(19) United States
(12) Patent Application Publication
(10) Pub. No.: US 2026/0014217 A1
(43) Pub. Date: Jan. 15, 2026

(54) COMPOSITIONS AND METHODS FOR BACTERIOPHAGE INFECTION OF STAPHYLOCOCCUS BACTERIA

(71) Applicant: Wisconsin Alumni Research Foundation, Madison, WI (US)

(72) Inventors: Charlie Mo, Madison, WI (US); My Tran, Madison, WI (US); Angel Hernandez Viera, Madison, WI (US)

(21) Appl. No.: 19/268,588

(22) Filed: Jul. 14, 2025

Related U.S. Application Data
(60) Provisional application No. 63/670,394, filed on Jul. 12, 2024.

(51) Int. Cl. A61P 31/04; A61K 35/76; C12N 7/00

(52) U.S. Cl. CPC A61K 35/76; A61P 31/04; C12N 7/00; C12N 2795/00021; C12N 2795/00032

(57) ABSTRACT

This disclosure provides Staphylococcus bacteriophages comprising a knockout and/or nonsense mutation in open reading frame 141 (ORF141), or a homologous position thereof. Also provided are compositions comprising the bacteriophage, methods of treating a bacterial infection, and methods of making a bacteria more susceptible to β-lactam antibiotic drug treatment.

### CROSS-REFERENCE TO RELATED APPLICATIONS

This application claims the benefit of priority to U.S. Provisional Patent Application No. 63/670,394, filed Jul. 12, 2024, the contents of which are herein incorporated by reference in their entirety.

### REFERENCE TO AN ELECTRONIC SEQUENCE LISTING

The contents of the electronic sequence listing (96029604714.xml; Size: 281,952 bytes; and Date of Creation: Jul. 2, 2025) is herein incorporated by reference in its entirety.

### BACKGROUND

Due, at least in part, to the over-prescription and misuse of antibiotics in humans and animals, the development of antibiotic-resistant bacterial strains has emerged as an immediate concern posing a significant threat to human health. Global estimates indicate that, in 2019, drug-resistant bacteria were the direct cause of 1.27 million deaths, and a contributing factor in 4.95 million deaths (Antimicrobial resistance. World Health Organization. who.int/news-room/fact-sheets/detail/antimicrobial-resistance). In addition to the staggering human toll, the long-term consequences of this rise in antimicrobial resistance-including an increased risk associated with other medical treatments (e.g., surgery, chemotherapy)—are expected to produce significant economic impacts world-wide. Thus, there is a need for the development of methods and compositions with improved ability to treat antibiotic-resistant bacterial infections.

### SUMMARY

Provided here are Staphylococcus bacteriophage comprising a nucleic acid having a knockout and/or nonsense mutation in open reading frame 141 (ORF141), or a homologous position thereof. The knockout and/or nonsense mutation may be located at or prior to a codon corresponding to amino acid residue 100 of ORF141. The knockout and/or nonsense mutation may comprise a mutation that replaces amino acid residue 77 of ORF141, or a homologous position thereof, with a premature stop codon, wherein the position is defined relative to SEQ ID NO: 1. The bacteriophage has a genome comprising or consisting of a nucleic acid of SEQ ID NO: 2 or a nucleic acid having at least 90%, 95%, 96%, 97%, 98%, or 99% identity to SEQ ID NO: 2.

In another aspect, compositions comprising the bacteriophages provided here with a pharmaceutically acceptable carrier for administration are also provided.

In yet another aspect, methods of treating a bacterial infection are provided. The methods include administering a bacteriophage to a subject infected with or suspected of being infected with a Staphylococcus bacteria. The Staphylococcus bacteria is associated with β-lactam antibiotic drug resistance and the Staphylococcus bacteria may be methicillin-resistant S. aureus (MRSA).

In a further aspect, methods of making bacteria more susceptible to β-lactam antibiotic drug treatment are provided. The methods include obtaining a sample comprising a Staphylococcus bacteria identified as resistant to a β-lactam antibiotic from a subject and contacting at least a portion of the bacteria with a bacteriophage to generate bacteriophage-contacted bacteria. The bacteriophage-contacted bacteria are then contacted with a β-lactam antibiotic; and a determination is made whether the bacteriophage-contacted bacteria exhibit increased susceptibility to the 0-lactam antibiotic as compared to the Staphylococcus bacteria in the sample prior to contact with the bacteriophage. These methods may be used to determine if treatment of a subject with the bacteriophage provided here in combination with a β-lactam antibiotic is a means of treating a subject with an infection.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show that infection by bacteriophage ΦStaph1N drives the loss of β-lactam resistance in MRSA. (1A) Schematic of experimental setup. Drug-resistant (Abx^{R}), phage-sensitive (Phage^{S}) bacterial cultures are infected with phage. The population of infected cells is passaged and allowed to recover. The surviving cell population is resistant to phage infection (PhageR), but have evolved sensitivity to antibiotics (Abx^{S}). (1B) ΦStaph1N infects MRSA strains MRSA252, MW2, and LAC (left panel). Following infection with ΦStaph1N, cultures of the three MRSA strains are resistant to ΦStaph1N (right panel). (1C) ΦStaph1N-treated, evolved MRSA strains show significant loss of resistance against oxacillin, compared to the parental strains. Loss of resistance is indicated by the area of bacterial clearance surrounding the antibiotic resistance strip. (1D) ΦStaph1N treatment causes loss resistance against different β-lactams. Plotted are the fold reductions of minimal inhibitory concentration (MIC) between treated and mock treated cells. OXI=oxacillin; CEF=cefazolin; AMX=amoxicillin; AMX+CA=amoxicillin & clavulanic acid; VANC=vancomycin. Top to bottom in the legend is left to right in the graph for each strain tested.

FIGS. 2A-2C show that Evo2 is a variant of ΦStaph1N with higher activity against MRSA. (2A) Evo2 shows comparable infectivity towards MRSA252 but improved infectivity towards MW2 and LAC, relative to ΦStaph1N. The same plaquing data is also shown in FIG. 9A. (2B) Similar to ΦStaph1N, Evo2 infection reduces β-lactam resistance in MRSA. Left to right for each strain tested is OXI, CEF, AMX, AMX+CA and VANC. (2C) Evo2 infection reduces the MIC against oxacillin in clinical isolates of USA300 (ADLs).

FIGS. 3A-B show that phage-treated MRSA strains evolve distinct mutational profiles. (3A) Coding sequences (CDS) with mutations from the three MRSA strains following phage treatment or mock treatment. For each strain, three isolates were sequenced and their mutations identified. Mutations are color-coded based on the number of occurrences among the three replicates. (3B) Categories of genes with mutations that arose in each MRSA strain and treatment condition.

FIG. 4 shows that phage infection changes the transcriptomic profile of MRSA. Differential expression analysis was performed on the transcriptomes of MW2 (top panel) and LAC (bottom panel). For both strains, Evo2-infected samples were compared to uninfected controls. 3 biological replicates were analyzed for each condition. Horizontal dotted lines represent an adjusted p-value cut-off of 0.002, while vertical dotted lines represent a log 2 fold change of −2 or 2 in expression. Transcripts with a log 2 fold change between −2 or 2 and a p-value >0.002 are labeled as grey dots (Not significant, NS); transcripts that pass either the fold change or p-value cutoff (but not the other) are represented as blue and green dots, respectively; transcripts that pass both cutoffs are shown as red dots. Genes discussed in the main text are labeled. Data for all the transcripts with significant fold changes is shown in Table 6.

FIGS. 5A-5C show that phage treatment of MRSA results in attenuated virulence phenotypes. (5A) MW2 and LAC strains display hemolytic activity on rabbit blood agar plates, while MRSA252 does not. (5B) Phage-treated MW2 and LAC strains display reduced hemolysis compared to uninfected cells. (5C) Surviving cultures of MW2 and LAC treated with either ΦStaph1N (blue) or Evo2 (red) show reduced clumping rates compared to mock untreated cells (teal).

FIGS. 6A-6C show co-treatment of MRSA with bacteriophage and β-antibiotic. (6A) Checkerboard assays of MRSA strains with gradients of oxacillin and Evo2 (top panels) or ΦStaph1N (bottom panels). The oxacillin gradient is a 2-fold serial dilution of drug concentration (μg/mL), while the phage MOI gradient is a 10-fold serial dilution of MOI. The rows and columns of each plate are labeled with letters and numbers, respectively. The black-white gradient in each well reflects the optical density of the culture and is the mean value from three biological replicates. MRSA strains co-treated with oxacillin and (6B) Evo2 or (6C) ΦStaph1N were tested for their phage resistance and oxacillin resistance. The letter/number combination reflects the well from which the cells were picked for analysis. Wells that could not produce a viable culture are labeled as NG (no growth). For wells that regrew, we calculated the efficiency of plaquing (EOP) of phage and measured the fold reduction in oxacillin MIC. Cultures that showed no detectable viral plaques are marked as resistant (R).

FIG. 7 shows phage sensitivity of MRSA strains. Efficiencies of plaquing of phages on MRSA252, MW2, and LAC. Phages were 10-fold serially diluted and spotted onto top agar overlays of each strain.

FIGS. 8A-8B show growth curves of MRSA strains under varying levels of phage infection. MRSA252, MW2, and LAC cultures were infected with either ΦStaph1N (8A) or Evo2 (8B) at the indicated multiplicity of infection (MOI). The optical density (OD600) of the cultures was monitored on an automated plate reader. Each condition was tested in 3 independent replicates FIGS. 9A-9B show isolation and sequencing analysis of Evo2. (9A) Individual Evo2 plaques appeared in larger ΦStaph1N plaques on LAC. Individual plaques were isolated and propagated in liquid culture. Evo2 shows improved plaquing on MW2 and LAC. Plaquing data in the right panels are the same as in FIG. 2A. (9B) Evo2 is a mutant form of ΦStaph1N with a nonsense mutation in ORF141. The A to C mutation (marked by the arrow) in Evo2 converts Serine 77 of ORF141 into a stop codon.

FIGS. 10A-10B show that phage ΦNM1γ6 infection of LAC does not drive the loss of β-lactam resistance. (10A) LAC treated with ΦNM1γ6 evolves resistance against ΦNM1γ6 evidenced by the reduction of plaquing from the parental to the evolved populations. (10B) Evolved and parental LAC populations show comparable MICs against different β-lactams and vancomycin.

FIGS. 11A-11B show that phage SATA8505 infection drives loss of oxacillin resistance. (11A) MRSA strains MRSA252, MW2, and LAC treated with SATA8585 evolves resistance against SATA8585, evidenced by the reduction of plaquing from the parental to the evolved populations. (11B) Evolved and parental MRSA show reduced MICs against oxacillin.

FIG. 12 shows types of polymorphisms in MRSA strains following infection by phage or a mock treatment. Plotted are the polymorphisms that were found in a gene with an assigned COG category.

FIG. 13 shows the plaquing efficiency of Evo2 and ΦStaph1N on MW2 and LAC strains with knockouts in mgrA, arl, and sarA.

FIG. 14 shows the effect of phage infection on biofilm formation in MRSA strains. Cultures were infected or mock-infected with either ΦStaph1N or Evo2. RP62a is a strain of S. epidermidis with known biofilm-forming capability, while LM1680 is a derivative of RP62a that has lost biofilm-forming ability.^{69,70} Biofilm biomass was assessed by staining with Crystal Violet. Solubilized crystal violet was quantified by measuring absorbance at 600 nm. Values represent averages and standard deviations of three replicates. Statistical significance was determined with a two-tailed t-test. Top to bottom in the legend is left to right in the graph for each strain tested.

FIG. 15 shows survival of phage-treated MRSA cells following H₂O₂ treatment. Bacterial strains treated with ΦStaph1N or Evo2 bacteriophage exhibited decreased survival following H₂O₂ treatment, indicating an increased susceptibility to oxidative stress. Top to bottom in the legend is left to right in the graph.

FIG. 16 shows the effect of Evo2 infection on the expression of surface protein, cell wall homeostasis protein, and staphyloxanthin synthesis protein genes in two strains of MRSA. Following phage infection, strains MW2 (left bars) and LAC (right bars) exhibited increased expression of surface proteins (ebh, fmtB, sasC), and several genes associated with cell wall maintenance (lytN, fmhC). Both strains showed reduced expression of genes associated with staphyloxanthin biosynthesis (crtM, crtN, crtP, crtQ).

FIG. 17 shows that Evo2 infection decreases the expression of genes associated with quorum sensing (agrA, agrB, agrC, hld) and the type VII secretion system (esaA, essA, esxA, esxB, esxD) in two strains of MRSA (MW2 (left bars); LAC (right bars)).

FIG. 18 shows the effect of phage infection on staphyloxanthin (STX) production. Consistent with the decreased staphyloxanthin biosynthesis gene expression shown in FIG. 16, MW2 treated with Evo2 and ΦStaph1N exhibited decreased production of staphyloxanthin, a carotenoid pigment with antioxidant properties that contributes to MRSA's ability to tolerate oxidative stress.

FIG. 19 shows the activity of Evo2 and ΦStaph1N when plated with human serum. Serial dilutions of Evo2 and ΦStaph1N were added to a plate containing MRSA (MRSA252, MW2, or LAC), BHI top agar, and 10% human serum. The presence of 10% human serum in the top agar exhibited minimal effect on the infectivity of Evo2 and ΦStaph1N.

FIG. 20 shows the activity of Evo2 and ΦStaph1N when incubated and plated with human serum. Serial dilutions of Evo2 and ΦStaph1N were prepared in phosphate buffered saline (PBS) or diluted human serum (10% or 25%), incubated at room temperature for 1 hour, and added to a plate containing MRSA (MRSA252, MW2, or LAC), BHI top agar, and 10% or 25% human serum. The presence of 10% or 25% human serum in the top agar exhibited minimal effect on the infectivity of Evo2 and ΦStaph1N. In addition, Evo2 and ΦStaph1N retained infectivity, though the number of plaques was reduced by 1-2 log following incubation with 25% human serum.

### DETAILED DESCRIPTION

The development of antibiotic-resistant bacterial strains has emerged as an immediate concern that poses a significant threat to human health. For example, Staphylococcus aureus (S. aureus) is a Gram-positive bacterium found on the skin and mucosa of humans and animals. It causes a wide range of human diseases, from endocarditis to pneumonia. Methicillin-resistant S. aureus (MRSA) is a form of S. aureus with a high level of drug resistance. The chief mediator of resistance in MRSA is the SCCmec cassette, which is a mobile genetic element that carries genes encoding resistance to β-lactam antibiotics, such as penicillins and cephalosporins. β-lactams act by inhibiting the synthesis of the peptidoglycan layer in bacterial cell walls and are one of the most commonly prescribed drug classes. Many β-lactams are designated as “critically important” antimicrobials by the World Health Organization. Due to resistance, MRSA infections pose a considerable public health risk since they are notoriously difficult to treat. In 2019, MRSA alone accounted for more than 100,000 deaths attributable to drug-resistant infections world-wide.

Bacteriophage therapy offers a unique evolutionary solution to re-sensitizing resistant pathogens. It has been shown that phage predation can select for phage resistance mutations in bacteria. However, we discovered that this resistance against phage infection can come with evolutionary “trade-offs” that are clinically useful. Leveraging these evolutionary trade-offs by treating antibiotic-resistant bacteria with a virulent bacteriophage confers several benefits, including (1) reduced bacterial burden of the infection, (2) reduced virulence of the remaining bacteria, and (3) increased susceptibility of the remaining bacteria to β-lactam antibiotics.

In the present invention, we performed evolution experiments by treating three strains of MRSA bacteria with Staph 1N (S1N; also referred to herein as ΦStaph1N) bacteriophage. We evaluated the activity of the evolved bacteria, and challenged samples of the bacteria with antibiotics or bacteriophage. We discovered that, following infection by a staphylococcal bacteriophage, MRSA bacteria demonstrated an evolved phenotype, characterized by increased sensitivity towards β-lactam antibiotics, reduced hemolytic ability and clumping (an indicator of virulence), decreased staphyloxanthin production, and increased susceptibility to oxidative stress and/or reactive oxygen species (ROS). We further evaluated the evolved bacteriophage, finding that the SiN phage had evolved into a more virulent phage, referred to herein as Evo2, which exhibits higher levels of infectivity against MRSA. In addition, we found that SiN and Evo2 demonstrated the ability to infect all three tested strains of MRSA (MRSA252, MW2, and Lac-Fitz), as well as a different bacterial species, Staphylococcus epidermidis (S. epidermidis).

Thus, provided herein are bacteriophage compositions, methods of treating a bacterial infection, and methods of making a bacteria more susceptible to β-lactam antibiotic drug treatment.

### Compositions

In an aspect, the disclosure provides a Staphylococcus bacteriophage comprising a nucleic acid having a knockout and/or nonsense mutation in open reading frame 141, or a homologous position thereof Δn open reading frame is a stretch of DNA, spanning from a start codon to a stop codon, that can be translated into a protein. As used herein, “open reading frame 141” or “ORF141” refers to an open reading frame in the genome of ΦStaph1N located, with reference to SEQ ID NO: 1, at nucleotide position 120,742-121,185. The sequence of ORF141 is highly conserved across many Staphylococcus phages, including, without limitation, Staphylococcus phages 812, K, IME-SA118, PM36, Sb1M_9382, vB_SauH-H33, vb_SaHa_Safa, PM34, A3R, 676Z, APTC_SA_4, vB_SauH-Ge, vB_SauH-Ge, vB_SauM_0414_108, PM22, Sb1_8383, Fi200W, A5W, IME-SA119, HYZ21, PM4, ISP, 812, SAML-229, IME-SA2, vB_SauM-313A1, and Sb1M_6168. Thus, in some embodiments, the Staphylococcus bacteriophage comprises a knockout and/or nonsense mutation at a position homologous to ORF141 as shown in SEQ ID NO: 1. Based on the information provided here, one of skill in the art could design a similar mutation in a highly conserved in Staphylococcus phage in ORF141.

As used herein, a “knockout mutation” refers to an engineered mutation that results in the removal or inactivation of a target gene or polynucleotide sequence within an organism's genome. Typically, a knockout mutation comprises a deletion, an insertion, and/or a substitution. As used herein, a “deletion” refers to the removal of one or more nucleotides relative to the native polynucleotide sequence, a “substitution” refers to the replacement of a nucleotide of a native polynucleotide sequence with a nucleotide that is not native to the polynucleotide sequence, and an “insertion” means the addition of one or more nucleotides to the native polynucleotide sequence. A deletion may remove a portion of the target gene or polynucleotide sequence (a “partial deletion”) or may remove the entire target gene or polynucleotide sequence (“a full deletion”). As used herein, a “nonsense mutation” refers to a point mutation in a genomic DNA sequence that causes the introduction of a premature stop codon during protein synthesis, yielding a truncated and often non-functional protein. Nonsense mutations may occur spontaneously, or may be the result of genetic engineering. In some embodiments, the knockout and/or nonsense mutation is located at or prior to a codon corresponding to amino acid residue 100 of ORF141. As demonstrated in the Examples, genomic analysis of the Evo2 bacteriophage revealed a single nonsense mutation in the codon for amino acid residue 77 of ORF141. Thus, in some embodiments, the knockout and/or nonsense mutation comprises a mutation that replaces amino acid residue 77 of ORF141, or a homologous position thereof, with a premature stop codon. In some embodiments, the knockout and/or nonsense mutation comprises a partial deletion of ORF141. In other embodiments, the knockout and/or nonsense mutation comprises a full deletion of ORF141.

In some embodiments, the bacteriophage is engineered to comprise the knockout and/or nonsense mutation. Suitable methods for engineering bacteriophages are well-known in the art (see, e.g., Lv et al., Viruses. 2023; 15(8):1736) and include, without limitation, host-mediated homologous recombination, in vivo recombineering, bacteriophage recombineering with electroporated DNA (BRED), yeast-based assembly using a yeast artificial chromosome (YAC), and CRISPR-Cas mediated systems.

In some embodiments, the bacteriophage exhibits increased infectivity of Staphylococcus bacteria as compared to a Staphylococcus bacteriophage without the knockout and/or nonsense mutation in ORF141. As used herein, “infectivity” refers to the ability of a bacteriophage to enter, survive, and multiply in a host bacterial cell. Bacteriophage infectivity may be assessed using any suitable method known in the art, including, without limitation, a bacterial plaquing assay.

In further aspects, provided herein are compositions comprising a bacteriophage disclosed herein, and a pharmaceutically acceptable carrier or excipient. In some embodiments, the bacteriophage comprises a nucleic acid having a knockout and/or nonsense mutation in open reading frame 141 (ORF141), or a homologous position thereof. In some embodiments, the bacteriophage has a genome comprising a nucleic acid of SEQ ID NO: 1 or SEQ ID NO: 2, or a nucleic acid having at least 70%, at least 75%, at least 80%, at least 85%, at least 88%, at least 90%, at least 92%, at least 95%, at least 96%, at least 97%, at least 98% or at least 99% sequence identity to SEQ ID NO: 1 or SEQ ID NO: 2. In some embodiments, the bacteriophage is a SiN bacteriophage or an Evo2 bacteriophage. The bacteriophage described herein may also be used in pharmaceutical compositions. Two or more of the bacteriophages described herein may be used in combination in a pharmaceutical composition in any ratio.

Those of skill in the art are aware of suitable, pharmaceutically acceptable carriers or excipients, such as, e.g., as described in (Remington's Pharmaceutical Sciences 16th edition, Osol, A. Ed. (1980). In some embodiments, the pharmaceutically acceptable carrier is a liquid suitable for intravenous administration, including, without limitation, as a suspension in Phosphate Buffered Saline (PBS; See, e.g., Nick et al., 2022, Cell 185, 1860-1874). The bacteriophage may present in the composition at a concentration of at least 10³ plaque forming units (PFU)/mL, at least 10⁴ PFU/mL, at least 10⁵ PFU/mL, at least 10⁶ PFU/mL, at least 10⁷ PFU/mL, at least 10⁸ PFU/mL, at least 10⁹ PFU/mL, at least 10¹⁰ PFU/mL, at least 10¹¹ PFU/mL, or at least 10¹² PFU/mL, at a concentration of about any of the foregoing, or at a concentration within a range bounded by any of the foregoing. In an embodiment, the bacteriophage is present in the composition at a concentration of at least 10⁷/mL or at a concentration of about 10⁷/mL. In some embodiments, the composition comprises about 10⁴ to about 10¹⁶ PFU, about 10⁵ to about 10¹⁵ PFU, about 10⁶ to about 10¹⁴ PFU, about 10⁷ to about 10¹³ PFU, about 10⁸ to about 10¹² PFU, about 10⁸ to about 10¹¹ PFU, or about 10⁸ PFU of the bacteriophage. In some embodiments, the composition is formulated to result in a bacteriophage multiplicity of infection (MOI) in the subject of at least 0.0001, at least 0.001, at least 0.01, at least 0.1, at least 1, or at least 10, or a multiplicity of infection within a range bounded by any of the foregoing (e.g., 0.0001-10, 0.01-0.1). The composition may be formulated for any suitable route of administration. In some embodiments, the composition is formulated for topical, oral, intranasal, or intravenous administration. The bacteriophage can be administered as a single dose or in multiple doses.

The bacteriophage may be produced and/or manufactured using any suitable method known in the art, including, without limitation, host-strain propagation, recombinant bacterial expression systems, and cell-free expression systems.

### Treatment Methods

Also provided herein are methods of treating a bacterial infection. In an aspect, the methods comprise administering a bacteriophage to a subject infected with or suspected of being infected with a Staphylococcus bacteria, optionally wherein the Staphylococcus bacteria is associated with β-lactam antibiotic drug resistance. In some embodiments, the Staphylococcus bacteria is S. aureus or S. epidermidis. In further embodiments, the S. aureus is a methicillin resistant S. aureus (MRSA). The MRSA may be any MRSA strain that is susceptible to bacteriophage infection, including, without limitation, MRSA252, MW2, and Lac-Fitz.

As used herein, “β-lactam antibiotic” and “β-lactam antibiotic drug” refer to antibiotic drugs containing a β-lactam ring, including, without limitation, penams, cephems, monobactams, carbapenems, and carbacephems, including, without limitation, penams (e.g., benzathine, benzylpenicillin, benzathine penicillin G, benzathine penicillin V, phenoxymethylpenicillin, procaine penicillin, pheneticillin, cloxacillin, dicloxacillin, flucloxacillin, methicillin, nafcillin, oxacillin, temocillin, amoxicillin, ampicillin, mecillinam, piperacillin, carbenicillin, ticarcillin, carbenicillin, ticarcillin, azlocillin, mezlocillin, piperacillin), cephems/carbacephems/cephalosporins/cephamycins (e.g., cefazolin, cefalexin, cefadroxil, cefapirin, cefazedone, cefazaflur, cefradine, cefroxadine, ceftezole, cefaloglycin, cefacetrile, cefalonium, cefaloridine, cefalotin, cefatrizine, cefaclor, cefprozil, cefuroxime, cefuroxime axetil, cefamandole, cefonicid, ceforanide, cefuzonam, cephamycin, cefoxitin, cefotetan, cefminox, cefbuperazone, cefmetazole, carbacephem, loracarbef, cefixime, ceftriaxone, cefotaxime, ceftazidime, cefoperazone, cefdinir, cefcapene, cefdaloxime, ceftizoxime, cefmenoxime, cefpiramide, cefpodoxime, ceftibuten, cefditoren, cefotiam, cefetamet, cefodizime, cefpimizole, cefsulodin, cefteram, ceftiolene, oxacephem, flomoxef, latamoxef, cefepime, cefozopran, cefpirome, cefquinome, ceftaroline fosamil, ceftolozane, ceftobiprole, cefiderocol, ceftiofur, cefquinome, cefovecin) monobactams (e.g., aztreonam, tigemonam, carumonam, nocardicin A), penems/carbapenems (e.g., ertapenem, doripenem, imipenem, meropenem, biapenem, panipenem, faropenem, ritipenem), and combinations thereof. “β-lactam antibiotic drug resistance” refers to decreased susceptibility to treatment with β-lactam antibiotics. “Susceptibility,” “sensitivity,” and “responsiveness” are herein used interchangeably.

The Staphylococcus bacteria may be identified as associated with β-lactam antibiotic resistance using any suitable method known in the art, including, without limitation, cell culturing in the presence of a β-lactam antibiotic (e.g., agar culturing, disc diffusion test, MIC test strip), polymerase chain reaction (PCR) amplification followed by detection of the PCR amplification product (e.g., by gel electrophoresis, sequencing), qPCR, or antibody-based methods (e.g., enzyme-linked immunosorbent assay [ELISA], western blotting).

The bacteriophage may be any bacteriophage capable of infecting the Staphylococcus bacteria, including any bacteriophage described herein. In some embodiments, the bacteriophage comprises a nucleic acid having a knockout and/or nonsense mutation in open reading frame 141 (ORF141), or a homologous position thereof. In some embodiments, the bacteriophage has a genome comprising a nucleic acid of SEQ ID NO: 1 or SEQ ID NO: 2, or a nucleic acid having at least 70%, at least 75%, at least 80%, at least 85%, at least 88%, at least 90%, at least 92%, at least 95%, at least 96%, at least 97%, at least 98% or at least 99% sequence identity to SEQ ID NO: 1 or SEQ ID NO: 2. In some embodiments, the bacteriophage has a nonsense mutation or other means of knocking out ORF141 in the bacteriophage and that mutation is conserved relative to SEQ ID NO: 2. In some embodiments, the bacteriophage is a SiN bacteriophage or an Evo2 bacteriophage.

As used herein, the terms “administering” and “administration” refer to any method of providing a pharmaceutical preparation to a subject. Suitable routes of administration include, without limitation, intramuscular, intradermal, intranasal, oral, topical, parenteral, intravenous, subcutaneous, intrathecal, transcutaneous, nasopharyngeal, and transmucosal routes. In some embodiments, the bacteriophage is administered topically, orally, intranasally, or intravenously. The bacteriophage can be administered as a single dose or in multiple doses. For example, the bacteriophage may be administered two or more times separated by 4 hours, 6 hours, 8 hours, 12 hours, a day, two days, three days, four days, one week, two weeks, or by three or more weeks.

The bacteriophage may be administered in any suitable pharmaceutical preparation, including, without limitation, any composition described herein. In some embodiments, the administering comprises administering the bacteriophage at a concentration of least 10⁷ plaque forming units (PFU)/mL. In some embodiments, the administering comprises administering about 10⁸ to about 10¹² PFU, or about 10⁸ PFU of the bacteriophage. The administration of the bacteriophage may result in a bacteriophage multiplicity of infection (MOI) in the subject of at least 0.0001, at least 0.001, at least 0.01, at least 0.1, at least 1, or at least 10, or a multiplicity of infection within a range bounded by any of the foregoing (e.g., 0.0001-10, 0.01-0.1). In some embodiments, the MOI in the subject is at least 0.01. In some embodiments, the MOI in the subject is about 0.01 or 0.1.

The subject to which the methods are applied may be a human being or any other vertebrate, including, without limitation, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human.

The bacteriophage may decrease/reduce the virulence of the Staphylococcus bacteria. As used herein “virulence” refers to a microorganism or pathogen's capacity to cause disease in a host organism. In some embodiments, the bacteriophage reduces the hemolytic activity of the Staphylococcus bacteria. As used herein, “hemolytic activity” and “hemolytic ability” refers to lysis of red blood cells by the bacteria, and is indicative of bacterial virulence. Hemolytic activity may be measured by any suitable means known in the art, including by incubating the bacteria on blood agar plates. In some embodiments, the bacteriophage reduces the hemolytic activity of the Staphylococcus bacteria by at least 10%, at least 20%, at least 30%, at least 40%, at least 50%, at least 60%, at least 70%, at least 80%, or at least 90%, as compared to a Staphylococcus bacteria that has not been subjected to the methods disclosed herein. In some embodiments, Staphylococcus bacteria that have been subjected to methods disclosed herein exhibits no detectable hemolytic activity.

In some embodiments, the bacteriophage reduces the clumping (also referred to as agglutination) of the Staphylococcus bacteria. As used herein, “clumping” and “agglutination” refers to the binding of bacterial cells to fibrinogen, and is indicative of bacterial virulence. Clumping may be measured using any method known in the art, including, without limitation, as described in Crosby et al (2021). In vitro assay for quantifying clumping of Staphylococcus aureus. Staphylococcus aureus: Methods and Protocols,^{33, 60}. In some embodiments, the bacteriophage reduces the clumping of the Staphylococcus bacteria by at least 10%, at least 20%, at least 30%, at least 40%, at least 50%, at least 60%, at least 70%, at least 80%, or at least 90%, as compared to a Staphylococcus bacteria that has not been subjected to the methods disclosed herein.

The bacteriophage may increase the susceptibility of the Staphylococcus bacteria to treatment with β-lactam antibiotics. The susceptibility of Staphylococcus bacteria to treatment with β-lactam antibiotics may be assessed using any suitable method, including as described in the bacterial methods below. In some embodiments, the methods further comprise administering a β-lactam antibiotic to the subject. The β-lactam antibiotic may be administered to the subject prior to, concurrently with, or after bacteriophage administration. A first dose of the β-lactam antibiotic may be administered to the subject within 1 hour, 4 hours, 6 hours, 12 hours, 24 hours, 3 days, 1 week, 2 weeks, or 1 month after administration of the bacteriophage, or within a range bounded by any of the foregoing. In some embodiments, a first dose of β-lactam antibiotic is administered within 24 hours after administration of the bacteriophage. Following the first dose, a skilled artisan will be aware of a suitable dosing regimen for the particular β-lactam antibiotic being administered. The β-lactam antibiotic and bacteriophage may be administered using the same route of administration or different routes of administration (e.g., oral or intravenous β-lactam antibiotic administration and topical or intranasal bacteriophage administration).

The bacteriophage may increase the susceptibility or sensitivity of the Staphylococcus bacteria to oxidative stress. As used herein, “oxidative stress” refers to a systemic redox imbalance characterized by increased production of reactive oxygen species (ROS) in a subject, and the ability to tolerate oxidative stress is indicative of bacterial virulence. The susceptibility or sensitivity of the Staphylococcus bacteria to oxidative stress can be assessed using any suitable method known in the art, including, without limitation, by assessing bacterial survival under conditions known to promote oxidative stress (e.g., in the presence of hydrogen peroxide [1H₂O₂]). The susceptibility or sensitivity of the Staphylococcus bacteria to oxidative stress may also be suitably assessed by measuring a Staphylococcal marker (e.g., a protein, enzyme, or metabolite) associated with oxidative stress tolerance. In some embodiments, the Staphylococcus bacteria exhibits reduced production of staphyloxanthin (SPX). SPX is a yellow carotenoid pigment produced by certain strains of S. aureus that contributes to bacterial oxidative stress tolerance via its antioxidant properties. In some embodiments, following administration of the bacteriophage, the Staphylococcus bacteria exhibits at least a 10% decrease, at least a 20% decrease, at least a 30% decrease, at least a 40% decrease, at least a 50% decrease, at least a 60% decrease, at least a 70% decrease, at least an 80% decrease, or at least a 90% decrease in SPX production, as compared to a Staphylococcus bacteria that has not been subjected to the disclosed methods, or exhibits a decrease in SPX production within a range bounded by any of the foregoing.

In some embodiments, the methods further comprise administering ROS or an ROS generator pharmaceutical to the subject. As used herein, an “ROS generator pharmaceutical” refers to a pharmaceutical composition that induces oxidative stress and/or increases ROS production in the subject. Suitable ROS generator pharmaceuticals are known in the art, including, without limitation, H₂O₂. The ROS or a ROS generator pharmaceutical may be administered to the subject prior to, concurrently with, or after bacteriophage administration. The ROS or ROS generator pharmaceutical may be administered to the subject within 1 hour, 4 hours, 6 hours, 12 hours, 24 hours, 3 days, 1 week, 2 weeks, or 1 month after administration of the bacteriophage. In some embodiments, the ROS or an ROS generator pharmaceutical is administered within 24 hours after administration of the bacteriophage.

### Bacterial Methods

Also provided herein are methods of making bacteria more susceptible to β-lactam antibiotic drug treatment. In an aspect, the methods comprise obtaining a sample comprising a Staphylococcus bacteria from a subject and testing the bacterial sample to identify the bacteria as resistant to a β-lactam antibiotic. The sample may be any biological sample that may contain a Staphylococcus bacteria, including, without limitation, a tissue sample or a biological fluid such as whole blood or whole blood components including red blood cells, white blood cells, platelets, serum and plasma, ascites, vitreous fluid, lymph fluid, synovial fluid, follicular fluid, seminal fluid, amniotic fluid, milk, saliva, sputum, tears, perspiration, mucus, cerebrospinal fluid, pus, or urine. A swab or washing obtained from a skin or mucus membrane may also be used as a sample of bacteria. For any type of sample, a skilled artisan will be aware of suitable methods for obtaining the sample. In some embodiments, the sample is a sputum sample obtained following expectoration. In some embodiments, the sample is a tissue sample obtained via swabbing or biopsy of a wound site. In some embodiments, the sample is a mucus sample obtained via nasal or throat swabbing. In some embodiments, the sample is whole blood sample obtained via venipuncture. The sample may be obtained from the subject directly, or via a third party intermediary (e.g., a healthcare professional or clinician). The bacteria from the sample may be isolated and/or cultured to grow the bacteria prior to analysis of antibiotic resistance. These steps are well known to those of skill in the art.

The subject to which the methods are applied may be a human being or any other vertebrate, including, without limitation, cows, horses, sheep, pigs, goats, rabbits, dogs, cats, bats, mice, and rats. In certain embodiments, the methods may be performed on lab animals (e.g., mice and rats) for research purposes. In other embodiments, the methods are performed on commercially important farm animals (e.g., cows, horses, pigs, rabbits, goats, sheep, and chickens) or companion animals (e.g., cats and dogs). In preferred embodiments, the subject is a human.

In some embodiments, the Staphylococcus bacteria is S. aureus or S. epidermidis. In further embodiments, the S. aureus is MRSA. The MRSA may be any MRSA strain that is susceptible to bacteriophage infection, including, without limitation, MRSA252, MW2, and Lac-Fitz. The Staphylococcus bacteria may be identified as resistant to a β-lactam antibiotic using any suitable method known in the art, including, without limitation, cell culturing in the presence of a β-lactam antibiotic (e.g., agar culturing, disc diffusion test, MIC test strip), polymerase chain reaction (PCR) amplification followed by detection of the PCR amplification product (e.g., by gel electrophoresis, sequencing), qPCR, or antibody-based methods (e.g., enzyme-linked immunosorbent assay [ELISA], western blotting).

In a further aspect, the methods comprise contacting at least a portion of the bacteria with a bacteriophage to generate bacteriophage contacted bacteria. The bacteriophage may be any bacteriophage capable of infecting the bacteria, including any bacteriophage described herein. In some embodiments, the bacteriophage comprises a nucleic acid having a knockout and/or nonsense mutation in open reading frame 141 (ORF141), or a homologous position thereof. In some embodiments, the bacteriophage has a genome comprising a nucleic acid of SEQ ID NO: 1 or SEQ ID NO: 2, or a nucleic acid having at least 70%, at least 75%, at least 80%, at least 85%, at least 88%, at least 90%, at least 92%, at least 95%, at least 96%, at least 97%, at least 98% or at least 99% sequence identity to SEQ ID NO: 1 or SEQ ID NO: 2. In some embodiments, the bacteriophage is a SiN bacteriophage or an Evo2 bacteriophage.

The sample comprising the bacteria may be processed by any suitable method prior to contacting at least a portion of the bacteria with the bacteriophage. In some embodiments, the bacteria, or the sample comprising the bacteria is cultured in growth media and poured onto a plate with top agar, and the bacterial lawn is spot-treated with diluted bacteriophage. In some embodiments, (1) the bacteria is plated onto agar plates and grown, (2) after growth, individual colonies (also referred to as P0) are picked from the agar plate, inoculated into growth media, and incubated, (3) the grown P0 cultures are transferred into fresh growth media (P1 cultures) and incubated, (4) bacteriophage is added to the P1 culture at early log phase (e.g., OD 0.3), and the bacteriophage-treated P1 culture is incubated, (5) the bacteriophage-treated P1 culture is transferred into fresh growth media (P2 cultures) and incubated, and (5) the grown P2 cultures are used for phenotypic assays such as assessment of antibiotic resistance and comparison to the P0 cultures, not treated with the bacteriophage.

The bacteria may be contacted with any suitable amount or concentration of the bacteriophage. In some embodiments, the contacting comprises a bacteriophage at a concentration of least 10⁷ plaque forming units (PFU)/mL. In some embodiments, the contacting comprises about 10⁸ to about 10¹² PFU, or about 10⁸ PFU of the bacteriophage. The contacting may comprise a bacteriophage MOI of at least 0.0001, at least 0.001, at least 0.01, at least 0.1, at least 1, or at least 10, a MOI of about any of the foregoing, or a MOI within a range bounded by any of the foregoing (e.g., 0.0001-10, 0.01-0.1). In some embodiments, the contacting comprises a MOI of at least 0.01. In some embodiments, the contacting comprises a MOI of about 0.01 or 0.1. The sample may be contacted with the bacteriophage for at least 1 hour, at least 4 hours, at least 6 hours, at least 12 hours, at least 24 hours, at least 3 days, at least 1 week, for a time period of about any of the foregoing, or for a time period within a range bounded by any of the foregoing. In some embodiments, the sample is contacted with the bacteriophage for at least 24 hours.

In a further aspect, the methods comprise contacting the bacteriophage-contacted bacteria with a β-lactam antibiotic, and determining whether the bacteriophage-contacted bacteria exhibits increased susceptibility to the β-lactam antibiotic as compared to the Staphylococcus bacteria in the sample prior to contact with the bacteriophage. The increased susceptibility of the bacteriophage-contacted bacteria to the β-lactam antibiotic may be determined using any suitable method known in the art, including, without limitation, by determining the β-lactam antibiotic MIC defined as the lowest concentration of the β-lactam antibiotic that inhibits visible bacterial growth, for the bacteriophage contacted bacteria and the bacteria present in the sample prior to contact with the bacteriophage, and calculating a fold decrease in β-lactam antibiotic minimum inhibitory concentration (MIC). In some embodiments, the β-lactam antibiotic MIC fold decrease for the bacteriophage-contacted bacteria at least 0.1 fold, 0.5 fold, 1 fold, 5 fold, 10 fold, 100 fold, 1,000 fold, or 10,000 fold as compared to the β-lactam antibiotic MIC for the bacteriophage present in the sample prior to contact with the bacteriophage.

In some embodiments, the methods further comprise administering the bacteriophage to the subject based, at least in part, on the determination that the bacteriophage-contacted bacteria exhibits increased susceptibility to the β-lactam antibiotic. Suitable routes of administration include, without limitation, intramuscular, intradermal, intranasal, oral, topical, parenteral, intravenous, subcutaneous, intrathecal, transcutaneous, nasopharyngeal, and transmucosal routes. In some embodiments, the bacteriophage is administered intravenously. The bacteriophage can be administered as a single dose or in multiple doses. For example, the bacteriophage may be administered two or more times separated by 4 hours, 6 hours, 8 hours, 12 hours, a day, two days, three days, four days, one week, two weeks, or by three or more weeks.

The bacteriophage may be administered in any suitable pharmaceutical preparation, including, without limitation, any composition described herein. In some embodiments, the administering comprises administering the bacteriophage at a concentration of least 10⁷ plaque forming units (PFU)/mL. In some embodiments, the administering comprises administering about 10⁸ to 10¹² PFU, or about 10⁸ PFU of the bacteriophage. The administration of the bacteriophage may result in a bacteriophage multiplicity of infection (MOI) in the subject of at least 0.0001, at least 0.001, at least 0.01, at least 0.1, at least 1, or at least 10, a MOI of about any of the foregoing, or a MOI within a range bounded by any of the foregoing (e.g., 0.0001-10, 0.01-0.1). In some embodiments, the MOI in the subject is at least 0.01. In some embodiments, the MOI in the subject is about 0.01 or 0.1.

In some embodiments, the methods further comprise administering a β-lactam antibiotic to the subject. The β-lactam antibiotic may be administered to the subject prior to, concurrently with, or after bacteriophage administration. A first dose of the β-lactam antibiotic may be administered to the subject within 1 hour, 4 hours, 6 hours, 12 hours, 24 hours, 3 days, 1 week, 2 weeks, or 1 month after administration of the bacteriophage, or within a range bounded by any of the foregoing. In some embodiments, a first dose of β-lactam antibiotic is administered within 24 hours after administration of the bacteriophage. Following the first dose, a skilled artisan will be aware of a suitable dosing regimen for the particular β-lactam antibiotic being administered. The β-lactam antibiotic and bacteriophage may be administered using the same route of administration or different routes of administration (e.g., oral or intravenous β-lactam antibiotic administration and topical or intranasal bacteriophage administration).

In some embodiments, the methods further comprise administering ROS or an ROS generator pharmaceutical to the subject. Suitable ROS generator pharmaceuticals are known in the art, including, without limitation hydrogen peroxide. The ROS or an ROS generator pharmaceutical may be administered to the subject prior to, concurrently with, or after bacteriophage administration. The ROS or an ROS generator pharmaceutical may be administered to the subject within 1 hour, 4 hours, 6 hours, 12 hours, 24 hours, 3 days, 1 week, 2 weeks, or 1 month after administration of the bacteriophage. In some embodiments, the ROS or an ROS generator pharmaceutical is administered within 24 hours after administration of the bacteriophage.

In some embodiments, the methods further comprise determining that, as compared to a non-bacteriophage-contacted bacteria, the bacteriophage-contacted bacteria exhibits reduced hemolysis, increased susceptibility to oxidative stress, and decreased staphyloxanthin production. The reduced hemolysis, increased susceptibility to oxidative stress, and decreased staphyloxanthin production may suitably be determined using any method described herein.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

The indefinite articles “a” and “an,” as used herein in the specification and in the claims, unless clearly indicated to the contrary, should be understood to mean “at least one.” For example, reference to “a β-lactam antibiotic” or “the β-lactam antibiotic” is meant to encompass a single (3-lactam antibiotic and any combination of two or more different β-lactam antibiotics.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, “about” with respect to the compositions can mean plus or minus a range of up to 20%, preferably up to 10%, more preferably up to 5%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” All references, patents and patent applications disclosed herein are incorporated by reference with respect to the subject matter for which each is cited, which in some cases may encompass the entirety of the document.

The present invention has been described in terms of one or more embodiments, and it should be appreciated that many equivalents, alternatives, variations, and modifications, aside from those expressly stated, are possible and within the scope of the invention. The following examples are meant only to be illustrative and are not meant as limitations on the scope of the invention or of the appended claims.

### EXEMPLARY EMBODIMENTS

Embodiment 1. A Staphylococcus bacteriophage comprising a nucleic acid having a knockout and/or nonsense mutation in open reading frame 141 (ORF141), or a homologous position thereof.

Embodiment 2. The bacteriophage of embodiment 1, wherein the knockout and/or nonsense mutation is located at or prior to a codon corresponding to amino acid residue 100 of ORF141.

Embodiment 3. The bacteriophage of embodiment 1 or 2, wherein the knockout and/or nonsense mutation comprises a C for A substitution at nucleotide 230 in the ORF141 gene, or a homologous position thereof, wherein the position is defined relative to SEQ ID NO: 1.

Embodiment 4. The bacteriophage of any one of embodiments 1-3, wherein the bacteriophage has a genome comprising or consisting of a nucleic acid of SEQ ID NO: 2 or a nucleic acid having at least 90%, 95%, 96%, 97%, 98%, or 99% identity to SEQ ID NO: 2.

Embodiment 5. The bacteriophage of any one of embodiments 1-4, wherein the knockout and/or nonsense mutation comprises a mutation that replaces amino acid residue 77 of ORF141, or a homologous position thereof, with a premature stop codon, wherein the position is defined relative to SEQ ID NO: 1.

Embodiment 6. The bacteriophage of embodiment 1, wherein the knockout and/or nonsense mutation comprises a full or partial deletion of ORF141.

Embodiment 7. The bacteriophage of any one of embodiments 1-6, wherein the bacteriophage exhibits increased infectivity of Staphylococcus bacteria as compared to a Staphylococcus bacteriophage without the knockout and/or nonsense mutation in ORF141.

Embodiment 8. A composition comprising (i) a bacteriophage selected from the bacteriophage of any one of embodiments 1-7, an Evo2 bacteriophage or a Staph 1N (S1N) bacteriophage and (ii) a pharmaceutically acceptable carrier.

Embodiment 9. The composition of embodiment 8, wherein the bacteriophage is present at a concentration of at least 10⁷/mL.

Embodiment 10. The composition of embodiment 8 or 9, wherein the composition is formulated for topical, oral, intranasal, or intravenous administration.

Embodiment 11. The composition of any one of embodiments 8-10, wherein the pharmaceutically acceptable carrier comprises phosphate buffered saline (PBS).

Embodiment 12. A method of treating a bacterial infection, the method comprising administering a bacteriophage to a subject infected with or suspected of being infected with a Staphylococcus bacteria, wherein the Staphylococcus bacteria is associated with β-lactam antibiotic drug resistance.

Embodiment 13. The method of embodiment 12, wherein the Staphylococcus bacteria is Staphylococcus aureus (S. aureus) or Staphylococcus epidermidis (S. epidermidis).

Embodiment 14. The method of embodiment 12 or 13, wherein the S. aureus is methicillin-resistant S. aureus (MRSA).

Embodiment 15. The method of any one of embodiments 12-14, wherein bacteriophage comprises a nucleic acid having a knockout and/or nonsense mutation in open reading frame 141 (ORF141), or a homologous position thereof.

Embodiment 16. The method of any one of embodiments 12-15, wherein the bacteriophage is the bacteriophage of any one of embodiments 1-7.

Embodiment 17. The method of any one of embodiments 12-15, wherein the administering comprises administering the composition of any one of embodiments 8-11.

Embodiment 18. The method of any one of embodiments 12-14, wherein the bacteriophage is an Evo2 bacteriophage or a Staph 1N (SiN) bacteriophage.

Embodiment 19. The method of embodiment 18, wherein:
(a) the Evo2 bacteriophage has a genome comprising or consisting of a nucleic acid of SEQ ID NO: 2 or a nucleic acid having at least 90%, 95%, 96%, 97%, 98%, or 99% identity to SEQ ID NO: 2; or
(b) the SiN bacteriophage has a genome comprising or consisting of a nucleic acid of SEQ ID NO: 1 or a nucleic acid having at least 90%, 95%, 96%, 97%, 98%, or 99% identity to SEQ ID NO: 1.

Embodiment 20. The method of any one of embodiments 12-19, wherein administration of the bacteriophage results in a bacteriophage multiplicity of infection (MOI) in the subject of at least 0.01.

Embodiment 21. The method of any one of embodiment 12-20, wherein the administering comprises administering 10⁸-10¹² plaque forming units (PFU) of the bacteriophage.

Embodiment 22. The method of any one of embodiments 12-21, wherein the Staphylococcus bacteria exhibits reduced hemolytic activity following administration of the bacteriophage.

Embodiment 23. The method of any one of embodiments 12-22, wherein the Staphylococcus bacteria exhibits increased sensitivity to oxidative stress following administration of the bacteriophage.

Embodiment 24. The method of any one of embodiments 12-23, wherein the Staphylococcus bacteria produces less staphyloxanthin following administration of the bacteriophage.

Embodiment 25. The method of any one of embodiments 12-24, further comprising administering a β-lactam antibiotic to the subject.

Embodiment 26. The method of embodiment 25, wherein a first dose of the β-lactam antibiotic is administered to the subject within 6 hours, 12 hours, 18 hours, 24 hours, or 48 hours after administration of the bacteriophage.

Embodiment 27. The method of any one of embodiments 12-26, further comprising administering a reactive oxygen species (ROS) or ROS generator pharmaceutical to the subject.

Embodiment 28. A method of making bacteria more susceptible to β-lactam antibiotic drug treatment, the method comprising:
(a) obtaining a sample comprising a Staphylococcus bacteria identified as resistant to a β-lactam antibiotic from a subject;
(b) contacting a portion of the bacteria with a bacteriophage to generate bacteriophage contacted bacteria;
(c) contacting the bacteriophage contacted bacteria with a β-lactam antibiotic; and
(d) determining whether the bacteriophage contacted bacteria exhibits increased susceptibility to the β-lactam antibiotic as compared to the Staphylococcus bacteria in the sample prior to contact with the bacteriophage.

Embodiment 29. The method of embodiment 28, wherein the Staphylococcus bacteria is S. aureus or S. epidermidis.

Embodiment 30. The method of embodiment 28 or 29, wherein the S. aureus is methicillin-resistant S. aureus (MRSA).

Embodiment 31. The method of any one of embodiments 28-30, wherein the bacteriophage is a Evo2 bacteriophage or a Staph 1N (SiN) bacteriophage.

Embodiment 32. The method of embodiment 31, wherein:
(a) the Evo2 bacteriophage has a genome comprising or consisting of a nucleic acid of SEQ ID NO: 2 or a nucleic acid having at least 90%, 95%, 96%, 97%, 98%, or 99% identity to SEQ ID NO: 2; or
(b) the SiN bacteriophage has a genome comprising or consisting of a nucleic acid of SEQ ID NO: 1 or a nucleic acid having at least 90%, 95%, 96%, 97%, 98%, or 99% identity to SEQ ID NO: 1.

Embodiment 33. The method of any one of embodiments 28-32, wherein bacteriophage comprises a nucleic acid having a knockout and/or nonsense mutation in open reading frame 141 (ORF141), or a homologous position thereof.

Embodiment 34. The method of any one of embodiments 28-33, wherein the bacteriophage is the bacteriophage of any one of embodiments 1-7.

Embodiment 35. The method of any one of embodiments 28-34, wherein the contacting in step (b) comprises a bacteriophage MOI of at least 0.01.

Embodiment 36. The method of any one of embodiments 28-35, further comprising determining that the bacteriophage contacted bacteria exhibits reduced hemolysis, as compared to a non-bacteriophage contacted Staphylococcus bacteria.

Embodiment 37. The method of any one of embodiments 28-36, further comprising determining that the bacteriophage contacted bacteria exhibits increased susceptibility to ROS, as compared to a non-bacteriophage contacted Staphylococcus bacteria.

Embodiment 38. The method of any one of embodiments 28-37, further comprising determining that the bacteriophage contacted bacteria produces less staphyloxanthin, as compared to a non-bacteriophage contacted Staphylococcus bacteria.

Embodiment 39. The method of any one of embodiments 28-38, further comprising administering the bacteriophage to the subject based, at least in part, on the determination in step (d).

Embodiment 40. The method of embodiment 39, wherein the bacteriophage are administered to the subject to result in a bacteriophage MOI in the subject of at least 0.01.

Embodiment 41. The method of embodiment 39 or 40, wherein the administering comprises administering the composition of any one of embodiments 8-11.

Embodiment 42. The method of any one of embodiments 39-41, further comprising administering a β-lactam antibiotic to the subject.

Embodiment 43. The method of embodiment 42, wherein a first dose of the β-lactam antibiotic is administered to the subject within 6 hours, 12 hours, 18 hours, 24 hours, or 48 hours after administration of the bacteriophage.

Embodiment 44. The method of any one of embodiments 39-43, further comprising administering to the subject a ROS or pharmaceutical capable of increasing ROS.

Embodiment 45. The method of any one of embodiments 12-26 or 39-44, wherein:
(a) the antibiotic is administered orally or intravenously; and/or
(b) the bacteriophage is administered topically, orally, intranasally, or intravenously.

Embodiment 46. An Evo2 bacteriophage.

Embodiment 47: The composition of any one of embodiments 8-11, wherein the composition comprises 108-10¹² colony forming units (CFU) of the bacteriophage.

### EXAMPLES

### Example 1: Bacteriophage Infection Drives Loss of β-Lactam Resistance in Methicillin-Resistant Staphylococcus aureus

Bacteriophage (phage) therapy is a promising means to combat drug-resistant bacterial pathogens. Infection by phage can select for mutations in bacterial populations that confer resistance against phage infection. However, resistance against phage can yield evolutionary trade-offs of biomedical relevance. Here we report the discovery that infection by certain staphylococcal phages sensitizes different strains of methicillin-resistant Staphylococcus aureus (MRSA) to 3-lactams, a class of antibiotics against which MRSA is typically resistant. MRSA cells that survive infection by these phages display significant reductions in minimal inhibitory concentration against different β-lactams compared to uninfected bacteria. Transcriptomic profiling reveals that these evolved MRSA strains possess highly modulated transcriptional profiles, where numerous genes involved in S. aureus virulence were downregulated. Phage-treated MRSA exhibited attenuated virulence phenotypes in the form of reduced hemolysis and clumping. Despite sharing similar phenotypes, whole-sequencing analysis revealed that the different MRSA strains evolved unique genetic profiles during infection. These results suggest complex evolutionary trajectories in MRSA during phage predation and open up new possibilities to reduce drug resistance and virulence in MRSA infections.

Staphylococcus aureus is one of the most notorious and widespread bacterial pathogens, responsible for hundreds of thousands of severe infections worldwide every year. Methicillin-resistant S. aureus (MRSA) poses a particular clinical threat, as MRSA infections increase mortality, morbidity, and hospital stay, as compared to those caused by methicillin-sensitive S. aureus (MSSA).¹ Part of MRSA's notoriety stems from its strong resistance against the β-lactam family of antibiotics, such as penicillins and cephalosporins, which inhibit the activity of transpeptidase enzymes during peptidoglycan synthesis in bacterial cell walls.² β-lactams are one of the most commonly prescribed drug classes, with many designated as “Critically Important” antimicrobials by the World Health Organization.³ Thus, MRSA infections pose a considerable public health risk as they are notoriously difficult to treat and are widespread in communities and hospital settings. Indeed, in 2019, MRSA alone accounted for more than 100,000 deaths attributable to drug-resistant infections worldwide.¹

### Phage Treated MRSA Strains Display Increased Susceptibility to Oxidative Stress

Hydrogen peroxide (H₂O₂) is a reactive oxygen species that is released by neutrophils to induce oxidative stress and fight off microbial infection. To investigate the impact of phage treatment on bacterial response to oxidative stress, we measured the survival of MRSA252, MW2, and Lac-Fitz following treatment with H₂O₂(FIG. 9). As compared to untreated bacteria, the bacterial strains treated with ΦStaph1N or Evo2 bacteriophage exhibited decreased survival following H₂O₂ treatment, indicating an increased susceptibility to reactive oxygen species/oxidative stress.

A chief mediator of β-lactams resistance in MRSA is the SCCmec cassette, a mobile genetic element that carries the resistance gene mecA. MecA encodes for the penicillin-binding protein 2A (PBP2a), a transpeptidase that has a low affinity for β-lactams.⁵ This lower affinity permits PBP2a to participate in peptidoglycan synthesis even in the presence of β-lactams, ultimately resulting in cell survival. In addition to mecA, numerous MRSA strains also carry β-lactamases, such as BlaZ, that degrade β-lactams, thus further contributing to drug resistance. Together, these mechanisms can severely limit treatment options against MRSA, with current clinical treatment options relying primarily on vancomycin and daptomycin.⁶ Both vancomycin and daptomycin are last resort antibiotics against MRSA, and a major concern is the increasing resistance of MRSA to these drugs.^{7,8} Developing solutions to combat MRSA is a major focus in academia and industry.

Due to its drug resistance and clinical burden, S. aureus is a prime candidate for alternative antimicrobial treatments, such as bacteriophage (phage) therapy. Phages are viruses that infect and kill bacteria, posing one of the greatest existential threats to bacterial communities, with some estimates suggesting that 40% of all bacterial mortality worldwide is caused by phage predation.⁹ In phage therapy, lytic phages are administered to kill the bacterial pathogen(s) causing an infection. Phages offer certain advantages over traditional antibiotics: they are highly specific to their hosts by reducing off-target killing; they self-amplify and evolve, enabling the rapid generation of new phage variants with improved activities; and they are generally regarded as safe, as toxicity has been reported only in extremely rare cases in animals and patients.¹⁰ Indeed, against S. aureus infections, over a dozen promising case studies and clinical trials have been reported in the past decade.¹¹

Despite these advances, routine use of phage therapy is still met with challenges. Chief among these is the inevitable rise of phage resistance, as phage predation exerts a strong selective pressure on bacterial populations. According to one meta-analysis that focused on phage therapy outcomes, resistance against phage evolved in 75 percent of human clinical cases in which the evolution of resistance was monitored.¹² Mutations represent a chief pathway by which bacteria evolve resistance against phage. To date, the best characterized phage resistance mutations involve alterations on cell surface receptor molecules that mediate phage attachment. In many bacteria, these receptors are often proteins or sugar moieties, which are recognized by phage proteins.^{11,13,14} For example, in Escherichia coli, mutations in the cell-wall protein LamB confer resistance against lambda phage infection, while in S. aureus, mutations that modify wall teichoic acid (WTA) have been shown to limit phage infection.^{14,15} Complicating the picture, studies have revealed that a plethora of additional host mechanisms, including dedicated anti-phage defense systems, can impact the evolution of resistance against phage.^{16,17} These problems highlight the importance of developing phage treatment strategies that minimize or capitalize on the evolution of phage resistance.¹⁸

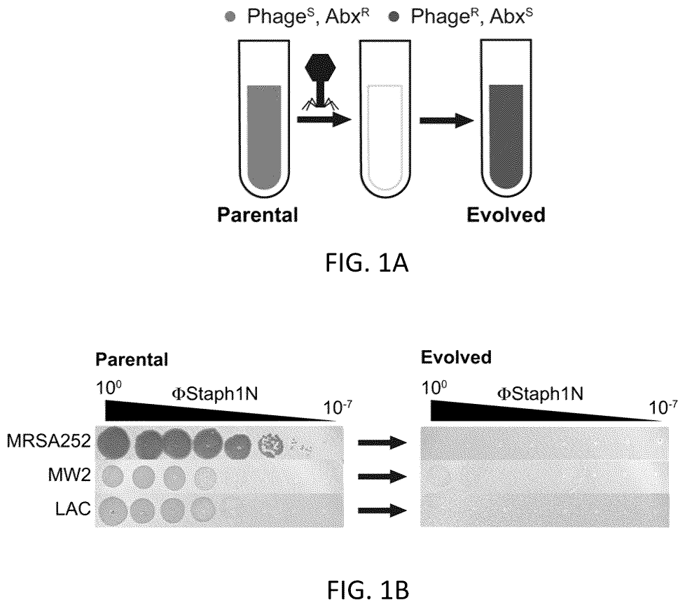

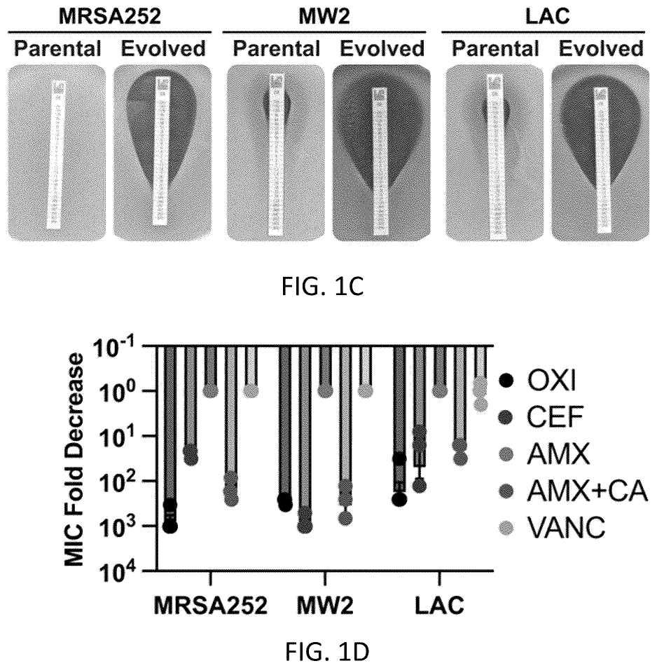

A unique aspect of phage therapy is the possibility to exploit evolutionary trade-offs to combat resistant pathogens. A genetic trade-off is defined as an evolved trait that confers a fitness advantage against a particular selective pressure at the expense of reduced fitness against an unselected pressure. Across many different species of bacteria, such trade-offs have been shown to occur between phage resistance and antibiotic resistance (FIG. 1A). Phages that bind to a virulence factor or mechanism for antibiotic resistance in the target bacteria are predicted to exert a strong selection pressure on the bacteria to mutate or downregulate the phage-binding target. These changes would confer protection against phage infection but could in turn reduce the resistance or virulence in the bacterium. As an example, in P. aeruginosa, infection by the phage OMKO1, which binds to the outer membrane protein M OprM of MexAB- and MexXY-OprM efflux pumps, drives the evolution of mutations in those genes, leading to the re-sensitization of phage-resistant P. aeruginosa mutants to antibiotics.^{10,19}

Little is known about how phage resistance can mediate genetic trade-offs in MRSA. Previous work has shown that phage resistance in S. aureus can proceed through genetic mutations that are not directly involved in phage binding. For example, studies by Berryhill and colleagues demonstrated that phage infection of S. aureus Newman, an MSSA strain, can select for mutations in femA, which is a cytoplasmic enzyme that catalyzes the formation of the pentaglycine bridge of peptidoglycans in S. aureus.²⁰ Rather than serving as a direct phage receptor molecule, femA maintains the integrity of the cell wall, which in turn could be vital for WTA maturation and phage attachment.^{13,21} Interestingly, a consequence of these femA mutations is increased sensitivity against antibiotics. We therefore asked how phage resistance might impact the physiology of drug-resistant MRSA, with the hopes of identifying genetic trade-offs of potential biomedical relevance.

In the work reported here, we show that infection by staphylococcal phages causes MRSA strains to evolve sensitivity to different types of β-lactam antibiotics and attenuate virulence phenotypes. We found that this loss of resistance and virulence is associated with distinct mutational profiles distinct in each MRSA strain, and that phage-treated, evolved MRSA populations display significant transcriptome remodeling. Unexpectedly, we also discovered a mutant phage with higher activity and a broader host range against MRSA. Findings from our work can help in the development of phage therapies that reduce drug resistance and virulence in pathogenic bacteria.

### Results

Identification of ΦStaph1N with Activity Against Multiple MRSA Strains

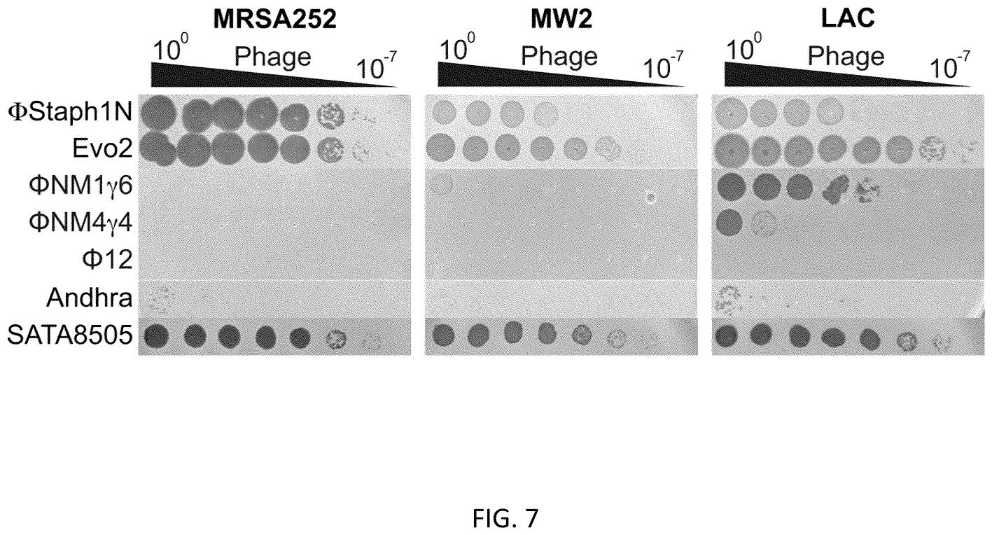

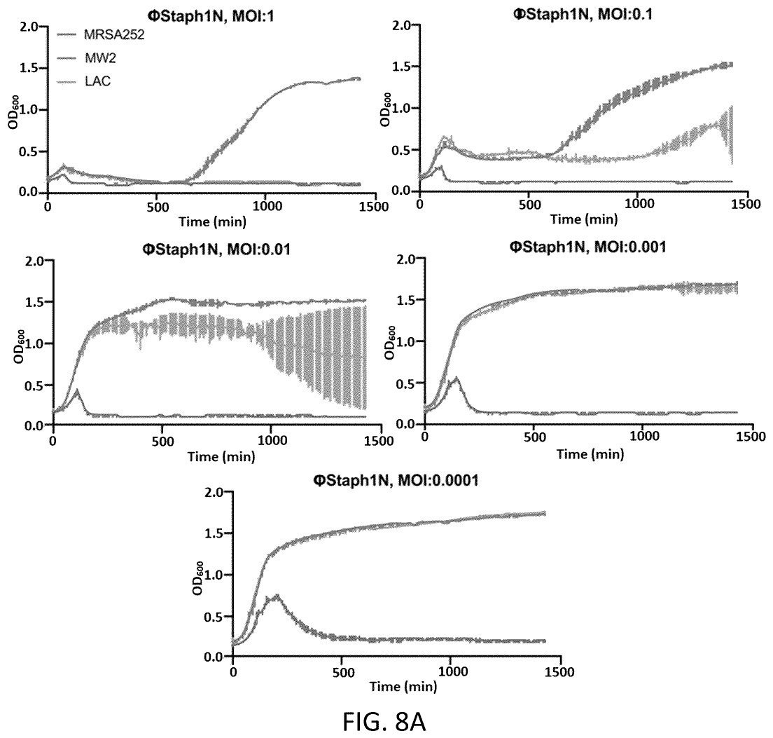

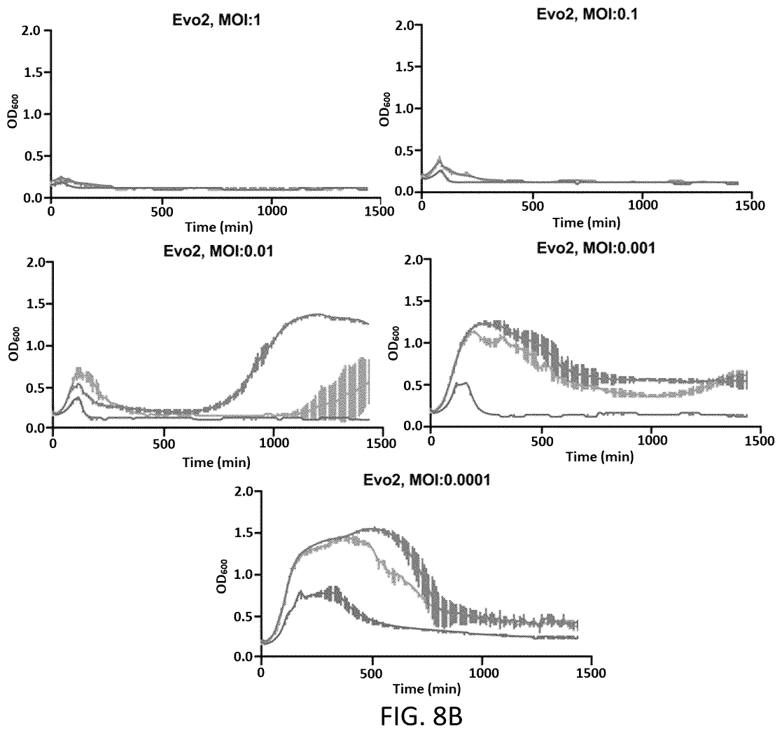

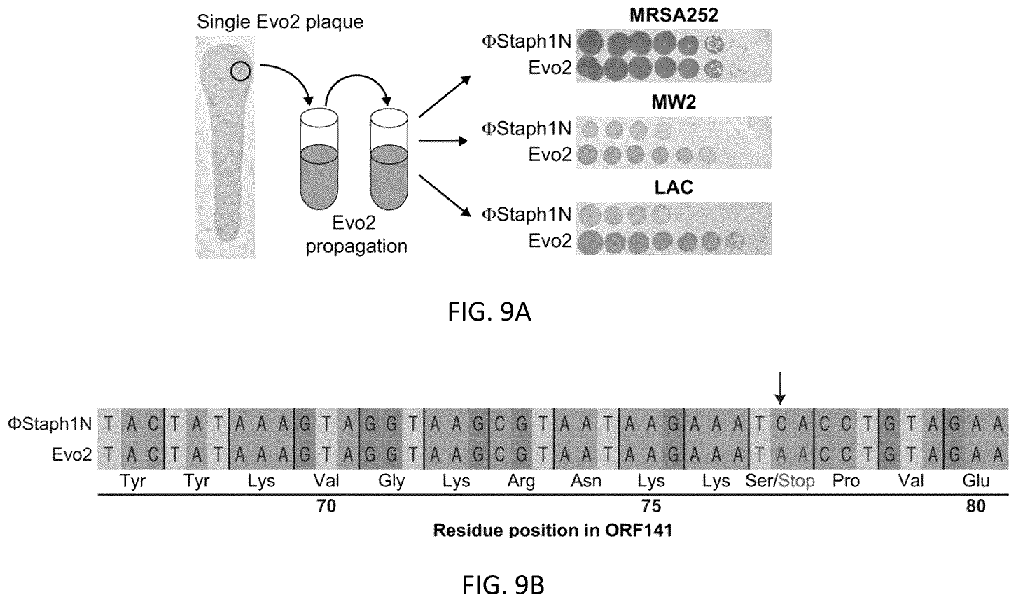

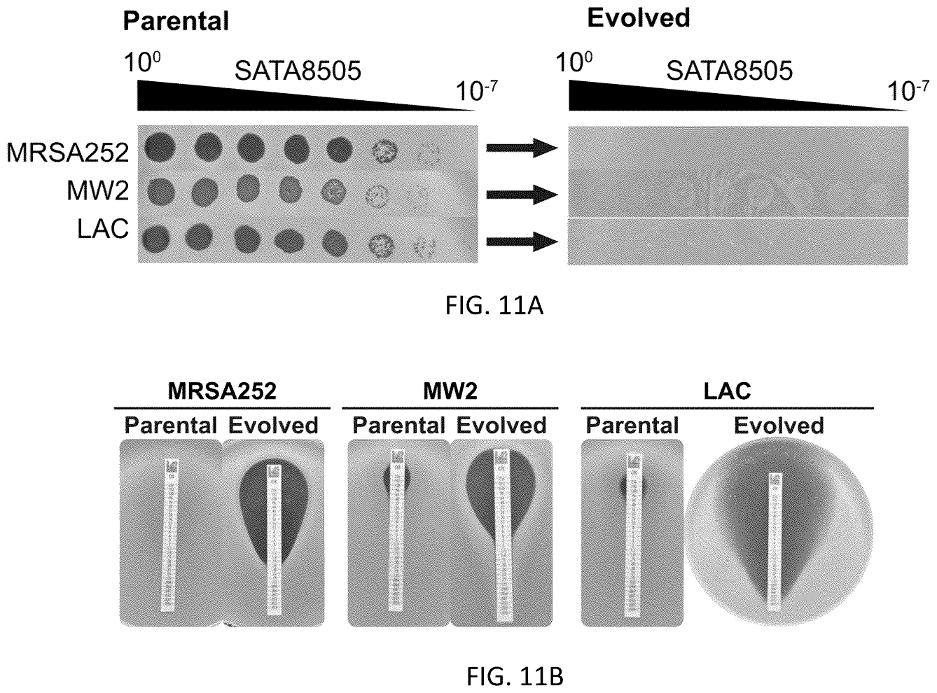

For our studies, we focused on three MRSA strains—MRSA252 (USA200), MW2 (USA400), and LAC (USA300). All three MRSA strains are pathogenic isolates implicated in human disease and are used as representative examples for studying MRSA.²²⁻²⁴ To test for phage susceptibility, we performed plaquing assays with a panel of staphylococcal phages (Table 1, FIG. 7). Of the phages tested, one phage, ΦStaph1N, which belongs to the Kayvirus genus, formed plaques on all three MRSA strains (FIG. 1B).^{21,25} Yet, despite its ability to infect all three strains, ΦStaph1N infection was unable to eradicate MRSA cultures. Both MW2 and LAC displayed incomplete lysis in liquid culture at multiplicities of infection (MOIs) of 0.1 or lower (FIG. 8A). Furthermore, infected cultures of all three MRSA strains could recover back to high cell density after passaging one percent of the culture into fresh media following 24 hours of initial infection. These results suggest that infection by ΦStaph1N selects for resistant mutants that could sweep the population. Indeed, ΦStaph1N was unable to form plaques on recovered MRSA cultures that survived in the initial ΦStaph1N infection (FIG. 11n).

TABLE 1
Bacterial strains and bacteriophages used in this study

| Strain Name | Comments | Source or reference |
|---|---|---|
| MRSA MW2 (USA400) | | Baba et al., 2002 |
| MRSA LAC (USA300) | | Voyich et al., 2005 |
| MRSA252 (USA200) | | Holden et al., 2004 |
| S. epidermidis RP62a | Methicillin-resistant | Christensen et. al., 1982 |
| | biofilm-producing | |
| | S. epidermidis | |
| S. epidermidis LAM1680 | Derived from S. epidermidis | Jiang et al., 2016 |
| | RP62a; carries genomic | |
| | deletion that inactivates | |
| | biofilm production | |
| ΦStaph1N | | Łobocka et al., 2012 |
| Evo2 | Derived from ΦStaph1N | This study |
| ΦNM1γ6 | Lytic version of ΦNM1 | Marraffini laboratory |
| ΦNM4γ4 | Lytic version of ΦNM4 | |
| Φ12 | | |
| Andhra | Infects S. epidermidis | Hatoum-Aslan laboratory |
| SATA8505 | Isolated from the | Environmental isolate; |
| | environment in this study | Pincus et al., 2015 |
| ADL1, ADL2, ADL3, ADL4, | | Levin laboratory; Land et |
| ADL5, ADL6, ADL7, ADL8, | | al., 2015 |
| ADL9, ADL10, ADL11, ADL12, | | |
| ADL13, ADL14, ADL15, ADL16, | | |
| ADL17, ADL18, ADL19, ADL20, | | |
| ADL21, ADL22, ADL23, ADL24, | | |
| ADL25, ADL26, ADL27, ADL28, | | |
| ADL29, ADL30 | | |
| AH1263 | LAC, Erm^{S} | Horswill laboratory |
| AH3455 | LAC mgrA::tetM | |
| AH1975 | LAC Δarl | |
| AH1525 | LAC sarA::kan | |
| AH843 | MW2 | |
| AH3456 | MW2 mgrA::tetM | |
| AH3060 | MW2 arl::tet | |
| AH5679 | MW2 sarA::Tn(Erm) | |

### Resistance Against ΦStaph1N Infection Sensitizes MRSA Against β-Lactams.

Because both phages and β-lactams interface with the bacterial cell wall, we hypothesized that resistance against ΦStaph1N infection could cause a trade-off in β-lactam resistance in MRSA even in the presence of PBP2a and BlaZ. We first tested the β-lactam sensitivity of the parental MRSA252, MW2, and LAC strains. As expected, all three strains displayed high minimal inhibitory concentrations (MICs) of ≥48 μg/mL against the β-lactams oxacillin (OXA), cefazolin (CEF), amoxicillin (AMX), and amoxicillin & clavulanic acid (AMX+CA), visually indicated by their ability to form lawns surrounding antibiotic strips (FIG. 1C, Table 2). The strains were sensitive to vancomycin (VANC; MICs=1.5 μg/mL), which inhibits cell wall synthesis through a different mechanism than β-lactams. Strikingly, phage-resistant MRSA that survived ΦStaph1N infection displayed a strong reduction in resistance against OXA, CEF, and AMX+CA, with fold reductions in MIC between 10 and 1000-fold (FIG. 1D); no change in MIC was observed with VANC or with AMX alone. These results show at a phenotypic level that ΦStaph1N-resistant MRSA loses resistance towards most β-lactams.

We next asked whether this loss of β-lactam resistance depended on the MOI of ΦStaph1N. We infected the three MRSA strains with ΦStaph1N at MOIs ranging from 10⁻² to 10⁻⁵, isolated the surviving MRSA cells, and tested their MIC against oxacillin (Table 2). For MRSA252, we still observed a ˜3-order of magnitude fold reduction of MIC at an MOI of 10⁻⁵. With MW2, the reduction of MIC was markedly decreased with lower phage levels, showing no significant loss at MOIs of 10⁻³ or lower. For LAC, two replicates displayed a reduction of MIC by an order of magnitude at an MOI of 10⁻⁴, while the third replicate did not display any change. These results show that for MRSA252, ΦStaph1N MOIs as low as 10⁻⁵ can still drive the loss of resistance, while for MW2 and LAC, higher MOIs of phage are needed to ensure the same outcome of reduced β-lactam resistance.

TABLE 2
MICs (μg/mL) against oxacillin of MRSA
strains treated with different MOIs of phage

| | MRSA252 | | | | | |
|---|---|---|---|---|---|---|
| | ΦStaph1N | | | Evo2 | | |
| MOI | Rep 1 | Rep 2 | Rep 3 | Rep 1 | Rep 2 | Rep 3 |
| 10\hat{ } − 2 | 0.25 | 0.125 | 0.38 | NG | 2 | 0.5 |
| 10\hat{ } − 3 | NG | 0.94 | 0.19 | 1 | 0.75 | 1 |
| 10\hat{ } − 4 | 0.5 | 0.25 | 0.19 | 0.75 | 1 | 0.5 |
| 10\hat{ } − 5 | 0.25 | 0.38 | NG | 0.38 | NG | NG |
| Mock | >256 | >256 | >256 | >256 | >256 | >256 |

MW2

| | ΦStaph1N | | | Evo2 | | |
|---|---|---|---|---|---|---|
| MOI | Rep 1 | Rep 2 | Rep 3 | Rep 1 | Rep 2 | Rep 3 |
| 10\hat{ } − 2 | 3 | 24 | 24 | 4 | NG | NG |
| 10\hat{ } − 3 | 32 | 24 | 48 | 4 | NG | NG |
| 10\hat{ } − 4 | 48 | 96 | 32 | 3 | NG | NG |
| 10\hat{ } − 5 | 96 | 64 | 24 | 2 | NG | NG |
| Mock | 96 | 48 | 32 | 96 | 48 | 32 |

LAC

| | ΦStaph1N | | | Evo2 | | |
|---|---|---|---|---|---|---|
| MOI | Rep 1 | Rep 2 | Rep 3 | Rep 1 | Rep 2 | Rep 3 |
| 10\hat{ } − 2 | NG | NG | 2 | 0.064 | NG | NG |
| 10\hat{ } − 3 | NG | 3 | 1.5 | 0.032 | NG | NG |
| 10\hat{ } − 4 | 32 | 1.5 | 1 | NG | NG | NG |
| 10\hat{ } − 5 | 32 | 16 | 0.38 | NG | NG | NG |
| Mock | 32 | 48 | 48 | 32 | 48 | 48 |

*NG: no growth detected

Discovery of a Mutant ΦStaph1N with Enhanced Activity Against MRSA

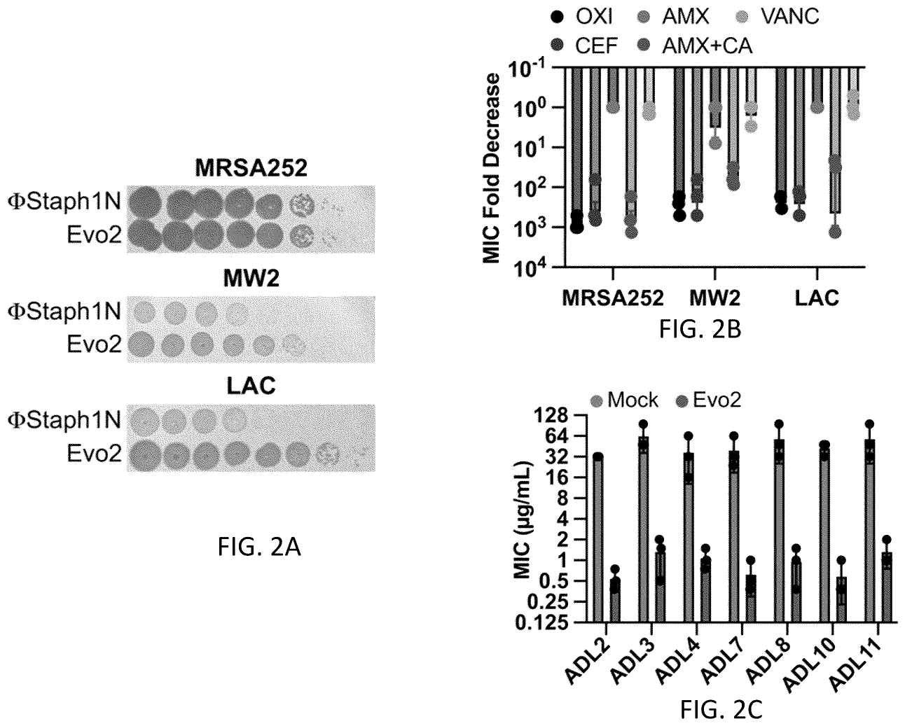

For ΦStaph1N, we noticed that while the phage could plaque on all three MRSA strains, its plaque-forming efficiency was reduced on the MW2 and LAC strains (FIG. 1B, FIG. 7). ΦStaph1N plaques on MW2 and LAC bacterial lawns were hazy, and the overall efficiency of plaquing was approximately two orders of magnitude less than that on MRSA252. Unexpectedly, we consistently observed smaller, clear plaques arising in the larger, hazy plaques of LAC (FIG. 9A); notably this did not appear in MW2. We hypothesized that these clear plaques were caused by a mutant form of ΦStaph1N that evolved higher lytic activity. We isolated phage clones from these single plaques and tested their activity against MRSA. This mutant phage, which we called Evo2, plaques on LAC and MW2 strains with higher efficiency, displaying comparable plaquing to MRSA252 (FIG. 2A). In growth experiments, we further observed that Evo2 lyses MRSA cultures at lower MOIs compared to ΦStaph1N (FIGS. 8A-8B). Evo2 exhibits lytic activity against MW2 and LAC even at an MOI of 10⁻⁴, a concentration at which ΦStaph1N does not show any detectable activity against the two strains.

We sequenced the genome of Evo2 to determine the genetic mechanism driving this enhanced activity. We observed a single point mutation in ORF141 that induces a premature stop codon (FIG. 9B). Sequence analysis with HHpred predicts ORF141 to be a putative DNA binding protein with an HTH motif (PDB: 2LVS, E-value: 2.5e-9). We speculate that this protein is a transcriptional regulator that when inactivated by a nonsense mutation, increases ΦStaph1N infectivity. Future studies will center on determining the mechanism of this mutation and why Evo2 only evolved in the LAC strain.

Given Evo2's enhanced activity against MRSA, we asked how predation by Evo2 affected β-lactam resistance. We infected MRSA252, MW2, and LAC with Evo2 at an MOI of 0.1 and measured the MICs against β-lactams after 48 hours of passaging. Similar to ΦStaph1N, infection by Evo2 reduced the MICs of the three MRSA strains against OXA, CEF, and AMX+CA, while MICs against AMX alone and VAN did not change significantly (FIG. 2B). Expanding on the different classes of antibiotics, we tested whether Evo2 predation could impact the susceptibility to the transcription inhibitor rifampicin; the translation inhibitors erythromycin and mupirocin; and the cell envelope disruptors teicoplanin, fosfomycin, and daptomycin (Table 3). We found that the MICs of these antibiotics did not change significantly, with a few exceptions: in some cases, Evo2-resistant LAC became sensitized to fosfomycin and daptomycin; furthermore, one replicate of Evo2-resistant MRSA252 evolved sensitivity to teicoplanin. However, overall the MIC reduction in these cases was not as dramatic as the MIC reduction seen against β-lactams.

TABLE 3
MICs (μg/mL) of mock- or Evo2-treated MRSA strains against different antibiotics

| | | Mock | | | Evo2 | | |
|---|---|---|---|---|---|---|---|
| Strain | Antibiotic | Rep 1 | Rep 2 | Rep 3 | Rep 1 | Rep 2 | Rep 3 |
| MRSA252 | Oxacillin | >256 | >256 | >256 | 0.38 | 0.75 | 0.5 |
| | Rifampicin | 0.047 | 0.032 | 0.012 | 0.023 | 0.047 | 0.023 |
| | Mupirocin | 0.75 | 0.5 | 1 | 0.5 | 0.5 | 0.38 |
| | Erythromycin | >256 | >256 | >256 | >256 | >256 | >256 |
| | Teicoplanin | 6 | 6 | 4 | 4 | 4 | 0.75 |
| | Fosfomycin | 12 | 8 | 8 | 8 | 8 | 6 |
| | Daptomycin | 2 | 3 | 2 | 2 | 2 | 2 |
| MW2 | Oxacillin | 48 | 32 | 32 | 0.75 | 1 | 0.75 |
| | Rifampicin | 0.032 | 0.047 | 0.064 | 0.023 | 0.023 | 0.032 |
| | Mupirocin | 0.5 | 0.25 | 0.5 | 0.38 | 0.38 | 0.25 |
| | Erythromycin | 1 | 0.75 | 0.75 | 0.25 | 0.5 | 0.25 |
| | Teicoplanin | 2 | 1.5 | 1.5 | 1.5 | 1 | 1 |
| | Fosfomycin | 1 | 1.5 | 1.5 | 0.5 | 1 | 1 |
| | Daptomycin | 1.5 | 2 | 1.5 | 0.75 | 0.5 | 3 |
| LAC | Oxacillin | 48 | 24 | 64 | 0.19 | 0.064 | 0.047 |
| | Rifampicin | 0.047 | 0.047 | 0.047 | 0.032 | 0.032 | 0.047 |
| | Mupirocin | 0.5 | 0.75 | 0.75 | 0.5 | 0.5 | 0.5 |
| | Erythromycin | 3 | 3 | 3 | 1.5 | 1 | 2 |
| | Teicoplanin | 1 | 0.5 | 1 | 0.5 | 0.5 | 0.75 |
| | Fosfomycin | 6 | 6 | 12 | 1.5 | 12 | 1 |
| | Daptomycin | 0.5 | 3 | 1 | 0.064 | 2 | 0.75 |

Finally, we examined how different MOIs of Evo2 impacted β-lactam resistance in MRSA (Table 2). We infected the three MRSA strains with Evo2 at varying MOIs from 10⁻² to 10⁻⁵ and measured the MIC against oxacillin of the evolved MRSA. Across all three strains, we found that replicate cultures across different MOIs were unable to recover growth following Evo2 infection (Table 2). However, cultures of MRSA252, MW2 and LAC that did regrow displayed a loss of oxacillin resistance, between 10- to 1000-fold. Thus, overall Evo2 displayed a higher infectivity against the MRSA and a greater potency in reducing β-lactam resistance.

Evo2 is Broadly Active Against Recent Clinical Isolates of S. aureus USA300

MRSA252, MW2, and LAC were isolated in 1997, 1998, and in the early 2000s, respectively. We therefore tested if Evo2 can infect more recent S. aureus clinical isolates. We compared the plaquing efficiency of Evo2 and ΦStaph1N against 30 USA300 strains that were isolated between 2008 and 2011 at St. Louis Children's Hospital (Table 1).²⁶ We observe dramatic variation in the plaquing efficiency of ΦStaph1N and the 30 strains, while Evo2 exhibited a higher plaquing efficiency in the majority of the 30 strains (Table 4; FIG. 21). We then tested how infection of Evo2 impacted OXA resistance in 12 (ADL1-12) of these clinical isolates. We infected the strains with Evo2 at an MOI of 0.1 and, if a phage-resistant population was recovered, measured the OXA MIC after 48 hours of passaging. Interestingly, after 15 independent challenges with Evo2, we were not able to recover phage-resistant populations from ADL1, 5, 6, and 12. This suggests that Evo2 resistance acquisition is a rare event in these strains. In the rest of the strains, we observed that similarly to MRSA252, MW2, and LAC, the OXA MIC was reduced between 10- to 100-fold after Evo2 infection (FIG. 2C). Overall, these results highlight the broader host range and activity of Evo2.

TABLE 4
Efficiencies of plaquing (EOPs)* of ΦStaph1N, Evo2, and ΦNM1γ6 on
clinical isolates of USA300 (ADL1-30)

| Strain | ΦStaph1N | Evo2 | FNM1g6 |
|---|---|---|---|
| RN4220 | 1.00E+00 | 1.00E+00 | 1.00E+00 |
| ADL1 | 1.70E−02 | 2.70E+00 | 6.70E−01 |
| ADL2 | 2.00E−01 | 3.30E+00 | 1.00E+00 |
| ADL3 | 6.00E−02 | 2.00E+00 | 1.70E−02 |
| ADL4 | 1.30E−03 | 1.00E+00 | 1.70E−01 |
| ADL5 | 1.20E−02 | 2.70E+00 | 1.00E+00 |
| ADL6 | 1.50E−02 | 1.70E+00 | 1.00E+00 |
| ADL7 | 6.00E−01 | 1.70E+00 | 6.70E−01 |
| ADL8 | 9.00E−02 | 2.00E+00 | 3.30E−01 |
| ADL9 | 6.00E−03 | 1.30E+00 | 2.00E−04 |
| ADL10 | 5.00E−01 | 1.30E+00 | 1.00E−03 |
| ADL11 | 1.00E+00 | 1.00E+00 | 3.30E−01 |
| ADL12 | 9.00E−04 | 2.00E+00 | 6.70E−07 |
| ADL13 | 7.00E−02 | 2.70E+00 | 2.30E−01 |
| ADL14 | 2.70E−01 | 3.30E+00 | 1.00E−05 |
| ADL15 | 9.00E−02 | 3.00E+00 | 2.00E−02 |
| ADL16 | 2.40E−01 | 5.60E+00 | 7.80E−02 |
| ADL17 | 3.30E+00 | 1.00E+01 | 2.40E+00 |
| ADL18 | 6.70E+00 | 1.40E+01 | 1.60E−02 |
| ADL19 | 7.10E−01 | 1.60E+00 | 2.30E+00 |
| ADL20 | 6.20E+00 | 7.80E+01 | 2.40E+00 |
| ADL21 | 5.20E+00 | 5.60E+00 | 3.30E−06 |
| ADL22 | 9.50E−01 | 2.00E+00 | 5.20E−01 |
| ADL23 | 1.50E+00 | 3.30E+01 | 1.20E−01 |
| ADL24 | 7.10E−01 | 5.60E+00 | 3.30E−06 |
| ADL25 | 7.10E−01 | 1.80E+00 | 2.30E−01 |
| ADL26 | 2.40E−02 | 3.90E+00 | 2.20E−03 |
| ADL27 | 1.30E+00 | 7.80E+00 | 1.70E+00 |
| ADL28 | 1.90E+00 | 8.90E+00 | 8.90E−02 |
| ADL29 | 1.90E+00 | 1.00E+01 | 1.30E−04 |
| ADL30 | 6.70E−01 | 1.40E+00 | 1.70E−05 |

*Phage EOPs on the clinical isolates are standardized to their respective EOP on the laboratory strain S. aureus RN4220.

### Effect on β-Lactam MIC by Other Phages

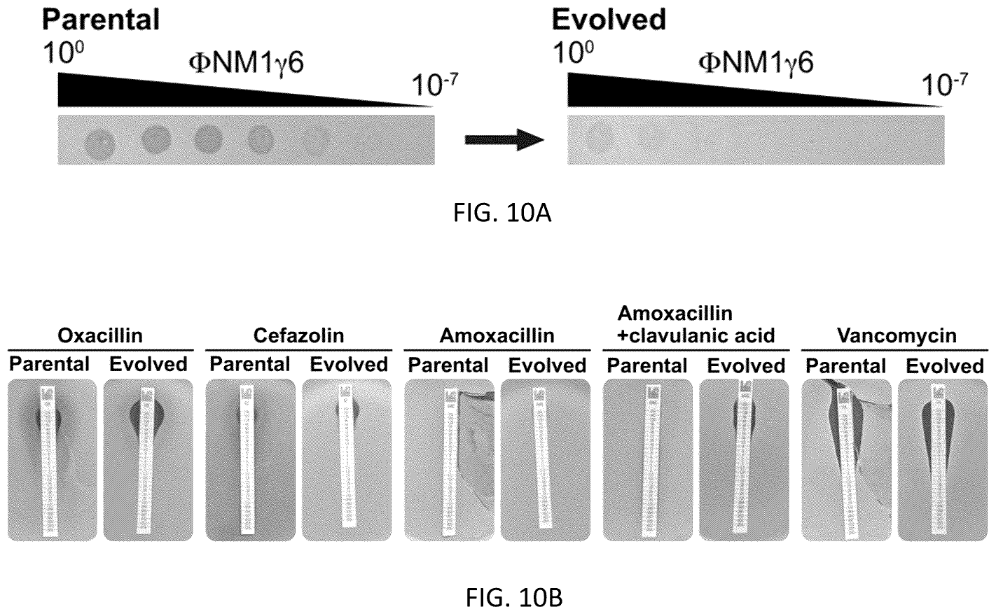

We asked whether two additional phages from our collection could elicit MIC reduction against oxacillin in MRSA. The MRSA LAC strain is sensitive to infection by ΦNM1γ6, a lytic version of the temperate phage ΦNM1 of the Dubowvirus genus, derived from the S. aureus Newman strain.²⁷⁻²⁹. In LAC, ΦNM1γ6 displays plaquing comparable to that of ΦStaph1N, while also showing activity against some of the clinical USA300 isolates (FIG. 7, Table 4). Therefore, we infected LAC with ΦNM1γ6 at an MOI of 0.1 and measured the MIC against β-lactams of the surviving cells. While the recovered LAC cultures exhibited resistance against ΦNM1γ6 they did not show a reduction in MIC (FIGS. 10A-10B), suggesting that phage resistance caused by ΦNM1γ6 is uncoupled from β-lactam resistance. We also isolated from the environment a second phage of the Kayvirus genus, called SATA8505,³⁰ and tested its activity against MRSA. SATA8505 is active against MRSA252, MW2, and LAC (FIGS. 11A-11B), and infection of the three strains caused arise of phage resistance in MRSA252, MW2, and LAC; (FIG. 11A). Similar to ΦStaph1N and Evo2, cells resistant to SATA8505 showed a strong loss of oxacillin resistance (FIG. 11B). Altogether, our results with these two phages suggest that the ability to reduce β-lactam resistance is not a universal feature across all staphylococcal phages and call for a more comprehensive analysis of staphylococcal phages and their ability to elicit β-lactam trade-offs.

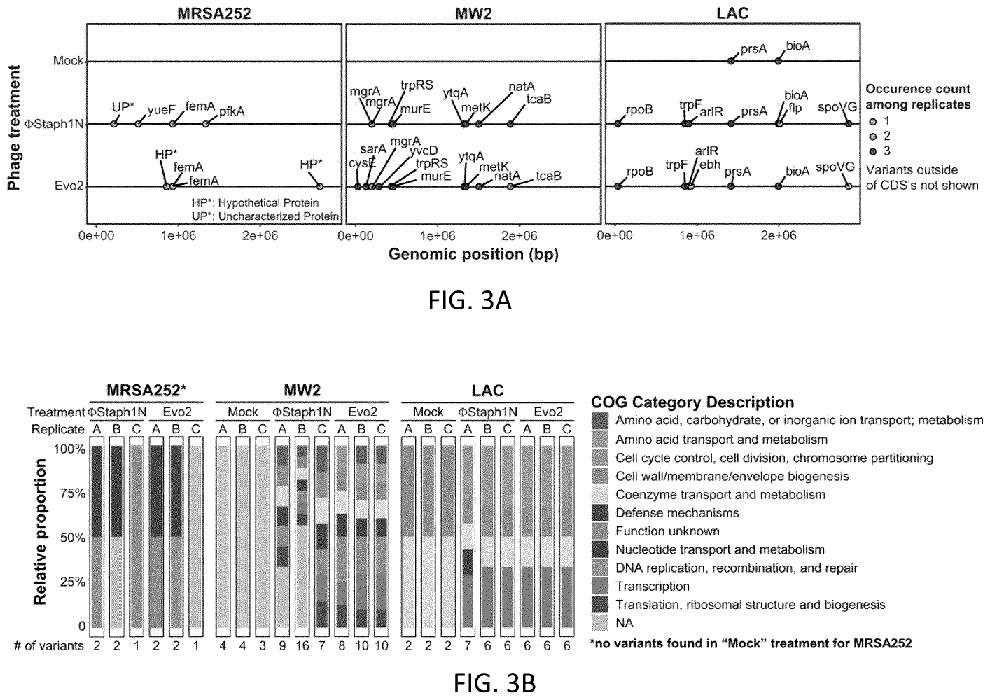

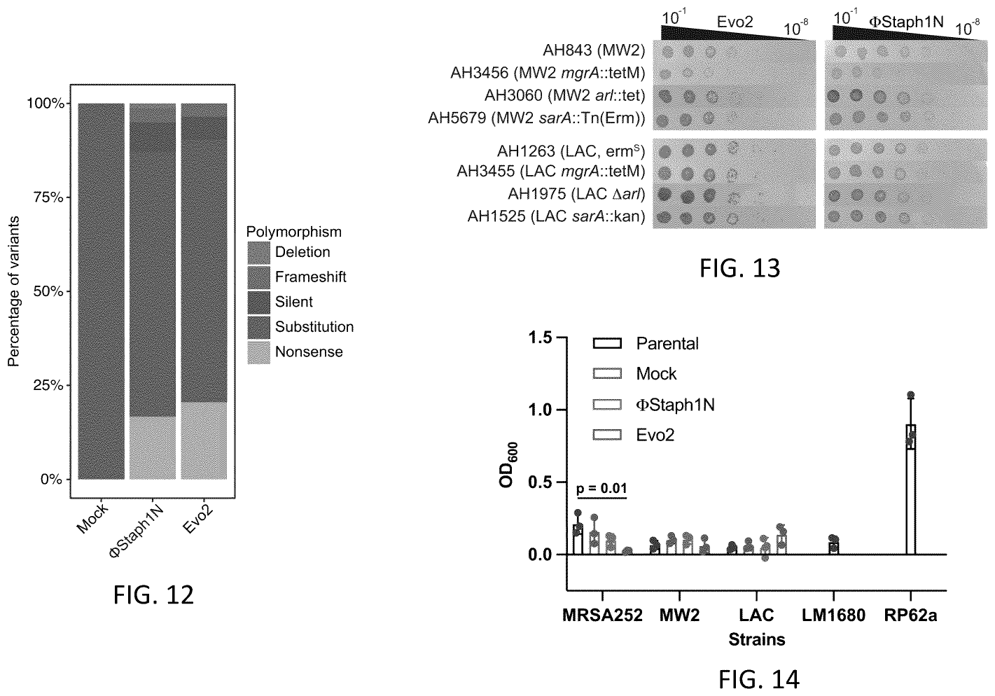

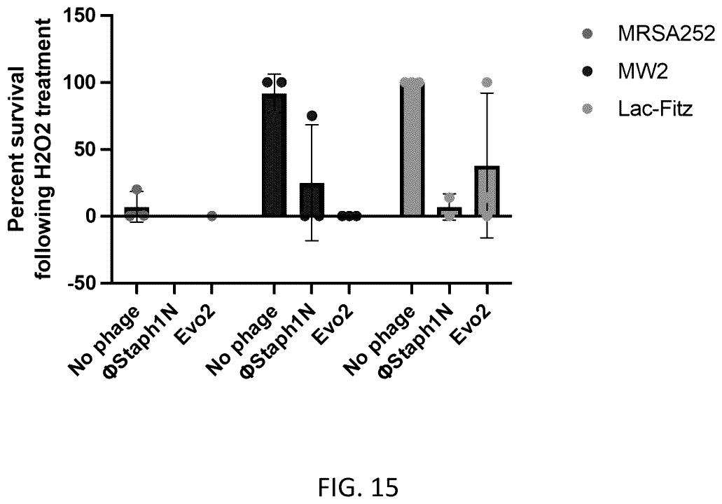

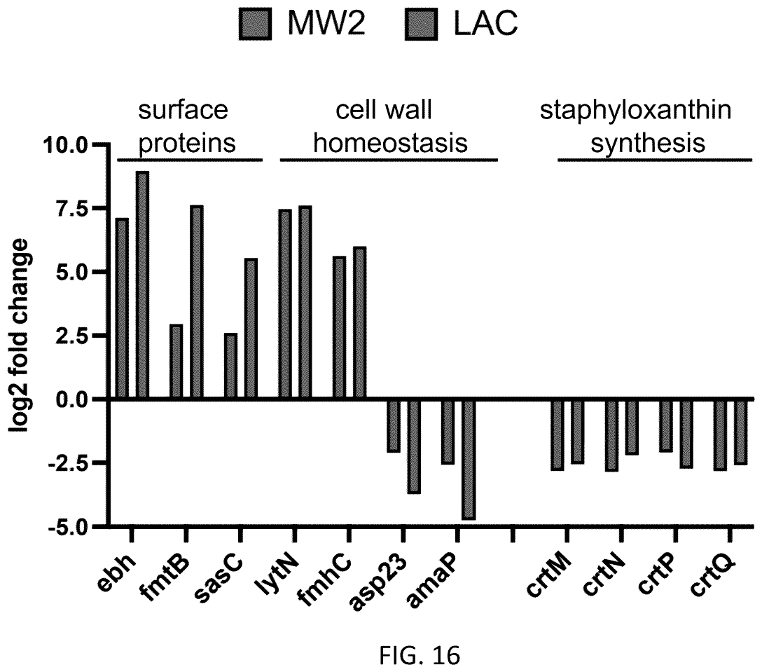

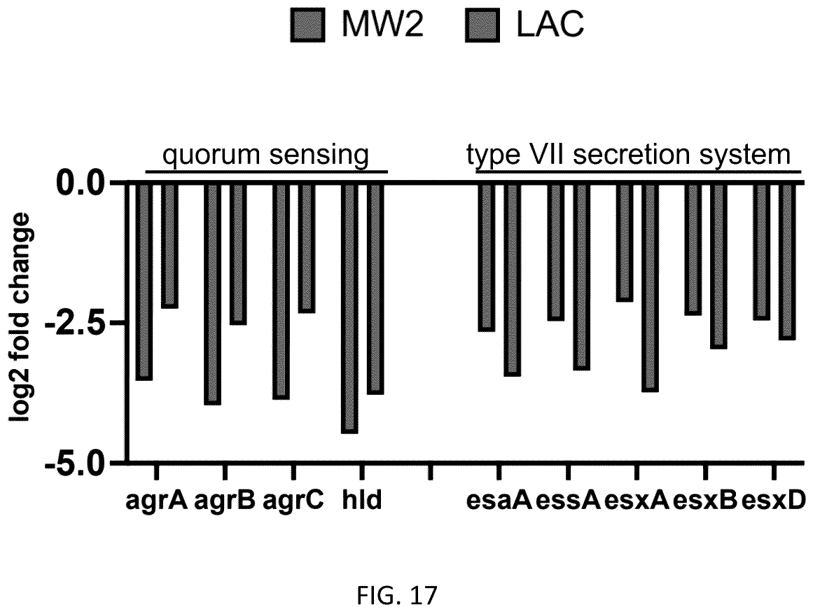

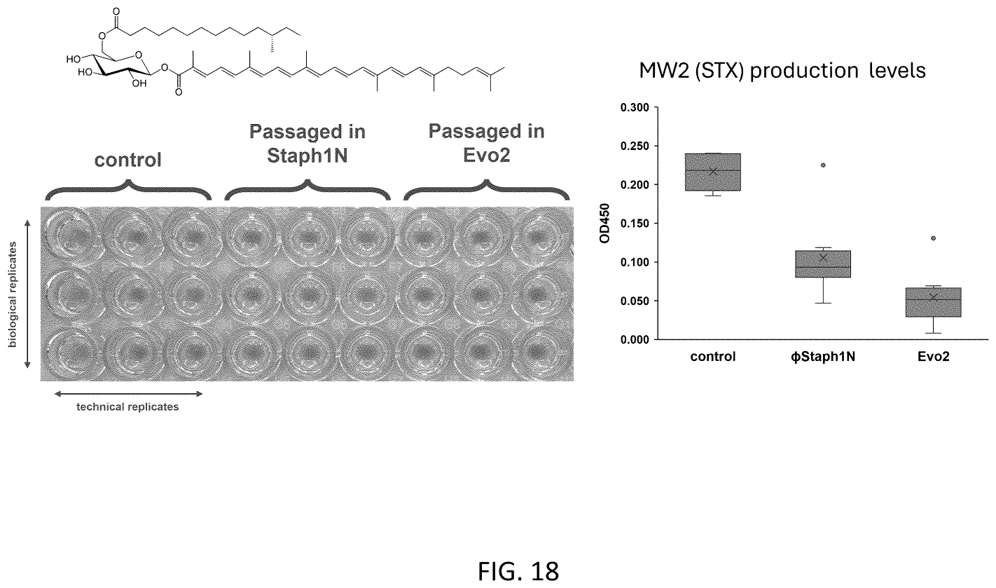

Genomic mutations in MRSA strains following phage infection Following our phenotypic analyses, we examined the genomes of the phage-resistant MRSA. We first sequenced the genomes of three clonal isolates (A-C) from each MRSA strain that underwent ΦStaph1N, Evo2, or a mock infection. We observed that each MRSA strain evolved distinct mutation profiles (FIG. 3A). Irrespective of the strain and phage treatment, most mutations were predicted to be substitutions, followed by truncations (FIG. 12). Cluster of Orthologous Genes (COG) variants associated with transcription, cell wall/membrane/envelope biogenesis, coenzyme transport and metabolism, and defense mechanisms were the most commonly found categories. Mutations in annotated genes that appeared at least twice across the clonal replicates are summarized in Table 5. Information on all detected genetic variants is listed in Table 6.

TABLE 5
Mutated genes in MRSA following infection with phages ΦStaph1N or Evo2

| Gene | Description | Strain | Phage infection | Reference |
|---|---|---|---|---|
| sarA | Transcriptional regulator of | MW2 | Evo2 | 31, 32 |
| | antibiotic resistance and virulence | | | |
| mgrA | Transcriptional regulator of | MW2 | ΦStaph1N, Evo2 | 33, 34 |
| | antibiotic resistance and virulence | | | |
| rpoB | Beta subunit of RNA polymerase | LAC | ΦStaph1N, Evo2 | 35 |
| | Transcriptional regulator of | | | |
| | antibiotic resistance | | | |
| arlR | Transcriptional regulator of | LAC | ΦStaph1N, Evo2 | 34, 36, 37 |
| | antibiotic resistance and virulence | | | |
| spoVG | Transcriptional regulator of | LAC | ΦStaph1N, Evo2 | 38, 39 |
| | antibiotic resistance and virulence | | | |
| cysE | Cysteine and methionine synthesis, | MW2 | Evo2 | 40 |
| | serine O-acetyltransferase | | | |
| metK | Cysteine and methionine synthesis, | MW2 | ΦStaph1N, Evo2 | 41 |
| | S-adenosylmethionine (SAM) | | | |
| | synthetase | | | |
| trpF | Phenylalanine, tyrosine and | LAC | ΦStaph1N, Evo2 | 42 |
| | tryptophan synthesis, | | | |
| | phosphoribosylanthranilate | | | |
| | isomerase | | | |
| femA | Peptidoglycan synthesis, | MRSA252 | ΦStaph1N, Evo2 | 43, 44 |
| | pentaglycine synthesis | | | |
| murE | Peptidoglycan synthesis, UDP- | MW2 | ΦStaph1N, Evo2 | 45 |
| | MurNAc tripeptide synthesis | | | |
| trpS | Aminoacyl-tRNA synthesis, | MW2 | ΦStaph1N, Evo2 | 46 |
| | tryptophanyl-tRNA synthesis | | | |
| ytqA | tRNA modifications, mnm⁵s²U | MW2 | ΦStaph1N, Evo2 | 47 |
| | synthesis | | | |
| yvcD | Unknown | MW2 | Evo2 | |
| natA | ABC transporter | MW2 | ΦStaph1N, Evo2 | 48 |
| tcaB | Predicted multidrug efflux pump | MW2 | ΦStaph1N, Evo2 | 49 |
| fmhC | Fem-like factors | LAC | FNM1g6 | 50 |

TABLE 6
Genes with significant fold changes in MRSA MW2 and LAC strains following Evo2 infection

| | | log2 Fold | | p-value, | |
|---|---|---|---|---|---|
| Gene | Strain | Change | p-value | adj. | Regulation |
| DBIAII_00174 = PBCCJM_00186 | MW2 | −4.48 | 2.86E−07 | 1.47E−06 | downregulated |
| DBIAII_00175 = PBCCJM_00187 | MW2 | −3.8 | 1.41E−05 | 5.71E−05 | downregulated |
| DBIAII_00176 = PBCCJM_00188 | MW2 | −5.23 | 4.97E−27 | 1.14E−25 | downregulated |
| DBIAII_00177 = PBCCJM_00189 | MW2 | −5.38 | 1.09E−32 | 2.95E−31 | downregulated |
| DBIAII_00178 = PBCCJM_00190 | MW2 | −5.91 | 5.99E−12 | 5.14E−11 | downregulated |
| DBIAII_00179 = PBCCJM_00191 | MW2 | −6.25 | 1.96E−32 | 5.22E−31 | downregulated |
| DBIAII_00183 = PBCCJM_00195 | MW2 | −6.53 | 4.92E−14 | 4.88E−13 | downregulated |
| DBIAII_00190 = PBCCJM_00202 | MW2 | −5.14 | 5.18E−33 | 1.47E−31 | downregulated |
| DBIAII_00256 = PBCCJM_00269 | MW2 | 2.47 | 5.12E−17 | 6.27E−16 | upregulated |
| DBIAII_00461 = PBCCJM_00477 | MW2 | 2.44 | 1.38E−40 | 5.35E−39 | upregulated |
| DBIAII_00467 = PBCCJM_00482 | MW2 | 8.48 | 1.72E−204 | 4.27E−202 | upregulated |
| DBIAII_00594 = PBCCJM_00608 | MW2 | 2.76 | 5.73E−20 | 8.68E−19 | upregulated |
| DBIAII_00595 = PBCCJM_00609 | MW2 | 2.43 | 3.40E−35 | 1.07E−33 | upregulated |
| DBIAII_00603 = PBCCJM_00617 | MW2 | 2.4 | 1.38E−88 | 1.24E−86 | upregulated |
| DBIAII_00607 = PBCCJM_00621 | MW2 | −4.61 | 3.87E−04 | 1.23E−03 | downregulated |
| DBIAII_00608 = PBCCJM_00622 | MW2 | −3.92 | 1.24E−11 | 1.05E−10 | downregulated |
| DBIAII_00998 = PBCCJM_01016 | MW2 | −2.54 | 4.40E−86 | 3.65E−84 | downregulated |
| DBIAII_00998 = PBCCJM_01017 | MW2 | −2.54 | 4.40E−86 | 3.65E−84 | downregulated |
| DBIAII_00999 = PBCCJM_00948 | MW2 | −3.8 | 2.56E−79 | 2.05E−77 | downregulated |
| DBIAII_00999 = PBCCJM_01018 | MW2 | −3.8 | 2.56E−79 | 2.05E−77 | downregulated |
| DBIAII_01132 = PBCCJM_01149 | MW2 | −2.02 | 1.78E−06 | 8.29E−06 | downregulated |
| DBIAII_01158 = PBCCJM_01175 | MW2 | 2.54 | 9.97E−26 | 2.07E−24 | upregulated |
| DBIAII_01189 = PBCCJM_01206 | MW2 | 2.02 | 9.13E−37 | 3.05E−35 | upregulated |
| DBIAII_01714 = PBCCJM_01760 | MW2 | −2.6 | 1.99E−10 | 1.50E−09 | downregulated |
| DBIAII_02017 = PBCCJM_02076 | MW2 | −2.01 | 1.65E−18 | 2.19E−17 | downregulated |
| DBIAII_02021 = PBCCJM_02080 | MW2 | 9.45 | 2.59E−257 | 1.16E−254 | upregulated |
| DBIAII_02211 = PBCCJM_02269 | MW2 | 3.52 | 1.80E−71 | 1.26E−69 | upregulated |
| DBIAII_02313 = PBCCJM_02405 | MW2 | −2.61 | 2.39E−47 | 1.09E−45 | downregulated |
| DBIAII_02313 = PBCCJM_02408 | MW2 | −2.61 | 2.39E−47 | 1.09E−45 | downregulated |
| DBIAII_02313 = PBCCJM_02409 | MW2 | −2.61 | 2.39E−47 | 1.09E−45 | downregulated |
| DBIAII_02340 = PBCCJM_02433 | MW2 | −2.15 | 8.68E−33 | 2.40E−31 | downregulated |
| DBIAII_02343 = PBCCJM_02436 | MW2 | 3.74 | 1.74E−123 | 2.79E−121 | upregulated |
| DBIAII_02346 = PBCCJM_02439 | MW2 | 2.42 | 6.84E−24 | 1.32E−22 | upregulated |
| DBIAII_02368 = PBCCJM_02462 | MW2 | −4.74 | 1.59E−29 | 3.74E−28 | downregulated |
| DBIAII_02391 = PBCCJM_02484 | MW2 | 2.2 | 4.47E−09 | 2.82E−08 | upregulated |
| DBIAII_02497 = PBCCJM_02580 | MW2 | 4.52 | 3.62E−133 | 6.24E−131 | upregulated |
| DBIAII_02505 = PBCCJM_02592 | MW2 | −2.39 | 2.13E−44 | 8.99E−43 | downregulated |
| DBIAII_02650 = PBCCJM_02740 | MW2 | −3.29 | 1.69E−73 | 1.22E−71 | downregulated |
| DBIAII_02651 = PBCCJM_02741 | MW2 | −3.11 | 3.35E−66 | 2.27E−64 | downregulated |
| DBIAII_02652 = PBCCJM_02742 | MW2 | −2.63 | 1.22E−52 | 5.95E−51 | downregulated |
| ebh | MW2 | 7.13 | 0.00E+00 | 0.00E+00 | upregulated |
| sarU | MW2 | 9.73 | 0.00E+00 | 0.00E+00 | upregulated |
| lytN | MW2 | 7.47 | 9.68E−298 | 7.23E−295 | upregulated |
| tarM | MW2 | 6.63 | 5.01E−292 | 2.80E−289 | upregulated |
| fmhC | MW2 | 5.62 | 4.11E−223 | 1.53E−220 | upregulated |
| efeO | MW2 | 4.03 | 2.34E−214 | 7.50E−212 | upregulated |
| sarV | MW2 | 4.15 | 9.15E−214 | 2.56E−211 | upregulated |
| sdrD | MW2 | 4.8 | 8.37E−185 | 1.88E−182 | upregulated |
| efeB | MW2 | 4.24 | 5.85E−181 | 1.19E−178 | upregulated |
| sarT | MW2 | 6.7 | 1.49E−179 | 2.79E−177 | upregulated |
| yut | MW2 | 2.34 | 1.72E−113 | 2.58E−111 | upregulated |
| spa | MW2 | 8.39 | 2.95E−112 | 4.13E−110 | upregulated |
| fTR1 | MW2 | 4.04 | 1.32E−104 | 1.74E−102 | upregulated |
| ureF | MW2 | 3.81 | 1.83E−99 | 2.28E−97 | upregulated |
| hysA | MW2 | −2.89 | 4.28E−99 | 5.05E−97 | downregulated |
| ureA | MW2 | 4.99 | 4.86E−97 | 5.44E−95 | upregulated |
| gLY1 | MW2 | 3.08 | 6.31E−96 | 6.73E−94 | upregulated |
| ureG | MW2 | 3.34 | 1.33E−95 | 1.35E−93 | upregulated |
| ureD | MW2 | 3.37 | 6.93E−92 | 6.75E−90 | upregulated |
| capA | MW2 | 4.03 | 6.45E−91 | 6.02E−89 | upregulated |
| wcaJ | MW2 | 4.05 | 1.56E−88 | 1.34E−86 | upregulated |
| ureC | MW2 | 4.28 | 3.13E−78 | 2.42E−76 | upregulated |
| fmtB | MW2 | 2.95 | 2.34E−75 | 1.75E−73 | upregulated |
| argF | MW2 | −2.01 | 6.12E−66 | 4.03E−64 | downregulated |
| lpl9 | MW2 | −3.67 | 3.24E−65 | 2.08E−63 | downregulated |
| sarS | MW2 | 3.2 | 4.10E−65 | 2.55E−63 | upregulated |
| msaC | MW2 | 2.85 | 4.73E−64 | 2.87E−62 | upregulated |
| ureE | MW2 | 3.79 | 4.19E−62 | 2.47E−60 | upregulated |
| arcA | MW2 | −2.86 | 4.87E−61 | 2.66E−59 | downregulated |
| sdrE | MW2 | −3.03 | 1.08E−59 | 5.79E−58 | downregulated |
| ureB | MW2 | 4.73 | 2.10E−55 | 1.09E−53 | upregulated |
| crtN | MW2 | −2.84 | 4.66E−54 | 2.37E−52 | downregulated |
| DBIAII_02627 | MW2 | 2.28 | 7.23E−54 | 3.60E−52 | upregulated |
| esaE | MW2 | −2.67 | 1.37E−50 | 6.51E−49 | downregulated |
| metN1 | MW2 | −2.11 | 1.05E−44 | 4.61E−43 | downregulated |
| DBIAII_02296 | MW2 | −2.24 | 1.66E−44 | 7.15E−43 | downregulated |
| cap8D | MW2 | −3.71 | 2.74E−44 | 1.14E−42 | downregulated |
| DBIAII_02618 | MW2 | −2.53 | 5.91E−44 | 2.41E−42 | downregulated |
| tatC | MW2 | 2.29 | 7.15E−44 | 2.86E−42 | upregulated |
| ywqE | MW2 | 3.54 | 1.46E−39 | 5.54E−38 | upregulated |
| esaA | MW2 | −2.65 | 1.16E−38 | 4.35E−37 | downregulated |
| dprA | MW2 | 2.81 | 3.29E−38 | 1.21E−36 | upregulated |
| sraP | MW2 | 2.25 | 1.12E−37 | 3.97E−36 | upregulated |
| cwrA | MW2 | 4.32 | 1.34E−37 | 4.71E−36 | upregulated |
| ldhD | MW2 | 4.09 | 3.07E−36 | 1.01E−34 | upregulated |
| cap8E | MW2 | −2.75 | 3.24E−36 | 1.05E−34 | downregulated |
| psmA4 | MW2 | −5.07 | 4.23E−34 | 1.26E−32 | downregulated |
| essA | MW2 | −2.58 | 2.92E−32 | 7.61E−31 | downregulated |
| feoA | MW2 | 4.26 | 3.43E−32 | 8.84E−31 | upregulated |
| sasC | MW2 | 2.6 | 4.81E−32 | 1.23E−30 | upregulated |
| arcD | MW2 | −2.03 | 8.96E−31 | 2.21E−29 | downregulated |
| cap8B | MW2 | −3.46 | 8.89E−30 | 2.12E−28 | downregulated |
| ceuD | MW2 | 2.22 | 1.85E−27 | 4.28E−26 | upregulated |
| sceD | MW2 | −2.28 | 5.16E−27 | 1.17E−25 | downregulated |
| crtM | MW2 | −2.82 | 8.48E−27 | 1.88E−25 | downregulated |
| lytR | MW2 | −2.24 | 3.47E−26 | 7.47E−25 | downregulated |
| ydhF | MW2 | −5.44 | 4.20E−26 | 8.88E−25 | downregulated |
| lrgA | MW2 | −3.86 | 6.84E−26 | 1.43E−24 | downregulated |
| cap8G | MW2 | −2.5 | 1.45E−24 | 2.93E−23 | downregulated |
| tenA | MW2 | 2.33 | 2.86E−23 | 5.49E−22 | upregulated |
| secY2 | MW2 | 2.19 | 2.90E−23 | 5.51E−22 | upregulated |
| ykoE | MW2 | 2.51 | 3.45E−23 | 6.44E−22 | upregulated |
| hfq | MW2 | 12.22 | 9.87E−23 | 1.80E−21 | upregulated |
| lip1 | MW2 | −2.76 | 1.28E−22 | 2.31E−21 | downregulated |
| cap8C | MW2 | −3.56 | 3.28E−22 | 5.79E−21 | downregulated |
| ppnN | MW2 | −5.45 | 4.85E−22 | 8.23E−21 | downregulated |
| ssaA | MW2 | −2.45 | 1.56E−21 | 2.58E−20 | downregulated |
| thiE | MW2 | 2.03 | 3.60E−21 | 5.85E−20 | upregulated |
| ftnA | MW2 | −2.51 | 3.89E−21 | 6.27E−20 | downregulated |
| pyrB | MW2 | −2.12 | 4.39E−21 | 6.98E−20 | downregulated |
| metL1 | MW2 | −3.62 | 4.72E−21 | 7.46E−20 | downregulated |
| esxA | MW2 | −2.14 | 9.07E−21 | 1.40E−19 | downregulated |
| thiM | MW2 | 2.03 | 7.79E−20 | 1.16E−18 | upregulated |
| essC | MW2 | −2.02 | 1.14E−19 | 1.67E−18 | downregulated |
| uraA | MW2 | −2.4 | 5.59E−19 | 7.93E−18 | downregulated |
| trpF | MW2 | −2.59 | 8.66E−19 | 1.21E−17 | downregulated |
| lytS | MW2 | −2.41 | 1.25E−18 | 1.72E−17 | downregulated |
| cntL | MW2 | −2.14 | 1.39E−18 | 1.88E−17 | downregulated |
| crtQ | MW2 | −2.87 | 5.16E−18 | 6.65E−17 | downregulated |
| essB | MW2 | −2.02 | 9.68E−18 | 1.24E−16 | downregulated |
| esxD | MW2 | −2.58 | 1.70E−17 | 2.12E−16 | downregulated |
| thiD | MW2 | 2.09 | 2.18E−17 | 2.70E−16 | upregulated |
| mtlD | MW2 | −2.08 | 5.25E−17 | 6.40E−16 | downregulated |
| lrgB | MW2 | −3.11 | 5.89E−17 | 7.13E−16 | downregulated |
| argG | MW2 | −3.33 | 6.39E−17 | 7.70E−16 | downregulated |
| ceuC | MW2 | 2.51 | 1.12E−16 | 1.32E−15 | upregulated |
| seh | MW2 | −3.15 | 1.58E−16 | 1.83E−15 | downregulated |
| asp1 | MW2 | 2.13 | 2.27E−16 | 2.58E−15 | upregulated |
| agrA | MW2 | −3.53 | 3.32E−16 | 3.74E−15 | downregulated |
| dapB | MW2 | −2.4 | 1.97E−15 | 2.17E−14 | downregulated |
| aur | MW2 | −2.13 | 2.68E−15 | 2.93E−14 | downregulated |
| ceuB | MW2 | 2.63 | 9.78E−15 | 1.03E−13 | upregulated |
| trpC | MW2 | −3.01 | 2.20E−14 | 2.27E−13 | downregulated |
| pstS | MW2 | 2.82 | 2.94E−14 | 2.98E−13 | upregulated |
| ehuA | MW2 | −2.65 | 4.21E−14 | 4.21E−13 | downregulated |
| agrB | MW2 | −3.97 | 5.39E−14 | 5.33E−13 | downregulated |
| cap8F | MW2 | −2.28 | 9.21E−14 | 9.05E−13 | downregulated |
| natK | MW2 | −3.9 | 3.47E−13 | 3.26E−12 | downregulated |
| yaiI | MW2 | −5.84 | 2.07E−12 | 1.86E−11 | downregulated |
| esxB | MW2 | −2.3 | 4.70E−12 | 4.12E−11 | downregulated |
| agrD | MW2 | −3.17 | 1.57E−11 | 1.31E−10 | downregulated |
| argH | MW2 | −2.72 | 2.02E−11 | 1.67E−10 | downregulated |
| lacA | MW2 | 2.03 | 2.87E−11 | 2.34E−10 | upregulated |
| asd | MW2 | −3.62 | 3.08E−11 | 2.51E−10 | downregulated |
| cydD | MW2 | −5.5 | 1.86E−10 | 1.40E−09 | downregulated |
| dapA | MW2 | −2.86 | 3.28E−10 | 2.37E−09 | downregulated |
| nrdD | MW2 | −2.34 | 3.30E−10 | 2.38E−09 | downregulated |
| amaP | MW2 | −2.55 | 3.99E−10 | 2.87E−09 | downregulated |
| fetB | MW2 | −2.37 | 6.18E−10 | 4.31E−09 | downregulated |
| pstC | MW2 | 2.13 | 8.52E−10 | 5.84E−09 | upregulated |
| uppP | MW2 | −5.41 | 9.33E−10 | 6.38E−09 | downregulated |
| vraX | MW2 | 2.08 | 3.19E−09 | 2.05E−08 | upregulated |
| psmA3 | MW2 | −5.12 | 3.85E−09 | 2.46E−08 | downregulated |
| cydC | MW2 | −5.11 | 4.28E−09 | 2.72E−08 | downregulated |
| hld | MW2 | −6.48 | 6.58E−09 | 4.13E−08 | downregulated |
| opp3C | MW2 | −2.27 | 1.55E−08 | 9.39E−08 | downregulated |
| psmA2 | MW2 | −7.28 | 1.71E−08 | 1.02E−07 | downregulated |
| crtP | MW2 | −2.04 | 4.01E−08 | 2.30E−07 | downregulated |
| asp23 | MW2 | −2.11 | 1.61E−07 | 8.57E−07 | downregulated |
| mgrA | MW2 | −4.95 | 2.13E−06 | 9.90E−06 | downregulated |
| DBIAII_00256 = PBCCJM_00269 | LAC | 6 | 3.39E−43 | 9.59E−42 | upregulated |
| DBIAII_00305 = PBCCJM_00303 | LAC | −3.16 | 5.34E−15 | 5.54E−14 | downregulated |
| DBIAII_00401 = PBCCJM_00416 | LAC | 2.5 | 5.60E−29 | 1.13E−27 | upregulated |
| DBIAII_00461 = PBCCJM_00477 | LAC | 4.54 | 0.00E+00 | 0.00E+00 | upregulated |
| DBIAII_00594 = PBCCJM_00608 | LAC | 3.83 | 1.69E−26 | 3.08E−25 | upregulated |
| DBIAII_00595 = PBCCJM_00609 | LAC | 3.2 | 3.32E−51 | 1.22E−49 | upregulated |
| DBIAII_00646 = PBCCJM_00662 | LAC | −2.09 | 1.51E−53 | 5.94E−52 | downregulated |
| DBIAII_00745 = PBCCJM_00760 | LAC | 4.52 | 7.89E−44 | 2.34E−42 | upregulated |
| DBIAII_00746 = PBCCJM_00761 | LAC | 2.14 | 4.23E−34 | 1.00E−32 | upregulated |
| DBIAII_00846 = PBCCJM_01758 | LAC | −2.18 | 6.03E−07 | 2.90E−06 | downregulated |
| DBIAII_00931 = PBCCJM_01018 | LAC | −3.45 | 4.80E−115 | 5.29E−113 | downregulated |
| DBIAII_00998 = PBCCJM_01017 | LAC | −3.33 | 1.32E−125 | 1.61E−123 | downregulated |
| DBIAII_00999 = PBCCJM_01018 | LAC | −3.45 | 4.80E−115 | 5.29E−113 | downregulated |
| DBIAII_01132 = PBCCJM_01149 | LAC | −2.84 | 3.00E−05 | 1.15E−04 | downregulated |
| DBIAII_01291 = PBCCJM_01317 | LAC | 2.21 | 4.45E−17 | 5.43E−16 | upregulated |
| DBIAII_01322 = PBCCJM_01351 | LAC | 2.04 | 2.20E−35 | 5.37E−34 | upregulated |
| DBIAII_01476 = PBCCJM_01506 | LAC | −2.22 | 4.34E−15 | 4.55E−14 | downregulated |
| DBIAII_01683 = PBCCJM_01723 | LAC | 8.29 | 1.40E−187 | 2.49E−185 | upregulated |
| DBIAII_01714 = PBCCJM_01760 | LAC | −4.62 | 1.36E−05 | 5.51E−05 | downregulated |
| DBIAII_01820 = PBCCJM_01874 | LAC | 3.93 | 2.68E−43 | 7.67E−42 | upregulated |
| DBIAII_01982 = PBCCJM_02038 | LAC | −2.44 | 1.92E−12 | 1.56E−11 | downregulated |
| DBIAII_01998 = PBCCJM_02054 | LAC | −2.33 | 5.45E−15 | 5.63E−14 | downregulated |
| DBIAII_02017 = PBCCJM_02076 | LAC | −3.66 | 4.30E−18 | 5.69E−17 | downregulated |
| DBIAII_02018 = PBCCJM_02077 | LAC | −3.2 | 1.28E−13 | 1.16E−12 | downregulated |
| DBIAII_02019 = PBCCJM_02078 | LAC | −3.51 | 1.08E−14 | 1.08E−13 | downregulated |
| DBIAII_02052 = PBCCJM_02112 | LAC | 3.2 | 3.42E−44 | 1.03E−42 | upregulated |
| DBIAII_02113 = PBCCJM_02173 | LAC | −3.5 | 4.30E−108 | 4.15E−106 | downregulated |
| DBIAII_02114 = PBCCJM_02174 | LAC | −3.36 | 3.74E−111 | 3.77E−109 | downregulated |
| DBIAII_02115 = PBCCJM_02175 | LAC | −3.51 | 3.56E−83 | 2.58E−81 | downregulated |
| DBIAII_02169 = PBCCJM_02227 | LAC | 2.86 | 8.92E−45 | 2.72E−43 | upregulated |
| DBIAII_02211 = PBCCJM_02269 | LAC | 4.04 | 3.27E−278 | 6.89E−276 | upregulated |
| DBIAII_02222 = PBCCJM_02281 | LAC | −3.3 | 1.51E−12 | 1.24E−11 | downregulated |
| DBIAII_02226 = PBCCJM_02284 | LAC | −2.02 | 3.66E−05 | 1.38E−04 | downregulated |
| DBIAII_02278 = PBCCJM_01758 | LAC | −2.18 | 6.03E−07 | 2.90E−06 | downregulated |
| DBIAII_02313 = PBCCJM_02405 | LAC | −2.43 | 1.01E−16 | 1.21E−15 | downregulated |
| DBIAII_02315 = PBCCJM_02405 | LAC | −2.43 | 1.01E−16 | 1.21E−15 | downregulated |
| DBIAII_02343 = PBCCJM_02436 | LAC | 8.48 | 0.00E+00 | 0.00E+00 | upregulated |
| DBIAII_02346 = PBCCJM_02439 | LAC | 6.82 | 0.00E+00 | 0.00E+00 | upregulated |
| DBIAII_02359 = PBCCJM_02452 | LAC | 3.58 | 6.36E−23 | 1.03E−21 | upregulated |
| DBIAII_02372 = PBCCJM_02465 | LAC | −2.6 | 9.73E−18 | 1.26E−16 | downregulated |
| DBIAII_02502 = PBCCJM_02587 | LAC | −2.22 | 2.65E−80 | 1.76E−78 | downregulated |
| DBIAII_02503 = PBCCJM_02588 | LAC | −2.44 | 5.23E−98 | 4.66E−96 | downregulated |
| DBIAII_02504 = PBCCJM_02589 | LAC | −3.71 | 6.75E−19 | 9.31E−18 | downregulated |
| DBIAII_02505 = PBCCJM_02592 | LAC | −3.59 | 2.99E−24 | 5.21E−23 | downregulated |
| DBIAII_02650 = PBCCJM_02740 | LAC | −2.12 | 4.99E−51 | 1.81E−49 | downregulated |
| DBIAII_02651 = PBCCJM_02741 | LAC | −2.06 | 3.29E−41 | 8.98E−40 | downregulated |
| ebh | LAC | 8.97 | 0.00E+00 | 0.00E+00 | upregulated |
| sarV | LAC | 6.03 | 0.00E+00 | 0.00E+00 | upregulated |
| fmhA | LAC | 4.1 | 0.00E+00 | 0.00E+00 | upregulated |
| ywqE | LAC | 7.62 | 0.00E+00 | 0.00E+00 | upregulated |
| wcaJ | LAC | 8.57 | 0.00E+00 | 0.00E+00 | upregulated |
| rfbX | LAC | 5.05 | 0.00E+00 | 0.00E+00 | upregulated |
| capA | LAC | 6.55 | 8.65E−305 | 2.00E−302 | upregulated |
| sak | LAC | −4.41 | 1.98E−215 | 3.83E−213 | downregulated |
| gLY1 | LAC | 5.45 | 3.62E−184 | 5.99E−182 | upregulated |
| marR | LAC | 2.23 | 3.49E−178 | 5.39E−176 | upregulated |
| ldhD | LAC | 4.54 | 7.20E−174 | 1.04E−171 | upregulated |
| fmtB | LAC | 7.62 | 4.46E−141 | 6.08E−139 | upregulated |
| lytN | LAC | 7.6 | 6.25E−128 | 8.04E−126 | upregulated |
| phnD | LAC | 6.78 | 9.07E−121 | 1.05E−118 | upregulated |
| ureC | LAC | 5.09 | 5.56E−113 | 5.85E−111 | upregulated |
| phnC | LAC | 6.31 | 2.22E−107 | 2.06E−105 | upregulated |
| splD | LAC | 5.29 | 1.21E−97 | 1.04E−95 | upregulated |
| ureB | LAC | 5.71 | 7.27E−89 | 6.01E−87 | upregulated |
| hfq | LAC | 3.04 | 1.15E−88 | 9.16E−87 | upregulated |
| fmhC | LAC | 6 | 3.49E−87 | 2.70E−85 | upregulated |
| mrp | LAC | 2.07 | 4.52E−85 | 3.38E−83 | upregulated |
| phnE | LAC | 5.12 | 1.82E−82 | 1.27E−80 | upregulated |
| yut | LAC | 3.93 | 1.39E−81 | 9.47E−80 | upregulated |
| ureE | LAC | 4.05 | 9.41E−79 | 5.89E−77 | upregulated |
| ureA | LAC | 6.43 | 5.77E−74 | 3.52E−72 | upregulated |
| opp4A | LAC | 8.69 | 1.05E−72 | 6.26E−71 | upregulated |
| essB | LAC | −3.08 | 1.79E−66 | 1.04E−64 | downregulated |
| msaC | LAC | 3.76 | 2.60E−66 | 1.47E−64 | upregulated |
| pyrC | LAC | −3.16 | 6.66E−66 | 3.68E−64 | downregulated |
| splE | LAC | 4.74 | 1.58E−65 | 8.51E−64 | upregulated |
| esaA | LAC | −3.47 | 1.03E−64 | 5.33E−63 | downregulated |
| splA | LAC | 5.4 | 1.32E−63 | 6.50E−62 | upregulated |
| ureF | LAC | 4.22 | 7.55E−63 | 3.64E−61 | upregulated |
| ureD | LAC | 3.53 | 1.32E−61 | 6.19E−60 | upregulated |
| splB | LAC | 5.48 | 3.08E−56 | 1.35E−54 | upregulated |
| ureG | LAC | 3.22 | 3.15E−56 | 1.35E−54 | upregulated |
| splC | LAC | 5.1 | 5.34E−56 | 2.25E−54 | upregulated |
| splF | LAC | 4.32 | 6.76E−56 | 2.80E−54 | upregulated |
| irrE | LAC | 12.27 | 4.75E−53 | 1.83E−51 | upregulated |
| essE | LAC | −3.11 | 1.70E−50 | 6.07E−49 | downregulated |
| sarT | LAC | −2.75 | 6.41E−50 | 2.25E−48 | downregulated |
| clfB | LAC | −3.75 | 1.93E−49 | 6.66E−48 | downregulated |
| fnbA | LAC | −3.58 | 3.25E−48 | 1.11E−46 | downregulated |
| lukE | LAC | 4.13 | 1.11E−47 | 3.72E−46 | upregulated |
| miaA | LAC | 2.5 | 1.08E−46 | 3.58E−45 | upregulated |
| PBCCJM_02590 | LAC | −3.11 | 4.78E−46 | 1.56E−44 | downregulated |
| brkB | LAC | −2.39 | 2.90E−45 | 9.34E−44 | downregulated |
| lukH | LAC | −4.05 | 5.98E−45 | 1.87E−43 | downregulated |
| isaB | LAC | 2.7 | 7.03E−45 | 2.17E−43 | upregulated |
| PBCCJM_02591 | LAC | −2.4 | 1.08E−43 | 3.17E−42 | downregulated |
| essA | LAC | −3.47 | 1.93E−43 | 5.59E−42 | downregulated |
| esxC | LAC | −3.3 | 1.51E−42 | 4.22E−41 | downregulated |
| pyrB | LAC | −3.26 | 5.06E−42 | 1.40E−40 | downregulated |
| lukD | LAC | 3.86 | 1.07E−40 | 2.88E−39 | upregulated |
| argC | LAC | 2.45 | 3.42E−40 | 9.11E−39 | upregulated |
| lukG | LAC | −3.61 | 7.62E−39 | 2.01E−37 | downregulated |
| esxA | LAC | −3.74 | 3.57E−38 | 9.30E−37 | downregulated |
| lpl10 | LAC | −2.2 | 1.49E−36 | 3.79E−35 | downregulated |
| yzzA | LAC | −2.54 | 1.11E−34 | 2.67E−33 | downregulated |
| tdcB | LAC | −4.27 | 8.20E−34 | 1.90E−32 | downregulated |
| sasC | LAC | 5.56 | 6.90E−33 | 1.58E−31 | upregulated |
| pxpA | LAC | 2.74 | 3.05E−31 | 6.79E−30 | upregulated |
| norB | LAC | −4.11 | 1.53E−29 | 3.17E−28 | downregulated |
| esxB | LAC | −3 | 4.26E−29 | 8.66E−28 | downregulated |
| ugpQ1 | LAC | −2.01 | 5.29E−28 | 1.01E−26 | downregulated |
| essC | LAC | −3.24 | 9.15E−27 | 1.70E−25 | downregulated |
| fetB | LAC | −2.71 | 8.96E−25 | 1.59E−23 | downregulated |
| ridA | LAC | −2.04 | 2.46E−24 | 4.31E−23 | downregulated |
| esxD | LAC | −2.6 | 1.69E−23 | 2.80E−22 | downregulated |
| gtfB | LAC | −2.42 | 2.71E−23 | 4.42E−22 | downregulated |
| gtfA | LAC | −2.64 | 7.59E−23 | 1.22E−21 | downregulated |
| mntH | LAC | 2.26 | 3.73E−22 | 5.73E−21 | upregulated |
| carA | LAC | −2.37 | 4.97E−22 | 7.58E−21 | downregulated |
| mtlD | LAC | −3.6 | 2.69E−21 | 4.02E−20 | downregulated |
| dprA | LAC | 3.02 | 4.72E−20 | 6.83E−19 | upregulated |
| msrA | LAC | 2.02 | 1.11E−19 | 1.59E−18 | upregulated |
| lytS | LAC | −2.18 | 1.19E−19 | 1.70E−18 | downregulated |
| PBCCJM_01714 | LAC | −2.51 | 2.26E−19 | 3.21E−18 | downregulated |
| mtlF | LAC | −3.52 | 2.45E−18 | 3.31E−17 | downregulated |
| znuA | LAC | −2.03 | 1.03E−17 | 1.32E−16 | downregulated |
| crtN | LAC | −2.2 | 1.24E−17 | 1.59E−16 | downregulated |
| lacD | LAC | 5.07 | 1.37E−17 | 1.74E−16 | upregulated |
| lacC | LAC | 4.91 | 1.40E−16 | 1.66E−15 | upregulated |
| crtQ | LAC | −2.82 | 1.58E−16 | 1.87E−15 | downregulated |
| cap8B | LAC | −2.78 | 1.81E−16 | 2.12E−15 | downregulated |
| cwrA | LAC | 2.6 | 2.74E−16 | 3.15E−15 | upregulated |
| lCB5 | LAC | −2.07 | 4.00E−16 | 4.52E−15 | downregulated |
| lacB | LAC | 4.99 | 1.29E−15 | 1.43E−14 | upregulated |
| metL1 | LAC | −2.45 | 1.32E−15 | 1.45E−14 | downregulated |
| lacA | LAC | 5.3 | 1.35E−15 | 1.48E−14 | upregulated |
| lacF | LAC | 5.1 | 5.02E−15 | 5.23E−14 | upregulated |
| phnB | LAC | −2.35 | 1.01E−14 | 1.02E−13 | downregulated |
| lrgB | LAC | −4.41 | 1.83E−14 | 1.80E−13 | downregulated |
| lacE | LAC | 4.38 | 2.52E−13 | 2.23E−12 | upregulated |
| crtM | LAC | −2.6 | 2.52E−13 | 2.23E−12 | downregulated |
| yocR | LAC | −2.31 | 4.87E−13 | 4.18E−12 | downregulated |
| cap8C | LAC | −2.94 | 9.84E−13 | 8.20E−12 | downregulated |
| lnsB | LAC | −2.27 | 1.30E−12 | 1.08E−11 | downregulated |
| wbiI | LAC | −2.63 | 4.99E−12 | 3.95E−11 | downregulated |
| lytR | LAC | −2.14 | 2.07E−11 | 1.55E−10 | downregulated |
| fnbB | LAC | −2.02 | 4.53E−11 | 3.26E−10 | downregulated |
| abgT | LAC | −2.48 | 6.66E−11 | 4.72E−10 | downregulated |
| uraA | LAC | −2.34 | 1.20E−10 | 8.35E−10 | downregulated |
| scn | LAC | −2.5 | 1.31E−10 | 9.08E−10 | downregulated |
| opp4C | LAC | 2.25 | 3.77E−10 | 2.51E−09 | upregulated |
| merR | LAC | −2.33 | 1.10E−09 | 7.10E−09 | downregulated |
| scb | LAC | −2.54 | 1.15E−09 | 7.39E−09 | downregulated |
| efeB | LAC | 3.49 | 2.28E−09 | 1.42E−08 | upregulated |
| lacG | LAC | 3.34 | 2.89E−09 | 1.77E−08 | upregulated |
| efb | LAC | −2.49 | 4.59E−09 | 2.77E−08 | downregulated |
| fTR1 | LAC | 3.32 | 1.01E−08 | 5.88E−08 | upregulated |
| crtP | LAC | −2.61 | 1.97E−08 | 1.10E−07 | downregulated |
| graF | LAC | −2.82 | 4.29E−08 | 2.32E−07 | downregulated |
| ssaA2 | LAC | −2.09 | 5.85E−08 | 3.12E−07 | downregulated |
| crtO | LAC | −2.74 | 6.48E−08 | 3.41E−07 | downregulated |
| yozE | LAC | −2.1 | 7.06E−08 | 3.71E−07 | downregulated |
| amaP | LAC | −4.74 | 9.86E−08 | 5.10E−07 | downregulated |
| lrgA | LAC | −4.81 | 1.92E−07 | 9.67E−07 | downregulated |
| feoA | LAC | 4.21 | 2.40E−07 | 1.21E−06 | upregulated |
| esaB | LAC | −3.08 | 2.43E−07 | 1.22E−06 | downregulated |
| pbp4 | LAC | −2.57 | 3.05E−07 | 1.52E−06 | downregulated |
| nikA | LAC | −2.24 | 3.78E−07 | 1.87E−06 | downregulated |
| ycjR | LAC | −2.01 | 1.78E−06 | 8.03E−06 | downregulated |
| sdrE | LAC | −2.12 | 2.54E−06 | 1.14E−05 | downregulated |
| efeO | LAC | 2.66 | 6.38E−06 | 2.73E−05 | upregulated |
| agrA | LAC | −2.25 | 8.01E−06 | 3.39E−05 | downregulated |
| opp3b | LAC | −2.13 | 1.05E−05 | 4.35E−05 | downregulated |
| opp3C | LAC | −2.22 | 1.17E−05 | 4.79E−05 | downregulated |
| sarS | LAC | −2.21 | 2.62E−05 | 1.02E−04 | downregulated |
| fpr | LAC | −2.06 | 3.23E−04 | 1.03E−03 | downregulated |
| asp23 | LAC | −3.72 | 1.43E−03 | 3.98E−03 | downregulated |

* DBIAII (MW2) or PBCCJM (LAC) prefix are the locus tag prefix as generated by Bakta (See Methods).

Without wishing to be bound by any particular theory, one plausible hypothesis explaining the loss of β-lactam resistance is that phage infection selected for a defective SCCmec or blaZ. However, we did not observe any mutations in the two loci. Instead, all MRSA strains exhibited mutations in ancillary genes implicated on the loss in β-lactam resistance. In MRSA252, both ΦStaph1N and Evo2 infection were selected for frameshift or nonsense mutations in the femA gene that would inactive the protein product. As discussed above, femA is required for the synthesis of the pentaglycine branch on S. aureus Lipid II, the peptidoglycan precursor (Table 5). Deletions of femA have been shown to increase susceptibility to β-lactams even when PBP2a (encoded by mecA) is expressed, thus providing a genetic mechanism for how some MRSA252 cells lose 0-lactam resistance after phage selection.^{43,44} At the same time, we found the presence of other uncharacterized mutations in phage-resistant MRSA252. For example, clone A of ΦStaph1N-treated cells carried 2 mutations: a frameshift in femA and a substitution mutation in an uncharacterized protein; meanwhile, clone B displayed a substitution mutation in pfkA, a predicted ATP-dependent 6-phosphofructokinase and mutation in an intergenic region; clone C showed a substitution mutation in a putative transport protein, called yueF (FIG. 3B, Table 6). The role of these mutations in mediating phage resistance or β-lactam sensitivity, if any, remains unknown.

In MW2, we found mutations in two transcriptional regulators, mgrA and sarA (FIG. 3A, Table 5). Both mgrA and sarA belong to the family of MarR (multiple antibiotic resistance regulator)/SarA (staphylococcal accessory regulator A) proteins, which regulate drug resistance and virulence in S. aureus.³¹⁻³³ In ΦStaph1N-treated MW2, only mgrA was mutated, while in Evo2-treated MW2, clones also showed nonsense mutations in sarA. We also found mutations in metK and ytqA which are both predicted to be associated with S-adenosylmethione (SAM): metK synthesizes SAM, while ytqA belongs to the radical SAM enzyme family and is predicted to be involved in tRNA modification.^{41,47} Phage-treated MW2 also displayed mutations in other genes, including tcaB and murE. Notably, each clonal replicate had multiple mutations in the genome, while by contrast untreated MW2 cells only displayed a deletion in an intergenic region that is not present in any of the phage-treated samples. These findings suggest that MW2 could be amassing multiple mutations during the course of phage infection.

For LAC, we observed a third, distinct mutational pattern (FIG. 3A). Of note, we found nonsense mutations in arlR, which is part of the arlRS two-component signaling system (Table 5). The activity of arlRS has been implicated in S. aureus virulence, pathogenicity, and oxacillin resistance.^{36,37} Further, we observed substitution mutations in spoVG, which is a transcription factor regulating the expression genes involved in a variety of functions, including cell wall metabolism (Table 5).³⁸ Indeed, spoVG activates the expression of femA.³⁹ Studies have shown that spoVG modulates β-lactam antibiotic resistance by modulating cell wall synthesis.³⁹ Similar to the other two MRSA strains, phage-treated clones of LAC showed multiple mutations in their genomes. We observed mutations in prsA and bioA that appeared in both the mock and phage treatment conditions, suggesting that these mutations do not arise due to phage selection.

Altogether, our results show that phage-infected MRSA strains acquire distinct mutational profiles. These mutations likely work in concert to promote phage resistance and β-lactam sensitivity, making it challenging to determine the mechanistic contributions of individual mutations. For example, we observed that the genes mgrA, sarR, and arlR evolved nonsense mutations, which would result in truncated, potentially non-functional protein products. We therefore tested if single knockout mutants of these genes alone are sufficient to confer resistance to ΦStaph1N and Evo2 (FIG. 13). In MW2, the mgrA knockout resulted in a modest reduction in plaquing of Evo2 and ΦStaph1N. However, none of the remaining mutants in either the MW2 or LAC background conferred resistance. Prior experimental studies have also shown that phage resistance in S. aureus can arise from the disruption of single genes directly involved in the synthesis and modification of WTA, such as tagO.⁵¹ Some MRSA strains also alter cell wall glycosylation through dedicated genes encoded on prophages.14 However, we did not see any mutations in genes directly involved in WTA synthesis. Our results thus highlight how MRSA can take on unique mutational pathways under phage selection.

Finally, we examined the mutational profile in ΦNM1γ6-resistant LAC populations Because infection with ΦNM1γ6 did not result in a decrease in OXA resistance, we hypothesized that mutations that arose in LAC following ΦNM1γ6 would be distinct from those following Evo2 infection. We found that the two genes were mutated across three clonal isolates from different resistant populations: bioA and fmhC. As seen previously, mutations in bioA appeared in the mock treatment, suggesting that that the mutations arose independently of phage selection. On the other hand, LAC showed a missense mutation in fmhC (H21D, Table 5, Table 6). FmhC and its homologue fmhA pair with femA and femB to incorporate Gly-Ser dipeptides into peptidoglycan cross-bridges.⁵⁰ However, the mechanism of the H21D mutation is unknown, and to our knowledge, mutations in fmhC have not been associated with phages resistance in S. aureus before.

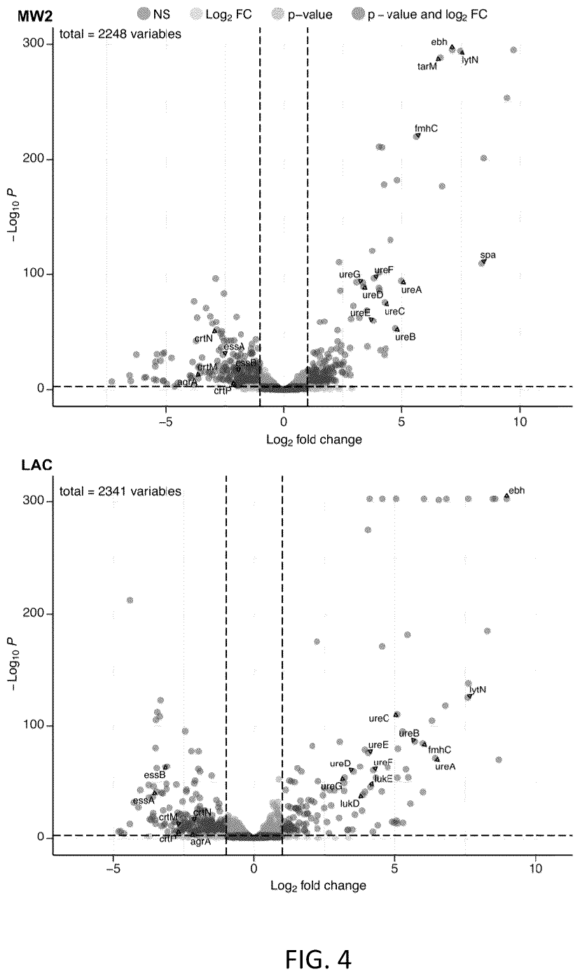

Phage-treated MRSA strains display broad changes to their transcriptomes Our genomic analysis revealed that phage-treated MRSA evolved mutations in a variety of transcriptional regulators, some of which are known to affect MRSA virulence. We therefore hypothesized that the mutations in these regulators would fundamentally alter the transcriptional profile of the treated MRSA. To test this, we performed bulk RNA-seq experiments on MW2 and LAC strains that were treated with the phage Evo2 and compared their transcription profiles to those of untreated strains (FIG. 4, Table 6). We observed significant changes in gene expression in both MW2 and LAC. Notably, mirroring the trend seen in the mutational data, we did not observe significant changes in the expression of genes in the SCCmec cassette or blaZ present in both MW2 and LAC.

We first compared our expression data against transcriptomic studies from previous studies. For example, Horswill and colleagues have shown that deletions of arlRS and mgrA de-represses the extracellular matrix binding protein ebh, resulting in significantly higher expression levels of the gene. In addition, these deletions also increased the expression of urease genes involved in the urea TCA cycle.^{33,37} In our experiments, both MW2 and LAC strains evolved nonsense mutations in mgrA and arlR, respectively. We therefore hypothesized that these mutations would mimic the effects of gene deletions and likewise yield elevated transcript levels of ebh and urease genes. Aligning with our hypothesis, differential expression data from both MW2 and LAC displayed a log 2 fold changes of >7 for ebh and >3 for ureABCDEFG genes (Table 6).

Additionally, both MW2 and LAC strongly upregulated several genes involved in cell wall maintenance. These include lytN, which is a murein hydrolase involved in the cross-wall compartment of S. aureus, and fmhC, which, as described previously, incorporate Gly-Ser dipeptides into pentaglycine cross-bridges in the S. aureus peptidoglycan cell wall. Overexpression of lytN has been shown to damage the cell wall, which in turn is alleviated by overexpression of fmhC.50 Interestingly, fmhC overexpression is linked to increased β-lactam sensitivity and thus may contribute to the loss of β-lactam resistance phenotypes we observed in the MRSA strains. MW2 and LAC also downregulated numerous genes, many of which are known virulence factors. Both strains reduced transcript levels of genes in the locus of the type VII secretion system (ess locus), staphyloxanthin biosynthesis (crtM, crtN, crtP), and quorum sensing (e.g. agrA) (FIG. 4, Table 6). Individually, these pathways have been shown to bolster the ability of S. aureus to establish infection and evade the host immune system.⁵²⁻⁵⁴ Our results suggest that infection by Evo2 can lead MRSA to reduce the expression of all of these pathways, which could reduce the virulence of S. aureus. Consistent with the decreased staphyloxanthin biosynthesis gene expression, MW2 treated with Evo2 or ΦStaph1N exhibited decreased production of staphyloxanthin. Staphyloxanthin is a carotenoid pigment with antioxidant properties that contributes to MRSA's ability to tolerate oxidative stress (FIG. 18).

Finally, we noted that both MW2 and LAC showed transcriptional changes that appear to be strain specific. For example, MW2 saw significant increase in the virulence factor spa, known to interfere with the host immune response and interface with other bacterial species. The presence of cell wall-bound Protein A has also been shown to decrease phage absorption, likely by masking WTA.⁵⁵ Further in MW2, we found that tarM, which adds α1,4-GlcNAc to WTA, was strongly upregulated (log 2 ratio=6.63). This is in line with previous findings showing that elevated tarM and α1,4-GlcNAc-WTA can lead to phage resistance in MRSA. The LAC showed an increase in the expression (log 2 fold change >3.8) of the hemolytic cytotoxin genes lukD/E, which lyses host cells and targets neutrophils.⁵⁶ We do not know whether the increased expression of these genes results in a greater level of protein production and secretion, but these transcriptional changes could represent a potential “trade-up” associated with phage resistance. Additional studies will be needed to fully assess the physiological and ecological effects of these upregulated genes in MRSA. Altogether, our RNA-seq results suggest that phage infection and resistance in MRSA cause significant transcriptional changes across a wide range virulence, metabolic, and cell-wall associated genes.

### Phage-Treated MRSA Strains Display Reduced Virulence Phenotypes

S. aureus is a highly virulent pathogen, relying on a vast array of toxins and immune evasion proteins to promote infection.⁵⁷ MW2 and LAC strains as models of MRSA virulence.^{33,34} In light of our mutational and transcriptomic data, we hypothesized that phage-treated MRSA cells would display altered virulence phenotypes, in addition to reduced β-lactam resistance. We first tested the ability of MRSA strains that survived phage predation for their ability to form biofilms in a Crystal Violet assay. We found that Evo2 infection of MRSA252 resulted in a significant reduction in Crystal Violet absorption compared to the parental strain. However, we show no significant difference in Crystal Violet absorption between parental, mock- and phage-treated MW2 and LAC strains (FIG. 14).

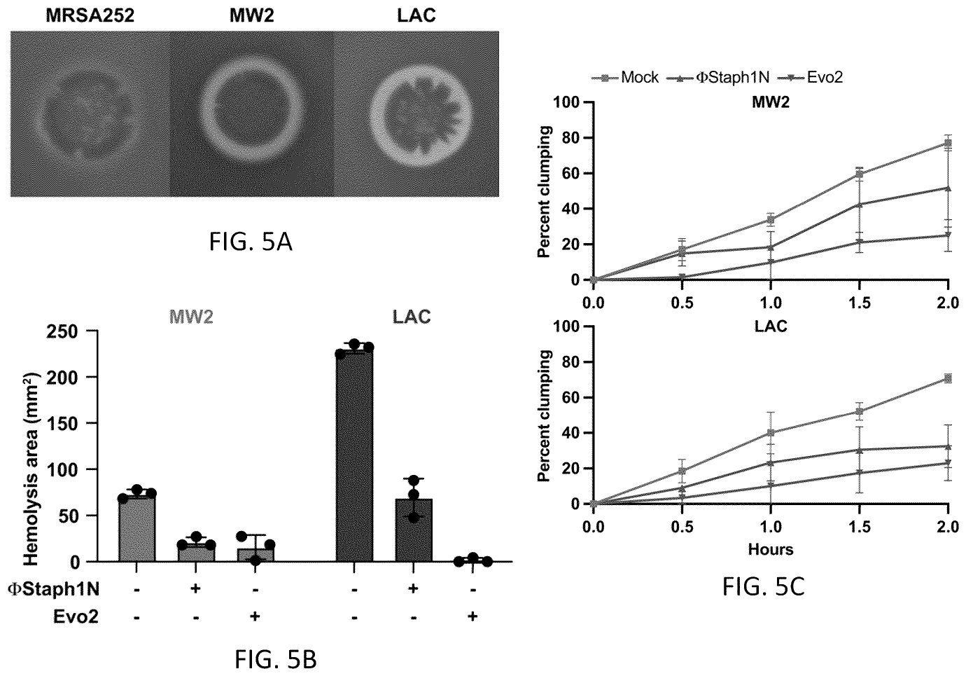

Next, we tested whether phage infection could affect the hemolysis of rabbit blood cells. Hemolysis is mediated by the secretion of toxins, notably alpha toxin encoded by the gene hla, and plays an important role in MRSA infection.⁵⁸ Expression of these toxins is regulated by virulence pathways that comprise numerous transcription factors, including mgrA, arlR, and sarA. Furthermore, in our RNA-seq results, we found that phage-resistant MRSA strains showed reduced expression of other cytotoxins. Parental MW2 and LAC colonies lysed rabbit blood cells on blood agar plates, producing distinct halos of clearance around the bacterial cells. For untreated LAC, the total area of hemolysis was on average 3-fold larger than that of untreated MW2 (˜210 mm2 vs ˜70 mm2, respectively); with MRSA252, by contrast, lysis was not detected (FIG. 5A). Following treatment with ΦStaph1N, we observed that MW2 and LAC displayed a reduced area of hemolysis by 4 to 5-fold. With Evo2-treated cells, we found that in MW2 the fold reduction was comparable to that of ΦStaph1N-treated cells. However, for Evo2-treated LAC, loss of hemolysis was even more pronounced, with two of the replicates showing no detectable hemolysis. We note that neither MW2 nor LAC showed a reduction in transcript expression of hla. We posit that the loss of hemolysis could be driven by an inability of phage-evolved MRSA to secrete the toxin.

We next tested how phage infection affected cell agglutination (or clumping) in MRSA. S. aureus binds to fibrinogen, forming protective aggregates of bacterial cells. Clumping is thought to have several functions in the context of staphylococcal infections, facilitating adhesion to the host tissue. Clumps are also likely to be more resistant to clearance by the immune system, partly because they may be too large to be phagocytosed by neutrophils.³⁴ In our transcriptional data, we noted that several cell surface proteins known to reduce cell clumping, such as ebh, were over-expressed in phage-resistant MRSA. Horswill and colleagues found that de-repression of ebh reduces clumping. Indeed, phage-treated MW2 and LAC displayed less clumping than the mock-treated or parental strain. For MW2, ΦStaph1N infection resulted in a modest reduction, while Evo2 infection resulted in a reduction of approximately 3-fold (FIG. 5B). In LAC, we found that both ΦStaph1N and Evo2 treatment resulted in comparable reductions of clumping in surviving cells. Overall, these phenotypic results align with our genetic and transcriptomic data, showing that phage infection can drive MRSA populations to reduced virulence phenotypes.

### Combination Treatment Between Evo2 and β-Lactam

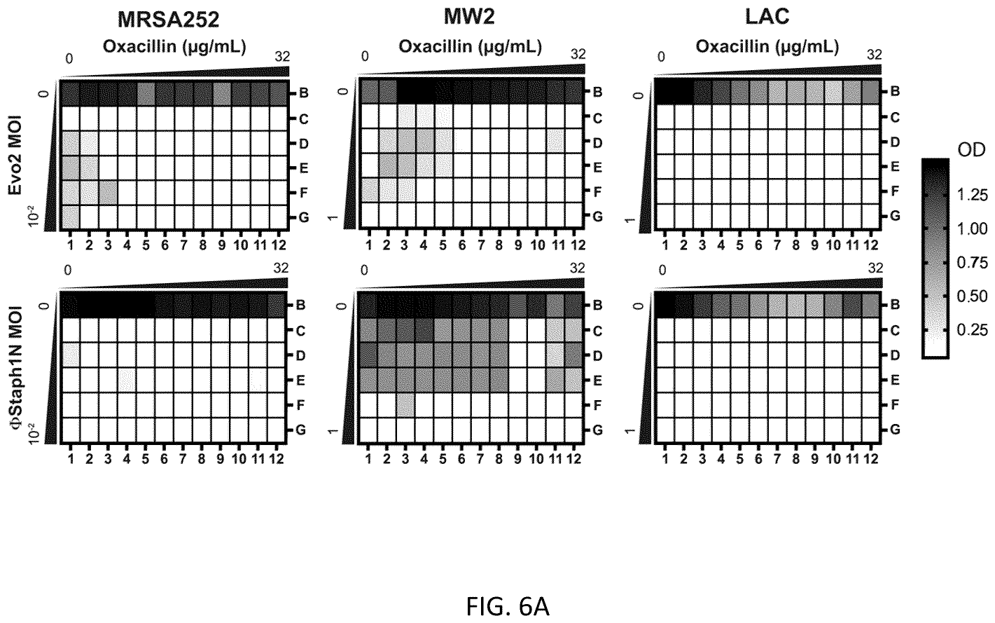

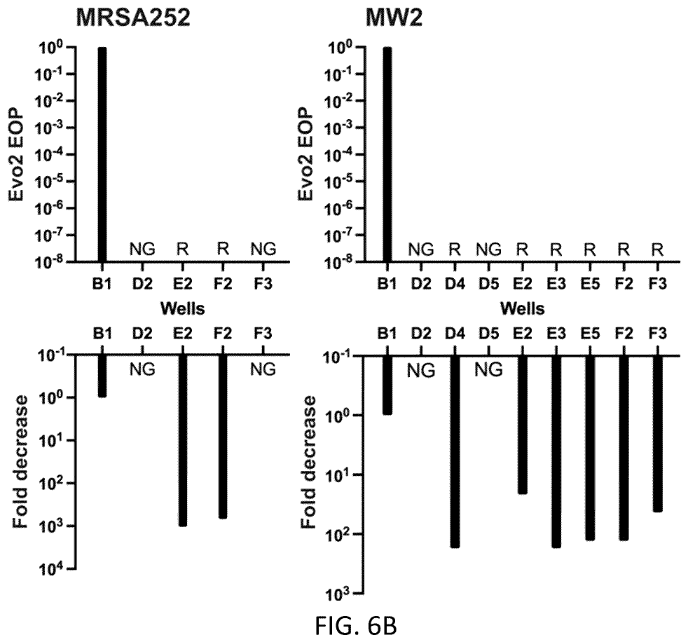

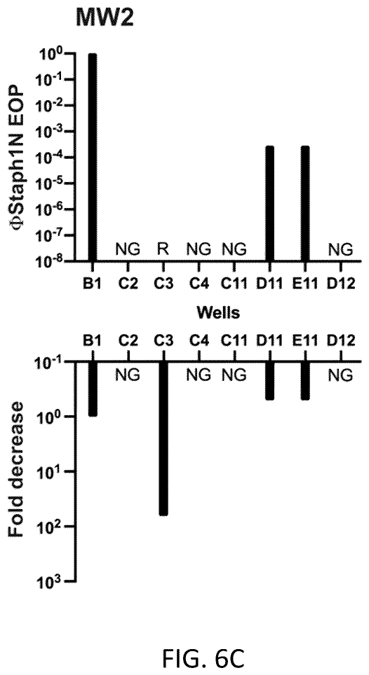

The aforementioned results suggest that MRSA cells evolve phage resistance following infection, which is associated with trade-offs in virulence and β-lactam resistance. We next asked how MRSA populations would evolve under co-treatment with phage and β-lactam. In principle, these two simultaneous selective pressures could drive the evolution of resistance against both the phage and antibiotic, negating the trade-offs in drug resistance. To test this, we performed checkerboard assays with phage and oxacillin on MRSA252, MW2, and LAC. Serial dilutions of Evo2 or ΦStaph1N were mixed with serial dilutions of oxacillin on a 96-well plate (FIG. 6A), after which MRSA strains were added to the plate and allowed to grow for 24 hours. Following 24 hours, one percent of the culture in each well was transferred into a fresh plate well with nonselective media, and the cultures were allowed to grow for another 24 hours (48 hours total). Throughout the experiment, the cell density was monitored by measuring the optical density in each plate well.

We first examined how MRSA grew in combinations of Evo2 and antibiotic (FIG. 6A, top row). For MRSA252, cells grew at low levels of phage (MOI of 0.01 or less) and oxacillin (<0.125 μg/mL) (FIG. 6A). LAC displayed greater sensitivity, displaying no detectable growth after 48 hours in the presence of phage, irrespective of the presence of oxacillin. For MW2, cells showed limited growth at MOIs <1 and oxacillin levels <0.125 μg/mL. For each strain, we picked cells from wells with an OD600>0.5 and inoculated them into fresh, non-selective media. As a control, we also regrew cells that were treated with neither phage nor oxacillin (well B1). For MRSA252, two (E2 and F2) wells contained viable MRSA cells, forming turbid cultures; for MW2, six (D4, E2, E3, E5, F2, and F3) wells picked regrew (FIG. 6B). We posit that cells in the failed cultures had reduced viability from the phage and antibiotic treatment. We tested these regrown cultures for their phage and oxacillin susceptibility. As expected, MRSA252 and MW2 cells from the B1 control wells were sensitive to Evo2, but resistant to oxacillin. In MRSA252, cells from E2 and F2 were resistant to Evo2 infection and exhibited a 1000-fold decrease in MIC against oxacillin. Similarly, in MW2, the six viable cultures exhibited Evo2 resistance and a 10- to 100-fold decrease in MIC. Altogether, these results match with those of MRSA that underwent single selection with Evo2.

We next analyzed how MRSA grew under ΦStaph1N/0-lactam combinations (FIG. 6A, bottom row). Neither MRSA252 nor LAC could grow in any ΦStaph1N/oxacillin combination; MW2, by contrast, grew across under a wide range of ΦStaph1N/oxacillin combinations. When ΦStaph1N-infected MW2 was treated with high (16 μg/mL) levels of oxacillin, recovered cells (wells D11 and E11) showed no reduction in MIC against oxacillin (FIG. 6C). However, the plaquing efficiency of ΦStaph1N was reduced by 3 to 6 orders of magnitude. Whole genome sequencing revealed that these cells evolved a unique set of mutations different from those seen in the single phage treatment conditions (Table 7). However, when ΦStaph1N-infected MW2 was co-treated with low levels (<0.125 μg/mL) of oxacillin, recovered cells (C3) displayed strong phage resistance and a 100-fold reduction in oxacillin resistance, mirroring phenotypes observed in the single phage treatment experiments.

TABLE 7
Mutations in surviving MW2 cells co-treated with ΦStaph1N and oxacillin

| Gene | Description | Reference |
|---|---|---|
| rsaC ncRNA | modulate oxidative stress response and | doi.org/10.1093/nar/gkz728 |
| | metal immunity | |
| nrdF | class 1b ribonucleoside- diphosphate | 10.1128/JB.183.24.7260- |
| | reductase subunit beta; beta subunit | 7272.2001 |
| | contains a metal-based cofactor; involved | 10.1074/jbc.M113.533554 |
| | in DNA synthesis | |
| fstAT ncRNA | Unknown | |
| rpoC | DNA-directed RNA polymerase subunit | |
| | beta' | |
| tRNA | Transfer RNA | |

We note that of the three MRSA strains tested, MW2 is the least sensitive to ΦStaph1N infection. We posit that the selective pressure exerted by high levels of β-lactam dominates over the selective pressure of ΦStaph1N, leading to the evolution of cells with continued β-lactam resistance and limited phage resistance. However, at lower β-lactam levels, the pressure exerted by phage infection predominates, leading to the rise of cells with complete phage resistance and trade-off in β-lactam resistance. This dose-dependent selection by oxacillin is not observed when a more active phage (e.g. Evo2) is used. While limited to one MRSA strain, these results suggest that different phage/β-lactam combinations can produce divergent evolutionary outcomes in MRSA, each with potential clinical implications.

### Phage Stability and Infectivity in Human Samples

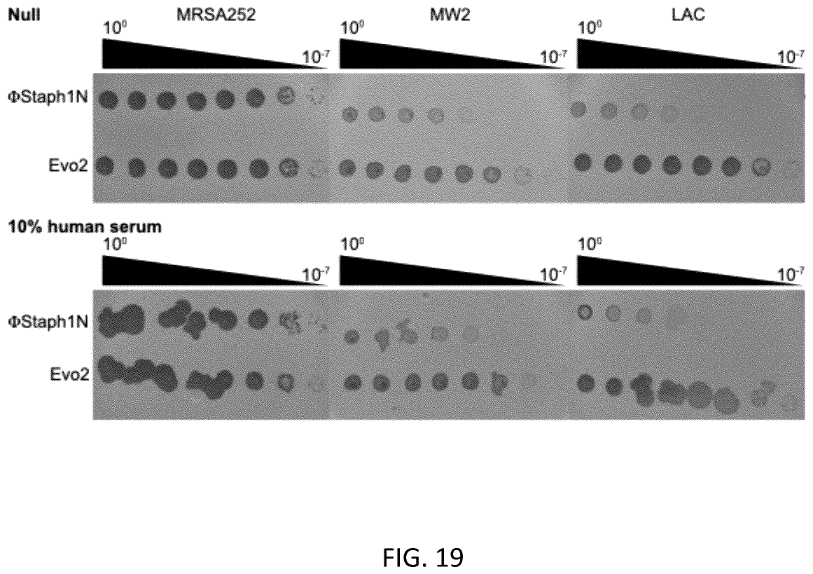

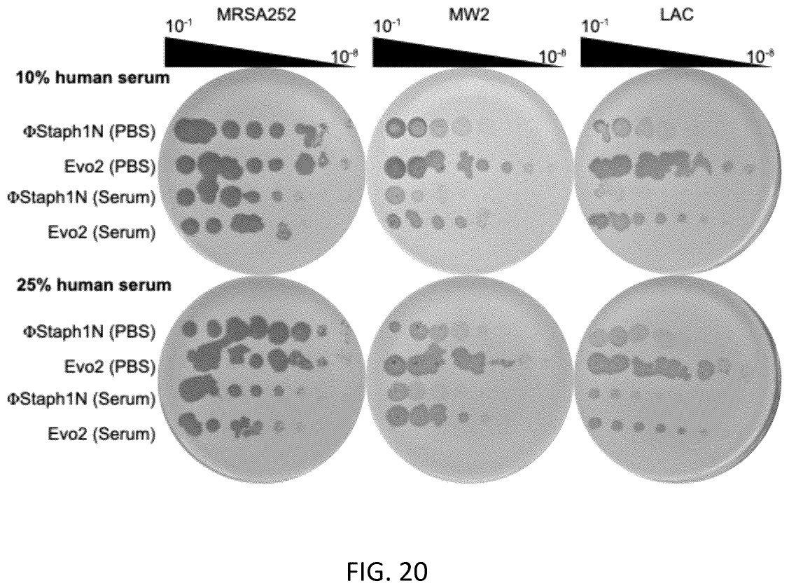

To investigate the compatibility of phage therapy in the treatment of human MRSA infections, we assessed the effect of human serum on phage activity (FIGS. 19-20) and the ability of phage to infect MRSA strains obtained from human clinical samples. First, serial dilutions of Evo2 and ΦStaph1N were added to a plate containing MRSA (MRSA252, MW2, or LAC), BHI top agar, and 10% human serum (FIG. 19). The presence of 10% human serum in the top agar exhibited minimal effect on the infectivity of Evo2 and ΦStaph1N. Next, we assessed the activity of Evo2 and ΦStaph1N when pre-incubated and plated with human serum (FIG. 20). Serial dilutions of Evo2 and ΦStaph1N were prepared in phosphate buffered saline (PBS) or diluted human serum (10% or 25%), incubated at room temperature for 1 hour, and added to a plate containing MRSA (MRSA252, MW2, or LAC), BHI top agar, and 10% or 25% human serum. The presence of 10% or 25% human serum in the top agar exhibited minimal effect on the infectivity of Evo2 and ΦStaph1N. In addition, Evo2 and ΦStaph1N retained infectivity following incubation with 25% human serum, though the number of plaques was reduced by 1-2 log.

### Discussion

Here we report the discovery that infection by certain phages can drive MRSA populations to evolve favorable genetic trade-offs between phage and β-lactam resistance. Exploiting genetic trade-offs has been proposed as a means to combat resistance in bacterial pathogens.18 Not only could phage treatments reduce the bacterial load of an infection, but also potentially resensitize bacterial populations to antibiotics against which they were previously resistant. We show that MRSA strains infected with Kayviruses ΦStaph1N, Evo2, and SATA8505 evolved resistance against phage, yet developed up to a 1000-fold loss in their MICs against β-lactam antibiotics. In addition, these evolved MRSA display reduced virulence phenotypes such as lower levels of hemolysis and clumping. Our findings show that phages can resensitize MRSA to β-lactams and even decrease their virulence, which are outcomes of significant biomedical value. However, not all staphylococcal phages can mediate these trade-offs: infection with ΦNM1γ6 a phage of the Dubowvirus genus, did generate phage resistance in MRSA, but did not produce a drop in β-lactam resistance. A major future direction will be to determine which types of phages elicit these beneficial evolutionary trade-offs in MRSA.

Our results also paint a complex picture of MRSA evolution during phage infection. Whole genome sequencing revealed that MRSA strains evolved distinct mutation profiles following phage infection, suggesting a multitude of evolutionary paths that different bacterial strains can undertake to evolve resistance against phage. Not only did MRSA strains evolve distinct mutations, but individual, phage-resistant clones accumulated multiple mutations in their genome. Nonetheless, different MRSA displayed a convergence of phenotypes in the form of phage resistance, reduced β-lactam resistance, and attenuated virulence. We posit that this convergence is caused by the involvement of the cell wall in all three phenotypic outcomes. Phages must interface with the cell wall, β-lactams target proteins associated with cell wall maintenance, and many S. aureus virulence factors are embedded within the cell wall or are secreted through it. Thus, any modifications to the integrity or chemical composition of the cell wall by phage resistance will impact β-lactam sensitivity and virulence. Cell wall maintenance is controlled by numerous genes, ranging from single proteins involved in cell wall synthesis, such as femA, to transcriptional regulators, such as mgrA, that control the expression of cell wall synthesis genes. Thus, the mutational patterns observed in each MRSA strain could reflect genetic solutions that enable the bacteria to adapt to the phage predation, while also maximizing the fitness for that particular strain.

Strikingly, MRSA heavily modulated transcriptional profiles following phage infection. We believe these altered expression profiles are a consequence of the genomic mutations that emerged in the various transcriptional regulators. We observed that evolved cells downregulated genes involved in quorum sensing, type VII secretion, and a variety of toxins. It is intriguing to speculate how the down-regulation of these genes impacts MRSA interactions with other bacteria occupying the same ecological niche and with the host immune system. At the same time, MRSA strains also upregulated expression of select virulence factors, such as spA, which could represent “trade-ups.” Trade-ups are thought of as non-selected traits that are enhanced following selection (e.g. cross-resistance between phage and antibiotic), which from a therapeutic perspective might be undesirable.⁵⁹ Future work will focus on assessing the risk of these trade-ups in light of the clinical benefit of reduced resistance and virulence.

Drug resistance in bacterial pathogens is an evolutionary problem and will require evolution-guided solutions to mitigate. Our findings highlight the ability of phages to dramatically alter the evolution and physiology of drug-resistant MRSA. Select phage treatments can force bacterial populations down evolutionary paths that make them vulnerable to antibiotics or the host immune system. Critically, this permits the re-deployment of agents that would otherwise remain ineffective, buying time for new drug discoveries. We therefore hope that our work may suggest avenues of research into new phage-based treatment strategies against MRSA and other drug resistance pathogens.

### Conclusion

These results identify a novel, more virulent bacteriophage (Evo2), which exhibits higher levels of infectivity against MRSA. In addition, the results demonstrate that, following infection by ΦStaph1N or Evo2 bacteriophage, MRSA bacteria (MRSA252, MW2, and Lac-Fitz) develop and evolved phenotype, characterized by increased sensitivity towards β-lactam antibiotics, increased susceptibility to oxidative stress, and reduced virulence, as evidenced by hemolytic activity and clumping.

### Materials and Methods

### Stains and Culture Conditions

The bacterial strains used in this study are listed in Table 1. Unless otherwise indicated, all MRSA strains were grown in Brain Heart Infusion (BHI) media at 37° C. with shaking (235 RPM).

### Plate-Based Plaque Assay

Bacterial lawns were prepared by mixing 100 μL of an overnight culture with 5 mL of melted BHI agarose (top agar). The bacteria and top agar mixture were poured onto a solid BHI plate. The plate was dried for 10 minutes. 10-fold serial dilutions (10⁰-10⁻⁷ unless otherwise noted) of phage were then spotted on the bacterial lawn. Plates were then incubated at 37° C. for 16 hours. Phage titer in plaque-forming units per μL (pfu/μL) was then calculated.

### Phage Infection Assay

MRSA strains were plated onto BHI agar plates and grown overnight. Individual colonies from the parental strains (also referred to as P0) were picked. Single colonies were inoculated in a round bottle tube containing 5 mL BHI broth. The cultures were incubated at 37° C., 235 RPM for 24 hours. The grown P0 cultures were then diluted 1:100 into fresh 5 mL BHI broth. At the early log phase (OD˜0.3), the bacterial cultures were treated with phage at an MOI of 0.1, unless indicated otherwise. The treated bacterial cultures were incubated at 37° C. with shaking (235 RPM) for 24 hours. The cultures were then passaged 1:100 into fresh 5 mL BHI broth. This passage was then grown at 37° C., 235 RPM, for another 24 hours. Surviving cultures were then used for both phenotypic assays and sequencing experiments. As a negative control, MRSA strains were passaged using the steps described above without phage treatment (mock).

### MIC Assay

Bacterial lawns were normalized to contain 1×10⁸ CFU/mL bacteria mixed with top agar for a total volume of 5 mL. The bacteria and top agar mixture is poured onto a solid BHI plate. The plate was dried for 10 minutes. MIC with increasing concentrations of antibiotics were placed on the semi-dried bacterial lawn and allowed to dry for 10 minutes. The plates are then incubated at 37° C. overnight. For analysis, the plates were imaged, and the MIC of the bacteria was determined. The MIC is determined at the edge of the inhibition ellipse intersects the side of the strip.

### Rabbit Blood Hemolysis

Phage-treated or mock-treated cultures were diluted to an OD600 of 0.1, 5 μL of this dilution was spotted on rabbit blood TSB agar plates and incubated at 37° C. for 24 hours. The area of clearance was determined by the following formula:

$$[ π {( diameter of clearance / 2 )}^{2} ] - [ {π ( diameter of bacterial spot / 2 )}^{2} ]$$

### Clumping Assay

Clumping assays were performed as described previously.⁶⁰ In short, overnight cultures were diluted 1:100 and incubated at 37° C. until the cultures reached an OD600 of 1.5. At this point, 1.5 mL of culture was washed two times and resuspended with PBS. Lyophilized human plasma was added for a final concentration of 1.25%. Resuspended cells were left to sit statically at room temperature. 100 uL were taken from the top of the cell suspensions in 30-minute intervals and the OD600 was measured.

### Biofilm Assay

Biofilm assay was performed using the crystal violet method as outlined.⁶¹ In brief, overnight cultures grown in BHI at 37° C. were back diluted 1:100 into a 96-well bottomed microwell plate. The plates were incubated without shaking at 37° C. overnight. The contents in the plate were discarded and washed with PBS. Biofilm fixation was done with sodium acetate (2%). Crystal violet (0.1%) was used for staining followed by a final wash with PBS. Absorbance at 600 nm was read using a spectrophotometer.

### DNA Sequencing and Genome Assembly

Following published protocols, genomic DNA from bacteria and phage was isolated using phenol-chloroform extraction. Purified DNA was sent to Plasmidsaurus and SeqCenter for Nanopore and Illumina sequencing, respectively. Reference genomes for bacterial strains were assembled using Flye v2.9.3 with default settings for long-reads. This resulted in 1 singular contig assemblies for 252 (2902592 bp, 125×) and MW2 (2820460, 600×), and 3 contigs for LAC (2907712, 645×). Phage assemblies for Evo2 and ΦStaph1N were done with the SPADES assembler v3.15.5.

Open-reading frames (ORFs) were called on the assembled bacterial genomes using Prodigal v.2.6.3,⁶² resulting in a gene-feature file (GFF), and translated genes as .faa and .fna formats. We used BLASTp (Accessed Jun. 4, 2024), against the protein BLAST database swissprot_2023-06-28, with an expectation value cutoff of 0.001. The top hit for each ORF was used as the final functional annotation. Additionally, we annotate the genomes using Bakta v.1.10.3.

### Mutation Identification

A total of 27 genomic samples were collected. DNA was extracted and sent for long-read sequencing using Oxford Nanopore Technology (Plasmidsaurus, San Francisco, USA). Reads were filtered using filtlong v0.2.1 using default settings (github.com/rrwick/Filtlong) and with the -p flag 95 (keeping 95% of the best reads). Quality-filtered long reads were mapped against the respective genomes using minimap2 2.22-r1101⁶³ resulting in 1 alignment file output per sample (.sam file). The read mapping software Minimap2⁶³ was selected because of its suitability to map long reads. Samtools v1.20⁶⁴ was used to convert the .sam files into .bam files, sort the bam file, index the bam files, and generate a coverage table for each position along the alignment.

Reference files (fasta and GFF files) and the 53 “sorted.bam” alignments files were imported into Geneious. Variant calling was performed using Geneious Prime Geneious Prime® 2024.0.2 (geneious.com), using the custom settings: 10% coverage, minimum 95% variant frequency threshold, and the option for “Analyze effect of variants on translation” checked. Variant results and genome annotations table files were exported as a tab-separated-table, and visualized using R v4.4.0, mostly with the tidyverse package. Sequencing data processing, quality filtering, and mapping were performed at the Center for High-Throughput Computing (chtc.cs.wisc.edu/). BLAStp was performed on usegalaxy.eu (Accessed Jun. 4, 2024).

### RNA Purification

Parental and evolved MRSA strains were diluted 1:100 in BHI broth and incubated at 37° C., 235 RPM until they reached an OD₆₀₀ of 1.5. 500 μL of culture were transferred into a microcentrifuge and 1 mL of RNAprotect Bacteria reagent (QIAGEN) was added. The mixture was vortexed for 5 seconds and incubated at room temperature for 5 minutes. The tubes were centrifuged for 10 minutes at 5000 g, and the supernatant discarded. Bacterial pellets were resuspended in 80 μL of phosphate buffered saline and 10 μL of lysostaphin solution (1 mg/mL stock). The suspensions were incubated at 37° C., with shaking, until the solution looked clear (˜30 minutes). 10 μL of 10% sarkosyl was then added and the tube mixed, after which 300 μL of TRIzol reagent (Invitrogen) was added. RNA purification was performed following the protocol from the Direct-zol RNA Miniprep Plus kit (ZYMO Research). DNase I treatment was performed as recommended, and the samples were eluted in 75 μL of DNase/RNase-free water.

### RNA Sequencing and Differential Gene Expression Analysis

Purified RNA was prepared and sequenced on an Illumina sequencing platform at the UW-Madison Gene Expression Center. RNA-seq data was collected in the parental and evolved MRSA strains to assess differentially expressed genes. Cleaned RNA reads were mapped onto the LAC and MW2 reference genomes using bowtie2 v2.5.4,⁶⁵ and featureCount v2.0.8⁶⁶ from the software subreads was used to generate a read count matrix. Two read count matrices (one for LAC and one for MW2) were imported into R v.4.4.0 for processing with DESeq2 (version 1.44.0).⁶⁷ Figures were generated using the packages tidyverse (version 2.0.0) and EnhancedVolcanoPlots (version 1.22.0). To generate the volcano plots, we chose an adjusted p-value of 0.002 and a log 2 fold change (log₂ FC) of <−2 or >2. Multiple “unknown” genes were deemed significant (adjusted p-value <0.002, abs(log₂ FC)>=2) in both LAC and MW2. To compare the results between the genomes, we performed a protein clustering analysis using MMseqs2 version b804fbe384e6f6c9fe96322ec0e92d48bccd0a42 between all the Bakta-generated amino acid sequences (.faa files) from LF and MW2.⁶⁸ Then we considered any protein sharing over 80% identity to be the “same” to generate a summary table showing up-regulation and down-regulation among the 2 genomes.

### Checkerboard Assay for Phage-Antibiotic Synergy

The overnight cultures of MRSA252, Lac-Fitz, and MW2 were back-diluted 1:100 in BHI and incubated at 37° C. until the culture reached mid-log phase. The culture was then inoculated into each well of the 96-well plate containing a gradient of oxacillin and phage (ΦStaph1N or Evo2). The oxacillin gradient was a 2-fold serial dilution, while the phage MOI gradient was a 10-fold serial dilution. The plates were then placed at 37° C. with shaking (235 RPM) for 24 hours. Following 24 hours, each well from the plate was then passaged 1:100 into another 96-well plate with fresh BHI and grown at 37° C. with shaking (235 RPM) for another 24 hours.

### Staphyloxanthin Assay

MRSA strains were infected and passaged with bacteriophage, as described above. Following phage treatment, the bacteria were pelleted with centrifugation and washed 3 times with 5 mL PBS. Following the washes, the pellets were then resuspended in 1 mL of PBS. Resuspended cells were then transferred into a microcentrifuge tube and spun down at 15000 rpm, after which the supernatant was removed. The pellets in the centrifuge tube were then weighed on a balance (tared with the weight of an empty centrifuge tube). Based on the pellet weight, 0.1 g/800 μL of 100% methanol was added to the pellet. The mixture in the tube was then vortexed for 10 minutes, and then centrifuged for another 10 minutes at 15000 rpm to separate methanol soluble from insoluble fractions. 100 μL of the supernatant was then added to a 96 well plate and the absorbance measured at 450 nm.

### REFERENCES

1. Ippolito, G., Leone, S., Lauria, F. N., Nicastri, E. & Wenzel, R. P. Methicillin-resistant Staphylococcus aureus: the superbug. International Journal of Infectious Diseases 14, S7-S11 (2010).
2. Tomasz, A. The Mechanism of the Irreversible Antimicrobial Effects of Penicillins: Howthe Beta-Lactam Antibiotics Kill and Lyse Bacteria. Annu. Rev. Microbiol. 33, 113-137 (1979).
3. World Health Organization. Critically Important Antimicrobials for Human Medicine. (World Health Organization, Geneva, 2019).
4. Murray, C. J. L. et al. Global burden of bacterial antimicrobial resistance in 2019: a systematic analysis. The Lancet 399, 629-655 (2022).
5. Zapun, A., Contreras-Martel, C. & Vernet, T. Penicillin-binding proteins and β-lactam resistance. FEMS Microbiol Rev 32, 361-385 (2008).
6. Liu, C. et al. Clinical Practice Guidelines by the Infectious Diseases Society of America for the Treatment of Methicillin-Resistant Staphylococcus aureus Infections in Adults and Children. Clinical Infectious Diseases 52, e18-e55 (2011).
7. Turner, N. A. et al. Methicillin-resistant Staphylococcus aureus: an overview of basic and clinical research. Nat Rev Microbiol 17, 203-218 (2019).
8. Barros, E. M. et al. Daptomycin Resistance and Tolerance Due to Loss of Function in Staphylococcus aureus dsp1 and asp23. Antimicrob Agents Chemother 63, e01542-18 (2019).
9. Suttle, S. W. Viruses and Nutrient Cycles in the Sea. 49, (1999).
10. Kortright, K. E., Chan, B. K., Koff, J. L. & Turner, P. E. Phage Therapy: A Renewed Approach to Combat Antibiotic-Resistant Bacteria. Cell Host & Microbe 25, 219-232 (2019).
11. Hatoum-Aslan, A. The phages of staphylococci: critical catalysts in health and disease. Trends in Microbiology 29, 1117-1129 (2021).
12. Oechslin, F. Resistance Development to Bacteriophages Occurring during Bacteriophage Therapy. Viruses 10, 351 (2018).
13. Azam, A. H. & Tanji, Y Peculiarities of Staphylococcus aureus phages and their possible application in phage therapy. Appl Microbiol Biotechnol 103, 4279-4289 (2019).
14. Gerlach, D. et al. Methicillin-resistant Staphylococcus aureus alters cell wall glycosylation to evade immunity. Nature 563, 705-709 (2018).
15. Meyer, J. R. et al. Repeatability and Contingency in the Evolution of a Key Innovation in Phage Lambda. Science 335, 428-432 (2012).
16. Bernheim, A. & Sorek, R. The pan-immune system of bacteria: antiviral defence as a community resource. Nat Rev Microbiol 18, 113-119 (2020).
17. Maynard, N. D. et al. A Forward-Genetic Screen and Dynamic Analysis of Lambda Phage Host-Dependencies Reveals an Extensive Interaction Network and a New Anti-Viral Strategy. PLoS Genet 6, e1001017 (2010).
18. Oromi-Bosch, A., Antani, J. D. & Turner, P. E. Developing Phage Therapy That Overcomes the Evolution of Bacterial Resistance. Annu. Rev. Virol. 10, 503-524 (2023).
19. Chan, B. K. et al. Phage selection restores antibiotic sensitivity in MDR Pseudomonas aeruginosa. Sci Rep 6, 26717 (2016).
20. Berryhill, B. A., Huseby, D. L., McCall, I. C., Hughes, D. & Levin, B. R. Evaluating the potential efficacy and limitations of a phage for joint antibiotic and phage therapy of Staphylococcus aureus infections. Proc. Natl. Acad. Sci. U.S.A. 118, e2008007118 (2021).
21. Xia, G. & Wolz, C. Phages of Staphylococcus aureus and their impact on host evolution. Infection, Genetics and Evolution 21, 593-601 (2014).
22. Voyich, J. M. et al. Insights into Mechanisms Used by Staphylococcus aureus to Avoid Destruction by Human Neutrophils. The Journal of Immunology 175, 3907-3919(2005).
23. Holden, M. T. G. et al. Complete genomes of two clinical Staphylococcus aureus strains: Evidence for the rapid evolution of virulence and drug resistance. Proc. Natl. Acad. Sci. U.S.A. 101, 9786-9791 (2004).
24. Kobayashi, S. D. et al. Comparative analysis of USA300 virulence determinants in a rabbit model of skin and soft tissue infection. J Infect Dis 204, 937-941 (2011).
25. Deghorain, M. & Van Melderen, L. The Staphylococci Phages Family: An Overview. Viruses 4, 3316-3335 (2012).
26. Land, A. D., Hogan, P., Fritz, S. & Levin, P. A. Phenotypic Variation Is Almost Entirely Independent of the Host-Pathogen Relationship in Clinical Isolates of S. aureus. PLoS ONE 10, e0129670 (2015).
27. Mo, C. Y et al. Type III-A CRISPR immunity promotes mutagenesis of staphylococci. Nature 592, 611-615 (2021).
28. Goldberg, G. W., Jiang, W., Bikard, D. & Marraffini, L. A. Conditional tolerance of temperate phages via transcription-dependent CRISPR-Cas targeting. Nature 514, 633-637 (2014).
29. Baba, T., Bae, T., Schneewind, O., Takeuchi, F. & Hiramatsu, K. Genome Sequence of Staphylococcus aureus Strain Newman and Comparative Analysis of Staphylococcal Genomes: Polymorphism and Evolution of Two Major Pathogenicity Islands. J Bacteriol 190, 300-310 (2008).
30. Pincus, N. B. et al. Strain Specific Phage Treatment for Staphylococcus aureus Infection Is Influenced by Host Immunity and Site of Infection. PLoS ONE 10, e0124280 (2015).
31. Li, L. et al. The Global Regulon sarA Regulates β-Lactam Antibiotic Resistance in Methicillin-Resistant Staphylococcus aureus In Vitro and in Endovascular Infections. J Infect Dis. 214, 1421-1429 (2016).
32. Zielinska, A. K. et al. SARA-mediated repression of protease production plays a key role in the pathogenesis of Staphylococcus aureus USA 300 isolates. Molecular Microbiology 86, 1183-1196 (2012).
33. Crosby, H. A. et al. The Staphylococcus aureus Global Regulator MgrA Modulates Clumping and Virulence by Controlling Surface Protein Expression. PLoS Pathog 12, e1005604 (2016).
34. Kwiecinski, J. M. et al. Staphylococcus aureus uses the ArlRS and MgrA cascade to regulate immune evasion during skin infection. Cell Reports 36, 109462 (2021).
35. Panchal, V. V. et al. Evolving MRSA: High-level β-lactam resistance in Staphylococcus aureus is associated with RNA Polymerase alterations and fine tuning of gene expression. PLoS Pathog 16, e1008672 (2020).
36. Bai, J. et al. The role of ArlRS in regulating oxacillin susceptibility in methicillin-resistant Staphylococcus aureus indicates it is a potential target for antimicrobial resistance breakers. Emerging Microbes & Infections 8, 503-515 (2019).
37. Walker, J. N. et al. The Staphylococcus aureus ArlRS Two-Component System Is a Novel Regulator of Agglutination and Pathogenesis. PLoS Pathog 9, e1003819 (2013).
38. Schulthess, B. et al. The σ^{B}-Dependent yabJ-spoVG Operon Is Involved in the Regulation of Extracellular Nuclease, Lipase, and Protease Expression in Staphylococcus aureus. J Bacteriol 193, 4954-4962 (2011).
39. Liu, X., Zhang, S. & Sun, B. SpoVG Regulates Cell Wall Metabolism and Oxacillin Resistance in Methicillin-Resistant Staphylococcus aureus Strain N315. Antimicrob Agents Chemother 60, 3455-3461 (2016).
40. Chen, C. et al. Characterization of serine acetyltransferase (CysE) from methicillin-resistant Staphylococcus aureus and inhibitory effect of two natural products on CysE. Microbial Pathogenesis 131, 218-226 (2019).
41. Markham, G. D., DeParasis, J. & Gatmaitan, J. The sequence of metK, the structural gene for S-adenosylmethionine synthetase in Escherichia coli. Journal of Biological Chemistry 259, 14505-14507 (1984).
42. Proctor, A. R. & Kloos, W. E. Tryptophan Biosynthetic Enzymes of Staphylococcus aureus. J Bacteriol 114, 169-177 (1973).
43. Maidhof, H., Reinicke, B., Blumel, P., Berger-Bachi, B. & Labischinski, H. femA, which encodes a factor essential for expression of methicillin resistance, affects glycine content of peptidoglycan in methicillin-resistant and methicillin-susceptible Staphylococcus aureus strains. J Bacteriol 173, 3507-3513 (1991).
44. Srisuknimit, V., Qiao, Y, Schaefer, K., Kahne, D. & Walker, S. Peptidoglycan Cross-Linking Preferences of Staphylococcus aureus Penicillin-Binding Proteins Have Implications for Treating MRSA Infections. J. Am. Chem. Soc. 139, 9791-9794 (2017).
45. Gardete, S. et al. Role of murE in the Expression of β-Lactam Antibiotic Resistance in Staphylococcus aureus. J Bacteriol 186, 1705-1713 (2004).
46. Xu, Z. J. et al. Tryptophanyl-tRNA synthetase from Bacillus subtilis. Journal of Biological Chemistry 264, 4304-4311 (1989).
47. Jaroch, M. et al. Alternate routes to mnm ⁵ s ² U synthesis in Gram-positive bacteria. J Bacteriol 206, e00452-23 (2024).
48. Kobayashi, K. et al. Comprehensive DNA Microarray Analysis of Bacillus subtilis Two-Component Regulatory Systems. J Bacteriol 183, 7365-7370 (2001).
49. Maki, H., McCallum, N., Bischoff, M., Wada, A. & Berger-Bachi, B. tcaA Inactivation Increases Glycopeptide Resistance in Staphylococcus aureus. Antimicrob Agents Chemother 48, 1953-1959 (2004).
50. Willing, S., Dyer, E., Schneewind, O. & Missiakas, D. FmhA and FmhC of Staphylococcus aureus incorporate serine residues into peptidoglycan cross-bridges. Journal of Biological Chemistry 295, 13664-13676 (2020).
51. Jurado, A., Fernindez, L., Rodriguez, A. & Garcia, P. Understanding the Mechanisms That Drive Phage Resistance in Staphylococci to Prevent Phage Therapy Failure. Viruses 14, 1061 (2022).
52. Cao, Z., Casabona, M. G., Kneuper, H., Chalmers, J. D. & Palmer, T. The type VII secretion system of Staphylococcus aureus secretes a nuclease toxin that targets competitor bacteria. Nat Microbiol 2, 16183 (2016).
53. Clauditz, A., Resch, A., Wieland, K.-P., Peschel, A. & Gotz, F. Staphyloxanthin Plays a Role in the Fitness of Staphylococcus aureus and Its Ability To Cope with Oxidative Stress. Infect Immun 74, 4950-4953 (2006).
54. Yarwood, J. M. & Schlievert, P. M. Quorum sensing in Staphylococcus infections. J. Clin. Invest. 112, 1620-1625 (2003).
55. Moller, A. G. et al. Genes Influencing Phage Host Range in Staphylococcus aureus on a Species-Wide Scale. 6, (2021).
56. Reyes-Robles, T. et al. Staphylococcus aureus Leukotoxin ED Targets the Chemokine Receptors CXCR1 and CXCR2 to Kill Leukocytes and Promote Infection. Cell Host & Microbe 14, 453-459 (2013).
57. Cheung, G. Y C., Bae, J. S. & Otto, M. Pathogenicity and virulence of Staphylococcus aureus. Virulence 12, 547-569 (2021).
58. Otto, M. Staphylococcus aureus toxins. Current Opinion in Microbiology 17, 32-37 (2014).
59. Kortright, K. E., Doss-Gollin, S., Chan, B. K. & Turner, P. E. Evolution of Bacterial Cross-Resistance to Lytic Phages and Albicidin Antibiotic. Front. Microbiol. 12, 658374 (2021).
60. Crosby, H. A., Kwiecinski, J. M. & Horswill, A. R. In Vitro Assay for Quantifying Clumping of Staphylococcus aureus. in Staphylococcus aureus (ed. Rice, K. C.) vol. 2341 31-36 (Springer US, New York, NY, 2021).
61. Feoktistova, M., Geserick, P. & Leverkus, M. Crystal Violet Assay for Determining Viability of Cultured Cells. Cold Spring Harb Protoc 2016, pdb.prot087379 (2016).
62. Hyatt, D. et al. Prodigal: prokaryotic gene recognition and translation initiation site identification. BMC Bioinformatics 11, 119 (2010).
63. Li, H. Minimap2: pairwise alignment for nucleotide sequences. Bioinformatics 34, 3094-3100 (2018).
64. Li, H. et al. The Sequence Alignment/Map format and SAM tools. Bioinformatics 25, 2078-2079 (2009).
65. Langmead, B. & Salzberg, S. L. Fast gapped-read alignment with Bowtie 2. Nat Methods 9, 357-359 (2012).
66. Liao, Y, Smyth, G. K. & Shi, W. feature Counts: an efficient general purpose program for assigning sequence reads to genomic features. Bioinformatics 30, 923-930 (2014).
67. Love, M. I., Huber, W. & Anders, S. Moderated estimation of fold change and dispersion for RNA-seq data with DESeq2. Genome Biol 15, 550 (2014).
68. Steinegger, M. & Söding, J. MMseqs2 enables sensitive protein sequence searching for the analysis of massive data sets. Nat Biotechnol 35, 1026-1028 (2017).
69. Christensen, G. D., Simpson, W. A., Bisno, A. L. & Beachey, E. H. Adherence of slime-producing strains of Staphylococcus epidermidis to smooth surfaces. Infect Immun 37, 318-326 (1982).
70. Jiang, W. et al. Dealing with the Evolutionary Downside of CRISPR Immunity: Bacteria and Beneficial Plasmids. PLoS Genet 9, e1003844 (2013).

We claim:

1. A Staphylococcus bacteriophage comprising a nucleic acid having a knockout and/or nonsense mutation in open reading frame 141 (ORF141), or a homologous position thereof.

2. The bacteriophage of claim 1, wherein the knockout and/or nonsense mutation is located at or prior to a codon corresponding to amino acid residue 100 of ORF141.

3. The bacteriophage of claim 1, wherein the bacteriophage has a genome comprising or consisting of a nucleic acid of SEQ ID NO: 2 or a nucleic acid having at least 90%, 95%, 96%, 97%, 98%, or 99% identity to SEQ ID NO: 2.

4. The bacteriophage of claim 1, wherein the knockout and/or nonsense mutation comprises a mutation that replaces amino acid residue 77 of ORF141, or a homologous position thereof, with a premature stop codon, wherein the position is defined relative to SEQ ID NO: 1.

5. The bacteriophage of claim 1, wherein the knockout and/or nonsense mutation comprises a full or partial deletion of ORF141.

6. The bacteriophage of claim 1, wherein the bacteriophage exhibits increased infectivity of Staphylococcus bacteria as compared to a Staphylococcus bacteriophage without the knockout and/or nonsense mutation in ORF141.

7. A composition comprising the bacteriophage of claim 1 and a pharmaceutically acceptable carrier.

8. The composition of claim 7, wherein:
(a) the bacteriophage is present at a concentration of at least 10⁷ plaque forming units (PFU)/mL;
(b) the composition comprises 10⁸ to 10¹² PFU of the bacteriophage; and/or
(c) the pharmaceutically acceptable carrier comprises phosphate buffered saline (PBS); and/or
(d) the composition is formulated for topical, oral, intranasal, or intravenous administration.

9. A method of treating a bacterial infection, the method comprising administering a bacteriophage to a subject infected with or suspected of being infected with a Staphylococcus bacteria, wherein the Staphylococcus bacteria is associated with β-lactam antibiotic drug resistance.

10. The method of claim 9, wherein the Staphylococcus bacteria is methicillin-resistant S. aureus (MRSA).

11. The method of claim 9, wherein the administering:
(a) comprises administering a composition comprising the bacteriophage and a pharmaceutically acceptable carrier;
(b) comprises administering the bacteriophage at a concentration of at least 10⁷ plaque forming units (PFU)/mL;
(c) comprises administering 10⁸ to 10¹² PFU of the bacteriophage; and/or
(d) results in a bacteriophage multiplicity of infection (MOI) in the subject of at least 0.01.

12. The method of claim 9, wherein the bacteriophage comprises a nucleic acid having a knockout and/or nonsense mutation in open reading frame 141 (ORF141), or a homologous position thereof.

13. The method of claim 9, wherein:
(a) the bacteriophage has a genome comprising or consisting of a nucleic acid of SEQ ID NO: 2 or a nucleic acid having at least 90%, 95%, 96%, 97%, 98%, or 99% identity to SEQ ID NO: 2; or
(b) the bacteriophage has a genome comprising or consisting of a nucleic acid of SEQ ID NO: 1 or a nucleic acid having at least 90%, 95%, 96%, 97%, 98%, or 99% identity to SEQ ID NO: 1.

14. The method of claim 9, further comprising administering a β-lactam antibiotic to the subject.

15. The method of claim 14, wherein a first dose of the β-lactam antibiotic is administered to the subject within 6 hours, 12 hours, 18 hours, 24 hours, or 48 hours after administration of the bacteriophage, and/or wherein the method further comprises administering a reactive oxygen species (ROS) or ROS generator pharmaceutical to the subject.

16. A method of making bacteria more susceptible to β-lactam antibiotic drug treatment, the method comprising:
(a) obtaining a sample comprising a Staphylococcus bacteria identified as resistant to a β-lactam antibiotic from a subject;
(b) contacting at least a portion of the bacteria with a bacteriophage to generate bacteriophage contacted bacteria;
(c) contacting the bacteriophage contacted bacteria with a β-lactam antibiotic; and
(d) determining whether the bacteriophage contacted bacteria exhibits increased susceptibility to the β-lactam antibiotic as compared to the Staphylococcus bacteria in the sample prior to contact with the bacteriophage.

17. The method of claim 16, wherein the Staphylococcus bacteria is methicillin-resistant S. aureus (MRSA).

18. The method of claim 16, wherein:
(a) the bacteriophage has a genome comprising or consisting of a nucleic acid of SEQ ID NO: 2 or a nucleic acid having at least 90%, 95%, 96%, 97%, 98%, or 99% identity to SEQ ID NO: 2; or
(b) the bacteriophage has a genome comprising or consisting of a nucleic acid of SEQ ID NO: 1 or a nucleic acid having at least 90%, 95%, 96%, 97%, 98%, or 99% identity to SEQ ID NO: 1.

19. The method of claim 16, wherein bacteriophage comprises a nucleic acid having a knockout and/or nonsense mutation in open reading frame 141 (ORF141), or a homologous position thereof.

20. The method of claim 16, further comprising administering the bacteriophage to the subject based, at least in part, on the determination in step (d).

21. The method of claim 20, further comprising administering a β-lactam antibiotic to the subject.


SEQUENCE LISTING (5 sequences)

SEQ ID NO: 1 - DNA, 138281 bp, Kayvirus G1
  source 1..138281: mol_type = genomic DNA
tagtagtagtaatagtagtagtaatagtagtagtaatagtagtagtaatagtagtagtaatagtagtacataacaaaggtacttagttttacttggttaggttctcattctatatcttcctcattagtcaacatactctcactctaaatattgtccttcctattactataatacttaatgattatcatggtaatctgtaattagctgtgcaacaataatagttaacaagggaacaactcctatagacataataattatttttgcaaacaagtccatatgtttcctccttataaattatatactttaaacatcttatccatagtaatctataaactctacttcttcaaaacctcttatctctgataatcctgaatcatctgtagattctattgttactgtttcatttttaaagttaataatttctacattaacaatttgttgagttatttcatcccataactttttatatttatttatcatattaattccctcccttaccatttaatatttaagtaatctaaccctgctaaattatttgtagatatagaagggtactctatatcattactaatgtggtttacactatatccttggttagtaagttcttctataactctatattctacttcattatctactcttaagtctctaagaacatattgataacttgtattacctttcatagcttcttcaataataccatcattaatattatctagtatatcttctacaatactatctatagtttcataatgtacatcatcttcataaatttctgataggtacttagcatgccctgcatttatcataatagtttactcctccttattatttaccatcgttcaatagatacaatatgtctagtatcaataataacttttttatctttcccatatttagttacttcaatacaatctttactcataatattttcatacaaagtaactagtgattcgtatttactataataagtatattttctattatctacagttcttattgtataatcatgttctatacttatcattataattactcccatacgaatccgaatataccatcattttgtacttgtcttacccattcctctaataccttatgaagaactgaactaatactctgcgtactagtaattttaacaacagttgttatataaagaccttgtggtatatgcatttctatagaatctgagtgtaacttatcaatatctatagctacaacttctgtattgtaagtgaaattaagtctcatagtataaggtgttacatcaactctatataactcaactttattttcatcaggtaatattagagatttaccttgtttatttgtaaagtactctactaattttgtactattcatattacttaaacacttccttcattttattataagcctctaataattttttatattcttgcttcgaaagagtaactttttcttttgaatctgattttttaaagtattccctgtagtccctctcgataggtctaataatataatcaatatctttctttggatataaatgctcctctacaggattagtagctttaccatctctattttgagtaataacagttacattgttaaaatcatggttatatacaataataccattaaaaccgaatacggagattctttctgatacaaatatatctccttctctataatcatctgttcttaacaaatctttggctttaattattttccaagaactaggtaatctttcgtatttcattttattagcatttaacttagttttgtacctacctagagttaatcgttgaacataattagatactaagtctgtagattgatatccataatagttatcttcctttataaatttacctacataatcatttaaatacatagttagtctcctttatatattaattcatcattcttatacactttctcacctacattagagaaatcatcacctttatcaaacttatgatagacctctttattaagatatacacggtcttctcctttatctgtctctaaatctacatagtatttcatttcaccaatgactggtacagtaacgctacctgattttatataagtagttggttctctgtattcacctactcctactactttaccttcatggttatcaggagagcaaccccctagtaatagagaaatagtaattgtaagtaatactattaactttttcatattttcatctccttgtatactattatataatagatacctacctaagtcaataggtagatactaatttatttatcagctacgtactcataaaacccaatatcttctctaagtttatcttctcttaactgcttaacatctttaggtggtactgtaactccttcaacatattctaccaggtcaatgaatgctttctctaagtattccctaggacagtcttcgttttcccaatagttaactccttctttttcccaatagttaactccttctttttcccagttattattcatgctatttctctcctttcaactctttaatttcaagtgtaactactttatctacatactcctctatagctttataagataactgatttaaatattgttcactaactcctaatatttcagtaaccttttcctggtttcttactacatcatctaactcagtatctacataacctagagattcaaacatttcatctatttcatttttgtcaggcttaatttctttatattcattataaaattcttcagtagttctaaagtactgttgtctatttcttttaatatgatggtagtctattaattgtctctcaccattaattgtattaaactttcttacactaaatgatacgacatgcccacctatttctaatttaactccatttaatttatctataatcattttatttctctccttttacttatggctttaaccattcccatttttcaaaccatagtatttttataatattgtacttatgacctactctgtagtcatccgggttgaactcatttacttttaaggtaataatctttcttttcctaggattatctacaccaataagaatcacatctacataatctacatacgcaaacccctcatcaggaggataagtggaatgctcaaatctactatagtaattatgttttgatattttataaatcatttctttcactcctttatttactatagccctactatataataaaaaactccctttgtcaatagggagtttaaatatttattaattatttttaattatttctctaatttcacttaacgactggtctctataaagtttaccatcttcaaatactctttctaattgattaacattttgtttgtttagccattgatgtaaggttaagttatcttcccatgaaccgtctttatatacagctactcgacctttatgtgaacgtttaccactatctgttttaggatttttatagataagtttttcttcatttttaatcttagcatacgttgctttgattgcaaacccgtgggtatcacgagtattaaattggtaagagaaactacctacacctaatacaacattaatacttgcaaaacctttttcttttaatccttcacaaatttgttgagcacgttcatagttaatagagtctccataaattaatcctacatgttcatctaatactttgtaccctttttcagtttcagtacctccaaatgtatcccaaagcacttctactgcacctttacgttcatattcagtgtctgcatcagggtctccgcaaataattttaacagggtctccactatcaggtctgattactactttaccattacgttccataataatatcctttaatctaggtaaagttttagtcatattaccccaaaagtcccaagtatcagacacaatagagaaaatgcctgttggatatatttctgttaacatacgtttatatgtttcatactcatccatggtttcataattaccatttgcacacattactgagtgctcagtagcagggatagaagcaacaaccatttccttctctacgtctgcattgtaatatgattctaagaaatccactacaggtactgtatcactacctttaaaactaattgcatgacctgctgaacttaattgagctgtttctaaagaactcataccacgcatactaaagtcatgaccctggaaatctactgcttcaatattacctactgtttccattgcatatttatctagcattttacgcatatcatatgctagtgtagcactagtcattgcttgccatgtttgagtactaataatagtttctaggtaattagttaaccaaaagaaatctgagtgagtattttcaattgtcataacaggtgtgttaggatgaacaacagtaccttcttttaaagctcttacatcaataggtaaataacctaagtcatgtaattgttctaagtgtttagcatcaggattttcttgtcctagtgtaaatttaactgtacgtttgtattcattaataacttcctctttaggtctgttaaagaactctttattaaacatatcaataaaatattttttaacaagtgattgaataccaaatactacaatattatcactgtgttcataatatttattacttctaggtactagtgtactatatacaatttctgtaccttcagggtattgttctctgtgacttagtttatagaagtcacacattaaagttggatttagcataatttatttttctccttttataaatatctttcgatatcttttatttgtaattcagttccgttgattctagctctatgtttagcaatagctttttcccaattatttttagggaacatagagtctgttacaataatatctttaaatcctgagctaagtaatgctccttctgcaaaagctcgttctgcatgagtcaatattaatgatacactacttaccttaagtttgtcaagggcttttttacaaccgacaaatgtcccaccgtaacttgttaagtcatctagtataatacaattatcatataaagtatttttatctttaattattttaatacctttaatcttacctgtttcaaaatctcgtttcttctcaccatatacaattgaatatgattcaatattagattccattaagatacgttctacatcaaatagatatctatcgtatgcccctttatcaggtagtactattgttacaggctcttcggacatttcaatatactgatttaataatttaagtgtaacatcaatagcaaaagaattagtaagtaattcttctgttacagggctatgaggttctgctacccataccgatttataattaaggttattaataaattctgtaatgtatttaagactgaacatattattatgtccttcttctactctatccattctgctataaggtagatatgcaatctctaaaaattcagctttatctttaatacctagttgctctaaatacttagtaaacatatacaaatgcattaagtctgcgtcattttcccatttaaagaaaacatcaaatcttaagtcctcttctactaaatactttaaatcatcatatttaaaattaatctcaccattaggaaacttttcagtaataataggtttgtattctttatctacttctgaaaatattttaatcattttgttgatagctcctttttatatgttatttggaatacattttctgccgatatattcttaattacatcaataaatcctttaccaatgcttaacattgtatctacatctacaagtgttaaattttctttatacattttcctcagtaattcttttacctcaatgtattcacctttaaaaactaacttacctgttttaagatgatagatttcataaatttctacttcttttgttgtaatgctctctaaatactctcttgtactttttagcttatacgctaattcttccacatctatttcaggatacataattgttcatctccttaatttcaattctaagctactttaaggtaccatagagagcacttaaagtaatttaggtactcttatggtaaaatcattactgctctttatcatacactctaacagtcttctgaacattgctttttttaatttcgaacataattttattataaatcatttcaatactaaataagaaagctccgaacattactgaaactaatcgaataacattttctctgacatttaaatagtctaagtagaagtaaagaccaatagccatccctaaaacaattaaaatacctgataagccagtactaaactttgtaacatagaaataaatagcatacataccaaccaatactgctacaatattaataaccaattccatttataacccctccgttgtaatttgacttccaaaattattcatatagtacacataagcttgtagttcatctaagttaatttcctttttagggtaagatgctgatgtcatctcagaaataatgtgtactcgtttttctgtaacactgatgtgtacgttgtaaccaccgctaatacaatctgtaataaaatctctataattcatatttacctctccttaaaataaaatagctcctgctttataatataaatcctccaagtagtaatgcaatcccaacaccaaatatggtaaatagtatttttaaatctacatcatcataatacttacccaataccaacttataaccgctagataaaaataatatgagtccaataagtataactattacgggtatcattatattacctcctttttctttatatctaaagtatactataaaaaatacctgctgtcaacaggctgtcaacaggtatttataaatttatttaatttttaatgatacccactaaaagtgcatcaacagatactttatcagcactatataactggtcaatatctacttcatctattagaacaatttcacctttaatatcataatttgaataatcttcagcagaagctactctaagatgagggaatttatttttaagcatttttctcataggttcatttgatactactaagggtaaatccttttctagtgctaaataagctaatgtgcttgttttacctgtacatctattgagtacttcagaagataaagctacaggtatttccttttcaatagaagatttaaacattgaataatattcattattgatataaggctttagtttttctaattctttagttgaattaatttctttaatagattcaataaaatcattgtcagtctctacctgttcttctacaatagaaaaaatcttatctcttaattgtaataaaatttcttttggattattactaccataatcaacaaaaatttcagttgtaatatatctcgttgttctagggtctttatcagctttataacttatgcttatttcaaaataatcccctttatcaccccaaccagtttgtttattgatatgagcaataatactatacgtttctccttttattccttctaaatttaataaataatttgtaatggttttattagacacttatttttcctccttaaaatatcttgatagttctatagttgttttagtttggtctcctacaatagcttgtagtttattactaatttctgtatatagctctttagcacttaatgtttcaggtctctcaattgtaatagctgtatctaataaatgtttacctgaaagtgtagatacttgagcttctaaaaacaatactgattcatctatagtatctatatctaaaatatacgatgaactagcctgtaagaatcttactttataaccctctatttgtagtactttgtcattaaaaaatcttgctattgattctgctttcataattaattttcctccttattatctttcggaataaaaatctgaggtctatatttatcttctagtactctaatagcttcctcatactgagatatcattgtatcaaagtgtcttagttgtaactgtgattcatactgctcctcacctttagtttggtactccgcaaagctcttttcttcagttattaattttgctagtgcctctcttaaagcttgtgacggtgttgcaatataattctctccattaatatttgattcatattttgccatttatagttcctcctttaatttctgctaaacatacttcctattgcactgattaaaataaatattccgatacatacccaaatacttactttaactccgaatgcagatagtactaactgtaaaatccatgccggtaaaacaatacctaatgcaataactagtattacaattcctaaaattaaaataaaatgcttcataaattaacctccttatttcttatactcttagtatataataaaagaaacctaatgtcaataggtttcttaaacttttttatttatttttaaaccctcttagagtaccactttcaagagtacctattttaacagattttttttctttacgttcttgctcttctaggtaaagaatacctaatgagttggctgttgtaactatagttcttgaaatttgacctgtgttataattctcttgcttaattttacctttcttgtacctaacaagtgctacaatatctaaagctaacattaaaaaattatttactgttagtgttaaaattttcttttctctactgttcatcttttgtttttcctccttcgtcttcaaaacgttctatttgttttttaagctctttattctctgcttcaagttcttttactttttgctctaactcttctttagtcatagaattcctcctactttataaaacctttaagttctaaaatagcataatactcagggttaattacatgtaaaccgccccaaataaaactagcaacaatactcactaacataattgagtacactacaaaagctgtcgggtgattaaaaggtacatggaatttttttactaatacccaaaccagtaaagataataccaatcctaataaaataaaccctaaccctactaatacttgatgccataccatagctgaaaatccttgacctaatgcattacttgcttgttctactgctttattcatattaatttcctccttattaaatatctaatttctcataacaatcttcacaaatcaaacctgattcctctttttcaatatactctttattatctcccatatatctatatccatcttgccacatatcaatataccctcgacacatatcacactcagtatcttcatgacttgtattattgagattaaatcactacttccacattcactacattcttcaggctcttcatcatgaccccagtcttcgaaatgcttacaatcctcacactcataagttaatacaaaatcctcttcaaattcaaagtctctttgtttcattttaatttcctcctttaacttgctcaattaaatcttttactgcataaacttccggattaattaatctcataatatcagttgggatacctgcaactattgcgtaaagagagaataacgttaatagtattaatagaattgtgtaacaccatccgataccacttaagtcatcgtaatcatcatacaataagctttcttttaaatccattttaaccttcttatattctttaaggtaaaccttacctaaaaatattgttagtgcaataactaaaatacttacaacaatatctataataccgtacgtaatttgatactttactaatgactccattacttgattacttaagttacctgctttatttccaagagaatcaattctctctaacactttatcttctaacttcattacaaccactccttatctagtcttctgagttccatccattcataaatccaagtacaaacagtactgccaatataaaaattacgaataacataggtcatacctcctctttatttgttaacttaagtataacctatgtatactcaattgtcaagacttatttcttctttttacttaattttttttgttgctcttttaatgaatccttttgttgaggtgttaacattaattatctttccttatcaaattcttttgttgttttaatcttgtattcttctttaggattttgttcttctaatctatcttttaaatcctctaatacagatacatccctgtgagctttaatttgtctaggtattttctttttacctattttgttataataacataaagcataccactcattcatttatataaccaccttacttaatattcttgatttctaactcaaactctactagtgagtttttagataaatctgtgatactgaacacattattatctcttttaataaagtgattactaggtgtaattctttccggttcatttttcttaatcaataattctaattgagagaataatctttcattaactaaattattaacatcctcttcaatatcttttctagttaaataagaatcatttaatttatagatatttttgttaacattacctttaccaataaacatattgtctacatgacaattacttcggtcaccatctttaaaacacacatgaccgttcttaaggtctcctcgtgattgactaatcatttcagggaatgtatgtgcaataactactcccctagaataacttgttttcttagggttaattaggtatacaatctcatgagcagtccacttaggatggttctttcttgtctgtgtacctttaattaactttttcgttactttatttctaattaaacccgtaggatgcgcttcataatacccattaaaaggtactgttctccatcctttctcaggctgtaatgagtggtctaaatgttttcttcttttaatcattatttttcctccttttgtaacttcttatctaaagttacgctataatcctctttataaaatcctttaattctacctttgcgccccctagcatgtttaacatacttataaacagtacctacaaattgaattctctttccttgaagctcctttagtttttcatagaaagagataccaacttcaatgttaacgtggtcaataggtataccattaatctttacatcagtaactgtgattttaacacacccatgttgattacctttatgtcttctacgctttgaatacttaacatcatctaagtaacccttaacattaacttgtttcctattaaaaggttttaactcttctctcatctttccacctcctactataattgtaatttatgatagttaaacatatctttagttaatttcttagcaaagtaattaatcatatattgtgttgaagttccttctagtttatcataatcaaaatcaatgtttgtaaaaactacaaagcttcctaaatttctactgccttctctgacttcaaatacaagtgtatcatttttaaaagttttaattttaaattctttattcgtatcaaaacttttagggaagttccaccattctttagttgttgccataatctcaattgcatttgaaaattgtcttctaattgtttctttactcatagtaatcatctcctgtacttgttatatactaagtataatacaaaagtacaaaggtgtcaatacttatttttaagaaaatgaaagaagatttattatcattcctagtgcacatattaatgaagcactggaatatagtattgactccttatcatctttaactttcctagctaatagtaagaaaagtaacattcctgcttgcagtaagaaaaaccatccataaaatcctgcccaaatatttattaagattgcagtaccagtaagcatatagctcatatcatcattaatcaaatcaataatgactacagctataaaagcaacagaagcaatagttaataagtccataggcattaatcccagttccagtcttccatcacaaaatcaatgtaagcttcattttcttcatctataatttggtctttaactacttctatttctgaaataacaaactgtaaagaatctgttaatctacctagcgcaatacgttctgttgttttaataggtaaacgtcctgagtgagggtgaactaagttatcaatgacttctactgcttttgtttcaccacttacaattttaatagtcccttcatcaaaaaatactttacttgattttcctgtacttgcgcttagtaaacttgcaatactattaatcatgtatacttttgtatcttccaggttttttagtttttcatttagcattctatttcctccttataacattaagtcttcatttagtttatttacagcttctataaaatcaggattcctatctagcatatctacaggattatcaacatcccattctattcctgcatgtatatctaaatatgaaattggtatgtctataataaaatctgcgtcttctacagcttcaccgtaatgataagaattatcagggtcatcttctgcaatgactatattatagttattaaatcttttctctatattctctctgctaagttttgttgctgtcatcttaaccaaaccatgttcatcatttaaatgattttgtattgtttgtattgtttcttttaaaacccgtttatttgtttctttataatgaataattagtatatccatctatacaacctcctaatacaagtatactactgtcatagcattatggatagatttcataatccaaaataagaatagtagtaaggctcctgctccgataacacctggcatccatctatctttccaaaactcaattccaaacatcaatacaaagtatgatacaaatccccatataaaccataaaattcctaataacataataatcattcctcttttctttattttatttatacttttagtatatattaaaatgcttcatctgtcaagcctttaatgtaaagttttttattaaaatattctaaagctttctgagccgtagatacttcttttgtgaactcacgtaaatctgtcatgttaagctctacagaacctatacgcattacaagctctacgccttctacaccattgtcaaatataataggtgtaaatgctgttttatgtttcttatctacttcagtaacaataatattccctgctaatcttacaactatagtataatctctatacttatctttgtaaagtacatttgtttctttagtcatttctttttctccttaaatcaatatataatgatttctttcttatctcttttaatttcttttccttatcttcaataggagtagaagaaggtagcttattctctaccttcttcttttgtttaggcttacctaccctatccgattcactgtttataaagtcattaaaatccatagtaaatctccttatagtaaaatattatttactgctagccatattaaaagtacaataattccaaacacaattgtactgatgactgccattatcatagagcaagcttctacatctaatgtagttttacttaataataacatcattagcttatctagtagaatagtcatacctttaaatactaagtagataattgccatagctactgcaaaagctataataatgtatatgacaagtagtatactatcaaatgagcttatcattgctttccacctctttaattactattgtattgtttatattcttctaaatcttcttgtttcctaatttcactttccttatctaatttctttaattttttatattctaaaattgttaaaggtctattagtaagcaattggtttttccagtggttaacatcttcaggtctttttacttcagcttgaaaagcccatgatatatttttttcttttacttcttctccattgtctctcttatagtaaaattctttaactcctaataaatctaaataccatttactatagtacttgtagttctcttctccaggtaatctgatgtaataggattcattccattcagtatagctccatcccggtgatacagctttatgaactagaataggtattaacgctgataggattccatatatagtaatgattatgaacatccctacgattatttgggctaaagtactctcaattgagtcagtaatagtacctattaaggttaaagttaaaataatagctattagtaaattaaataagtacactccgataaatttactcaccatatctctcctcttctagcaagttagtaacgtattcgttattttgttctaattcttggatgtaatcaaaagctaattctaacattttactttcactaatgtgaccttgtttataatactctaactcattcttgaatttgttttgtgctttaatactaggtataaccataatatctctcctcctttagtaatactatactacacttatttgttcttgtcaacagtttctttatcttttttaaaaagttctttagataatttagtatgttgttctactgccttattatatctcatatatttaagatgaagtatattaatctcactaaaagttatttgtcttaagttatgttttgatacaatatctacttcatcatatgaaaccaaccataatagaatctaatacctttgtagaaatataatttcttgttcctttagaacgtatgctgtccttataattatctacttcttttaactgtaactgtatgtctctattagcctcttcaatatctttaattaatttttccctttctgtcattgtcctatctcctttggtctatttgagctatcgacttaagtaagaatttatagtttcgtttaaaattttttgcttctaacttaccaaatttactcttaatacgttttttagccttgtaagctgtacctccttctagtatcgggtttagtaagtctacgcttaactccagtattctttcctccgaaagctcatcatctgaaattagtcttacaaatgcatcaaatctttctaaatctgccatttccatagttaatctctcctttctatatattaatataccataaaacaaaaagagagtcaagaattaacttgactcaatcactgttccataagtaataaatgatagcaataaagatgacgacaattaatgttgtcatttaagtcacctccttattactttttggttggtactgcttcaaacctatacttatgaattggattatcttctccatcttcagtatataccctaataagcttgaaatttaccccttccttaaatctacttgttttgtagtacctcatatctatgctgttatctcctgttctacctggtaggtaagtaggtaatgtttccataaaataagctttattatcttccacttttttcaatgtgtagtcaccgacaaattctacgttcttttcttctttaatctttcctgtgttcgataactcagtagagacttcattttttgctttgtcgtgatacatgaaagaagatatataataagatgagatagagaaagccaaaacaataaccactagtatatagaatatacttgcttttcttgacataattaacacctctatacaaaaatgtcccctcttacaattataatataagaggagactactattaatctataattttaacacatttatcgaggagtgaacaataaatcattttgtcactacctactaattctcgtaatttactatagtacttaagtaaatcattggaatccgttagttcagtgtatcggtcaagaacttcattaatacgactttcaaaaaacttattatcaacaatttctaaatgagaaacttcttctcctgttgctaatcttaagtcttctaactctacatcatatatctttttgtcatccttaatgtcttcaagtaactcatgttgttggtattcaatatttgtattattaatacgtatcaaaggttttaaaacatgagaagtttcatataagtttctaggcatttgacctcttaaatggctgaagtcttctgactctgataataatccagtactaataccaatatattgtcctggttgtacttcggtagactgatatcctttaggtattgtacagatatataacttattaggttcatataaatctttagatgcaggaaaatcagctacaacatcttttaatccttgagcatctagtaagctctcttttaactcatttaaagtatatagtttttcttcttctacttcaggttcttcaggaggagccacttcttcagtgaactcctcttcagatacccatttctctttattacgcatgtcatttaactgctcttgaaatgattttttatttctcatattataacctctttacttactcaacgacaatattatcctcttctaatgctttgtccttaagttcagattttctctccacatggtataaatctaagatgtaatataaaccattcttatgaagattatatttactataaatgtcctgtaaattcatatattggtaatccttaataatctctttgactaagtttttatcttttaagatatgtgcaattctttcttctacaggtgatttcttattacgcttacctactttatagtagtttagaactgtgtaaattaatcccatacttaccttagcttcttttgcaatgtaatctaaaggtttaccttcattatagcttgttacaatgaattcttctttaggtgtgttaaactctcttgatgtttctaatggtgtgtatttactttcctctttagttaataattctttctcttcttgatttaatgaaataaacataattgttagctcctttacttttagtatacatatactatatcataaactataggagctgtcaaccctaatctttaactttatttaatttattttcatcaaagagtattgtttcccctgtaagtgcatagaaataattaccattattatctgtaagtatttctttaatcatagcaacatctttttcttggtcttctcttgtatattctacaatagtatctttaggatacttaaccttagttgtttctgaatattcttcatctagataaacatcaaatgttataagagagttaacagatacattatagagaacccaatgccttttatagtagaatgattctttatctaagtctctagatacttcagtaaatttgtgtttccctggactcattctaacaaggatatctccttggttgtcttgtttaactctcagtactttcccgttttttaagtatgtcatgatatctactatatcgtcaattggcttagcatcttgtattggatttaaactaatctcatctgccattactctaacctcactatctaaattgtatagcctactctatcaagagcttcttttaattttctactttcctcatctaaatctagatttttgtagaattcattttgtagttcttctaatcgttgtaatacctcttcatgtttctcttcaggtataaattctaataatacttcactcatagctgtatcttgagctgtttgttttaattgtaattctctaagtccaataaatgacataccttttaattgatttccggtaatcttatctttagtttccattgcacgtatcattaaagcagggtctaaagtttccacaaactctaatccttttgcacctttagaaataatcatatctagtacttgaatatcatcataaattgtttgtgtgtgatgttttgattgttcgaatggagaaagttgctcctcttctaatataggagtttctttttccttaatatcttttacacttgtttttttacgtttatcaattaattctcctaaatcccaaccattttcaatagcttcttttcttttacttttatatcttgtaatagctgatttagacaattctaaatcatattctttacataattctataatatcatcataactcatatcttcatctaatgcattatctacctttgaacgtagtaacttattatggtataacttagataatatactttctttttctttatcaggaacaacttttatatcactgtttttatttcttaaattcttttttctagccattatatagctctccttaactgtcatttatttatggtaactgatgttctgaaaccagtttaacgaacataaacaaaccctacaacatcaactgtttcaaggttacactatataatatatcacaatttctgttattatttcataatatataaatgtaatataattttattaactactttgtataaaattttaatatattttttaactatttacaaagcctttacctaaaagatattttaacttttcataaatataatttcacagaaactagagcaagtagaaaaaacttaactagtttctgtattaaaaacattatgaaaacttcttatataacgtatatttatacttaattatcacatatttataacattttcctagaataaaatagttaatgtgtcgtcaccctttaggttaattactagctcagttaatttatgagtcttatcttcaattttaacataactagacttaatatctttagtgttaactttaacatatgactgaccacaagcaatagtaataacatctgtgtcttctaaaaagaatatccctgtctcatgtgtattatataaattaaagtagtagtctttacttaataacttaacacaaaatacacttttacttattaaactattgtttctgaagaatgctaataatttatcagtatctcctttagataacttatcttttttagagagcataccgaagagtgtttgaccatcatatagtttttctgtagtaagtaagtatacaatatgagtgtaaccatttaatggtgattcttcttctactttatacaaacgtagtttaatatctttagctatagtaatattttggtatgttacctcaggctcaataaaagtacttacaatatctactttcttagtagaggttaagttagtacctctagcaagtaaatctgtcattagtccagtctcttccaactcttttttaagttgttcagttctcacatagccaagacctgaagaacctaattgattattcatacctactacagcctgtttcattgattctactacatttaccttattaacaaactgtagaccctcatcttcattacctatatcctcaatgtaagattgtactgtaacacctgaagggaagtaatcactatcgtaagatacacctagtaattctactttaccacttgttaaggtcttgtataaggtcttaatagttgtaggtttgtctacatgttctgaaaatccttttaacattatttatctttcctttctgctttaactctatgtcttgttttgaatataaatatcaaatgtttacccttaaatgttctttgttctctgatactacactttccaataagttttttacctctatgaacataataaactctatctgtatgcaaaatagattctgttttttttcctttttgcttaagagacaagtaagtttctttaacatcaagtgcttcactaataagatttagtgcatactttttctgtaatttcacatctgtacttgttacgttatcaaatcttttaataaatagtatacgatattcaataacagatgttgcatctttaggctctgaaggctttttagcaaagaaactattattagctcctgttaaagtattcgtaggggcaaagttaacaggttgatgctgtatctctttacgtctatttggtttgtctttgctcattaattactcttaccccattcttcatagtattaaagaaatcttcttctataacttcccacctattattataaccatagataacacctcttgtaaagagtttataagtattatctacccctacataatgaattatttggttaataatagcaggttttttcattcttttactttgagttgtttgggagaaattaccttcccctccactatatctaccactaacattatagctataccctgttctatacgtatgagggtctctaatcaatctaagtattgtcattaagtttgtcttccataatatattcattgtatgataagggcttctatgtggtgatggtgtagcatctttgtatttacctacaacaggtttgcctttaaaactaatgtaactcattatacctacatgattatgtttgtaccttttaagtatttgttctactttaggtacatgtttgtcatggcacattacatatacatacttacatactagactataatattttaattggttatttaatctttgtgtagagtctcgttctgtttttatctctatacccattactgtaccattacggtctaacactaaacaatctgttctacatttaccttgacaaatgactttctcattgaagattcgtatatcgtttatatcactagttatttcatcgtctttgaacatgtgttttttagttctgataaggtctttaatatctttttcatagaacttaactacatcattagacatttaacctatctcctttaacccttttgtagtactcaacaacactttctaagggggtatatgttctattccacttaacacttgttagacctataaaaacacctgtgtattgaccatgaacgactttagttggttccttttgagaatagatagttctatccttatctctttcaaaaaatcctttaccggttttacctgttactgtatctgttcctaaaggaataacattagctacaaatatagggtcttctgtaaccttcttaggatacttacgttgaggcatcatactcttaattaaattactaagaggtgttcctcccggtattgtacgttggtaaggttctattctatatttcttctgaggattttctttttcctctctgtcatgtatgtcatatacaataacatactcagacatttcttcttctacatgctcaataaagtaataaggcgtatcattgaaatagtatacaccctcactaattctcattgctaatgagctattatcaaaagcactaaaattaaaatccatttttaagtcacctctatattaatataccatatatagtaaaaagagtcaagtaaagacttgactctataagaaatccttttccatttttctaatagtaggtacaatatcttctacatctgttaaatattgtttcactgacatactaggtgtttctttattgattaaacttactgaatatggctgaccttcaggatggtctaccataccatttgtcatttctttcaagaatccatcattaataacattaaataaatacttatcattacctttatcaaactgtattgtataaatatttcctttgtcatcttgagactcatgaatgttttgtactgtatcatttttaattaattgctcataaatatcattgaacttcattattatcctcctgtgtttgatactgtctagcttttctagcttggaatattgtaagtacaaatgaccctacaagaccaattataacataaactagcatgtaaataaagcttgtaataataacaccgaaccctgattgtgcggaatacaaggaatccctaaaaataatactcagtataataacaataacacctactaagtaaggtactatcacatgatagtactcaaagttacttagtggaggttgtccttgacttaccatttttttctctgtagtccataaatcaaataatctaaagactatgttaaatgttgcaaaagttataaacgctacacctataataaaaattgtatccatttattaaccatccttaatctaaaaatactgttttaatatcaatatctttattttccttatttacaaataataactgctgtgaaggtttagtttttgataaatgtaactctttagcatagttattatatcccatcggagatgaagctacgatgtgcattctattaaaatcttcttgttttactgagaaatgatgtacatgacctgtaattaataagtcaatatgactatctaagataaatttattgatatgattacctttaccttttaacccatctccgtggttaatgataatagatttaccgccaaaggtatctctaatagtataaatatcttcacgattatcaataatatctacaccatttaatagcccttgttcttggaataacaataaagaatctaacactacataagcaatgttatcattataaatcttctgattcttgttaccttgcatacggtcatggttaccaccaataataccaaatcttagttctcctgaaactacattagatagtacatttaggatatcaataagtaatcgagtacctttagagatttgttctgctaaagtaaactctgtttcaaatgcttggttaacatctctcatattaatatgttctactaagtccccaacaaagtaaacagttacattagaaatttccctatcttcaatttctttaactgttgtattaataaaatgatttaatcttgatttaagaacttcaaaatcatatttaccattagtcatatcagatacaatacttccaatatggaaatctgatagtaacacaattagctctgtatcattaatattagctttacttccgtgatgttctactaatgtttccccttgtaattccttaaccatatgttctttaagttcatcaaacataatttgagggtatgcagtatgcctattgtgcttacgctgttctcttaagtgagacaattctttttctttagctactacataagtagatacttcttcatggtagttattaccttcgaatacttggttatctgcaacatcttttaaatcacctaacgttactttacctttaattgctttgtttttaagttcatttagataagcgtcttgtctaatatcctctacaatttctttaaggtcatctttaccgattctatctaaaccaaactctttagtaattttattaaacttagatgttacaattttaccttcatctttaaataataccccaataataactgcttgtacttcttctattgttttatagttactcataactgcttcctttctctcttataaacttagttatttgatttcctaaaatattaatgtgactgtttacaaatgtatggaaaatattagaatcataatcatgttctacattgaacatactagctattgccttagcatccatacctttaacataaaaagtcagtatatcattattagaacgtgtctgtgaaccgataataacaataccatgctccttgtttgaaagtaaactataagctagaggtaatttataatcaggcagaacctcatataataaatatacattaccattattatagcacttttctcttaccatgtcaacttcttttttgtaaaaactatttaaatcaaatacatcttcaaaatcttgaaagttataatacttagttaaaccttttgcatcatcctctacccatttaggaaaagggtctttacttaactcagttatctgttgtaataatgaataatattttctatcagaatcaacagtgataaaagtatcaggaagtaatcctatatcagagacataataaatatcattattcttaatataatcatcaatatctgaattagaattaatgaccttattaaatgtgatatcatcatagatgaatcctgctaaaacagttggctcatcctgtattgaaagcaatccttcttcaaatgtatctactttctcaatatcaaaaaagctatcatactcatataaagtgtctgattgaatatggttggagtttactataattcccataccctactcacctactctttcatataagtagcctttaacgtctctttcatcatctttagaagaattttcttgtccgtactctttaactctgtaaccatagatgtgtaggtaatcttggatgacattcatcataccttgaacatttaaattatgaacagaaacagaatcaaactttactacaatatactgctctttgttttccattaatttttttactttcgtataatcatatggatttctaaataatattgttggaatatccattactttcccctccttttcttaattcttatatagtgtatcatagatattataaattgtcaataaaaaaagaccaactttttagttggtcttaattattatgcgtctcctgaaggttcttcagtaggagttttagttgtaaattcaggaacatctactttttctgattcacctgaatcattagcaaatgctactttaaaatctcctttagcataagtttttccggcttctaagcctgtaatagatactgtagtagctccatcgttattagctacttgagagcccacaacttcaccattactataaacttttaatgtcttttccattctttattgtcctcctatacagtaatatctgcgctattttcaccaggttcaactgttacgtctgcaggtgcttcaggaataggttccttaactgttacaacaacaatatcagtgtgactaccatcttcagtagttacagtaacattagctgttcctgcactaactgcttcaattaagccttctgatgttacgctagcaacacctgatttgtctgattcaaatgacacatttttgttagatgcttcactaggcgtgatagttgttgatagttggtgagtatctccaactgtaaggtctaaactatcaacgtcaagggtaactgaaataactttaattgcttttgtcttaaactgaggaacatcgaccttctctgactctcctgaatcatttgagaatgatactttaaaagtacctttagggtaatccgtattagcatctaaattctcaatcgtgattgttgattttccttctgttttttctacgctgtttagtaactcattacctttgtataaatttaacttagccaaatttaccatccttatctatatattattctgctgatacgtctgctccgtcttccttaggagtaacttgaacgttcttaggtgctgatggttctgaagtgtctccttgaccttctcctgaatctacgcctttaacgtccttacgagcagggctagcagggaaatccgcaattacatcttcatgtgcgtgctctaaactatctttaacttcatctgcatcattatctgtagggtctacaatagataatacatcctctttgttgtttgcaacaaatacgttataaacaccaaatttgaaacggtcttcaacgaaagctaagaaagcttgaataagttgtttacctttatctaatcctttaatatcttcagtacggataaatgagtgattataagggtctactactgtgtttactacgaaatggatattgtctccatcagtgtaagcaataggtgatttcttcattacatttaatgtacctttaccatctgtttgtacagggtataagaataattcttctccctctgaaactactttgtacgcatattgtccttcatgtgtacgagtataaccgtcacggttaactttaacttgtaaatacttatctgctactgtaacagattcttgttgtaatacatcattaacattctttttagccataagcttaatctccaatctttattttagtaatctattttaaagcattctataagacatagaaaaaagccttccctatgctttattattgaattctcgataataatataacacaaggaaggcactgtaactagttttcccaaatatgattttcagtattaactctatggttatttaattctttcttagcatagtactcatggtatgcatttattttaaatctaacatagtctctgagttctgtcaactcacttgctacttcttttcttttcatatcaaacttttccgctacttgagaaacgataaaggcatcatcttctctttcagggttaataagtaattctttaagtaactcactttgaacctctgtaaattctatatcatcaaatacataactcataatttgattatcctcgaagtcttcatttaaatcttctgttaaagactctactgtataatcttttttaccgataatttcagtacgtttatatttttcattcttcttaacataactattttgaactcttaaagttagtttagctttaatatatccaggaaaatccactttacttctaatattatattctttaactaatttaataaattgctcatcaatatattctcttagttcttgtctttcaaaatcattagataaaatccctgcgtacctatgatataacgaccatctaaggtttttatacctattaagtaaactatctaaatccctttcaaaatcaacaggtataccatctatatctattacatatctattaccattattagctttcttcataaggattagccccctcgaagattattcttactttgtgtgatttatctttaacatatatatcagagaaatggatagtcttaggtactttaacttcttcaccttcatgtctaattaaggaatatcctcctgcccatctatctgttacatgttctatatacatcactgtagaatctgtataagggaatccaccttcttcaatgataattgattcacctttttcttttacttcttcaaatttctttgctatatctttataaggtaaaggaagaacatctgaggaatttgcattccccaaagattcttttacctgattcatgtttttatctattaaattatccatctattattcagaatccttttcttgagattctccttttagttcttcctcttgttcctcttgtttattatctgtagcccattctttatagtagttttcaagagcttgatactcttctaattgagttacgattactttgttattaagtaatggtgagtaaccattagggaagtgttctccataaactctactaaacaattctagatataactctttattatctttaagtacaggaacccattcagcatctctaagtttaatttcttcaccattcaatgtaacatagtttctccatgctcctttggtaattaaacctctttcaactgcttctttatatacagtatgttctaagtcgataccatttaataagatagggtcatcttctttacctaccatataatcagatagtaagtctgcttcagctttttgccctggtcgtgatagtttagatttcttagtttcaatacgcataatgtgaccatggtattcatctttaccagttaactcacttttctgttttaactgagatgctttatgaaccttaatacgtaaactagcactatgttcaaaagctctaccacctgtagatttaatagggtcaccatacatacctgcattcatatcatcacgggcttggttaataacaattaaccctgtgtttgtatcatttagtttaggtgttactgcattaattactttttgggtagctgatgccttaagacccatttgcttctcatcaataccgccatcaatctctttctgagttcttgtagcccctaatgagtcccaaataaatacaataggtactcccggaatcttttcattgaaagtgtcaatccagtactctaattctttacctacttgctctacagataattctactgtatttttaagtctaccttctcctgattgaatagagaatagttttgaaacatctacaccaagttgctccatacgattgttatctgctgttccttcaatatcaatccaaatagtgataacacctaattgtgttgcaattcgagataagtgaactgcaaaagtagatttaccactgccagttaaaccgtaaacttctgttaaccgacctaatggaatacctccacctaaaatatagtcatactgaggaaccattgtaggtataacgtttttaatatctgctctgtttgtatctgatagcaatgttaatcctaattctttacctaaatcaattgtatttaaatctgttaaatcgacttctttacctttttttgctcttgccatttaatatcctcctagtaattatatctcaaataaaagagtgctctaaggcactctatatcttataggttatctaagtctagaccacctaatacatcgtcaatactttgcgtttgtttttgttttggaggctcttgtgtagtttgttgtggttcaggtgcttgttgtgtattttgaggttgtgtctcaaaaggtacctgtgaatcatcaacactgctaacatcaaaatcattgaatgggtttgaatcaggttgtgattgttgttgcgctaagttatcccaatcaacagaacctgtgttacttggttgttgtcctgaaggagttccttgaccaaattgagtgttattgataggttgttgtgtaccttgtaactgctgtccttgaccattactttgaccttgttgtgcatactgacctacttgaccttgctgaggctgattaggttgtcctcccatattacttggcatttgactatctacatcttgttgtgtgggtgcttgtttaggctctgaaggttcttcacctaagacatttgtttcacggttaaatttaaagttatcatgacttaactctgtgttattaacgttattgattaagaagttaacaaaattaggattttgttcttctgttggttttgctaattggtctaagtcagataattgttgttcccaaccttgaggtaacgcacctaaaggggcattaggataaacacttactttccatgacatttcacctttcttagctttaacaatattaactaggaatgcttcagttgctgagataaagctatgaggtgcattaggtgatggttttaacatagtgtcttttaagttagctaataattctttatatcctgtattagataattccataggttgaatcattacatttccttgagcatccgtttttggttgaatttgaccattgttattaaagtattcaataacatgaatgtaatagcgtactcctggtttattagggaatctactgaactgaacaccatttgttatccaatcctgaatgtatgggtcaaggacagatgaaccctcttctgcaggtaatgttaaccctgtgaatccctgagaaccatctttcttagaatagttaatacctgttgttctaaattctttaaagaaaacatttgttccttgtgcaggaggtaatactcgtacaaaagcattttctttaacaaccttatttccgtttgcatctttaatattacctaaacgtagtacagggtttttaggtttgtagctctctacattattgttaaaagaactgttattgctttctaatttgcttgcctcattgttaataaattgattgaaatccataattatcttttctccttttaattaatttacttatttattataacatagttttttatatttgtcaattatattctttactttattttttaatgtgaaaatggattagtaattccaccatttttatttgtttgtcgtaattctgcacctaattgaatcatcatatctcttcttgtctcaaaagcttttacaacatattgaagttgtttaactttataattccaagcctcaacaactttaagttgttgttgataagaatcgtgaatataaacactagattcaatcatatctttagtaggctttttaccttgttgttcgtactcttgtctaattgtaaggtttagcttactaccaattgtttctaggtttctttgttccatttcagaatatgctcttaccttctctaatatagaagcccaaaaagcatatttagctgattgttgcataaaatcctcttgtattgtatactctgtaattcttaactcatctgtaatatcaaactcttgtgagttcccatttctatctttaatagtaaagtttgtaaaatctagggaatcaatgtgtatttccatttatcagttcctccttttaaatattaactgagatactacttaaaggtaaagctaaaggatattcttcttcaagacttgccaattcctctgttacttctccagtagatgactctgttggtaaaactgtagaaccatgtttattttcttctctattatagaatgtaacaacaatattcatcccacctagctcatcctggtacatgcttgttataatccattcatccatagaacgattaaatctaatctcccctgtattcactctttttcctccttatatttattacttatatagtataacataagttaactatattgtcaatacttttattaaatttcttgataaatatgtttttcacttttaacaacattagtagccttttcaaattgttcatctgttagtttacctgactctttataatcttctaaggtctgtaaattcatcatatacttaatataaccttggtaagaattaaatgtatctatttcttcctcatcatattcaaccatatcattatagtttaacccaatttccatatcggcttcaataggatattgtacttcttttccatcaatttctgcttttaagaaatcaaatggtaagttttccataatatgaattgtcactttagccataattttagcttcttcaggaggacaatcaattaagatactatcatgtactgtggcaataacttttgattttaaattacgagattgaataaaatcatctaagtaagttaaagacatgtttgttaagaaactacctgaaccttggatgatagtgttaaatgactgtcttagaccttcattttttatctttttatctgttgattgggctgaacggataaatcttctatgtccgtgcattgtatcaacataaccatattgttgcacaaactcatgtacattgtcaatagaagtttttacacttggttttgtttggaaatacttttcaaatatttcttctgcttcacttacttccatattatttttacctgcaaatgagaagggtgattcaccaaaggctaatccgaagttaactgctttagttgcttgtcgttcttctttagttacttcttctactggtttattgtaaacaatacttgcagtagccttatggatatcttccccatttaagaatgattgtagcatatcagggtctttagtaaataatccgataatacgcatctctagggcgctatagtcggaccctagcagtactccattttcaaatctagaaacaaatgaacgtttaattggatgtttgtaatcaaacttatttacatctgatgtgtgtgcaggtaagttttgtaaattctataatttgtaagctttttagtttacaatctgttactgtttaggtaacacaatagctctcactattgagtagactatatcatatatcatttctgtggtaggttttaattattatttttaaatttatatcctctgtattcaacaaagtcatctgtatttcttagtttatctcttaatgttttatgattattcatttctagtatacccataattttattcattgtcaaatcttcatataataaattattatttaaatcatacatattaatagacttatttaaacttctctttgaatccttatatacgtattcccaagtataacttctatgatgcttacgtttaccgtgagcaacggaactaatcttagtacttttaaagtacccattagattctttttcagcttcacttggtgaatcccataacttaatgatgttaccttccatatctttttgataaatggctttcttagatacagaactaagtttttctcttgcactataagtattcaatcttccttgcttattagcgtgagcactattacctttgtaacttacccattctaaattttcaacacagttatttaatttattattatcaatatggttaactatactatagctgtcggtaggtataaatgtcatagccactaatctatgtactaatacattagcaatacctatagatacttgctcatatccggaatttattatccacggctttaaaatatagttagtttttatattccttacttttcctttattacttacctcgtagttttcaaatcctactactttcttccaaatctctttcatataactctccttattattccctaccacagaaataatacctttgctttcgtgggtgtgtattgattgtattgtctcaacacctagtcgttgaaccttacccatacttttatatactttcagggtcttggatgcggattctctctattcttaacctttttactatatcaaacacgttactgtttgcccttatatatgtttccattataagttagtagttaagacctaacgagtctcttcccgcagttaacaaagttttacaaccccaatttaagggttactactacttagtcttgatgtctctgtgcctgtctcagaaaaagaaccatgtaatgttcgtttttgtttattaatcatattaagtaatttcttagtaaatgaattacgttttgtctgcatactagcatgatatttaagtaattcaagaagctctttaacatcatcttttaattctaaattatctaacgcataaccaatagctttcttgtctgttttatagtcctgccaagtaaggtctgcttctttaacattagcattaaatggtttttccttgacatattctttatcataaggtaattgaattcctagaatatcataaattactctacctttatggtctccggaacttggggaaaacatccatttaccatctttaaatttatctctgtactgatggatatctttatctctatcttttggctttttctcatgttcttctaacgccatttggtaaagattgtattggtattcttcaaattctttaacagcccagtgctctctcattgtagcatggttcttagccatctcagactcgtatgactcatcattttctttcatataatctaagtcacaatataaaccatttgattcaattctagctaaagacttagtaagtcttgggtaattaacttctaataaatgcattgacttaggtctatcttgttctttaagtttcttaattacatcacaataaattcttctacatacatcagtatctcctgatgcgtatggatgcattaactcaatagggaaccaatcataattaaagttacctccgtcaatatcattaactacatcttttacatctctatactcattaattaaattaattgcagtatttaacgtgtaagacttagcgtagtcagataaccccatgtactcaggtgattgttctgcaatatttatgaattcatcattatccataactaaattcatatttacaatttcaggtgaaagacctagttgaatatactttttctctaattcactaactctaaattttttctcagtatcatctagttctactactgtttcatttaatttattctctaaccattctttatattcaggagctttaacatcatactctttcttagcaatctttttattttctttctgtatctctttaattttatctgagaagaatcttaataacttagtaacaaaccataatttaaagtcttctaatggtttatcatagcctccgacatctgtaacctcataagctaaatcagataatcttaaagattctttaacttcttgggtaacagctaggtaccaacctactttagtatcctgaatactctcaaaatctttaaagttttcagtactcattaagaacttaatatcatatttcgttttagcctatatatttctatatagaacggactataccttcataagcccttaccattaggtttgtttgtagttacctaaatgggcttgatgtccctcatttacatagtgtgattattttttatgatgagtatcttcatgacaggatttacataaccattttatttctaaaggtttagtataatcaggatggtgagcttcaggtatgcaatctttattacactctgaacacttatcaggtcttatgagagaaccatttcttaatgcggttcttacagagcttctagctttcgattttaaagaattgttttctctgtatttcctagacctatttttattaagctctctacctctgtcagaagtattatactcattactttgtcttctatatttatctccgtttttctcataacattttttaatagtatttttagttttatctaacttattatattccttccgacaaggtatacacatactttgataataagttttatcattcttcttatatgttttaaagttagataaatctaattcttcgttgcatcttctacatacttttttgttatttttaactgatgtcatataatcttttttcactgtaccacctctattaagtcgatacactatgtctcaccttggtctacaaaacctcggctcagtctctacaccccatctattgtcaccaataatgtaagctcgggattaccactatccttattacaggacttaggttctcttatcagctacttaaagctatgcccgatatgagagggattttacaacagcaaatcatttaccgttatgtgctactttaatatcttctttactagctaaccaattcttaagcaattctaagacttcatcaatatcatcttgaccgttttcccagttaaagtctgatttgtataagggtatagttacaccttgaccatttctccatgatagagatagtactaaaggtttacttccttctttatcaggttttaatgagttagtctctaagtcccatgcggtaatatctaccccatcataattatcattctttacttccttattgaatatttctcttactctttcaatgttatctaccaactcgtaagatacttccttaggtttaaatgcctcttctccttgctctacaaacttaccaactgtttgtaaatctgatactacatgacgttcactatttttatttacattagtatattcaatgctataagtaggtaatacccacacatcatgagaagatgttccattttcaatagttactttacttggtacacctcttactttaccaatagctgtaacatttaataggtatttaacacctagtttacctacaggaattaccatatcataagaattatcaataataatattattcattctctcatagtatggctttacttctgcttgtttaacatcttggtatttaatgattttaccgtagtcatttctaataggtgtaggtactgcattgtagagaaaatcaatatcataatctctgtttgttttatctctctttaagttagaacacttctcaagtaatttcttaagtgtttttccgttagatgtatttagtacattagatttcacactcccatctttacttacagaaaaatgctcttctctaatgtggtcaaataagattaatactttcatttaattcctctccttttttaacttaataatactataccatatattttaaaccttgtcaataaaaaaagaagagaattatttctcttcttcattaagtctatcaataacctcttctagttcttttagtggtttgaattttatagcttttctattaggtaaatggaagtattctttattcagaccatcccacgcttttttaccttttctttcaataacttctatttgcataaatttatgattctttactttagtatacccttcttctagcaagtcagaaatagcatctgtctctgcacttagtatttcctctacatcttggatatagtaacctgtattctctgatatctttcttgctatatctcttctatttgctgtactcatataatcaccttatatcttaatctttcgttgttgttttaatttcgtaattacattgtctacatcctcaaaatcacgactacttgttaagatatagatatacattttccatactgataaagatactctaattttatctgcaaaatcgactttatattcgggttttaaatcataagcttcaccatcaaagaaataaaacttttcttgtttttcatctacccctaatattcgtggaaatgataaatgaacttcatctacatttgttttaatatcatgtaaacttctaataatatcataagggtttacataaggaaatactacagaaatatcaataataacttttgtacccatgaatgataaagctatattatctacttcttttgcagtaaaaccgttgcgcatacgataaacaacttctgagtcagggtgactagataagtcttttaaaacatagtaaggtaatactgaatcagtatagtatgtaactttaccaccgtgtttacatactttttctataagcctatcattattgtacttctcattaaatagcatgtaatgcttttcatccctcatgatatatcttctagacttagatattaattcatccatattcttttcaatttcttcttcattaccgtgtgttactttaatagcctcattaattttatctttatcttttgtgtgtatattaaaatatctatcactattatatatcgttaatgttttattctttgcgatatgtcatcttccttccattaaaaaatcctactaccttgtactattatagtatagcacagatagtaggaaattccaaattaaagtgactctaattcttgtaattgcataggtgtaacttggtcttcatactttttaactaactgaccatctttatacccaattaatactggtgtactcatgatatcgaataacttaacagctttttctctatctacatcttcagcatctagatttaatgttaagataggttttttaatgtcacctgactcttgaaacatagggattacactttttaaaatctcacacttaccacaattatcttgtgtaatcatgacaataacatctttcttttgtctaattgctgtatttaaatctactaaactattcattttttccatcttctgaatcatccttttctaatgttttaataatttcgggaaatgagttaactattgtagctttttgtgcttcataaccttctgctaaatcaataagttttagctgtcctgagccataagataaagtatacatggtcatatcatgcataaactctagctgaaaagaattagacgtatcatcagctttagcatccttagagttactgaataacttcatagggtcattgatatcacttacacatgaaaactcatcatgttttttaaataatatttctgctttacttagtaaatccattgcttcttttctatccataatatcttttctcctttttataggaattccttggcagtactgtattctttactatctaagaatgagaaatcttcatctttaatatctttgatatttgtagatttttcatagtctgctttaccactaaagaaatcaacatttcgtaatctgtctacattagtttgattttctacaatagggttataaggttcaggattaaaatagtcctctcttccaaggttttgaagagctctattaaaattatactgaacatagttaattacgtcttcacttaattctagtgggtcatatagactatgtgtatacgctacttcattagtgtaaagaatatctaataattcataagtttctttatctgcttgttttttctcttcttctgttagaagattataatcatattgagctgttaatcctactgccgaaccatggtaagcttcatcttgagtaattttataaataatagctcctgattgtgtcatctgacctcttcctgcaagaagtaaaggataataaaatcctgagtagaataatgcactttctaaaaaggcactagctactttagccatgtatctatcaaatatagtaggattaggttttaatagttggtcgtagtaatatccgataaactgagcttttacttttaaaaagtcgttttcttctacccaagtatctaataaataacttgtttctttattacttagtaatgttgtaaaaatatgactataagatttagcatgtatctcttccataccacccataaacgcaaatacagcttggtatttaggtcttggttcgtggtaggatactaagttcataccttctcctccttgctttgtatcgagccctgtgagccctgcaaggactttcttataagtgttcttttcactttcagttaaactattccaagaacttaaatctcttgatacatcaaactcttcaggagtccaaaattgggagattccttgctcccaataaatatcagacatcccgtcatcttcaatattccagtttgttgcttttaatacagcatttttattatgttgttttactttttgtgtaatatccatatctatttcctttctaaaccgaacatgtttcacattcaataacagaaagtttacgtgaacgtgtatagtataatgattttaatccttgttcccaagcataatagtataatgatactagtttattagtagggatttctgattctacataaagtattgtagacactgcttggtctgtatgtttctgaacaactgcgcttgtattaataatacgtttattgtcaatcttataagctgtttctccttcatagaagaactgagttataggacttaggtaaggcattgggtaatatgtctccatatctccatattgtctattctctatagcactaggaactggcataatagaacttgtagcattctgtacataagatatagattgagtaggtgcttctgcttttcggtaaccattatacaaaccatgtactttcactgctttagctaattcatcccaatcatcttgtgttggaatataaaccttatctaagactttcttagctttatctgtcttaggttgattggattctctaatgtatcttacaaagtattcacctgtagcgtaatcggacttctcaaagcctttaaatggttttcctgtttctttagccattaacatagatgtcttaatagactggaagttaatagcactatataaactgtttactaggtctaatgcttccctagaaccataactaatcatattcttagctaaaagtccatgtaaattcattgaacctaaaccgatagctctaatatcatcatttgctttttgaacactaggtaagtaaggtaaatccataatatcagtaacttttgttaatgctcttgtaccaatatctacagattcattcaataaacctttttcaaccacattaaccaagttaagagaacctaatgtacagacaacatctctattaataacattctgattactgtaagagtacggataaatttctgatacctcttgtaattgtgaaatttctgtacataagttactcattttaactttacctaggtttttcaatgggtgattatcattagcattatcaatatagaatacataagggtaccctgattctaattgtgttttagcaatatctgtcataacttctctagcatcatgttttactttaccgatactatcattatttagtaacttatcataccattcatcaatgtttagctcatctaaatacttaccgtacactttattaatgtcgtaagtatcaaaagtatagaaaggtttaccttctttaactaactccataaatttgttagggattgtaacaccaatagataatttatcgagtctaacagactcactagcattgattttcttagaacttaataaatccaggatatcattatggaatatatttagataaacagcaccactaccctgtcttacaccaccctggtcataaatactaactttatgttcaatagctttagcaaaagcaataacacctttacttgaattaggtctattacgtactggagctcctttaggtctaattctagttaaatcaattgcaactccaccaccattagaactagcattagctacactatcttcaacaaagtttaaagactcaattgaatcatctactacaaatagataacatgagcttagttcacctctattagcttgtcctgagttgttatacgtaggtgtagatggttggaatgtttgctccataagttgtactagtaattctctagcctttacatagtcattttgaaataaatataaagctactcgaacattatgttgttcatagtcttcaacaaaaataggttttttgttttcatcatactgttttgatgcgtaagattcataaaacttttgccctgccatgaaactagggaatttaaaattaaaactgtaagctaacttagtcatttcatcaataatagtgtcaggaacgttgtcaaatattttatcatatgttccttcttttgtcaatactctaattctttctacttcactattaaactttcttgtattgtcttcaatatattttaaatactcatctaaagcttcttggtctttatagagtttattttttccattgtcatctaattgagttatttcattatttaactcaatccattttccatatgttgccatatatctctcatcctttacgctactttatatttattactaaagtgttctaatattggttgtatctcgttaaagtgtcctgattgctctactttagcaattaaaggaatcttaaacatctctgaaatctttttacttgctccacagaaattagagccccattgtttatttcctgtgccaataacttctttagggctcccattaatttctaggaatctttctatttgtttaggtattgcacctttcttataagtaggtgttattagtataaaaggtttatttactttttcttttccactcttaaccctaaatgtttcaaatgaattattaatattaattttgtttactaatctttctgtttgtcctgtacctgaaaaatatacgattaatggtctttctttgcttgtcatacttatcttaccttccttatatatcactaagaaggtcattaatcaattcatcttcatgcttacttacctcagataaattagactcgaccttcttgtatggttttatatcttttttgtcgtagcaagatataatataagaaggtgtaaatgaggtcaacccattcatggtagttataaggacatcataaaaaaatttatcttttctaatattatcaataataaagtctgttctcatgacagggtattcattatttcttaaatcttcatatactttctcatcataaggtaaagcaacataactctgagctctatttctagtaaatattaagcaaggtgtcttattcaccttactactatcattaatgacttgctcccaccatgtgtgtggctctctgttgtttaaaagaacgttatctatagtccattcttctctatgtttacattctactactaaaggaaaattagcttcctgaggtactactatatctccgacagcattattatctttaccccatgaagcaccacctgattgaggagacctattgaattggtatccccaccaagcagttaattctttagctattttacgttcgaatgtatccccttttttcttactgttcgtcatttttcttcaactgaattactttttcattgctgtcagattcagtttttttctgtgcctcttcttgttgtttttgacgtaattcttcttgtttctcttttaggtaagcttctcgtttttcatttagtttatctttagctttacctactttttcttgagttactcctaactcatcttccatgataatcatcattaagtcaattgcattaaatgcatcattaaagtctcctgtaacctcttgagctgttacagcattaagaactttagcaatacgagttgcttcttctaaagaaacacttcgtgaaccaattctatctaattgtttagctgttttctcatcaacttgtttaccatcaacagttccttgtactacagcttcaatccatttctccatgtctgcttgactaataccttttgtgtgtgtgctaatatttttttccattattcttgttctcctttttcttcttcatttttaggttcttcttgtgatttatcaattgattcttgaatgtagtctaatacatcttcattaataagctctgctttatataaagcatcaattaaaatatttaagtttaactcgttattatattctaagtctgtcatctgctcattaacacctaacataaggttagtaatagctttagctacatctgttaatgttgctgtttcttctttacccatttcttctaattgatgttgtcgttgctcattaccaactcttaaagctgtaatgtattcatctacaaattctaaaatatcttttttattatttgacattattttttttctcctttattaattacttaattactagtataacataaaacttctctaaggtcaagcctaaagtttaagtttttgttgtattttgtaactctgtatactttcaggtgttactagcacccggttctgttttaataactcaaaagctttccttgttcccatatcatttgcatcttcatcaccatgaggtacaaagtaaactttattaaaatgtgtttttaacttataagctaagtctatattattatctaatgcatcagtgtctaacataatatatattgatgtatctatactaacataatcaattattttttttatttgattctcagttacttgtttacctaatgttgctacgccatactcatggaaggttaaagcatcaaatacaccctcagttataactaagaactttttctttcttgctatattcaaattaaatatgacatcttttctccctacttcatcttgtttagcaggagcattaatagatttaatatacgggttcttttctatacttcgtgtattccaatactggtaatttccatcattatcataagtaaaaaatataatactattccttaatgatactttggattccccgtcaactttataaaagctcccattaataacataacctatattattattaataatttgttctaaagttatacctctgccttttaagtattttaaaaagggtataatctctttattatttaagttatcttttaataatttatacccttcaggtaatctaggaggtttaatactagtatttcctttatcttggtgcacacctctaagcattaatataagtttctctgattctgtcaaatccttattattggtcgtaagtaaaggggctctctctatatctatattcttagactctaataaatcaaaagcttgtttacctgtaatgttataataagtcttcataaatgtaatagggttacctgattcatcacattttttacaatgatactgaccattactagagtctagggcttgcttaacatagaacttataacttttttctccacaaaacggacaacagtatcttagctcacctatagtattttcttttggttctcctaattcttgggttaaaaagtcttcaaacctcataatatccttccttactatttactaatttttttcattaataacaacatattataaatttgtcctaagtcactataagttaaatctacagaaaaatcactctctttaagtgcttcattcagttgtgactcttctgatttcatgttaaatattcttaattcttcttctgttttaccttctaggtatctagtaacaacaatagttttaagactttggaatataactccaatatctctaaatccttgctctacagtattagcatcatattcttctgttataataggtaatccatattgtttagctttctctataatctttaaattatctttgcgtactgcagggttgtaccaataacctattttacatctatggttattatctctaataatatcttggttattatcatctaaaccataagatgaaggtaaagtagaacgtaatctttttaattctaagtattccccgtttctacttaacttgtactcataagcattacttagagggtaccaatcaactaagttagcagttggtacattatctattgttttgtgctccttattacgatgaaacacattaagtgttgcattatctttatctaaaatctttaatttcattatttttcctccagttttgatactccattttcttttacgattttaattgtttgttcaaacagtggtttaagctcggtattatgagtaattacaaatattgttcctactgtattaagtctatcttttaataatttaatcacgttttcacaaccgatagtatctaatccatcaaaacactcatcgtaaagtgcaatattcgtagatatctcatctttactcataattaaatcctgaattgcaaaactaattgctaaatcaatacgtttttgttctcctgctgaattggatttgtacgaacctccgcccttgctattcttaacaataacatcaaacttatcttttagttctcctttagcattcttcacttgagtttggaactctatttcaatatctgaacctgataaagtttgaaggtactcatttgctttttcatttaagaatggtgtaataaagtctaacactactgaacgtatacctttattactaaaagcatctacagcttgctgatatttacctttcttagtttctaattgtactatattctcttcatgtttgtcaatagattcttgtaattctttcttatcttcctcatgtttttctttacttggttcttctctctctaatccttgaacagattgatgttgatatttaacttttaaatacgttttattattctctaatgaactaatgtctgatttaatcctagaagcctcttgaattatgctgttaatcttattattgtgctctcgttcttgttggtcttcctgtgacattttatctttaatttcttgtgatttatcttttactttatcaatagcttctttatacgtatctttcatttctaatacactagtaatctcttgtttcttattctcaatctgattattgatattttcctgttcttttaatttatgagaattatctataggggagccacaaacaggacaatggtcatttgtatctaactgatttagactattgcttaatgtagctatttcagattttagtacttgttctttggtattatactctgagtaaacttgatttaactccgttaatttattagaaagtaaatctaggctttgagaggctttattatactcctctgagtgcttaaattcatcttcaggtattgttggtatctgtgaagctagttgctctatttgagcttctatttgcttctcctcttgttctcttaatctatctttttccttattactgttttctatctgaaccaattgttctttatattgattgtactgctctatttctctttgaaagtactcatcttttgtcgataactgataacctagtttatagatttcctgtcttatgttattttgttgttcttccacttctttaactttctcttttgctacatcttgtgcttgtttatatacgtctgtcttagtaatagattcaagaatttctttcttacctttatctgttgcttgagagaacatagggatatctccttgaccatacatgatggcattaacgtaagtattaaactcaataccaaataaatcttgtatttgtttatcggtaacgtcatttgttgaacctgtaatctctttttcattaacgaataattttactttattcttattctctttatctttacgataacgttcaattaaataactatcttccccaatatcaaacttaagtttaacagatgtatttttcttctcaatattatttactacatcatctgcttttagtcctttttcagttttaccatataaagcgtaagtaatggcagatattaaagttgattttcctgagccgtttgaatgaaatgactcattagatttattctcaccttcaattagaattaatcctctattgtctaactctagcgtagcttctttaattgctaaaaaattattaacttctacttgtttaaatacaaccattacttatcccctttcgtaactctaaagaaacaatgatgagagccataatcaatccagtaatactcatcatggtctactactctattgtaataatgcttttcaaaattactatcatcaaaaatctttttcattttacttagaccatcttctaaaccttcaacattacaaatattctctattactccatctgaatcttggaattgtaaaataggtttacttttatctaagtctttacttactacattactgatatctaatgtattaattttaggtttcatatctttaaacactttccataaatcttcggttactttctcccaatcctctgctaaattttctgcatctgtcttatttagatattctctatattttttcattataaaacctccttcaaacactcaagtatttcttgttctgattcaggtgagtattgtttagcataggtactagcaattgttaaagggtcatcagacacatcactatcaatacgtttctctacagtatactcttttttaatttgaacttcaacattcttatctttcatactgtcatccatttctaaaaccttagcatgatttgctgtaccgataaccctaataaaattatcttcctcgattagctgttcaaagttctcaggaatatcttctccttgaatagtaataaatctacgtgtatggattgggatgaattctgtagtcattttttctgtatctattaaatgaacaccattagcttcttgctcatcagaaaatgattgttgcattaatgaaccaccataaaaatgattcggattttggaaatattgtctacggtgataatgacctagtaaaatgaaatcatatttatcaggtaataaatcctggtatccaaatgccccttctaatctatgagacccttttccagtcaaagagccttctacacctaaatgacctactaagatatttactctatcttctacatatttatttttaataaactctttaatttcttcagtctcgtctccataaggagccataacaatattaatatcctgttcttctcctaaaaaccctaaagtatcaaggtttttacaaacctctacattaggtaagtaaccgaaaggttctatactagaatctgtatataaactatttgtaactgaatcatgattacctcttaacattagtacttctatatctctattaagttggaatgtttcaaataccttattatatactctagtatctactgcgttacgtttgtggaataaatccccaccaaatataactgttgcatcctcttctcttgcaatatcaaacattttctgtaaagcttgtatctgttctctaaatctatcattcacatactgctcatcaggtttagcatagttagtaaataagtgaaagtggctatctgtaaagaatacaaatttcattattaatctctcctttattctttataaaataattataccacctaacatataaaatgtcaagtggtatattttaaaaaattaaactcccggtaatcctccgaatgtgttatttatagcttctattttattatctttattttgaaatttacttgtgtcttcttttccattatctgatagcaattgtatatgctcttgtttttcttcaggtgtttcatctcttacaatcatcttagttggttctactttaagattaacaaatcgttctcctgtgttagagctattacgaattttatctaaatacaatcttaagaaaccgctcttgaattcttcatctttttggtttactgctaaagacacctcaacagcattgacaatcttacgagaaccttctacatgctcacttgtaataacatctgaaccataagcaccacggttagtttgagctaacgtccaacaaacaaatccatattgctgtgataatctacgaatatcttcaaacaatttccctcctgcatctgattctgaatgatatttagcataaggatttctcattaagtgaggatagtcaataataacaacatcaatatttttatccttcttaattgttgtattgacaataatttgttctaattggtttggtgtaacttcacctggcatatgtttagaaatataaaagtcacctaataactttctattcttctgataatgattttgtattgcatcataaactttattatttaaagacatatcctgatttacaatttgactcttttctgctcctgccatttgttgctcagcacgcaaaaccattctatccattttttcctctaaggcaatatataaaacacttaatccacttttaacataattcttagctaagtttgaagccattaatgatttacctctaccggtaggagctatgattaatccaacctctcctcttgctataccaccttcaatatggttgtcaatagaagtaaatccagtagagaatttatttgtagctaaattactcaatagttcttgtttcttttctgtatcttcgaagaagtcaataaactccccattacctcctgagatgtcacttacttctatttgctttagtttgtctacaagttcatgtagattatcagagtcttcttgtttattttctgcaataaatttaactaaaacttcttttgacatttctgttttaatatacttctctatttcataattaacagattcatctttttcattaacgtctactttatataaatcttctaagtaagttaatgtcttggttactttctcttcatcttccttattattacccataagctgttctactttaattgctaatgcttcatttgacatcttatcggaaatatgtgctgttctctttatagctgtaaataagtaacccatgtcctcagactctgctgaaaatagattcttaggtaagttatctaatacttctcttgcaaaatgtatatccttcattgatttatgaaggataagctcctttatcttcttactcatagttattaacctcttcctctactaaatgtataagtttattataatccaacataccctcttttgtcaatggtattttatcaattaaattatatttctctacaatatattttacttctcttaagtttacctcataccctaaatagtcaccaaacattcttaatatataataagttgaagtatgttgtctcatatataggtattcattaccattctgttctaagttttgtatatccataccactcaataaatctttattcactatacctcctagacttaatggtaataaatcctctctcttgattcctttatttaactgcttattgtaaactatatgctctttctgcatagtaaacattgataaccttgcagaaggtgaaatacttgttggtgaataatcattaattatgcattgttttacatatttattaataatatccttttcttctctaggtaaattcttaatttcttcttcaatcatagaattatactgtagtaaaataatatgtttcatatctccaaataatccataggaggcattttcaaggtctaaggcttgttcaaacatagtatcaatatcatgtaaagtagaatttaatcctgtagtatatagttggtgtagatgtaatacagcagggtttttcacatagtctgatgaattaatgatgctgtctgtatcacctaggtgtctattagtactgttaacacctttttttattcctttaatataattattatagtttttaaaatacttatctgagctaaagaagttaggagaaggtattggattaggttgcataccgttctcgtaataaaatgttacatttttaaatacattttgcatatacttaaatacattaatatttttatcttttatttttaaataaaaattataaaaagtattaaacattttactaccaaagaaattatcactaacgctatttctaggtaaattttgaatcattttatttctatagtattcagacatatgttgatggtagtcgtattcatctttagttgttgccttactcattgctttaagatgacttgtatgtttagcatccatatgagaaatagcatattggtcgtataacttacataagatgtacgctttataaaacccttccgggtcataggacatattaaaaatatctttagaaggataaggaagaccttctgaatagaaattcatatctgctataattcttctcttctcatccttaatggcattgtattgtgccatttctatcttagtacgtctacgttgttcttttctctcatatctataagaagggaagtgcttttctcttaatgccttagcatactgtaaatcggaagtaataatattatctgtagaattattgaattctttcaattcttctgtgtcatactctctaactaaagttattattaccccgccgttttttcctcttttagaaacagtaacgactttattttcttcttctaactttttaaggttattggataaagcagagatggaaacacctagagaatctgctagctctttcttagtagaaatatgaaatttattttctgtagcattttcctctagatattggtatagctttctttgtgtttttgttgccatctcttaccctcctattttcctaagtcttttatttcaaaatcttcctcttcataaattttcctacgctcattagcatgagtatataaaaatctatttgtcatatcattaaaatcaaatatttgtgttgtattatcgtcttttttcttacgtaaagcacgaccaatacgttgcaatgtttgtcttaatgacttacctcctgcacctaatattagtgcattaatacctgatatatctacaccctcatcaataagtgatgtagctatcattactttaagcttaccacttctcatatcgtttaatttttctctcctagtttcagagtctatttctccatgtaagaagtaatgctctacatctaaatcatttaacatttctgatattgtgtctccatgttcaatgaagtttacaataatcaatgtacctttatcttgattataccacttttctgttagttttgcaataagtttattcctaaaatcattatttgttatacctttatcgtaagcttccctataatcatcaattctatctatgtcattaggattagctacaggtataatatttattgttggtctagcagaatgaccttcatcaattaaaaacttattagtagttcgtgcaataacattaccgaatagcgcctgcaatctcatccaaagtaattcatcttttttatctatagaccctgttaatgcaattcgatataaagctttttcacatgtcattaaattattataccaagaatcagatttagaatggtgtgcctcatcaactatcataactgtaactgagtctaaaaaatctctcatatcttgatatttatcatatttcttttcgttcttttctctaacaattttttgaaaatgtgcattatgatttcttaagtttaataaaacttctgcatctgtcttactattttggtaaattatctctaaggcacttaatacattttgttctacttttgttttaggtgttgtgttatcaagtaatacttttagtaatttcttttgatttgttccaccttcaaatttaggtaatatctcttgagcaatctttttactaatattttgtttaggcgtaacctttaccccttgtgttgggtctttaaggtttgcatttaaagtaggtatcattacaactgtaacctgcttaacatcaaacttacctgcacctactttaccaataggaatatttaaacgctcttggagcctatctgcagactgatggaatatctccgtagagcctgtgaagaatgctactctctcacctttttctaattgaggtaataattggtctataataccactagcaacctcagttttaccaccattagtagctaagtgagcaataccattgtaaaatgttaagctattgtacactgcttcatattggtaatctcttaaggtaatttgaccgacattattatcaagcaatgttatctcatcatcaatatcttcttcagataagaaactttcatcacgctcatcaattgtttcaaactggaaattataccttgattgtagctcacctaataataactcaattttaaataaaagtccagtggggaatttatcttcctcataaacataaaaatctacataaccatcccaactacctcttttgtacgcagggctgtactgaaatccttcttgtcttgctcctatagtcgtatggactctctttaaaaatatatcttttaagtatttatcatcctcctcaaattctacataggtatataagttcttaattcttaatctcattttaatctccttatatccttacacgagttatagtataccacagaatccttttcaagtcaagaaaaaaaaagactaggaaatttctcctagtctatggcatattaatacctataattcttgtaacgtattgagttttactattttgtactgcactatctacaagagtccatgtagaacctttaaaagatacacctactctataaaactccgtgatagggggttttgtatcggttctatccccggaaacaaatgtacctgaacaaacgtaagttccactaccatctaaataaaacttatgacatatagttcctgtattgtcattacctttagtcttctctgttgtcttatgtgtagtataaacttctacataagagtagttactaatatcatctgataaattcttagtagttcctgtagataagaatgattgtgctcctgtccataattctttatgagaaggggatgtttttggtgaagtccaattactacctaccactgaacctgttacatttctaccatctgtacctatagcattgtaaacaccatgtaaactagaagtattaggtgtgtctgacatgaataaccctcgacaactattcgacataggggaaccggatacaccgccttgaacatagaaagtaaaggttgtttgacctgttctatctatatggtctataatagcttgttgaaatgttttactatcactagtataaactaggagtgggaatgatgcactaccatcgtctataatactacttagtagatatttctccatatcttttggctcaaattcttttttccaggctgtgtatgttcctgtaggtttcttaatgctagtatattctttaccggtttcagcatcagttagtctagctaatctattatcattagcatctacacttaagtctaaaaatccggagcttactccactaggcattcctcctgtcattcccataacaaagtaaacacctggtgaagctatatctaaaatattagttcctgatataacagcatttttattactaggtaatccttgaataaattgccattctgaactagagtttcctgagacaaatctatataacattttaatttggttattgttagtagatagtgtatataaatgttgaactaccttactagaagtatcaagaggtactacatgtaaaacaacatcaagagattccatatcaggtctatctaataatgttgctaatgttctagcacctatatagtaataaccaggttcttttatatctctaagggctgttatgtttccataagatttagttaatcctttatcatcagttagtttaattttctgtgatttattaaaattataaactaaatcatcggtaatgtcaaatatatttctttcttttgttaattcttctccaatgttattgacctgagtagtcaggtctctaataacattgttttccgttattgtagtaaaatttaatgccatttataattaccccttttgacctaagagcttttgaagcatttccatcatagcttcttgttgttttctcataagctctagttcttcttttaaactatcatattgttctttaggtacagtaatttgttgggattcctcatcatcatcattgatttgtggtggtacatagtttttattttctataacctcttcaggtttactataatacaaatatctttcataggcaaaattatcaaagaagtaagcaggtaagatttctctattaacttttacttgtcctaactccttatcataccctcctactgttgtaaaccctgttatatatttaccttctcttaaatgtaatactatctcattattacctatatcatatttaacagccatagtttttttattccacctttctttatcctctattaattcctataatattgtatacctttatacgttccttttcattccatgatgctacttttgttgtatttgtagacccgtctaaagttatagattttaccataccaggttgtactgatgttctagtaggaaaagtgcaatacccttcaaaaaagtctacactagaacctttactatcatttgaaatattaaaatctctaatatacagtaaatttgatgtattttttatatctaatctttttgtagaaaaatgtcctgctgaacgagtccaataagttacctcaattaaatcgtaattatctaatgagtcatttaagttaacagcttccgtagacccaaaatctaaagtacctttccatagtaaagtagaagtttctgattgctttaagggtttccaaccctggttataaccaccataatataatgtgtaaggaataccctctgtgtcagtaaacaataatttaacaaacttattagctctatatgggtcacctttgtcataatctacctgaatggttcctctaatagaacgtcccttaggcgtcccggcaatagagccttgacaataaaatgtatgatatccttgaggtactgtcatagctaaatcttttaaactcttttcattaacttcagatttccaccactgatatttaaaataatcattatattcaatatctacaggggagtgaccttttaattcatctttactaataaattcttgccaattagaccaaatctcatcatatttacgaacatcattttcatgtgtaataaatggtgtaactttagatgatttttctaatttaggtaaggcatagtatactacagcaccattacctttactagatgaacaattcattcttagtagtaaagatacgtaattaactttaacgcctgatgctccgattgtattttcaggtatagtgaatgtgaaagagtattttttccactcagttgttatctcacttgctgttatttctctacgtcctcctgtataaggatttttactagtatcatctacagtatcgtatcctgctaattcaaagtaaactaagttatctttaagtaaagcagggtcattaattttagcatatactgataccgttacagtatcacctacagaaagttctttacccattttaaacctattagaaactactgaagggtacgtatcagacccatcatctatctgtactgtattctctcctctaaatctaactcctgaacgaccactattagaagaatatccccaaaatggaggcgtatcccagttatctgttttaatattagaatagctagtttcaggataaacaaataaactatctttcaataagttaggcttttgttcttgtactggtaactttcttatcatcttaaaagttgaagtggagttaaccgggttaaattctactaaagaagaaacaccatttttactttctttgtaattaacccaacctgtatttaagttctcttttggaggattaactagtgttgcatattgagttgtggattcagtaagttcctttacattcaaagtattaccttcattgtataagctaccttcatctaatttaaaactaatccaatcatatacagtaccattagcataagtcttgatatacattttgttactgctatatggtgaatagtaaattttataataatttacatttcttttatccaatcttacaaatcctgctgtagcttctccactaggagcaccttttgaattagttacataatagttaccactatcaggtatagatgctaaaatattattaaagtcgggttgtgatatctgttttacatcactaaggttaacttctgtttttaaaaagttacctatatcattaactttaggatacatatcttttaacttctgtgtttcagtaagaggcgtgtagttaaatgccatctatttttctcctctctctttctttaattcattgattaacttagtagcttcatcaattttttctttgtatgttttttctaattccttaattgatttttcctcaactgtaggtataaataatttagcttgagaataagggtctctaacttctctaggcatataaatctctccttattaaataaagaggggtcaatcaacccctctagtttattcatccctagtagtaaccataagtctacgaacacgaggacgtaaaaagctattttcagttgataaatctaatctaacttgtagtttagtatttgttcctgatgattttactttctcgtcaatgacatagcgtgtaaactcattattggctctagtagttgtaggggatttagtaaatgttttccaagtttttccatcatccgcagaatacttaggaacaactttagtacctttaggtaagaaagcttcataactaaatcttactgtattgtaaggagcctctgtcatatcaatagctctaccaacatatgaacctgttaactctgttaagaatgtagtgaatgttaaatcactagagctcattaatggtgagatatatctattagattcgaaagttgctcttaacttaacttgtcttgctagacctaaaacatctaagtcttgatagttaccgataggctcccatttcaattggtcgaatgttgtagaagatgccatatcatctagaattaatttcatttcccacgtacatcctgttctctcaggagttaagtacgtagacataagaactattctatccgctgatacatctttaattggttcgaattcaatcgttgctgtctcattaaatttagaagtataaataccaaatttaaggtcagagttttggtgaggagtccatgttgatgcgtttgatgaactgaataatacaccttgtaggtatggattacctgaaataacctcattaggtttatcaattttaggcttagttctagtacctacccacattgtataatcactgttctcagtaataataacaatagcgtactccttaccgccttcagccatcatagggtcatcaaagtatactctagtttcagcactagcattattagatactttaatatcatcagcattcataactgtttctgcatagattgttttattaggataaccttggtcacccatacctctaatttggataacaacattagattgtttatcacctttagaagcaaagtataatcctaatgatgatatagttctattctcatcatattggaatgattgtgctaacgggtctactaagtttactgttacacgagttctgataataatatcttgagcggtttttttacgtccttgggctgtgtaagttgtagcacttgtagagttagcattcttaagtgtaacttctcggttaccacaacgaatacctgcaggaatagtaaacttacctttagctgttcctttagcatctgtcattatcgtaccatcttcagagccttttctatagccagttgcaggtgttatagcacatcttactccatcaaataataaatataaattattatcattagggtttagtcctttaacttcgaaggatacatctctgattctaatgaattcaatcatttcttctagagtacgttgtcctcctgattccaataaagtaccggtacgaccatgctctctatcataagcgtaagtttcacctttccacttttgtcctgcatctaactgtaagttagaatacaagtaatgctcagtctcaccatagtaactttcattatgtctccaaaatcgtttcatagttacttttttagttttttgttcagtgattgtaacattttcagtatcaatccagttatcctcactaggtgttaattttaacacaccttgtttgttaggaatattataagggttaacatttaatgtttctgatgcttgaccttggtagattgtacgttcctcagtaaatggtgctgaaattaatctaccccaaatatgagctgttgtatctcctggaataatcttaggttggttaactgcttctgtataagctagagtagcttctgcatcttcaaaactaaatacaattccgaagtcaggatgtgtaatgtctgctttgtcaagactaatgaaaccttcactgaatactgaacgtaatgttagagggttctgtccttccatagcaccatcatctagagcatttactgcttggttatactctaagttatctactcttgttttaactttctgtaagtcttccatagacaatctagtgattgcaaatgaaatacatacggcttcatctgaatcaggtaatactgtaactgtacctaattgtaaattctcagggtctgtgtttaatggtggtgtaactaatctcataatattaggttcaccaggtaatattgcaatatcaccatacttattaataaacactgaatctttacgagccaagtagtaagtatagtctactaatactactgtttggtcaataggtttagcaccatttgaaggtgtaaagttaatgtaccatttcttacttaacccttcaccttgagttgttacttcataatccttaccggcttccatacgtttgttatatttataagaaacgtagtatgaagtacctcctgaaggttcttgaccttgaggtgaccaatcaattgtttgaccatctgttaatctgaagtcttctccttgtttatactcttttgtagtaactccagggcttgtttcagtccaaacttttacaatttcaaatgctgtattatttgaaagaaaatcttgaccatcaccttgagctcctcttgtaactcgttctttttcaataagtacttgacctgtaacacgtctaatttcttttacagggctattagctaaactaatagagttattagacttattaaagatagtactttcattttctgctgttcctaagtcataagatttaggtacactaattcttgttgatacgggtttgtctactttaaaaccttttacataggctttacctgcatctacaactacagaaacgtggtcatcatcttcagcattaccttctgaaaatagttcaaaaccatttactttatatgaacctgactcatcataagtacgttcagctaatactttgttgattttatccatctcagcattagttgattgaatatataaatccccatccatgaaagtataaatagttgctgatgtcgggtcattaactgttaatgacatcttttcttctaatctatctgcacctttagagaagtaacttggtactccactagtttggtctaataggctagcatcttcatcaggtgtaacaatacgttctgttaatttaataccaatagtttctttacctacgccagttattttaactgaatcgtcattatcgtaatatcttattttaccattgatatatacataaccaggatttactgtcaatacattatcttcagacaatgtgaatccaagccctgattgtttatctccgtctttaaaaatagcgtctcctagattttttaaataatattggtctatagactgcatttcatttaattctgcctgttgtagaggtctatcaggattaaataatacttttgttctatctttagacgggtcaaatctatctaaataaggtgaacctttaaaattaattgccatttagaattatctccctttctttaaacttctacaataaatctttctttagcagttgtctgctcacttctattttggaattgtttattatcaaagaataataatgttccagttgattctacttcactaggtactaagttaaatttactaataccactttttgctactaagtccataacaaatcctacttgtctatatgttccaagaggtagttcgtcaccaacaatactactttctaagtaaacccatttagctccttcagctttagcattttcaggtgttacttctacccacgatttattaccataagaaattaaattcttattatcatcttcaggtgatttagaaggtctaactaaagtaactttagtagcttttttatatccaataacctcctgtaatactgttgcattttcatcaggttgcggtgggtttgtttcattagaccacggtgtgctctttccaattgttaagtaaactgaatcagctttactaactagatattttgctaactcaacatgagaattatacgttgcaattgccatattttttaattcctttcatctgttgttttctttattaatataacatggtatacccactaaatattactttataagtataccatattattaactttctgtcaacttatttattaaactttccttaattctactcttcttactttagagatagttgaatcataaggtcgtatatatattctaacatctgtatttaaccttggtttgtcttcagtagatggttttgcaataaattctttttcagcaactccattaattatctctgatggatatataaatgtttcattaaagttagggttagaattgtataaagatataaattctatattatctttaaattctccttcgacaataagtttatatctagtaccatcttctataggctcatataaaccgtaatctaaatatttagaacctgtaacttctctttcaacaaatccatttttaataatgcttgtatttggtaactcctcatatattgagtcaattactttaattctattatcgacattaaatgattctgatgttagtagttcttcttttagctcaattattaatcgactagagcctaacgttaacggttcagtataattagtcaaagtataggctaacatatctaaatcagtataagcaccgacattaaatagagtagccttaataatttcacctgtagctccatcgaatactccgccctcagtgtctttaaccattctcatagaacctagctcaatatctgataatttatctatatcctgccatacagcattaaagtaatcattaggcatattatataaagaaccttcttgattgttaaattcacctagcttaacattccaaatggaacgtaatgatactttagacattggatttatattagaataatctactgtctgtaatccatataaatctattaagttattggtttctgttgtttctccagtactaatatttcctagagtctttagttctacccaactatcaccatagtaagaataatataaagttacattttttaagatagtatccgaattaatacttaaagtctgtatattagttaggatattattaaatttaattcctttttctattacgttaggtttatcttccggagcaggtgagtactctagcttagaagaaccctcagtcaattttaattccgtaattttaactgtattaaagaacactggaggagttgatgctttaccatcagatgtatatcttccagtatatattagtaatctatggtcagtaatagtaggggctgtaaaagtttgagttacggatactttgttacctatagtacgttccatagatttaacaccactaacaacaattcccttagtattactgtataaatatataccatgactatcagaataagatttaagagttgggtcattacctgttatttccatatcaaaagataacgtatacactttacctggttttaatactttagaaagataattagttgagtatatctctttagcccatcctgatacagtaaactcttttgtagaagttatttctctagctatcgtatttacacctttataagattgtaataaatttctattctctactgtattatatacatctaattcattagattctatagggttattatctgtatctttagctactatatttattgtgctaggtattgtattttcataattaccaactagttttaaataccctataggctgaaaacctgttttagttataatatctccattagataatgatgctattttaaatgcttctacataagtctctctagatatatctaaataattttctattaatgctttatacggttttattgtataaatacctcgttcatagtaataaaacattaaatctaagtaattaactttaatatctactgaatctcttttacctgcatttatctctaacctagtaaacatatttaattcactgtttagataatcttttatatatcctatattgctcccgatatttttttcatagaaagataaattattaattgatactgtaagccatctattaatagaaaaatcataaacttgtagtttaatttctatactttcatcaggaggtatcacagcactaagatagtaatcaatacttaattcttttatataatcagatacaattcttctagcctcatcagattgtaaatctacttgaggtctatatttactcatatagtccctaaaattaaagttattatataaataagacacaccggaagtagtttgcatactaagttctactgtattgttattcttagtactagtatacatataatagtcagtaggtacttcttgacctctaccttttgtactagcgcttatttgatttacagaagatgtcatacctggattataaacatatgatgttacatatccgtagttaatatactgtctccctacactagatgaacctgttaaaacatctaaactattaattaagctatggtttgttttaaaaatatctgatgaactgttatcagtatctttactttgattcatattaatatgaccatagaatgtatcatcataacctgtaaatctatcaaactcttggtatgtttctattttaggtaacccatctaaccacttaataattgctccacctctaatagtagaagccccatcataagtgacatatagagttacacctgcaggtttgaattcattaattacatctataatctctacagggaaataatcacctatagagacattaataacagcaaatctataatagtatcccattaagtggtcttcaccatttaaatgtgatttgttagtatagaaaatatttgtaaaaggttcatatacacttacatcaatatcatctctacctaaataatcttttatagcatctattatagcattattagttcctcttttcaagagtaaatattttataattcttgctctataaacatcatctttctcatcggtcttacgataaactccgaaccaatccccaaacttatctaaatattcactggtagatgtctttagagaagattgtaacttactttctatagcatctttctctacttgattcatctcttcattgagtgcatctatcagagcaaagttagggtcttgattatcttttttatttctatctcttcttaataatggatgaagattctttaaaaaattagccacgacttaagtttcacctctctttactttagttctacttttatttctcctgctctaataattccttgtggcggtactataatgttctcatctaggttatcaaatgacacatcatatattaatacatcatcaatattcattatagcttgaataaggtctgtaattattaggtcatcagaagtttttaaattatttaaatagcttctaataacactttcgatatgtttttgtaatgtatcaccaattctagatttattagatatagttactgtagcagaaacattaacttcttctttttctacacctgtaacatctaacattataccacttggtctatagtcttgtaaagcatcaattatatcttcttttaaggtatctgataaattaccgtttctatcatgagcaaatactgtaatatgtcctgtttcttcataaacataaacaccttctacatcaggtatctgcagtgcaccatatcttactgatttattagttgctctacctctagattctacaaaagagtggaatctgcgcttaaagtcttcttggctctcttctttagttcctgtattaaaagaatactcgttattaacacttctaatcaaactagaacctgatgctatagtattaattgttccttcaggaacattacctgcaacccctgtttctttacaataaacttcaacaacaatctcagtagaatcaggctctgcataataatcaactaatgtttcaaattgctgagggtattcttgtcgtgttgaagtaaaagttgttcctgcgggtatatacattctcatatctaagggttggtagaattgaatagtaacatcaccataagctcttttagattgtcttttttgaaaatcaaaagcttcaatgataccttcttgtataccccaatcaatattttcttttgttagaatatagaattgctctatctctaatgatacagcttctaataatgaacgagaagctgaaccaggagtaaagtctgttatcttgcttgtacctgccattgtcttatctattaattttgatagtatgttagttaattttctagttttcataatttaatcctttcctattcaaataaagcaaaaattccttcctcatcttgtcctaagacaaaatttattgattcttcaacagattttatttccactgaaaattgacctgaataaacattaccttgaattttccaatcaattagattagcagattttactctattatctgatgttaatgttctcaatacttccatttctattaatgtagcttgttcaggtatattaagaccaaataaattatgcaaatctgaaccgtaattaggatgtagcattaaagaacctctaggagttaataaacgtgcctgtaattgctgtttcatattatctatgccttttacagtatctaaatctccattaccatttgtgctgaatgctagtatattatcactagtaccatgttcattaaaatacttttcatctgcagtaatatttaaatctcttcctaaagctaattcaactagtacatctttatctctagaattaatttcttttgctgatacatctgttaaatcagattctataggtataatcagtgtatcacctgtagaagccaatcgttcagggtctttcattttttcttcatcagtttctactaaataggggtactttaaattattatgctctactaggtctatccaataactcacatcaccatagtatctctgtgctattgcttgcatcgtttcttcatgttgaactacgtgcttcttaaatctcattatacgttagcctcctttttgattagaggtaattgtaattctatagcactaaaacataagtctaaatctcttaagtctaagacaacactattatagtcatcatagttacctaagtagtcagctacataatttaaattttcccgtgttatactaacatcttcttcagttaagtattttaaagagctcggattttctacaaagcaattaacaatagcatatgcttcaagtacaatcgactgtagaatcatatacattcttggagcctgtacttttagtaatgtatcgttagctttatggataattgtattatcattaaattctctttctaatacaggtatatcatctgtctttatatttgtcaatactttttttgctaattcacttaattgtagctgtggtgtataaaatttagatacaaatgatacctcttcatccagtaaattcatatgagaagaagttaaatctgtatctagggcatttaaaaatctaagtagaccttgaggttgtggtataaaattattcattagctagatacacctccatcaccgtaacctattatcagcgccatattgtcaatattaccttttaatccgttagtagattgtctaggattatatatagaactactagggaagctttgcttaagtctattaccctcgttcgtattatctcctgtagatttaggtattgtattttctcttctagatatttcttgattgtttctatctctcgttttacgagcatttttatctaattcttttataccctcatctactctttgagaagcattaggtttaacgttaccaaactcttctgttgttacagcacttctatctgcttctgttaatgaaccaataactactaatgttatttcatatctaaaaagtaatggttcatctttagacctagttatctttaacccctgaggagctaaatgaactttataataactatcatctgtaaagttaaaaaattgtaagtaagaaccacttacattaccactgccaccttgcatagcatattcactaactctactttgtaactcttccattttctgcttacctgtttttaatccatctgcttctctaacaggtctaaaaccagttgtacctgtgaagttaataacttctatgtctttaccataatcttctattacaatatctgattttgttttaattgctgtcgtacgttgtggtgaatctattgtgtagttctcagggttaactttaaatctatacatatcataaccaccgccttctttagggaagcgtaaagctattctatgaagattacttattccatctgattgtggcattttatcctctccttttcagtatgtatataaatctcatactactaatatatcacgaactagtttaaattacaactaaaaaaagacctaaattaataggtctttaatattaaatctttttatattcatctaggtagtttgtaaagaatccatctacacccatttgtattaagctttgcatttcttcttttgtgtttaccgtccatgcatggactttaagccctttactatgagctaaatctactaattctttagttatagttgtatatttaggcgcaatagcataaaaaccataacttaaacaatcatccatttcactttcagaaaatgtacttgttaggtaagctaaaggtatattagagaattgattatgaatattaattaaagattctctagcaaatgattgaataattacttggaatctctctgaacctattcctattaatccttttgcttttaattgagttaataattctctatccatattagcatcaaacggacgtttagtttctatataatatttaacttttgttccaaaatgatttagtacatcatcaagagatggtatcggctcaccattgagagatgtaaagttagtttgtatataagataaggtcatatctcctaccttacctgttcctgttgttgttctgtctatagtagaatcatgcatacacaataacttattatcttttgttaattgtaaatccaattctatataatctgcacctttatctaaagccatttgatatccttttatcgtgtgctcatctgtgtatccggttgctcctctatgtcctataagcataaagtctttctccatagtttcagtttctaattctatagcttcaatactaaaccatgtataagggtctgctctgaattgtctgtcaccttctgagtcttggaaagcttcaaactcaaagtaatctccttctttaacaggaataactccactagtaccattctgaggtgctgtagatatagctacatctcttgtataaggtaatcctatactataagtaccatttttcaatattctcaacatacgttgtcctgtggcgttagagtcccacaaaacattacctgcaactcttacttttgttattcctttaggtactactaatcgagtaggattattagaatcccaaaactctgcattattataagtaactttgctccatggtattggcatatattttctcataggtactgtgacattttcctctaactgtatcatagcacctatatgttctcctctatctgagattattttatcagcattgttattattgttctctagttctgttaatctagttttaatatttggtatttcagttttctctaggttatcaactttagtctttaaggtcttaacatctgttttcatagtagggaaattctgatttacttcattaataacagtagaagcatcttcataaaaatctttaaaagtctcatctctaaatttttggaactgtgttataataccttcagttctaccactcacttctgctatctgagttgtaagtctattgacatctgaagttgcttttttagaagcttctatagactcttttgtagactctattaattcttctaaattgtctttacctacaccctcgagaaggtagtttatatcatcgagttctttttgtatttcattcaaattcttcatagcctctgctatactctcatctaagttttctaacataggtttatcatctatatagattccctcatcagtaaattcaaatttatgcttgtcatttttaatataaaatttattatcttcactaataccatactcaatccaaacttgagcttcatccaacattaaatcgtcatcttgtttgataactctataagacccatcacttgacatttcctgtgtagttcttttctgtgtttctgtatttaaaactgaggctcttatatcccctctttcagatataaataaagtagttatatgattatccggcgtgccatcatcataaaaaactccttgatgtttaaataacatattaggagccttttgtattctaggctcaattaatgtcttattaccataataagaagtaaataaatcttgatactcagttccagtataaaaatctgttgcctgaggtttctcttcttcacctgatgtcatagagaaaaatgatttaccattataagtcctaatacttgttccttctccatcatcatatttataatttaaagatggtaaaatttcatatagtgaactactatatttataaacactttctgtatcaaacttacctccatctagaggattggtattaatcattttattttgttcattatcaccataaacacttaaaataataggactatttatatcatcatttagaaaccctattaatactacagaaccttcagtaataagaggttttgtaccgaatacgcttccttcaggtgttcttcctatgaaagatttaggataaggtacagctaagctaccatcatcacctatacggcttcctagagttaaattgttaactttaacttctactgattgatatttataattaattttactaactgtagctaaaactaatccttttacatttaaatcatccgaatctactctttttaaacttctccccatggaagattggaatcttactaccattaaatatacacctcttcatcgaatctcataactcttccgttaaattcattccagtaatcatcaagtttatattttcctataggtgtttcatctttagggaatttacccatcatagatataaactccccatctcctacataaatacctatattagtatcacttctaccaaagaataatatatctccaactataaatttactaacatctaccttatacttatgtcctttaccgaatatagtaaagaatcttccctctgtaaagaagtagttaatattcataggatatgctaactcttcatcttcacctaaatataaccagtataataagttataagtatctatataaacatctttatctgttccaaatggattctctttaggagcacctttgaacatatactttatatttgataaggatttaagattatcatttaagtctaatgctagtatcgtacttaatggtacatcttcatcaaatacatattcttcttccatatattctccatacaacacatcttgtatctcatcaaagtaatctattaatagttcttgatttcgttctactaaatgaactgttaaagtagataaagaatctgtaggtatagaccctttatcaactaaataacttactttcctgaaacttaattctgcctctatatcataaccattattagaaaatgattcatactgaacatgctgagggttaaggctttgtattactccaacataaggtcgtatatacacaaccttttcatacatatcatctgtagttatttttttaatttcttcatctttttcttttattctttcttttgtaagtacatcaaaattaaatacattgtctataaatttatcaataacatctttagcttctttatctgttgccatagactatcctcctttatttcttatcgtaaatgaattgtgcattcttacctatggctgaactcatatctatcgttctagtaccatatccattaggggtagcatagttatgttcagagataaatatttttttacctccatctaatactttctctacaaacgctacgtgaccatattgtgggctacctccttgaactcctctttgccatataacacaagcaccttgtttaggtgttctacctacaccataacctgctcctttagcaccacctatccagtcggcggcgtccccccataaaggcacaggtatgcctaattgacctcttctattataagcataccatgtacactgatacttataatgtcggttaccaggttgaacaaaacttgggtcatgcttaggaagtttgccgttatattttttaagtgaatctaaagagccaccatcttgagctccaccactatcagaaccaccatctttatctccactactttgtttctctgctacacctttttctttaagttctttagaagtatcttcacccataagacctcccatgaagtctgatgattgattccataatcctgcaaatctatgcggactacctttaccatcttctagaatagcgtcttttaaacctctagttactcctacagttgtataatacccctgtttataatcgaatttatgttctacagattcaatatagaactcccaagtgtctcctcgttgcttatcttcaataaataatcttttacctaggtcatacttagggtctcctaaaacaataatatctcctgcatagaagttagggttaccatgataccaattaaatagcattctagaaaacataactaaagggctatcacttgcatctgaacctgtgtctgtagctacattactaacaccttcaatagctttaagatatttatcaaaagcctcatcgttaggtggctctcctttatatttaatatattcatctaataaagttgtagcatgtgttttattaccaaaacgatatttagttgtcaattctttaagtgcatctgtcttagcttttttctttttcttagaattattaaaatcatcttgtgttttaaacttctcttttagtatactctttaatttatcttttgtcaacttcggtctgttatctgatgttaattcatcatctaccttattacctgttattttttcatattctttttctgttacttttccttctttaacaaacttctctataatttttttagcttgggctttttttaagtttttatattttgaagataatttacttgtatacttatctatacctttagatatattatctcttccatagttacttaaatctttcataattttagaataagttcctcgttctgtgccattatcatcacctgaagaatctgaatcctcagtagctgaacctgattttgtactaagataaatattttctacttcaaatttagtataaccatatctatcagttaattcagggtggaattgtggtttagaaaatacatcaccgttaagctctttcaacatacctgcaggtgttgctgtaaatatagaatatgtctctacatcacttttacctacatcctcttctataaaatcctcagtaggtactttaatcatatctaaagctctccactcagtaggattaaaaggggtctttcttaatacaagttgagcctttccaggtgttttttctgaatttttgaagaataactcattaaaaggtctagctgttaccatatccattaactgttttaatgacccatcaaaattagtaaaggctgaaacttctgtaagtttttcaaactcatcccaactacttaaatcatcataatcaagatagttatcaattgtattatatgttttttcagtatagttatatttcatataaggtataaatctacgtataataccagtcattacttcatgagctgagctaccagtaaattttacttcattatctccatcaccatcaataagccaacctacttcaggtaatacagcttgaacttcctgaataacgcctaatccaaatttcataaaaggttttacaaaagattgacctgttattctaaattgagtttggtcattaccgtaactacctacttttgatacttgagaaaccatacctacctggataagtcgttcttgttttccttctttatcgttagggtcatcattaggtgtaataaatattcttatgatatcattagccataactagcttatcccaataagtatctcctgccatatttatttggaaaactgaactatcatcttccatagagtttttagtttggaatcctaaaagtttagctccaaactcatcaccattatagtcacgtgtatcttcaaatctcaatgtaaatgtattatcatctgtaacgatttctattcttaccttaggtcttcttattctacgcatatacacatacttccttttattttttatatgtataagagtagtatattatgatactactcttgtgtcaatacaaaattactaataacttctcttatgttcatttgcaaagatatccatattactatctagaacttctctaattcttcctccgataatgtctccagtttcttcaggattatttccaccttgaacattaacattgactgttattctgttagttgtagaagcattagaagcatcattagagcccctataattagaaccagtagttaatacaggattactcatagcttcttggtaagtagaggatagggctcctccaccacctccgccaccggagcctccgtatttcttcttgaattccttagcattgttaacacgagtacccatcatagcctcgtttgctcccatacgttcaaatccagtagcaaatgcttttgtattctgctctaagcttccacctttgctccaacctgcatttttaagattattgctttcataatcagactgcatttctttccataggtaatctaattgaacatccatgtcactagattttttacctttagatttagcaaaattttctaatcctgtttttctagcccctaaccactgagcaataccaaatgctccactagaagcattcttagcattagggtcaagattagattcttgttgtaagttccccataacagctcctacctgattatcagatagtccttttcctttaaggaacttgtacatcttctctgcgccttctccacctacatcagatgcagagcctccactatcagaaccactatcagaagttcctccatctatattaatacctttagcactctcaataattttctgagctctgtcaagtaaatcactatagattttaagattcttagtttcagattggttattcttttctctaagttgttctgctgtcattttttctttcttcttagtatctcctttaacgcctttaacaccttttttcttatctttactatcttcttcggatgcttcagcgtcattatcaaagagacctgttataccgctccatgctttagatataggttttcctaataaagaatcttggaatttattcttaccatccttatctccgttagtccatcctaggttccatccttctttagtgttagcttttttagccatgtctccgactttttcaccaacagctcctcctccgaaggaacctatagctccacctattaaagctccaggtattgcacctacaccaccaaataaagctcctatagaagctcctgctgatgcacctgccattcctccacccatagagcctattcctccgccgatagctttactatctccggaagctaaagcaggtattaatgataaggcagacgtagcaacacctaaaactccggctccttttccaagtccacctgcaagttttccaagtttccctatgccacttccacctgcttttggtgattgtgaaagaataccatttttacctccgtcaccaaacttatcagcaaacttaccatagagacccttagctttgccaccaaaacctttagctttttctcctaggttttgtcttctttctttaccaaatatactatcattatagatatctttaagaccactaccggtacctttagctatatctttagatttatccatggcttttctaccattatctctattaaatacttttctagtacctgaccaggcttctttagatgtttgtttaaagcctttacctttagactccccgccaaacatacctttccatcctgcgccacctactaggaatcctccaagacctttagcgctatcaagtatttttcctccagtagaacgagtgcccccacctttaggtgagcctccacccttaggtgaaccaccaccactaggcattccacctgccatagggttaccaccggagctaccgcctttatttctaccaaatttatttctaagtcctttggctcctttaccaattaagtgacctgctccaaattgacttgcagatgcaattagtgaagctgtaaatgctcctatagctcctatagctaaatatataggggcaggaagacctgctaaagcactatgagcatctcttagtggttgagccatatcataagtatcttctgctttatcatcagtcttcgatttattttggtcattttttcctgatttagattctttataatcagtggcgttatcttctccttcttttttaccttctttttccattttcttagctttcttagctaactcttctttagatagttttccggattgagcatctttaaatatttcatcagattgctccatagttaggttagcgcctatttctttcatacttctattaaataggtatttttgttctttttcactgccacccatttgagtagctatatcagccatatctgtcaagttttcaggattagatataccttcatccatacgtttttgtaaatcatatccaccttcaagaccttggtattgcgttccccatcccattgctatacgagcataagaactattcataccatttttaagtccttggtctatactgttaatagcattggcaccttgttcaccttgtaatcctttacttcctgactctgcaaaagtagattgcatggcagtaaggttactcatttggtctttagttagagttcttccttctcctgattgttccgctatagaacctagtgctttaagttgttcatcttgacgaccaaccatgcctgattgtttaataccacctaagaatgcatcttgcatttccttcatgttatcagaatttactccaccggtatgcatgatttgacccatagactcttgataagcttcttggtctttaatgcctaaagaacgtcctccaatagcaagctgtttagttcctgccattgtgtcctcatcacttttatgtcctactgatgcttcatatgaagtagccattttcaacatgtcagtactattataacctaatttgttatcaattgacaagtctccatatgcatttcttacagaatctatatccatattatcggaattttgacctaatgatgtaaccatcggtctattagtttcacttaatgaggcacccttcatgtataaacctgtagccatagctgtagtacccatcatagcatgagaaccaatagatggcaaacgttcttgaaccattctaccaaatgttcctctttgttggtctacgtctctagattggaaatccttttgaactgaacccttataatactgaactgttttatcaagttctgctcctaatttctcatagttagatataagttcttctctagctttcatttcctgtttaatagaagctaactcttgttcatattgttccttattaatttgaccgctagctctacgtttagtcaagtcttgttgttgatttctatagttgctatattgctctcttgcttgtcctaaattacgttggttttgttttctttggctctcataagaacttattttacctagctcacttctaactcgtgcaccttggtcatatgttaatcgtcctgttccttctgaacgctccatgtaacctctagtcatattacgattagctcttaatcgttttgtataatctttagttgcgctagaatctttacttattttattgtattcttcagttacttgatttaaggttttcatttccttaattctattttgaatactggcatcagaacctattttttgtgaagacattttctcaagtttttcttgagtttcatctacaactttacccatcttagcatatgctttttcaacttcagggtcaggtgcaatacctttagatatattctctttttgaaggtcatctaaagctttctttaattcatttatattctttgtagcagattgagtggatttaatagattcatccatgtctttagaagatataacagatttaccatcttgtttatccattgcgttctgtacatttttaacttctttagcaattacttttaatttttttaagaaattatctaaaggagcatcaatattcttagactctaaagaatccatataaagttctattgcctttagactattctctaaatccgaactatcaccggacaagaccaatctataatcgtcattcattgccatcattattcaccatccattttaaaaaaataaggatggtagtatcacctaccaccctttacatataccagtcatcatctaggtcttctagtgctttattaacgctatcttgtgatatagtagcgtcatcttcactagtcttaccatttcctacttcatcatctaattcttgttgtttctgtctgatgtattccatcatagctagatgttgtgtagtaagtccttctgtttcatcatttaactctgcatcgtttctcttagctctttcttccttatctctatcggataatttagcttccatctgttgtgctaaatcatcagcatctaagaaatcaggtactgggtcaaagtcttcatgagattcattccaccaactactatcatcatcttcgaattgactatcgaattttccacctttttgagcttgtttaagttgtttattatgttcataaacatctctattcatattcccaataataaactctatctgataatcgtctaagtccttaaatctttgctcggtaggtaaaacattaaactctttcataatagcccatagatttctagataaaggttgtttaacaattgattccataccgcccaatcgttctatatctttatctaatgtttctatactattagtaacgaaaggagtttaagaatcctaaccaatcttcatacataacagttaaaggatataagttataaatatcgtcagggtcttggaattccttaggaacttcaatacctacttcctgtaaggtagctaacatttgatatgctctaatcacttggtctgtttggtacatatccataccacctaagtaagcagaacgtaacgcaaagatattaccttgttctcgtgcattaggtaatttaatcttaactttaaatcttaagtctagttcttcaaagttatattctttttcccaagtatcattaacgcctttaataactttgttaataacttgttctttttcttgtttgtcttgatattttaatttatctatttcttccggtgtcattttactaacatcttttttttcttctttagttgtttcttgtacatcttgttcctttttaatttcttctgccataatatgatataccttccttttttaaatattaataaagcctacgcttatccgattcaatattaatataacacggactagtgtaggcttactactttaaacatagtatatcatattttgtttttaaatcaaaattactttacatattcacctgtacgcttagtaatttcttgtaaactttgttcaatcttttcactatttctaagtaggtaaatatagtattcttgtaatgaatcataatactcataatcaatagttaatatttcttgaccttctaaatattttctagtaatctttcctgcttgttctgttaatgttttgaatttatctaaaatctgattgattgcaattgcagttgatttacttgttatatctacctctttaggaaatttgtgtaattctactttcatttctgtgtaaattgctcttaatttatcttgtgacattttatatcttctcctttaatttgttatacctttattataatttattagttttaatttgtcaatacttttattctagttctactacataaattttagtgaccaataaataccattaataattaaacatgctaaaaattgaattgatgttattactactaatattttactcatagttaatgattcccttgtaactgtacccttaccatttaatatggttaatatgatgaggaaaatgctagacagaataaaataactatttactattgttatacccattataatacctccttattgaattgtcaagtctttactataataatttaaatctagtgttctatgtgtatcatctacactgttataccacaccgtaataactctagcatcttcaagactaattgtcaaaaactgatagctaggttttccttctacagttataactttaggatgtttaacaacaattctcctctcaatattaccattataatctgtgttattgtactctatgatattttcgtaggcaaacttatttttaaaaataaaacctattaatttaggtatatttgttgttggtttacctttaaccttttttgcactatgtcgtgtaaaattaataattctattttcagtaattgtctcttgtaataactctacacatttctttctttcatacatactcatttgagtataatgttttttaacttgcctatctttattagttgttcttctttctgaccaaaatgtactcatatacccatctcctttttttgttatactaataatataccttagttaattataattgtcaagacctttatgtttattttttctagaatattttgttttatcttttttaactttagttaccggattaatattccatgttgctcttgtgttactcatttttcctattgtttttctcttatttgccatctagtacatctcctgtttaaattattatatttataataacatataaattttaaagtgtcaataaaaaaagaccaactttttagttggtctaatctattaagtacgtgctttgtcacttgctgttaagtaactaaactcgatttcttctgttacaatttcatttgtctgccaagtttcattgtagttatttgcagagcaaccatgatatgagataataacttgtttcgttaagttatctactactaaaatatcaatgatgtctttcttaagaatttcttcaccaagtgaagcatatcctaaatctgcaaagttttcttttttcatacgtaaacgttctactgtaattgtaccttcataacgtaagtatacgtgttcttgaggcatgatagaaccgatttcgtatacaccagttgtaccgtattcacgttgacctgatgctgattgtgctcttcctaccggtttacctttaatcataagtaggacggtattaccagtatggacggtttgtttagcttctgatgccatctgtattcactctcctaaagtattcaaattatattaataggagtacctaatccaggtactccgtcacctagactaggcttgtaatgtctgttgcttgtaaaccaagctaacagagattttcttgaagcttctgattgggtaaactgtcattgaaattcttgcttcgttaccttcaacaataacttgtacgtcttcagcagggaagtcttgaatttcattatcacgtttcttacgacccaagtaagattggataaagtctttaatgattgaagcacttgtattaatagtacgagtaccaataaactggtcttcaagttgaactttaagctcacttactaagaagtcattagcttccccaacagccatttcagccttaactgggtctgatttatcgttgaatgtagttacatcgtcaacgattctgaagaatgtattagtacggttacgaacaaactctatactaataataccattttcatttaattcatctaagtctattgactcatagatttggtctaatgaacttacacgtagtggtttgaatgtgattgattcaccgatttctaaaccacttgcaagaccacctagagctacggctaccatgtaagcaggtacgtggtttttacgtccatcatccataacaaaagtacctgagttagctactaatgatactcgtggattagataatgatgcttgtctaccgaacaattgttctttagattcattgaatcctccaccaacaatagctctcattggttctcctgcatcagaacgttctttaacaaaagaagctacctctgcatgaactgattgtttagatgataatggaacaatgtagtatccgccttcatgtgcaaatttatctaacttgtctgcccatgtagcaggtggttcaccattagtaccgccttttaacttagttaactcaaacggttcaatagttttaataggtgaagtagcagttactgtagctgattcttctcctgcttcaacctctacattacttggtacttctccttctgcattaagttgctcgaaagatacgataccattgtaagctgtttgtttttctaagtcaccaaaaactgcttttacatatacagctttatcttttatatttgcattttcaattttatctaatttgctagattctaagttcttatctccgaaaggtgataatttagcttcgaaatcaggtaattgattaatgtctgtaataatagcattagtgtagtcataagctccaccagttaaatcatatgacttaacttcttggtctccaacttttaatactaaacgacttgctttttgagtttcttcatcatgttctacagagaaagttgcgttagcttcttctcctttgtacttgattgtgaagatattaccgatattatcataaacctcattgaaacggtcatcttggaatattactcttaaacgtaatgaatcacttagtgtattcttttctaatcctacttgaatgttgttagcaacattaccgtagattttagatgttatttttaatccgccaatttccgctgaagcaggtttagcatcttctatacgcatagctaaaatacgtcctgctgtatagttagggttagaaccccatgctaattctattgcatcaagtaattctcctgaacggaataaacgtttagcttgtgaatagttacgtaattcataaactgtatttggttctccgccttcagcctgaccgattaagcaaaatactttttcacttgaacctgctgagccaccgatacctgaagtatctacttcaatagatgcatgaggacgggtaataggtcttcttgggaatggttctactgccatatttaattctccttatctgaatgttattatatatctaatttcctacctagatacttctctaatgatggtaagaattcttgctcactaaacaaataatgactaccttccatataagctttaaatcctgctacttgtgaatccctcatattaaaaagggtttttgcagtatcaaggaatgtatctatatgaacataaccattaaatttaggagtgttattttcaggtgtcttttttctagccatcctacttccaatcctttctttctttaaaagttagtttattaatatcttgtgtaatagtataatccaaatctagagaacttgtgtacttaataattgttggtctaccaaaaatcattgagtcaccatcttctattattggtgcaatatcaccaaaagacaaattctgtaattggaatgtttgttgctcttctatactatctctcatagatattaaaatcattttcagtacagcatccaaacatcttgcaacgtctacattaaatgaaagacctacaactgttacttgttcttcaactgtgaatccttttactaaacctttagaatcatttttcttttcggtaaatataatgttagcattgtagttctcataatcttcatttgtttgatacttaaatgataccttattaccttcaacctgaagattatcgtatttagcaaactctatatcttctacctttattaactctcctattggtttagaaacagtaaaaactaacttatcatcttctcttattgctgtagaagattcgactaaagtatctcctgttgcctcaaagtaagatgactgaatactccctaaagagttttttgtctcttgtccttgacctaattgtatcaagtaatgagcttcatagttatttttaaattttgggaagttaaatcctaccgttacttcatgttgtgcattcttaccacagaaagcttctttaaagctctctctagtttgataatcaaagtcttttaaaacttcatctataatataacaattctctagcacagtgttaagtctaggctttatttctgataataaatatgaatcaactgatgttattgccatttaatactaaccctcctcttaatttaaatttttcatcttccagttcattaattgcttaacagtttttagagttgttttagagaagttatcttcattaactttatctctgttaagtatccaagaactagcaggtgacttactagaaacagttctaaatataaagtaagaagattgctttcctcttttaactttagtcatattatgagtcataggttcaggcttcattgaagggtggcttacattcttcctacgtccttctaggtaatcagttatcttcgaaactgaacctgttcctttatctacttttaaagttctcatatcttgataagtactgttattcattctactagttttgatgcggattggtacagttaaataccatccgccatctttactagttttctttttagatgagtgagcaaaagcttttttaaggtctactactccttctttttcaagctttttctctgttatttgtagataagtaggcatacgtttaatactagtattctctacttgctgtgctgttgcttgtaatttatcaagaacttctgaacgcataccatcaattgttctcttacctacctgcttatagtttttatttttaaataaattaggtctctttccatgaactggcattatttaaatgcacctccgaagaatccacctaaattactaggattagatgccgaagccttagggtcatctacttgaatttctaggtcttcttctataccatcattaactttataagggtcttgtagtacaatgacatcttcccttttaagaagtaatttttgaggtaagttttcaaattttgatttaggttgattaaacttagtatattgataacgactttcttttagtatatctgatactacatatcttaaagtcataagtatctttaaagttactgtatgattctcatatttttcatttaaatacaatctattattcttgatttcataatcttcttcatagactgtaccgtcttgagtggctatataagttatttcttttacatcgtagtataaaggtataccttttttaattctatctttattcacaaaataaatcatttgttggggcatcaatacctcaggaacagtaaacctgtctctataggaaattctcttttctaattgagtggtacctattgcagtacctgtatctaatatacctatatctaactggttagttcctttctcttgagactgtattgccattatcgtctctttaggaggtagatatgcaatacctttaccatgacatctagggcaatctactctaggatgtcctgtatcaggattaagacaaggacagaaatatgcttcttcccatagtacttttatacctcggtcattaataaacctaatcatgtcaatcgtatcaaactctagtctagtagtatacttaggtttatcttgttcatctgcttgtgttgtagtagtatatgttgttgacttattaataacattagggttagagttagctcctatcatatacggtttttccattttctaaccctccttataatcctatcatattatatccaaagtaagctcttaaaccatctaatagttcttttatatcttcatttatttgaagtatctgagcactagctccaccatacatagctgattgagtagtacctattgtctctgtaataccgtctacttctaatgttttattagcaataccggcaccaataattaagttaccccatacttggtatatttctttcaatgcatattttataactaactgttctaactcagggggcatttcccaaggtttatttcttcctgcttttttacgtggaagcatacctgatacatattctaatcgtatcatttgaggtgcaaatgttgctcctgatggtgggtatacacctgctaattgagggtatccattgaatactgcatcgtatgacattgattgtcctgtttgcataagtgctgtagggaataattgaacatgtcctgctagatgctctactttccaccagttagcagggtatttatatataggtctaccattaaactgtagctgtaagttctctacctgtaatataggttttctgtaagcatgtacaaacatgtaactattaaactctgtctcatgataatctctcatctcatgttgagtgtcaggtagtatagatatatctaatgctctttcagctttacctaccgctctttccaatatatggttatagaactcgtcacccataggctgtcctgtatcaggattttgaactacaataccgaacatatatgctttaactgcatccggtgaccatccataatcagctaatgttaaattatctatctcacttacatctatgtgtttaggattaccactaggatgataaggatattcatagtttaatgatttttcataagggtctaagtcccctccaaacattgagttaaccatttaattcaccctttacttattttcatcttttttggctgttgttttacgtgctgtagattttctagtagtatttgtagtttttctagtagaggctttcttttcctcttccttaggctcttcttctttactagtagaagctgattgttttctagtagttttcttttcttcttcaaattcaaaacctggaacattggcaaattctttttcttgttctttagttaatcctttgactactccatctttgtcaatctccactgtaccataaacagtagccatttttttatctaatagtttcttatagtataacatatttattaaatcccttcaagtttattttaataaaaaagggataaaccctaagtttatccctattctcttgtattcagtttttcttattctattaaactgcgatataacgaacgtttttaatacgagcccattttttaggagcacgtaatgctaatgcaccataccataatactgcaaatgtaatagaagcattaatttgagctaatggtaatttcatcattggaagtaattcgaataagtgaactacttgtggtgacatttcaccaacaaatacgtctgctgtttcaggcaatgtttcgttcttatctacgaatacgattgttccgtcttcttgtgcatctttaactggtacacgtttgattaggaagtacatacctgtttctttaccttgacggtagatagaaacgaattgtggttgttgttggtacatagcgttaacattaattgaaagtttaacaccatcgtctacgttagatactgtagctgttacttcttcagaaggagctgattgagcgtcatctgagttaactactactttatatgataatcctgcacggtcttcttcattttcaaaagcacctttttgcttagtttcaacagtagctgtaactttagcaggttgtggagcatttggtaatggttgtaatgattcatctaagattagttcattttccattactgtagaaccatgtaatttaatgaatccacgagatgagtagaaaccatttacgctgtaaccagtgttaacgttaccgctgttgtcttgcattaattgcatttgacgacctaagattgagttaacgaagtctgcgtgtacaccgataggcatgtaagcatctgtagctgtaccgaaacctttaccgatacgtaccgccgcctcatttaagtgtttctcagttaattgattacctttagcgttaattacgttatttttgtcaattaatttagctaaaccatcaaactctagaccttcaccttcaacttcagaagttaatgaagcgtcaccgtagaatgaagcccactcaattgtttttgcaacaactgcgatagcatcttctgtaaggatttgtgatgggtcagcaatgttatttactaaacctgatgcaattgacatatttttagtatcagaaacgtatttcattgatacagttttttgacggatatttgggtcagatactggtgctactccgatttctttaacgaaacgagagtgacctacgttaccatgacgtaaatattggtcgtattttactactgtagattgagcaggacggcgtgagatatcacgatagaagattaagtcttcattagtccatgttaacattgtgatttggtcatctaaaatttctctacgtaaagctcctgcgtcaatttgtgtatcaggagtgattccataaccagtttggaatgactttactacgtcttcttggaattggtcagcgtacttttgttgaacgtctgacaggttcttttcgatagtcatataaatttatcacctttcatttgtgttctttttattttatgtagggtacagtaaaatgtaccctatttatattctttagttacaccgtataatataacacaactttgtgcattaattaaatttgtgcaaagtcttttacagttttaatatctttctctgatgcattagtagggtcagtgttaatgttcaagtaagattggtaagctgattgtaagtcatgtttagaagctctagggtctttagcttgtgctttgtaagaatccatgaacttagttcgttcttcttgacttaatcctttaaatgtgttatcttcttgtacttcttcagcttcttctgattttgcttcaccagtttcagcttgttcttcagtgtctactgatttagaaacgtaaccttctactttttcttgctctccgtttgtagatgttactgatttttctacagcttcttcatcagtgtctactgatttagaaacatcttcagcttggatttcagaaatttttgctgataactcattaatagatttactaacttcacttaaatcttctttagtagcaaagttttctttctctttatttaagttttcaaaagatgttaagatagatttaaatactgttgtgatatcttcatcagaaattgatttagaagttttttccttcttatcttctttcttatctttatcatcgtcagtatctttatcttcatcgtcagaatcagaatcttcttttttatcttttttatttttacgtttctcgttgtctttattttctgtcttagtatctttttggtctacagggtcttttgattctttagctgatttttcaacttcctcatcttcattgccttctgctacctcttcagattcaactttttcaccttgctcttgtgcttctttagcatcttcctcagatactttttcaggttctttagcaggttcttcagttgtttctgctacttgctcttcggtagattctactgtattcttttcatcatcttctacagatttcgaaactgcctcttgagcatttaacttatcatactcttctaaaatattttgcatttctttactcatagaatgtattctccttacctattctagtttttgtttgtttatatccattactgctagttctgcatctttacgagataaacctttagctagttgtaacgtaaggactgattcctcatatcccatactattagatttactcaaatcttcaacaacgccattccatacatcattaaactcttttaaatctttaatcttagtgacgtaagctaaatttgtaatgctagatgctatttcttcttttcttaaagctcctgcatcaacttgagtgtcaggtgatgtaccatgaccagttaaaaatgatttcataaagctttcccatgttgcttcaggattagcagggtttttaactaatgcaactccggttatcataacttcatcaataactcgattgtcatttatattacgttttttaactgcaccttcaatagaaaaacctaaacgtcttccactacctgatttttctaatttctcagctaaatcaagcatcttaacaacattttcgtcattcttccatagctttgcttcaataaataaacctttttctatatccacatagcaattctctgtaggtatacctacaactttatcactttgatgctcataattaatgtacccttgtgacttaaaatactctatatctattcctttaggattaactatatcattctgtaaatccatacttggagtactagcccatcctgaaacgatagaatactcattagtatcagaagctgttgatacagatttcttcaaatccataggtacaaaagcattaaattttatttcttccaaaggtgtctacacctccacttgttattttatttacttttacgttaataatataacaataataacttgtaagtgaaatattaatattttttatgtattaatatcaattacatatagtatatcatagttattacatttatcaaaattaaattacgaaaaaaataaacaaagcctagatttattctaggcttattaattaatgtttaaagtcagaagatttttctccttttcttccttgacctttattagatgtttgagtacgataaacattatcgtcaccttttatttgtgcatctgttcctatctctttatcatcattactagaatctgttgattgcccttcttcagaatcatcattgtctccttctagtaaactcatcatcatttgtaaacgttctttttgtttaccatcattatattgtttatcttgttgtaattgggctgttccttgtaagaatgaagcatctagaataatgtctccaccttcaataggtttcttaccttgctcttctctagcctcattaactgttttaaatatttgagtctctagtttaagaatattaagtttatcagtagcactcttagtatctccacctacgaattggaatgtatacttatctccatattctgatataatatgtctattaactaagtcttcaataaatctaagtaaaggttgtaaacctttattttgagattgttgttgttttttacccgggtcagcctcatttaaagtagaaccacctttagaacctgtagctcctcctctattagggaaaccaatttctgcagggtcaataccatataaagcagatataatattgataaggtaatttaaccatttctcaaattgcatatcgttagcagtaggtgtcatatttacaaatttaatatcatctgccattaccactggaatttgccatgaaccattaatacctgataaactagatttccattcacgcttaaagttctctaatgcatgttgtgattgttgttggtctgaccgtatctgtaaaatacctctagtagtaccaccatgtgagaaaaatctatcattaaatgattccgtattattgtaggcaataaattctttcatagctatttctacttctgataatccgtatcctgaagaagataattcagttctagggtttcttatacccatagctaattctctagaagtaaaactagctactactcttttatctactacttgaacaaatctcttaccacccttaataattttaccttttttatctgttgcataaaaaatagtagaagggtctactgctatgaatttttctaatttagttttattattcttattaaatactttttcaaagttaacttggtcatagatataagtatctctaacaattttcttacagaaagtttgaaatgaatctctatctacatctttatctttacctgtattaacaataaaatcttctatgcgtttcatttcctctttttcttttctaccgggttccgcatctaggtctcttaatcttacctcaaaacctaaacctttctctgaatatcttgcaggttgacaatacatagctacttgatttgaacgtgtaagtatgatagcattaaggatagggttattaccaaactttttcaaaacatcatgtaagttatgttcgttcttcatgtaacttctcttatctctaaattcaggattcgtatccatcatttctataaatggctctgcataagcctgttgctgtccgtataaagacttagttaaatcttgatactctttattgtcttgttctatttttttaatgttagcttgtaatccttcatcaataggaacttgagcaatggtactattattaccatagtctttacctaatctgaattgcttaaataaatctgccaattactagtcacctctactccctcttactgtagttaatacaacagtattattataagttacatctctaatatgttgagggatatttattttaacctcaaacggttcttcaatgtaataaaacatcttcttactttccataacaatatcataatcttctaatagtatattattatctttatctactaatattactttattgtcttttgttgcttctagaatcatgtattttactttgtttaaagcaataaaaacagctacgtgttttccacctatctgtctataataatctacaatctctatccaattatcttgcataccttgacctttgatgaaatcttttgcttcatcccattttttatctttagagaatatcaacctattcaccgtcttttttattttttatctttaaagttactttttttaataattttcatcatcttgtaccaaattcctccaagtattgcacctataaaacttataataaatataattatagggtcattgaatccaatataatgttcagtattttctatggaatctattacatcattcttaataagtacagatgttgtttgaaacatagttaaaactatgtatgtacctattcctattatagcataaattacatacaaaacaaaaacgtttttactagagaacttactatgaacaaaaatcattaaagttaaaagacttaaacaggtaaatactactgatagtgacgttataatattcattcctaacaaagttttcaccaccgtaatgttcaatttttttatatttagctcttatttaatatagcaaacaaaaaaagaggagtaattactcctcttcagttttatcttcattatctatgttgttatcgtccggttgttcagtgggctgtttatcatctgtagtatcttcatttttatcctcttcgtccactttatcgtccggctctgaaggtgactcaggttcagttggttccgtttcccatcctccattttcatcaggattgtcagacggttcagatggttcaggttcaggttcaggttcaggttcaggttcaggttcaggttcaggttcaggttcaggcgtaacttctccaccatttcctcctgtattaccattatctgaaggttcttctcctgggtcaggtttttcttcccaatctcctcctccggtgttaccattatccgaaggttcttccgttgatggtggttcagaatttcctccaccaccggagcctcttccaccatcttcatctgtgtaccctggagggggaggatttggattatctgtatttccttgattattatctcctgggttctcaggagatatcggaactgaaggattatcaataggaggtataatatctgaaggtatagttggttcaactggatttgttataataggattatccgttggtaagttaatagaaggttttgtatcttctactatgctatctactgtatttggataaacattatcgttaataacacttaaaccatctatactatttgaaccaggtgataggaaaggagaacctaatacactagtaataatagaacctactgtttgtttttcatacccactatctacaggaacagttatcgtaccaattccatcttcactaataagtatttcgggcttgtctactttatcactaaactcaatattattttttactaatattaagtcatcatcaccagttacatcatatattttctctctagtttctttatcttctttttgtctttctttacttgacttcttattataatctttataatttgtttctttttttacttttttaagttcttcttcagccttatttaatcttttaacatatttctttttagtctcaccatcattatcatctgcttttttataaatctctactaatctttgatatctatctttatcttctttagcatcttgatatttattaaaaaatatggtcataatgactacaaatactgctatcataaccgttattatggttatgattacctcattgccttgtggtttctttttcaattatgatacctcattttatatagtctcataattcttcccttttataagttattaagtttctcttcttcttcttttctaattcgttctttttcttcaaagtcttttctaagcataaatgaaatacttccttggaactcattgcctgaaaggatgttacttaatgctgtatctataatagctacatcacctaactcaattgctttacctgcttctacatcttttctaaattgcttattagcttcgaaaatatcaatgcgttttgaaaaagcaagtttatacatcttatcatgtaaatctcctgttaaagggtcatttaatctactttgaaagaccattaaagagccatctagacgttgagtttcatccatagggtcttgtctaaatacattataagacttgtatagtctatttaccatttcacataataaactactaaattctttaatactatcttctttctcctctttactaagtggagttattgattcttctaatgttaatttattttcttctaaagtcatttatttcttccttccttttttatcttaccttcaatatcttcctgtaatatttttaataattcagttgtgttatctattctactcatattagactttatataaattgtttttaatacaatatataaaataaatacaatagaatagatgactatagtgctaaataaaatcactataatcacctaagaattgttttacatctttttttcttaaaatgtatccatctttattaggtttaaatcctaaagtttttaaatactctattgcttcaggtaattctatgtcatacacaactgcatttttattttctttaatacttaaacgtaataattccataatagctcttgctcctacaccatctgaatcaggtgaacttgcataagattctaatattatatcattttcagttcgtttaaaagatgtaattcctattgtatcaccgttatcatcaacaaactctttatacaatgatgagtttgtattaatatgggatacatctacaccttttttacttaaattagataatgaatttgctacttttttagggtttacctttccttcttcaccattagaaatagctcttgctcctactgcacttctactaacttcttcttttccttcttgtttttctttactatcgttctctttattaccatcttcgtaaactgtcatttccatttctttaccattacgaataacagtacgtttaaccttatttagtccaccaagatttttatctccacctttatggactatttcaggctctctattagcatataaaaaacaatcaaagaaattatcgaatcctagagatttataaacatgttcatatttaacatctaaaatatcactatttttagaaacataatctaaggataattcctcacccaataacaaattcaatacacttttattaatttcttcattactaattgtaccataaaataatttatctgtcaagtctatttcacctctattcatcaaagtagaattgtttatttccttcttgattgtaatcagtagaaacaaatgtagaaccaaagcttgtaccgtttccttctttaagaagttcttttattcttgttaatcctatataggcgtagacacttgcttgtgcatagtggtcgtcacctttacgtttgataacttggtacatttctccagttttttcatcttcttcatccataataacaacattttgccaatgttttaagaaagtttttaaatcatcatctactgtactataaacacttatatcctttgtcttaagtgcttgtacgtatcttttattctgcattaatttatccactgtaaccctgttattgttctcattaaattcaggtcttaattgtccggtagatttaggagaagatttataagtacatccaaatactttatcttttccaaaatgattaatgagttttaaaacattattacctgagtccccgttatctgcaattataatatcagggtcgtacttagatatttcccaaattattttttctaaatctgcttcaactaaatcaggtcttgtcatttttttaacagagaataatcgtattaagtctaccttaccattaggtaacataccatgaacagttatccagtgagtatttccccaatctataccaatagctatatgagaatatctatctcgtttcattaattgtgtttctgcaataggtgatttgttaccataaacatcttcttcattaactctaagtttaacatcttcaaaaggataacctaaaatatagttgtagaatgcttgcttagattctgtattcatttctttctcttttaattcatcagcagaaatccatacagcgttcatttgtgttattagataccctcgtacccctttattaccttttgtacgctcagggtacttacaatgccactcaccattataccatctatctagtggtttaccacatttttgacaaacaaattggtaactgccattttgtactgttttagcttgctcatctacaccttcaggattaacacataacatatttccactttcttcaagattatcagggttgtaatcattataactcatttcatttaagtaatcacaatgttgacatctatgaccgtaataccactggtctgattgttggtataatttgtgtatacccatcccaggtacagaaggtgtgctccatcttctcacaatcttaaaaggtgatgaagacattgattctaatgcagacgattctgctaataagtttaccctgtcatactcatctaaagataaataatcaatatccacaccctctacggtacttgctttagaacttgttctaaagaataaactagagtttcttatctttttaaaacctaacgagtctttatcccaatcaacaatgtctctaaaatattctttctctaaaacagggttcaaacgtgactgaacaaattttttcatttgttcatttgtagggaatgtatacagacactttgcgttagcataactatgcatatctgcaaaatgaaccatttccattacacccatttcactaagacctaactgacgtgattttattacagccttattaggatgagtatcatttacaatcctagtttgccaaggtctatgagcctgtgctttacttctatctctattaggaatactaaaagttataggatgccctctcagtgtatgatacttaagcatataggtagaaggatttagcatattgattatatggtctatctgttctcttgttattttttcagtttgaaatgtttcttgtgctatcttaattagttcttttccatccatttatatctttctccttagaatgtttctgaattttcctcattcattactttttccttttcagaaatcattgctgtaatatcttccgctgacatttctgatatcttctgtaaatcaactgtagattctccattactatcttctgttgtaatctcatcaaataattcctgttgaggtctagatagttgaggtattgctcctccttcagaagtttcattgaccatattttgcatttgagagaatatcacaaataatttgtataaatcattcgggtctttaacttctatttctttgttatctactttcttattgaattcgtacattaatttaccgataccattcataaacccatcagctatatattttcttgtatcctcttcttctaactttttcttgttaattatatctctaatattttctgcactcaatccatttcacctcgttttcttaaatttttataacaattgttgatatctgcgcaaatatcaaaataaatatacttagaatactgtaaatgaaaatgattaacttttgataatgcttttcctttagtgtaattcatacctgataactgctgaccacatagactacagattctactagggctagctattctcttatctcccttttttactaaagaaacagatgtagcaccatccttaagttctttcctttgttctttaatttctttctttgttttcattgtaatcttcctttttactatcttcctctactcttctttgtgctttagccctaacttttatctcttctgctgttataccataattactttctattattttcttgattattcttatttctttatcatttatctctacttttttaagaacatattcaggtagggggtcttctttcttcagtaagaacttatctgttacagtaagtgcgacattagaaacaataccaccaaagataaagtatgtcataactacactcggtcttattgagtaaataagggacaaggaattaataacagacaaaattagtagtaagaatataatgataaccagtgttttgaatatgaatatagctatgttattattcaaccactttaacctcctattgtctttttttattatattattaatctaacaattgacttttatgactggcttttaatgttatattagtggtgtaatctaaaagttctagaaataataggtcaaaaagaaataggtgtttaacaatgattatcgtatctttttttctgtctatacctcttttttgtttctattattttatacttagtggtgttacaacgtctttaggttattctatctatagaaaagataaaagtaaggtaactcttaatatagtatatctatcagctatctatgctttatctgtttttagtatttggcttattttctcagaaaaaataattattatcattattcttaatataattataataactgcctttatcataactattataccacaaaataagtaaatttaaaaattaatagtaaggaggttaccactttgactaaatataaggatatccttaagttagagtttaaagatgcactagcccattttaaaagagacagacgctatttccacgtctatagaatagacagagtattaataaatggctcaattatatacttcgactattattaccttccttctgatgaccctaatattgtaataaaagaattagacctccaaagttttggtaagcttaggtttgaaattgatacaaaaacatcttacggaaaagtcgttacggataattatatggagattatcaatgatttcttagaaaattatgatatccattctgaatctgaaacggttagaccttagaggtgattctagttggataaggagataaacaaccttgtttctcaagtagaaactattaagtctaagatacaagaaggtaattatatagatagaggaactttcaaagatttagaagttgaggtagctgaactaagaaaaatgatagttagtatagataaagatgtagctgtcaatagtgaaaaacaatcagcaatttacgtccaattagaaagacttgatgaaaagatttcagaacttgctgaaagtacaaaaactaaggatactaagaaaaaggatacaactgagaaggttcttttacttgttctaggagctatattatcctttgtctttaacaaatttgcataggagagattgtagatgtcaaaacatattgaaataacaatgtcaagtggagctaaatactttttagtatctactgatgaaaagagttacaataggcaagatatagattatatgttaagaggaatggatgaaacctctataaaagtatatactgaaagtgctataacttcacctcaagtatatattaatccaaacagaatagaatcttttaaaatagtattctaattattgagtgactttaagtagtcacttttttatttatactattgttgactttctaacattgttaattataattatatatttaaatagtaattaataatagtaataaatatatataactataataggagagctccttattttctaaatacgtacttttttgcctgtaagtactgatactaagggattttacttttatactgagtatctataagcagtatcagttatgtttatatcaattttcccaaccttattaattataattatatatttaatattatttattactctatatcagaatataattatatataataggagtactccttattttctaaatacgtactttttctcttgtaacccttgatattaaagtattcttcaaaataatttaaattatttttagaaaactgttgacttatttatcaatatagtatatagttaaattatcaaataaaagagaaaaggaattgattaattatgaaaagacaaaaaatgttttactcaagtttaatatgtaaagaatgtggaaatgtattcaaagtaccaagaaaaagagcaaataaaagagaagaaggtcatatcaaagatatatattgtatcaaatgttgtaaaactacaaaacatatagaagataatcgtagtgaagcagaaagaagatgggatgctattcaggaggaactaacaaaagataactaatgttttaaaaaaagtattaaattaacttgacaaaggtatcagtatatgctataatataattaacaataaaacaaataccttacacatcactgtttttattacattgttttccagtataaaacaataacaatgttatcatccttgtgtaaattaaatagaagtaggtaaacgtccctacttctataatttttttgaaattaacttgacaagtaataaaaattatgttataattaatatataaataatacaactaaatatctgagcatagtgactgaataatgatttggtagtcataaggtacaagataatcaaacgaatcaaaacctatcggagatatatcggtaggatgggtcgtcattagtattgttaaatacgattggtatacttggtacttacttataaggtctgtagagtatgataacggctagggtttaatttctttttagggtcttagcggaatcctagaaagatacgctgtaacgactaactgtagttggagcacagaaatgtgttagataatagaggttatcagtataagtagcataaatctacaggttcaaaagagctatatttgataaaaataatttggtaatcaaataaatttacttttcttcgaccatgtgaaagttattaactaatatgttaatatcacattatttaataaacttattaccgttttacggtttgtaagtcccttttaaattgcgtgagatggacttaaggtagctccttaatagagtcaatcgtagtgattcacttaataacaggtcttataggtaggttcctcgtgagacgggttatgcttggatatttagttgcattatatcagaatgtgactataaatattttcctctttatagtttattattaacaattatcagtttcctcctttgattgataattaaaaaaagaatctacagggagtaaactttcactctttcatgtatttattgttcacactcctttatgaaccaataacttactccttgtagattatacatatttcctatataagtaccaattaccacgttttgttcatttcacctcctttcacttaatatatagttgttatgagtggtaataatcgttgcttcgagcttatataaaaacctgctatacattaaggtaaggttagggtatagcattataattttggctcattggtgtaactggttaacacactgccctgtcacggcagagagtacgagttcgagtctcgtatgggtcgttttaaggtttcttagctcagatggtagagcactagattgaagctctaggtgtcattggttcaaatccaatagaaaccataataaacacccttagtataattagtagtacaagggtctccaaaacccttagtctttgtgcaaatcaaagagggtgtgttttacataaatgaaatctgagaattcatatgtgcggttgacgaaccggtttgctgtgtcctcgaatgagggtaggatttcattgagtatatagcgcaagtgtttgttcctaggatacgatactagctatatactttacatatttaaaaggagagatacaaatggcatcagcaaaacaattatattatacggaaagtttagttggtaaggcaattattaataataaagtatctaataaagaagaagtttgggataagttagagctacttcctgaaactaaattagaagatttagataacaaacaaatgtctgaagttatcaaaaaactaaaccaaattaatgagtaagtgttaaattaatatagataaacatgaccgacctactgttatattattgttagaaataaatataatagaaaggtcggttttttaatggctaatgaaactaaacaacctaaagttgttggaggaataaaccttagcacaagaactaagagcaaaacattttgggtagcaattatatcagcagtagcattatttgctaaccaaattataggtgctttcggtttagactactcagctcaaattgagcaaggtgtaaatattgtaggttctatactaacactattagcaggtttaggtattattgttgataataatactaaaggtcttaaagatagtgatattgttcaaacagactatcttaaacctcgtgatagtaaagaccctaatgaattcgttcaatggcaagcaaatgcaaataacactagtacttttgagatagacagctacgaaaacaatgcagaacctgacacagatgatagtgatgaagtacctgctattgaagatgaaattgatggtggttcagcaccttctcaagatgaagaagataccgaggaacatggtaaagtatttgcagaggaggaagttaagtaatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtaaataaattgccctgttatacagtaatgtataatgaaagttcttttaattgactgggaggctaaggctatcaagctatgctaatcagcatccaagaatattgttttaaaaaactacttgacaacataataactttcctatatacttaagtaaaggagatgttattatggaaaagaaattaaatgaaatacctggattagaaatatatgaaaattacactattactgataaaggagaagtaatatcttataaaggtaaagagcctaaaaagttaaaacttcaaaaaaataacaagggttacttgtttgtaaggttacgataccattcacctaaaatacatcgtttagttgctatggcttttatacctaatcctgataataaagaacaagttaaccatttaaatggtaaaaatgataatagtgtaggaaatttagaatgggtttctaattccgagaacagagaacatgcaataaagacaggattaaaaaatgaaataaattataatatagctcagtatgacttagaaggtaatttattgaatgtcttttacacagctcaagaggctttagagttcttaggtatttctaataaaagaagtggtaatataggaagatgtatcaaaggagagagaaaaacagcctacggatacatttggaaacaatattaaggttcaacgactatccctcggacgagtgccaataaaaataaatcgtcaataggagtagggctcaagttaatggagtgggtgagaatcccttaaatcgaaatggagaacccctaacaaaattagggtgaagatatagtctagtcttataggaaactataaggagttcataagagaaccggtaaaggagttacgtcttttattgaataatcggatggaacaggagcaaactcaggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcaagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaatagaatataaatttagacggattttaaatccgtctattttttttgcaaaaaagtgttgacaaaattaaatacatagtgtatagttatatatgtaatcaaataaaggaggaattacatggcactacttttaacatattttgctatttttattgtttttctagtccttgtaggttttggtataagttatttatttgattttctttcaatgaaagagaagaagagtaacataagaaaacaatacagggaattagttaggcaaggtacattagatgaatacggtttagaacaatatgtaaagtataaaaaacaattcttaaatgaccgtagacaatcaattgtaactagagccgataaacaagaaatagaccaagaggaaaaagctttaaatagcttaataaaagaaatagaaaaaggagaaatgtaatgagaaagtcagtagttatttcaggagtattagggtttttagcaattatagggtttattattttattaatgtgtattactaagattccacaaggtcatgttggagttgtatactcagtaaatggtgttaaggaagatactaaatcaccgggttggcatttaacagcaccttttgataaggtaaataaatacccaactaaaacacaaacacataaatataaagatttaaatgtagcaacttcagatggtaaaaatattaaattagacattgatgtatcttataaagtagatgcaactaaggcagtaaacctttttaatagattcggaagtgctgacatagaagaacttgaaaaagggtatcttcgttctagagtacaagataatgttagacaagcaatttctaagtactctgtaattgatgcttttggtgtaaaaacaggagaaattaaacaagatactttaaataaacttaatgataatttagaaaagcaaggttttattattgatgatattgcattatcaagcccaactgcagataaaaatactcaaaaagcaattgatgagagagtaaaagcaaaccaagaactagagcgcactaaagttgataagcaaatagcagaagaaaacgctaaaaagaaagaaattgaagcaaaaggtgaaaagaaagccaatgacattagaagtgaatccttaacagaggaagttttacaacaacaattaattgaaaaatggaatggaaaacaacctattagtataggttcagatagtgttattacaaacttaaataaataatatgtttaaccacctattgacataggtggttttttatgctatagttttattataggaggagatattatgattggagaaactattaataaactaaaagttataaaagagtctagtaagagggataaatcaagatgtaaaatgtatgaatgtttatgtgaatgcggagaagtaataatagttagaagctccactttaaggcaaggtaaaataaaatcttgtgggtgtgagagtaataaaattcatagtgagttaatgagagaaaggaatactacccatggattatccagtaacccaatgtaccaaagatggttaggtatgaaacagcgttgttacgatgttaacgctataaattataaaaactatggaggaaggggtatagaaatatgtgaagaatggaagaatgattttaaaaaattctacgactatatgggagaccctccgaatgaaaattatcaaatagatagaataaataatgatggtaattatgaacctggtaatgttaagtggtctactaggtctgaaaactcaactaatataagaaagaaaagtacacataatatatacaaaaaatcaaataatgtatataatatacaaattgtaagaaaaaataaagttaaatattttagtgcaaaatctttagaagaggctatagaattaagagataatgtaataaataaatataatgaaactggagagtggtaaatataattacctctctttttttgtatttaattatctgataactgtaatgtttgtaataaaaccttaataatattacactaattagtgctatattatacttgcgttatttaaagatatatgttatgattacttatgtcggtttacaaataaacaattctaaaaaattatttaaggagttttaaatttatgaagaaaacaatttttgcaacattagcattaggtacagctattacttttggaggtattgctacaaatgaagctagtgcagacgaaattgattataataagttagcagaacaagctaaatcaaattcagcagaagtaaatacaaaaccaattcaagcaggtaattatgatttctcttttagtgatggtgaatttacttatcatttctataattataatggtaactttggttatgaataccattcaggctcaactcaagtagataatacagtatctagattagcaggagaagaacaaacacctgaacaaaaagtagaccaacaacaagcacaatttgatactcaaaataaacaagatactaaaaaagaagtacaaacaacatcagcaccagttcaaaaggaaactaaacaacctacacaatcaactagttctacaggaggctctgtagcagaacagattagacaagcaggtggagacgaggcaatgattgaaattgctatgcgtgaatctacaatgaaccctaatgctgttaatgcatcatcaggagctcaaggattattccaaggattaggtaaatcatggagtggtggttctatagcagaacaaactaaaggtgcaaaacaatatatgattgaccgttacggttcaacatcaggagctcttgcataccacaacgcacataatagttattaatatattaagactaagattaatttcttagtctttttttgtatattatagttgacttcataagttaactatgctataattagtttaacaaataaatagaggaggataatgaatgaataaatttaaaagatggtttcgaattaatgttttaaaaaaagaaacactactttttaaagtttattggagatacgagtcaccgtctttaaaaaaacctcatgtatttcatatagagttatatgctaaaagtaaggcagaggcaagaaataaatcacaagagtatatactaaaaaatgcaaaagcatctgaggattttaaatttttaaaagtagaggagaaataattatggatgaaaaaaaggaaagtaaacctctaaaccttcaaaaaattagagtagaaaaaggacatacgttaagaagtctagcttctgagataggtgttcattactctcttatatcttattgggagtatggaaaaaagaaaccaagaagtgctaatttaatgcggttagagaaagcgttaaatactccaggaaaagagttatttaaagaattggaggaagacgatggagaataataatttagttaattttttaatgactacagatgatatagatgatactattgaaatggtagattcatttgaattacaagatataaacaaagttctaggtgaagatacgtttttaacaataatggaaattacagacagtcttcctgataaccaatataaaatagtattgttgtcctctttagacaagttattgaatacagatagaaaagaattagtagaatatgatgaagaatttcctactatacgaaaacataatgtatctgagctaaaaagagatacagttaactctgtaattgatagttatatgaatactaatgtagaaatactttatacagagtatcctactattagtaactacagtgtagttgtagattctgttaaagtgttaaatactttatatttaattgaaagtaaaaatggtaaaatagaagcaacactgtcagaagatggagaagacttacatgagtatatatcagaagagggttacagtgttacagacatattaaataaatttgatgatgttgaagatttatttgatgaggatgacagtttaattaatttcttttcagatattgatgaaggtaaaaataaaactattaaatcatttattgagttagttattaatttaaaataaaattagtggaggtatttgttttatgaaaaaaggagtatttacagtaatagctgatggttttaaatttaatgtgattgctaaagataaaaaagaagttcaagaacactgtttcaaatgttttgattttaactatatctcagtatctttttgcagagaagtctattcagattgtgaattccctcaatttatggaggattataagtatgcaggatagtgtaaatatatacacagacggtagctcctcatataataaaggtaaagtaggctcaggtgctgtcttggtaagtaaagaaggaaatataatatcggaaattagtaaaagtgttgacaaaccaggattaataaagtataataatgttgcaggtgaaatattggcttgttgttatggtattgaagaggctataaaactaggatacaatcaggcaatagtttatatagattatattggtttaatacattggtatgaaggtacttggtctgcaagaaatattctaagtaaaacatatattaatatgatacgagaataccaaaaagtaatagatataaactttgtaaaagtaaagagccattcaaatgacaaatggaatgactatgccgataatcttgcaaaaaaatcaattgatatataaaaaggagaaaatattatgaagaaaattaattcagtaattaaaggtgaaggtaagaaagtacaaacagcagatgttaggaaaattagttattatgttaaagattataacccttgcatgacagtagatgacgcaaacgactataatgcaactagtcaatatttggtaagtgacaatggtaaatttattgcaaaatataataaagatatgaatgcagtaggattctatgaagaatcaggggatactgtaaaacatttaacacatactacaccggaaagattagaaggaactgtattcactattgaagaagaaacagagattgatttaattaatgataccttacctcaaggtgatattttaattaaattttcagacggtagtatttatttacctgataatgaatcagtactagatagtgtaaattacttggcagataacgattgggattctgtggatgatattatttatacaggattatctaaaggtaatagtgaaaattgtattgtagattttaattataataattatgatattggttatgatgatgtagaagatgaagatgtttgtgataactaccctgaatgtgaatgtagtaattattgctcttcaacaggagaatatatcggaaattagtactatattaaaagagagtaaaactatttatagataggggaactttaaaatatggcaatacaactaaaagagttagactttaagttaaaagattatcctaatgtaagatacaacatgggagaacatctagtctttaatgaattccttgaaaaagctacaaccgagcagttagatttctgtgaggatttctttaatgataatgttgaaatactttggaatgagagtcaagccggtacaggtaaaacaatgtgctcagtagcctgtgcttacgcagactatcttaataaaaatagaaagctagtatttataatttcaccagtatcagaagatttaggaagcagaccaggtaatcagacagaaaaagaaatggcttatttcatgggattacatgatgcccttattgaacttaatatgaatcctgaacaacaaataactgaaatgttaatgatggaagataatgttaaagaagataaactaggagattgttgggtatctcaaatatcccatctattcctaagaggtggaaatctaagagatgctactataattataaatgaagcacagaactttaaacgtagtgaacttaaaaaagttcttacaagggttcatacaaaaaattctactgtaatagtagaaggtaattttaaacaaatagatttaaagaacgaaagtaaatcaggttttggagattatatggaatactttaaaaattatgaaggagcagtatttcataattttacagttaatttccgctctaagcttgcacaatatgcagataattttaaatggtaaaataatatgttgacaaatccccttagttatggtataattattctataataactaaggaggatttttttatgaattatttagctaaggtatttattaataacaattggttggtgaaacttataacaattgtattattaactttatttctaagcggtctagtttatgttataagtgcaatatcattattcttatcaacagttttaaatttacctggtttagtagtattagcatttttagcaagcgtaagtcttattttgttttctatagtacataattcaaaggaggataattaatgaaatcgtatactaaagtaaaaaataaaggtattgtactagataaatttaaagaaagaggtctagttgtacaagaaaaattagatggaagtaatgcaagcttcacagtagaaaatggtgaattagtatgtttttcacgtagaaaaaaattaaatgagaatgaaactttaaatggtttttatgattgggtacatgaaaatataaatgtaagaaatacgtacgtatcagccttagaaaaatacattatttttggtgaatggttagtcaaacataagattcagtacaaagaagaattttacaacaatttttatgtatttgatgtttatgataaagaaaatgaagtttatttatcagtagaagacatgaatgtaattgcacatcatttagggttaaaaacagttaaaactttgctagtatctaaaccatctcactacttaaatgatttaaaacctgaagaaattcaagaattagtaggaaaatctgacatgacagttaaacctgataagggtgaaggtatagtaattaaatacttagatggtaaatcagaatatgatgactactttaaattagtatctaatgagtttaaagaatttagtcgtcaaaaaatgaaaacagaagtaaaaaagaacgagtcagtggcagattatgccattacaagagcaagaatggaaaaaatgatttttagggctatagaagaagatagattatctgaagatgatttagaattagaaaacttcggtctaattatgaaacaagtaggtcaaaactttgttgatgatattatggaagaagaaaaagaaaatatactgaaaatagtagataaacaaattaagaaaaaaatgccacatattttaagagaaattttagaagaaaaaggagatactatagatgggtaataaaattaaagacaaagtaatttatatgggtggacatatcctaaatgaagctatggtagattacagagataaacaacataaagaagtagatggcattgtaggagtaactccttatagccctcacaaagataagtcaataaatgataaagctaatgcagaacaaactaagctagcagaacgtattttaactaatgactttaaggctatgcaagaatcagatatttttgtatttgatatccttaatgaaggattaggaacaattgcagaactcggtattttattaggtatgaaacatcaagcagaagaaacaattaaccatatatatgataatggagaagagtattttaattattttacaaataagtttgaaacgtcattaaatactgaagaagaattaatagtagataaacttgaaaacattgtaaataaacccgtattaatctactgctcggatattagacaaggacatggtaaaccatacaatgaccctgacagagctgaatttagtacgaaccaatttgtgtatggtatggttttagaactaacagacggagaaggatttatttcatgggaagaagttattaatagattagagaaattaggggaacaagatggataatttatcacattacttaagtatattatatgctatattaattacagtaggatatataccaggtttagtagcattagttaaagcagaaagtgtcaagggagttagtaactatttttggtatttaattgtagctacagtaggtataagtttttacaacttattattaactgatgcttcagtatttcaaatagtatctgtaggtcttaatttaaccttaggtattgtttgcttattagtagcttcatatagaaaaaaggactatttctctataccttttattattgtgttctcactgttactatttttattaagtgactttacagccttaacacaaactgtagcaactattactattatccttgcgtatgtaactcagataacaaccttttataaaactaaaagtgcagaaggaacaaatagatttctatttcttattattggattaggattagcttcattaattgtaagtatggttttaacacatacctatgttcatattatagctactgaatttgtaaactttgttcttatacttatatgttatctacaagctaattattactcaagagggtaggtaatattgaatgaccttatcaaagagggaaataaatattatcacaaagtaagagcaggagagacattatggactataagtaagaactatgatgtggaaattaagaaattacaagaattaaacaatattaaatcagtttctttaactaatttagaatacgtacttgtttgtgtagagtagaccaggaggtttataaatgagcatagaaaagaaagaagaagttatagcacataatgaggtagtatttaggagtttaactcaaggtctatatgtaaaagaagtagatatctattcagatgttgtaagctatactaaagatgttgatgaagctcttgctatgccaaatactatcaattttaaaaattcaagaaagtataaaaaacttattatgaatttagatttagaaccattaaataaaattcaaaaagttatatacgaaactcatttagaaggactttaaaaagttcttctttttttgttgacaaatattatttactatggtatgattagtacataacaaataaggagtgattttaaatggcaataacttataaacaaaaaggattaacagaacaagaaattattaatttacctaaggttaataaaggatgtatctatataggagaagaagatgtatttcttaagaaaaagaaaaataatataattaacttaggttctaaagaattatttagagatattcataatatatttagttttgatacagccacagaaatacatttatttttagccctatgtggtaataaagaagtaacaaactttgaaggtaacccttatgaaacagttgaaaaattagttgaaggtgtagttgatgataataaaggaagaagttacaaagagtatattgaatctaatagagaagaaagaaaagattttcctgtatatggtgataagagtagaagacgtatacaatcaaaaggttatgttgaagaaaaaattaaagaactagaaggtaatgaccatttatggaggaatgaatctagacaattagaagaatataaaaaagtagtagatagtttaaataatgatattatggatgtactagaccaaggtaaatatggtcttataaagtcatctattattgttatgaatgaagatatagaaaaaggttctagtgagtactatagtgctatgacagatgaattatatagccgtgtttggtacatgcatccaagtactgaaaactattcatcttttggtcttaaagttaaacatattagagataaacataatatgggtaacaaatgggttttagaaaataaaagttcatttgacgttaaaacaggagaagttaaggttttcttaacagatagtcttgttaataaagaaataactttaaacctatataaagatgatattagtaaaagtgaatataagaatgaattaactttatctgttttattaaatgttattttaaaaaactatgcacaacctaacttaactagaggaattataataaagataatagaacaaacattagagcaccataattttgatttttctagttggtgtcctgataatacagatgtttatggtcatataaattatagaggagataaatataggatttttataggagaaaattcaacttctaattacttaataactttaacagatattgttaaaaatattgataaaataaataacttagaagaatttgggttatttgaaagaaatgcattactattccatatacctaaaaaacctaaatggaaagttcatgaagccttcaatcttacaaaacagacttataaaaagttactaactttaaataaatttgagcaaggtaattacttaagatttgctaatattctctataagcactataatcatttacacaatgaagttaatttacaccaattgtttgacgatacctttttaatggttagagattcaagagatgttacagatgctttaaaagttaaacctattgtgaatcaaatattatctatatcttttgctaattacaaaaagatgacgcactatttagatgtagatgctcaagacagacaacgtataacaggatatgcactagataactattacttagattacttacatgatttatcaatattaataagagaaggttatagaacattagagagtgttagtttaacaccattttcactaaaactagaacatgatatagttacagatgagaaacaatctatacaacaacaattagatgatgcagaacttaaagctaaatatgataataaattagaaaaaataattgataaaacttataaattaaaagatggtagaaaagtaaaattccttcctgcagatactgtaagtaaacttaaagatgaaggtaaaatgttatctcattgtgttggtgggtatgctaacagaattctaaaaaatagttgtttaatattattagcaagattagaagaagatttagataattcatggtttacagtggaaatacgtattacagataatggttatgtattaggtcaacaacaatcaattgatgcctataaattacctaatgaattaaaagaagcattagaaaaagatatcaagaaaataaataaagaagaatttaaggaggttgcctaatggtaaaaccagttataactttagaacctgaggatgtaaaagtattattagattaccttagtttcttggaagatgatatgagaaactatgaaggtatgagagaattatatgaagaattacacaaaaagtatcaacttgctaaaggaaactactcagattaattgacaatagtatcataatatgatataataaaggaagttagaaatggaaaaattccaagaagattatgttaatatagatatcagagttaaagcttatgttcgtgtaggttataggtatgaagaagatattactaataatctacatgaattagttgaagataatttaaatgtaacaagtgactctgataacctaattatcaaagatacagaaattaaaggagatatagaataatggaaaaagtagaacttattaaacaatgggcaaaagatagaaatttacaaacaggtaaacctgaaggtcaaatgttaaagttattagaagaagcaggagaattagcttcaggtattgctaaaagtaatgaccatgtaacaagagatagtgttggggatatttttgtagtattaacagtactatgtttacagttagatatagatattgaagagtgtattgatatggcttatgatgagataaaagacagaaaaggtaagcttattaatggagtgtttgtcaaagaagaagaccttaaaaaataagtaataactagcaaggaaaactgttatattatgaggtgtacttagatagtacacttccataatatctttcctttttcaccttgcttgtagccaagcagggtgtttttttttatataatttaggagggtattacatgaacgcaaaagaatttatgaaaacacaagctcaagtagaagattatttagataaattaaaagtaacaattatagaagatgcactatcagtatctaaagaatggtctaatgattcaaatgatttaggttacgctttatctagtcttggtgaaagtataggtcttttagaagattattataatatacaagtagatgcacatttacctgaacactataaaggtagtaaggatgttatttcttttctagaagaacatttttcttatgatggctttgttgattctatgatatttaatattgtaaaatatactacaaggttaggaagaaaggatgcagtagataaagaagtacaaaaaattaaaacatattatgtacgattagaaagaaatataaagtacggagatagtactcgtgtctaaaagaacagacaattttatatatttctgtaaatattacttttcagaatatttaccttcactaggtgtagaagtacttaatcataatgaaacttctcatggaacaatggaaggtgttaggaaatattatatagcaaacatactttatgaaggtcaagaacttacggtaactattgatttagaggaattcaataatgcaacttctatgcataacatgttagaaataatgaataatcatacatataattgtatgtttatgtatgatatggatacacatgaaactaaagatattgatgatttttttaaattaatgtatttttaataaaagtgttgacaattaccttaccctatgttaagttataggtgtaaggtatttttttttattaacaactaaaaaaagttttagaaagttgttgacaaatacaaatacttgtaatataataaatgtataacaaatcaaaggagaatgataaatatggcaaagcaagatactattgaaagattagagagattggtagaacaacaaatggaaacaacaaaagacttggcagacaaactaggagagaaaaactctaatccgtatgaacaagcaattgtagatgcaattgttgagaaagcaggaactgagagtagagaaattattattactgacgttaaaaaacaaattgaagaatatgtagaagaacaacttagtaatttaccagttaaaattgaattacaacaagaaggaaaaacaattaaagatatctcaggaatcttccattatagataccaagatatactaaagttagttaaccaaaatattccagtatttttaaaaggtggagcaggttcaggtaagaaccatgtattagaacaagtagcagaagctctagatttagatttttatttcagtaatgcaattactcaagaatttaaattaacaggatttattgatgcaaatggtaagtttcatgaaacccaattctataaagcatttacaaaaggtgggttattcttcttagatgaaatggatgcatctattcctgaagtactattaattcttaattcagctattgcaaataaatactttgacttccctattggacgtgtaacagctcatgaagatttcagagttgtgtcagcaggtaatactatgggaacaggagcagaccatatttatgtaggtagacaacaattagacggagctacattagaccgctttgctcaagttgaatttgactatgatactaaggtggaacatcagttatcaagcaatgaagacctagttaactttgtacaacaattaagacatgagaatgatgaaaaaggattaccttatgtattctcaatgcgtgcaattattaatggtagtaaattagatggagtaatggaagatgagtttgttgtagaaagtattatctttaaatctgtaccgaaagatgagattaatcaatttattagctctttacctgagggtaacagatacacagaagcaacaagaaagcttttaggtatgcaacaagagcctaagcaggaacctagaaaatctgatagtacatcaaaagattcaatggactttgatacaattatggataaattaggattagaataggaggaaaatcaatgggtagacgattaatagataactcagaattaaatgtaattaaatatgatggtctacctgatttcttttctgctttaaaaaagaatagagtttcaggtagagataattcatcagatacaggtagctatgattttacagggactcatagttttcaagaagcctataacttaatggttaagggtgatagagagtcatatgatatggtagttaaacttaaaaaaatgacagatgcattatttagaatggataagtcagtaaaaagaaaacctgtcgtagctccggaagggtatcaacctcacgtacctaatgctataaaagggttacctaattctatgatgtctcagcaaagagttaaagcagagaagaaagttattgatgtattttataattctagtattagttggatggaagaccctgaaaatcttgcttacagaggggctataatgttaagtgctattcaaacattagaaacaaaaggatatagtataaacctttacttaggtaagttatcaaattcaggatatgaagataagttaacagggtttgttgttaatataaaacattcttatcaaagattaaatgtttttaaatcttccttttacttagtaaatccttcattcttacgtagaatatcttttagagtactagaagttgaacctgatatggttgacctaactaatcatggttatggtagtgtggtaagtaaaagtagttatggtaataaattaacagagcatatacttgataatgctgtaatttttgattctagcgtaggaattgatataaataacgattcatctgaaaacttaagagctgtaaaaaaactattcggaggtaggttgtaatggctaataatatttgggctgttgtattgagcattgttattttactcatcattttattaatactttggtttctttttagaaaaaaggtaaatggtagtagtaagaatgtagaaattcaaaaagcagaagaagataacgataataaagaacaagaggtagaagaagctcagtatagagaacttaatgaagaagagaaagaaaaaaatgaaaactctagtaaagattataagtacgataaagagaaagtaaaaaataaacttaaggagttagagtaactatgaaaaacctgattaaacttttatcaatggttgtagtaactatcttgactttttcactaacttatgttatacttaaaaaagaaacaaataataaaagaaatggtgtagcgccttttgatttttcattagaagaccacattcacctaaataaggagattaaataaatatggtatacgatagtattatttctcgaacaatggcagtatcaattttaaataaatggattgcagaattaattacagatgttgatttagataaatgtaagtttacagaagaagaatatgggaaagtagttacaaattcaatcaataaaatacaagatgtattaattgaaaagaactatgaggttacagatggtgaattgtatgatattgtttgtacagaattaattaacccaattaaaaataatacagaagaagaaaaacataatgaaaagaacgatttattagaacatttagaagatttggcttttagacatgatattgatttaggatatgttagtgatgggtcatataacttaactgtaacccattggttaatgcaagatgagtttacagatgttaacatcaaagttaataatgatgaagacttctatacagttacaattccggaaagtaaatatttttggttacctattacaaaagagaacttagaaatgttcttaacacaagaccctattaataaaggagaggttaagtaatggatagaattattggtaaacataacttaactcaagatttgagattaggtgataaagtagaagtttatgatgctcataaatttaaagaaaatgaagatggaactattgaattaggagataaaataactgaaggaattgttgtagattataaaggtgactttacaggtaatacaagtggtttagttacccttgactcttctgaaaaagaattaatcattggtgaatataattttaaacttattgaagaaggtaatttacaagcagtttatgattctgtatctaaaaacaaagtagaaagtctttctgaagattatgacatgtatagaaagttacttggagttaaatcaggagaactagcaggtatagaagatgaactagagtacttagtcagacaatataatagtaaagtagataattataatggactactaacattatctaaagaaaaagctagagaattatctctattaacaggagataaaaaaatgattcctcatatgaaaaatagaagattagaattaggtacagaagcagacttttaataagtctgcttttctcttatattagtaatagatataaggaggttggtatatggcaaatgaaaaagagattataagaatggttaattatcttattgataatatgtctatgtggcatataaactatgctagggctgtgttaataccaagtgaggtagaaaagataattaaagagcatgagaagtttgatgacctccttaagaagagaggagaatggttagtaaaaggttcagatacagataacattgatgatttagaaacttataatcaaataatgaataatcaaaaggatgaaatgatgatacaagaaattgatatctatacccaaggtaaaacaataacaatagataatgaacactattcttcagatgatttaggtgaagtcttaaataagctagaacaatcagaagatattaagataaaatctaattataagtcattatatgtaggatatactaacgtagtgggttatgaggttacttacgctagttcttacgaggaaacttttaaaaatgacttagaaaaagacctatgatatattagtactgtaagaataattaggaggaataaaccctatggaaaaagtaaatcatgagtttctagcagaattggcaaagagtaatagtcctgtactaaattcaaaaccacttcaagatggagactataatattgaatttgactatgatggttttcactttgagttctcacagaaaaatggttattggcgttggtcttataacgctaaataattaatatttaggctactttaattagtagcctttttttgttgacaaaatataaaaaatagtgtatagtaaagatataaataaaaaggagatgactaattatgtttaaaaaagcacctcaatacattatggaaaaagtagaaaaagaaaacaatattctaggagaagatttaagtcttgatatttattataaaggagttaaactaactgttaagagacaccctgaaactggtcatctaaatggatatataactttaccttcagatatcaatgaaaaagaatatgactccttagaaagacgtgcccatagaggtatcacttacgatgattatgactatgagggtaagagagtattagggtttgactgtgcacatgcctgggatatgacaccatatgctattataggttcgttagatgaccaatatagagatttagagtatgtactaagtattttaaaagatatggcagaatacgttaaaaaggatgagtaatatggacttctaccaatttctaaatcacgaaaatgtaagagttaattctataacaccaagtcaaaagaattttattagagaaaacttagaattaactaacctagaggatactgatattgattttattagttctaagcaagcaaaggaagaaatagagaaaattataagaattaaaaatgaagaagaatatgatatagctatggatgctttagcagggtgggtaactaaacatggttattaaggaggattgctatgaaatatattttaggattaataacactagggattatactatttaaagtctatgaacactttaagtataaacaagatgaagttgatacagaagaagatatataagtattgacattattatcaatatatgttattatttaaataacaaataaaaggaggatgttataatgaacttagaaaaaagtttcttattatcaacaatagaatttggaagtacttatcaaggaacatcagatgagcattcagataaagattacatgagtttagttgttcaacctttatcagatactatatttaggaataatgaaaaagcaagtaagcatacagaagtatcaagatactatgcagtagaaagatttatctctttagttctaaaaagtgggtttgataatgttcttaatttatgtgctcaattagagcaagctaaaaatactagattcaataaaactgttttagatttattttatgatgattttatatttttaacttatgttagagctaattttaagcctatagcatattctgttatcggtaatattaataatatactaaaaaaaggagaattaactggtaaagaccttgttaagttttatacattctataatcatttagaatactataatgatttattagatgatttagataacttaaatgttagctataaagactttgcaaaagttaaatatatgccaaaagaagtattagacaataagagaagcaatgtaagtattgaaaagaaaaaagatttggttaataaagtagagcccctaattcaagaagtaaaagataaacttaagtctaatgaatctaatatcaaacactataaagatgctatggaattagtagaaaaatctttgaaagacaaaactgtagaattccttacggaggtctataatgagagataaacgaatacactcagaattattatatgatatcataggtaaacacatacaagaagaggaaaatattacaccgtatatagaagctatatatgtagatatgatgaatattattgtagtagaatatactttttataatgaaaatggaacaagaatgctaggacaatatccgataggagaggttatgtaaatgaaaaatattattaattttttagtagactataacataaattttagttactcagaagatagtttaaatgttatgaataactcatacttagtagataaacatggtacacaagattatgaaattgtaggtaactatgaacatattacaggggtattttcttatcaaacagaagaagaggttatagctaagcttaaaaatcttatcggggtttgggagtaatatatgaaaaaaatttatatattagaagaagaaatagaggaaatggactatgacttgtgggaagaagatacagtgtatacaacaagttatgaaattttaggttatactgattccctagaggatgcagaatatattagagataactatggaacaagtaatcctatattcataaatgaatatccttatataacaaaagagaagttaatagaagaacaacgttactttagatacaatagttatattgaacttaaaagagttaatggttactttgaaatatctgaaataaatgacttacaggttactgaagactttagtattaataaagatgataaaaattttgattcacctttttctataaatatgttttcacataatagaaatagtataggtatagaattcattatgttttcagaatatgatgataaagaagatattatagaaaaagaaaaaaattcttttttaatgaaattaaaatatctccttaaacatagtaaagaagcagatatacgtagcacatcaaaaattatagattcaattgataaattaacttgacattaagtataatttatggtatattattagtatagtagttaactaaagcctcttatataatgctaaacacagttagcctttttaaccttataatgagaataagagcctaactatatgggtactttgaaatcattatattttacctatctaccaagcgtagaaaaggtctacggtaacgttataattctatttagcgttactagcaatcctgagtaatggcattggctagatgtagctacctagtcgttgaggttcgagtcctctctcaggatatttctaggactcttagcttaaaggtaaagccaaccgctcataacggtttgactgtaggttcgaatcctgcagagtccatataccaggctagcaaaaaacttataggtgttagacatatgtgcaatcacttgactatgatttaatattgtatgtggatttattgtttcatatctactaagtaaaacctatttctcttaagttactggttataagagaaggagtgaaaaactctaaaggtctagtcaacctgatgtacgagagataccgtatgttgcccggcagaggctttgtatctgaccgtagactccggatggtagtgcccctccattccgtatctatagttaattaattgattttggcttctagagcactcaagcttaattttagttttaacaataaagagtaggtaattgagaccgtagaacctttattgacgtaatagttagtacggacatagtgtcctgggtatcgaataggcttaaaaacgctaattgtacctttaaaaacaatcttcttccacaagctaccttctaaaatatcttgtggaataacgcagtgacaagagtttcaacgacagagggtctcatatagttgtcgtaaaatattattaaccgtaatctaagtacgggagctctgtcggacacctatttaaactaataggtgtttttttgttatattaattgtacaaaatatagtaaaggagataaaatttgtggagagaacattaaacttatatgattccaaaggtaaattattaaaatcttcagaaaaaattacaggtgcatccgctaaaattataattgaaaaactgacaccgaatacagtgtatagccaagggtcttttaaaatatcatggactattaatggtaaagagtctatattaacagatgttcccgaatttacaacaaaaagtaatgaagacaaacaagaaattgtttttaatactttaaacattgattctaactctttcgtagttagtgaaacagaaccaagtgataagagtaaattgtggtttaaaccaataaattaagaagaggagatttaaatgatggtaaacgtactacctagtgtttatgacgcagaaaaaggtgaatgggtaacactattagcaaaacctatagcagaagaagtacttaaaattatgaaagcagattatttagagcataaaggaaatattggattttttatatctaagtataaagacggagattctagtatagaacaacctaatgttgtagtattctataacgaaaaagattatgatacaatggagttaacagaatcagaacttactaatgcattaaatgaatatattgactataccttagatggcaagtataaaccattttctcttaataactttattaattatttggaagattatggatacagactacctgttaactttgaagttgatgttactattattttatctgacggtcaaaaatttacttaccctagaacaagctcaataactaataatgcttctatagtagatgcactgaaaagtgaagaccagtatatagaggttaaatatatttataatgaccatgcaatagatgataaaaaattagcacatggtaatgatactttaaaataacattaaatgataaacacctattaatttaataggtgtttttttattgactttttttactaagtatggtaagatagttatatacaataaggaggagattagatggctatatacgttgttcccgatatttacggagaataccaaaaattattaacaattatggataaaataaataatgaaagaaaacctgaagaaacaatagtatttttaggagattatgtagatagaggtaaaaggtcaaaagatgttgttaactatatatttgatttaatgtctaatgatgataatgtagtaactctgttagggaaccatgatgatgagttttataatattatggaaaacgtagaccgattaagtatctatgatattgaatggctctcaagatattgtatagaaacacttaactcttacggtgtgagtacagtaactttaaaatatagtagtgtagaggaaaatctaagaaataattatgattttattaaaagtgaactaaagaaacttaaagaatcagacgactatagaaaatttaaaatacttatggttaattgtagaaagtactataaagaagacaagtatatattctctcattcaggtggggttagttggaaacctgtagaagaacaaacaattgaccaattaatatggtcaagagactttcaacctagaaaagatggttttacctatgtatgtggtcacacccctacagacagtggagaagtagaaattaatggagacatgttaatgtgtgatgttggcgcagtatttagaaacattgattttccatttattaaattggaggttaagaaatgaagaaaaaatattttaaaggtcttaaattgaatgactttgaaaaagaagtttttgggataaaaaagaataaaaaatataagaaaatgaagaaaaaactaggtagaaatgaacctaagtattggaattatgacatgtccttttttattcagctatacgctgacctaaatgcatttatagagagtagtaatcatgtggatatggaataccatacttttgtagatgtagacggcaaagaaagaacacaaatagatatgataaaacatattttaagcttaataaaatattatcataaagaaatggatgattttgatatggataaatatgatgagcttgaacaggtacaaagtaaaatattagataattttaaaattgtattaccatcactatggaattaaaggagaaatattatgagagaaacaagggaatatattatgttttggggtaaagaggatatttattctaatttctaccctataaagtttaaacaccaaggaagaacatttaataactcagagcaagcctttatgtggcgcaaagcaagatactttaatgactttcaaatagcaggtgaaatactaaatgctaagaacccaaaccatgctaaaagtttaggtcgtaaagttcgtaattttaatgaagaacagtggaataaagtaagatataatattatggtagaagtagttaaagataaatttatgactacacacctaaagcaaagaatattagacacagatgtacgtaaagattttgtagaagcttcaccttatgataaaatatggggagtaggtcttaaagcaaatgaccctaaaatattagagcaaagtaactggaaaggtcagaatctattaggcaaagtaatggaagatgttcgagtacattgtatctacaataagtaggtaattataaaatggataagataaatcttaataaaaaacatgagggctctactgtagttaacatatcaaataatattacattaaaaatacaatgtacagacctaagaaaagaatgtgatgattcagaagcacctactacctatacccattttaaagcttatatcgtatataatatattcattgtagttaatgatagaaaacaaaagaaaaaagttaaatatgattgctataatgaccatgtaggcagaggtaatgttaaagacctattgaaagtaaaagatgttatcttccagttatccactcaattaaatactaatgaaattattaaaatatcaggtgcagatgaaagaagatataaaatatataaatattttatagaaaaagatataagatttgaagacaatatgtattatagtaaaagtaatatatggattataaataattttagcttattacaaaagttccaatggaacactgtagtaactaaagacggggactataataaaaaagaacttaaaaaggttgataaagaatggaaagaattattaatataaaaggagagttaaaatgaatatcaaatatattgatttagtattagaaaattgcgatgttgtaagattagagcctaaagacgtaagcaggttccatatatcaggtattacagaaggtatagattactatggtacatataaagggacttcaagtataagtcgaacacgtcactgtacttatttcggtattcttattgataagcctatggaaatacctcaagttggttttgcttatcctgataatacgaacgcttatgaaatgattacagcatattcagatattacagctatagatattatttatgaaaatgacgcaaatgaatatatttatgtagactttaatgaatacaatgataactataatatcaatcaaaagaatgattattacaataatatgttagaaattacaattacagaaagtaattccaaggaggaagaagatgaataaaacattttttaagttcctaggtaaaaacacattagagtattcaaaacaggggctaggttttcttgtagccctccctataatgttaattatattttctgtattcctagcattcattataggtattcctgcagttattatttacgctctacatgcattaaatgtagacaatgattttattatacagttagtacctgttatgtggttcataatactttatggtattgtaagaacaggtgagcacaaaaaaccatttgttaaactaaaattaaaggattacttattatctatattatatctaactactattacagctattagtgttttagaaaactatttgctctttcaatcattaccttttacaggggatgtaagagcagttataacactattatcatttatagtatttgttgctgtaaatagaggtatttgtaaaatagccattaagagctataaagaatataaggaggactcacaatgatagaaattagtatctcatggacttatttaatatcattcttgctactatggtcagccggtatcttatacattaactaccttgtttatagaataaggttaacaaataaggaacgtaaagaaatgagtaaggagcaccaccgcaatagagaagagataaaacaacggatagaaaataggagggataaataataatggaaaactataaaaactttattatagaggaaatgaataaagcccatatcttagtaactaaagcagaacaaattaaaaggaatagaaaattagcagaaacagaactagaagaagtatataaaaaagcagaagccttcgatgaaattgtaaatgagttactttatcaattacaaaatctagagagttgggatactctagaccaaaaagactgtcagacattaaaacaaatactagaggaaaacataaaggaggaaaaacagttgaaaagatacaaagtaaaacgtactattactacagaagaggtaagatatatagatgcagaaacagaggaggatgcatggtatagtgtagaatatgaagatgaaggtgcagatacagcacactataatgcagaatatggtacgtggtcttatgaagaggaggaaaaataatgggactagactttgaagtaatcggtgtgacattaagtaatagaaaagtggaacagaaaggcttacagcactttattaataacgctaggtatagacatatattagaaaagaattattataaaggatttaactttgaagatgactttagaaaaccaggatattttatggacttacttttaagagatgcagagacatattatgatgagtttgaagaatggtgtgaaggtgtattcgtactaactaaagacaagttagttaatctaatgaaaaatgagtttaatgaaaaaacgtttaaaggtacacatgatgcagaatattattatagattaatgtctcatatatacaatgtagaacaatatgaaggtaaattctatgacttttacttaatcatgagtgtaaatgtataaggaggattaagtatgaacaaagaacaagccaaacttaaactcgaaacaagtattattaattatgagaaccaaataaaattcttagaccctgcaagtatgtatactagagggcttatagatgcaaaaggttactcaaaattggcgttaaaagaattagaagatacaggtaaacactcttatgaagatactacatggaaagatagttatgcaaaggtatttacagatgaagaaatactagaattcttactatccaaaccaagagtcacatttaaaggtaatcaagaaaaactagatgaaattaaaaaagaaagagaaaaaatacaaaaagaagctactaaagacttacctaagggcagtccactaggtgacttatcaaaagaaaattatgagaaattttggggagcattacaatggtctagagaagaaagagaaaagttaacacaagaatcaagagcttattatgaaaactatctaaaaaaaatcaaggagaataagtaatatgaaactataccaagtagaacatgataattgtgaaccttatgaagataacttccattttagagaagataagatatatacagataaggagaacttaattaaacgtattaaagaagaaggttataaagaggaaacaaaccatagaggtgagcaagagttcattaaaggagacccaagagatttctatggaatggatatgattactatacatgaattagagtttgttaataatacctaagaggagggatttaatttccctctttttttattttaggtatgttgactttatatttagaggttgtctatttgtactataaattattgacttacagggaggggttataatgttgacttttaatatagggtgttgacttactttctatattttaggtgttgacttttaaatatttattttcataatttttcccaaaaaataagtacctgggtagaccataggggtatatagacccacccccattgagtggcattttacccctgcccccgtataatataaaaaaattatataataggataaaaataaaagagggattttattccctctatttgttatttaaaatattccattttgtttttttatttctacgattcttttttcttgtctttctaatttgttaactaaaatatcgtaagcttcagtgtattgttctcctttagcaatcattctatctaatacccaaataatgtcttttacatctttttctaattgttcctgctctaataagtttctcatgttcatcatttccttttcttttaatttgttaagttcattataccatacaaactaaaagattgtaaagcgttttgataaaataatttaaacaaatgtttgtattagttctttaatctcaccagtagataatgacgataggtcataagcttctctaagctcttctaagctaccatatgggctattactatatgaagtagtattattgttcatctgtattgttccgcttctctgtgtgcgtctgtgtagctcatcattgatgtactgctctatgaggttatccgttgggtgagcctgctcctgcaggagtttgctcttagcactcttaacgacattgtgaacctctctaatgttcattactcatcatcaccattcataacatcaactaataccattagctcttgcttagcctttatttgcttctctagttgtttattcaactcctgcatagtcttttgagtatctgctatttgttcatctaataatctaactgcttcgtttaacgctctgtttacccgtcttgtattcatcttcaacatctcctgattaattgtattagagaagcggttaagcttctctgtgtgctttctatagtgccattctttgagctacaatctcaaccgctagattaacatattgaaccttgtcagtttcttgtagttctaattcaacatcatccatagcctcatagattgcttccgcccggtctgcttcgtccatgtcttcaaatccttcaacctcatcagaagcaatgttttctgctaggtcaaattgaatgttattatattcgtcttcgtcttcaaattctacatttgcataattctcgaggtctctcataagctcatagttcaataagtcataatattgttgtcctgtaacctcttcaatgtattcagtgatgaccgcttcaatgtcctgctcgtaactatcaaagaagtttactacgtctgatgtgtagattaatttgccaattgctccgctttgtactccatgagttgctacgtccttaactgtgcttgcttcttgtccttgtaaaaatgcttgtattgtatttgtcataataatcaaatccttttctttttttattttttctatactcttatattaccatctatatgttaggatgtcaataccttttctaaactaatttccaaccgatgtcgtttacatattcttctagttcttccatagtgtccgcaaatttgagtgattgcaaagtgtctgaagtaataacaaccgcttcctggtcatgtatttctttgtctaggatgttattatctaagtggtcgatgtactcccaaattagggcttgttttacactctcaagtgttgctatgctaattacgatttcattatctctattgattaatttcatttgtatcaattccttttcagtttattttgtcgttgtgcttgattaccttacaatttaataataacattgtagggcttgaaagtcaagccctttatttaattaattttcgtaaatgaacaagtctttgcttggtaagtatctaaagtttggttcaattcctaagcaatatacaagtgtgtttaaactgtcacggtctgagttgctttcaccgtaatatgaaacaatctcatttatgcttaagtcaatctcaattgtttgtgtaccccaatgacgcataactaatttgttgccatcaatttcaattgcatatttttcttcaagttcacctttactatatccgtcaaagtaacgaccatttaaaattgcttgccctttgttttctgctttttctaataagttttcgatagttttgataaatttcattataatcaattccttttctgtttgttttttgttatcttaataatatcatggtttcaagtgactgtcaatagttttttttaaactttttttatttaggttggaagcggttgaccgcttcccttaccttgtatcttaatattaacatctattgtttaagttgtcaagccttattttgaatattttacaattttatttgctttcaagaaagtaacatcttgacggtagttttttaactcttctgtaaacctttcattatttttcatgccgattaagtttaaagttgttgtgatttttttcatttttatcatttcctttattgatttatttgttatcttaatattaacatgttgtttctaggttgtcaatagttatcttaaaaaaatatttatattatcttgtagtagtaaaaaccagtttacaagtttaggtgtgactgttgcaactaaaccccctataataagtaagagaatgttatctgttttttgtgttgatttgattgttttgtttttccatttaaattttatcattttgttcaatccctttcatttggtatacctttatattaaactataatgatattaaagtcaatagatttaatgaaattaatttaaaaaacttttttacatttatctattgacattaagaccgaattattatataatggaagtgataccaaaaaaggaggagtaaaaaaatggttgaaaataaaattaatgaaatttggaaacctataaaaaaagaatactttaataaatataatttttatgtttctaatttaggtagggtaaaaataaatgataggttgagtaaagtacatcaagaccgtgacggatatttaaccgtaagggtcaataataaaaaacacatggtgcatagacttgtatatgaatattttggcaatgattttattaaatcaaaccatgtacaccatatagacggtaacaaacaaaataattgtattgataacctagaatgtattagcccgtcagaacacaataaaaggcaccataaagacaatactttcaataggtataataggggttatgcattaacagaagatgaaagaaaagccatagcaagcaaatacaagcctagaaagtacactcaacctatgttagcaaaggaatataatataagtgaaataaccgttagaagaattataaaaaaatataaaaaagattaaagaaaagggttgaccttttcttttttctatagtatactataagtatagtaagaaagcggtaaggagcctacgtgtccgctatataagagaagaggttccgggaaagagtggttttaaaaccatttaatataaattttttatataataatttttttatactaaaaaaggtcttcacttcattataccataaaaaatacaaccttgtcaacagtcaattttacactaatttatttattaaataatttttttatataaattaaaataaataaaaatccctagaaatctaatcctagggaattgtataattttttttatattattgtattgcacttctattatatcttaatttcataaatttattataagtttgtttactgttaaatataataaattcatctgattcatttttaaatatattgtgtacagtttgatagctcatatatccattttttgctaaattccataaagtttttatatcactattatttttttctattttaaatttcatattatcatatcctttttatataatttttttatttaattcagaagcggttatattcaaccgcttcatatttaattttattgactaattctatttgttttaacttgtgtgtaagcatattctagagtatccgctatattataactttggaatactacaccaccgccaaaccatttagcagtatattttttaaagccgtatagtttattgatgttgtcatagtctgctagtttaatatctaaatccatgaaatgcactacataacgtgggttgccgtttgcgtcatttggaattctgaaagccgtaatgtcttgcccttgtactgtgtgagtttcttcgtataagtaatttttgtttgtcataataatcaaatcctattctataatttttttatttaatttcgttatttgctaggtggcttgtcttatttgttatcttaataataacatgtatcttgtaaggtgtcaacacttttctttaacttttttaaaaagttttttgaggtgtgttgctatcccctacacctattatattactatagatgcttgaggattgcaacccctatttttaaatttcttcaataaattttatttctttgttttgaactccgtacactacattttcatatacgattataattacatcgtcaatcacattaactaacatgctagccggttggctttgccctctcatgctcattataggcgttttgcggaacatctgcactgtgtcatagtttccattaatagaaacaattaacgccgtatatgtgcgtgtatggtttagtgtgctttcatataatggaggttctgcacatctgtagccgtccatttgccacaactccattatagtatttgcaatttcttttacatccatttaaatcatctccaattaaataatttttttatataattctaatatcttgttgctgttcccttgctgtacctttatattactaccatatgataataaagtcaataagaaaatgcaaattcttttaaaaacatttttagggctaaaaactactattatatagaagaaaaaagaatttaaaaaaagttactcaaaagtattgacttctgaataactatactgtaatatataaagtgtagtaaggcaagggacaaaccccaaccgcttctacaaatattatataaaaaaattataagaaaaggatttgatttaaatgaaaataaactatattccaatgtgggacaatgaagatgtattacaatatgcaaaatcacaattattagtaaatgaattagaaacaaaagaaattatattcaaaaattatcaaatatcagatgatttagatggcggaacagataaaaaatattatgaaatatatgaaagtaaattttatgtagatgaggaaacaacaaaagaagaatttaataatttaattatagaaaatgaaaaactaataaaagagtataaaacacaaaacggattaattaaaaatttaattaaatcacaacatgaagtaaatgaatttgaatacaatgttataaatattctataaaaaaattatataatacactaggaataatatcctagtgtatttatttttgcggtctgatattattccaaccgtctatatatatatagacacctgaagcggttaaaataaactagtagaaaacagagggagctacttagggtaaaatcataccgagctacttaggcaattcctagggagctacttagtgaaattagggagagctacttagtcaatccccacccatacgttgccaagcttttaacctaacaatttctcctaggtcatgtttactaatactttctcctctataatagaaataattactataattaccaaaaaactcaggaacacctaataaagctacaggttcattataactattatcaagaacaatgtcttttctttctaatagaccttccatatccttacagtatacaactgatacaatataagaggaatctatatcctcaggtaattcatctttatagtattctaagtatctatccctttcattaagccaaggtaagaactcgtcttttaaataatccttagaaacatctgtatatataactatcatcctatccctcctatcctacgtgccaaatgtgattgaaatcaaatacgccttgtcttctaatatctactagtaaattgactacatctatctttccatgtggtctgcgtttataatcatctaatgatatatcagtaactagattagtatttatcatcctttgaactaagttcatagtttctgtctcatttaaaccgctagctattaccctatcagattctttatgaataatagtgtatgttaaagtatccatcctaacttaaccccctatcctatcctatcctattgtaactcatcctaaattattttctatcctaagtttatcctatcctaccattctcttattgtaacataattacctctattgataagtaatataccttcctcattaaccgcttcaataagtttatctatatctatatcatcactcttactcccaaactctttattatagtgattagggaatacgttactaatatctttataccaagtatcatacaagaagtatattaaactacttctatctagcttatccttaatagttttcatactacctctatggtagactgtataatataccttaaacattagttacatctcctttacatactcttccatactatctacacctattgattcacccttaagatattcatgtatccacgtctccgctataaatacaagtatatcatcttcaaatggaggaagtgtactccattcttcaacttcgtctttagtacacctatagggattaacataaacatccaatcctggacttctattaacatcataagttagttctcttgaagcggtctctttaatccattttaattctgcttttagtaaattcttatcataattttcactaaggtatatctctatcattataatctctcctttacgaaggaaccaatattacatgatatacctctatagtagctacattctaaaccatgtctatttattagtatacaaagtttaccttcatcaataatgtttgagttaatccttacatcatctctttctctaaatacatttttctctatacatacatccgcttctccttttcccattaaatagctaagacctctactactacaattatcaatatgttctaatactttctttaaagccttggcatcatatttttcagttaaaaatattataatcattataacatctcctctacatacttacgtataataacaggttcaccattaacaaatgccttacagtatagctcttctataagaatacataagtcatccttgaacatgtttgtacctgataactcaattgtatcctcctcatctatagggtcatcggtttcaatcacgacatccttatcatagtctgtattaataccataagttaattccctacctacagtatcattaatatactcaagtaaactccttaatgtttctttattataatcctcttttacaaatatatgaatcattatatctctcctttcaattctaagacctctcagccttccatatagaagcggtatactattttaggacaaatcccctctaactacttaaccgcttcttaaatgagctctatattccaagtatagctaaaatacctgatgctattataactgtcataagtacaaatgttatgacgttaattaccttatcaaaactattatatctgattgactgaatcaagtctactaaaaaataaatatacatagagatagttgatatactcattatcgtaattaatattgttattcctatcatgattacttatcctccttaaaatatctcttctagtattcttaaaatactaatacctgaaatctgctcaccttggtggtatacaatagcatcatcatcaactatacaaataattaaatcacccgggaagtaataggatatatccttatatataaatctatcttcaggtttaacatataacattgtgtgaatattaacatccgcttctacattattaaaatcataacttaactccctaggagcatggtcattgattaatctgagtgctttagacaagtattctttattataaccttctcctaagtatatatcaatcatctatttcatctcctaaatttgactatacctaattatagcatacccgcttcatagttgtcaacaattaattacaattgaagcggattatctctcaatattctcaattatagaaccctaaatgataagtagctacttactattttaagtacattaaaactttgccctagggaaaacctaatactttttagtaattttaaaaataagaatcccatacctatagggttttatattttagtaccacttttcctagggtctaaagattaaaatcctagattagaatgattctaatgtaatatactagattaatgtaccattatataagattattatataataaccatagattaagaccgcttcagcatagattaacagtagaatactatagtttaagtattgtataataaatgaatattataactatagaataagtagttagggaagtcggtaataagtaataggctgagtgagagaggtaaaagaattcccaggtacttagtagtagtaatagtagtagtaatagtagtagtaatagtagtagtaatagtagtagtaatagtagtagtaatagtagtagtaatagtagtacataacaaa

SEQ ID NO: 2 - DNA, 138352 bp, synthetic construct
  source 1..138352: mol_type = other DNA
tagtagtagtaatagtagtagtaatagtagtagtaatagtagtagtaatagtagtagtaatagtagtacataacaaaggtacttagtagtagtaatagtagtagtaatagtagtagtaatagtagtagtaatagtagtagtaatagtagtacataacaaaggtacttagttttacttggttaggttctcattctatatcttcctcattagtcaacatactctcactctaaatattgtccttcctattactataatacttaatgattatcatggtaatctgtaattagctgtgcaacaataatagttaacaagggaacaactcctatagacataataattatttttgcaaacaagtccatatgtttcctccttataaattatatactttaaacatcttatccatagtaatctataaactctacttcttcaaaacctcttatctctgataatcctgaatcatctgtagattctattgttactgtttcatttttaaagttaataatttctacattaacaatttgttgagttatttcatcccataactttttatatttatttatcatattaattccctcccttaccatttaatatttaagtaatctaaccctgctaaattatttgtagatatagaagggtactctatatcattactaatgtggtttacactatatccttggttagtaagttcttctataactctatattctacttcattatctactcttaagtctctaagaacatattgataacttgtattacctttcatagcttcttcaataataccatcattaatattatctagtatatcttctacaatactatctatagtttcataatgtacatcatcttcataaatttctgataggtacttagcatgccctgcatttatcataatagtttactcctccttattatttaccatcgttcaatagatacaatatgtctagtatcaataataacttttttatctttcccatatttagttacttcaatacaatctttactcataatattttcatacaaagtaactagtgattcgtatttactataataagtatattttctattatctacagttcttattgtataatcatgttctatacttatcattataattactcccatacgaatccgaatataccatcattttgtacttgtcttacccattcctctaataccttatgaagaactgaactaatactctgcgtactagtaattttaacaacagttgttatataaagaccttgtggtatatgcatttctatagaatctgagtgtaacttatcaatatctatagctacaacttctgtattgtaagtgaaattaagtctcatagtataaggtgttacatcaactctatataactcaactttattttcatcaggtaatattagagatttaccttgtttatttgtaaagtactctactaattttgtactattcatattacttaaacacttccttcattttattataagcctctaataattttttatattcttgcttcgaaagagtaactttttcttttgaatctgattttttaaagtattccctgtagtccctctcgataggtctaataatataatcaatatctttctttggatataaatgctcctctacaggattagtagctttaccatctctattttgagtaataacagttacattgttaaaatcatggttatatacaataataccattaaaaccgaatacggagattctttctgatacaaatatatctccttctctataatcatctgttcttaacaaatctttggctttaattattttccaagaactaggtaatctttcgtatttcattttattagcatttaacttagttttgtacctacctagagttaatcgttgaacataattagatactaagtctgtagattgatatccataatagttatcttcctttataaatttacctacataatcatttaaatacatagttagtctcctttatatattaattcatcattcttatacactttctcacctacattagagaaatcatcacctttatcaaacttatgatagacctctttattaagatatacacggtcttctcctttatctgtctctaaatctacatagtatttcatttcaccaatgactggtacagtaacgctacctgattttatataagtagttggttctctgtattcacctactcctactactttaccttcatggttatcaggagagcaaccccctagtaatagagaaatagtaattgtaagtaatactattaactttttcatattttcatctccttgtatactattatataatagatacctacctaagtcaataggtagatactaatttatttatcagctacgtactcataaaacccaatatcttctctaagtttatcttctcttaactgcttaacatctttaggtggtactgtaactccttcaacatattctaccaggtcaatgaatgctttctctaagtattccctaggacagtcttcgttttcccaatagttaactccttctttttcccaatagttaactccttctttttcccagttattattcatgctatttctctcctttcaactctttaatttcaagtgtaactactttatctacatactcctctatagctttataagataactgatttaaatattgttcactaactcctaatatttcagtaaccttttcctggtttcttactacatcatctaactcagtatctacataacctagagattcaaacatttcatctatttcatttttgtcaggcttaatttctttatattcattataaaattcttcagtagttctaaagtactgttgtctatttcttttaatatgatggtagtctattaattgtctctcaccattaattgtattaaactttcttacactaaatgatacgacatgcccacctatttctaatttaactccatttaatttatctataatcattttatttctctccttttacttatggctttaaccattcccatttttcaaaccatagtatttttataatattgtacttatgacctactctgtagtcatccgggttgaactcatttacttttaaggtaataatctttcttttcctaggattatctacaccaataagaatcacatctacataatctacatacgcaaacccctcatcaggaggataagtggaatgctcaaatctactatagtaattatgttttgatattttataaatcatttctttcactcctttatttactatagccctactatataataaaaaactccctttgtcaatagggagtttaaatatttattaattatttttaattatttctctaatttcacttaacgactggtctctataaagtttaccatcttcaaatactctttctaattgattaacattttgtttgtttagccattgatgtaaggttaagttatcttcccatgaaccgtctttatatacagctactcgacctttatgtgaacgtttaccactatctgttttaggatttttatagataagtttttcttcatttttaatcttagcatacgttgctttgattgcaaacccgtgggtatcacgagtattaaattggtaagagaaactacctacacctaatacaacattaatacttgcaaaacctttttcttttaatccttcacaaatttgttgagcacgttcatagttaatagagtctccataaattaatcctacatgttcatctaatactttgtaccctttttcagtttcagtacctccaaatgtatcccaaagcacttctactgcacctttacgttcatattcagtgtctgcatcagggtctccgcaaataattttaacagggtctccactatcaggtctgattactactttaccattacgttccataataatatcctttaatctaggtaaagttttagtcatattaccccaaaagtcccaagtatcagacacaatagagaaaatgcctgttggatatatttctgttaacatacgtttatatgtttcatactcatccatggtttcataattaccatttgcacacattactgagtgctcagtagcagggatagaagcaacaaccatttccttctctacgtctgcattgtaatatgattctaagaaatccactacaggtactgtatcactacctttaaaactaattgcatgacctgctgaacttaattgagctgtttctaaagaactcataccacgcatactaaagtcatgaccctggaaatctactgcttcaatattacctactgtttccattgcatatttatctagcattttacgcatatcatatgctagtgtagcactagtcattgcttgccatgtttgagtactaataatagtttctaggtaattagttaaccaaaagaaatctgagtgagtattttcaattgtcataacaggtgtgttaggatgaacaacagtaccttcttttaaagctcttacatcaataggtaaataacctaagtcatgtaattgttctaagtgtttagcatcaggattttcttgtcctagtgtaaatttaactgtacgtttgtattcattaataacttcctctttaggtctgttaaagaactctttattaaacatatcaataaaatattttttaacaagtgattgaataccaaatactacaatattatcactgtgttcataatatttattacttctaggtactagtgtactatatacaatttctgtaccttcagggtattgttctctgtgacttagtttatagaagtcacacattaaagttggatttagcataatttatttttctccttttataaatatctttcgatatcttttatttgtaattcagttccgttgattctagctctatgtttagcaatagctttttcccaattatttttagggaacatagagtctgttacaataatatctttaaatcctgagctaagtaatgctccttctgcaaaagctcgttctgcatgagtcaatattaatgatacactacttaccttaagtttgtcaagggcttttttacaaccgacaaatgtcccaccgtaacttgttaagtcatctagtataatacaattatcatataaagtatttttatctttaattattttaatacctttaatcttacctgtttcaaaatctcgtttcttctcaccatatacaattgaatatgattcaatattagattccattaagatacgttctacatcaaatagatatctatcgtatgcccctttatcaggtagtactattgttacaggctcttcggacatttcaatatactgatttaataatttaagtgtaacatcaatagcaaaagaattagtaagtaattcttctgttacagggctatgaggttctgctacccataccgatttataattaaggttattaataaattctgtaatgtatttaagactgaacatattattatgtccttcttctactctatccattctgctataaggtagatatgcaatctctaaaaattcagctttatctttaatacctagttgctctaaatacttagtaaacatatacaaatgcattaagtctgcgtcattttcccatttaaagaaaacatcaaatcttaagtcctcttctactaaatactttaaatcatcatatttaaaattaatctcaccattaggaaacttttcagtaataataggtttgtattctttatctacttctgaaaatattttaatcattttgttgatagctcctttttatatgttatttggaatacattttctgccgatatattcttaattacatcaataaatcctttaccaatgcttaacattgtatctacatctacaagtgttaaattttctttatacattttcctcagtaattcttttacctcaatgtattcacctttaaaaactaacttacctgttttaagatgatagatttcataaatttctacttcttttgttgtaatgctctctaaatactctcttgtactttttagcttatacgctaattcttccacatctatttcaggatacataattgttcatctccttaatttcaattctaagctactttaaggtaccatagagagcacttaaagtaatttaggtactcttatggtaaaatcattactgctctttatcatacactctaacagtcttctgaacattgctttttttaatttcgaacataattttattataaatcatttcaatactaaataagaaagctccgaacattactgaaactaatcgaataacattttctctgacatttaaatagtctaagtagaagtaaagaccaatagccatccctaaaacaattaaaatacctgataagccagtactaaactttgtaacatagaaataaatagcatacataccaaccaatactgctacaatattaataaccaattccatttataacccctccgttgtaatttgacttccaaaattattcatatagtacacataagcttgtagttcatctaagttaatttcctttttagggtaagatgctgatgtcatctcagaaataatgtgtactcgtttttctgtaacactgatgtgtacgttgtaaccaccgctaatacaatctgtaataaaatctctataattcatatttacctctccttaaaataaaatagctcctgctttataatataaatcctccaagtagtaatgcaatcccaacaccaaatatggtaaatagtatttttaaatctacatcatcataatacttacccaataccaacttataaccgctagataaaaataatatgagtccaataagtataactattacgggtatcattatattacctcctttttctttatatctaaagtatactataaaaaatacctgctgtcaacaggctgtcaacaggtatttataaatttatttaatttttaatgatacccactaaaagtgcatcaacagatactttatcagcactatataactggtcaatatctacttcatctattagaacaatttcacctttaatatcataatttgaataatcttcagcagaagctactctaagatgagggaatttatttttaagcatttttctcataggttcatttgatactactaagggtaaatccttttctagtgctaaataagctaatgtgcttgttttacctgtacatctattgagtacttcagaagataaagctacaggtatttccttttcaatagaagatttaaacattgaataatattcattattgatataaggctttagtttttctaattctttagttgaattaatttctttaatagattcaataaaatcattgtcagtctctacctgttcttctacaatagaaaaaatcttatctcttaattgtaataaaatttcttttggattattactaccataatcaacaaaaatttcagttgtaatatatctcgttgttctagggtctttatcagctttataacttatgcttatttcaaaataatcccctttatcaccccaaccagtttgtttattgatatgagcaataatactatacgtttctccttttattccttctaaatttaataaataatttgtaatggttttattagacacttatttttcctccttaaaatatcttgatagttctatagttgttttagtttggtctcctacaatagcttgtagtttattactaatttctgtatatagctctttagcacttaatgtttcaggtctctcaattgtaatagctgtatctaataaatgtttacctgaaagtgtagatacttgagcttctaaaaacaatactgattcatctatagtatctatatctaaaatatacgatgaactagcctgtaagaatcttactttataaccctctatttgtagtactttgtcattaaaaaatcttgctattgattctgctttcataattaattttcctccttattatctttcggaataaaaatctgaggtctatatttatcttctagtactctaatagcttcctcatactgagatatcattgtatcaaagtgtcttagttgtaactgtgattcatactgctcctcacctttagtttggtactccgcaaagctcttttcttcagttattaattttgctagtgcctctcttaaagcttgtgacggtgttgcaatataattctctccattaatatttgattcatattttgccatttatagttcctcctttaatttctgctaaacatacttcctattgcactgattaaaataaatattccgatacatacccaaatacttactttaactccgaatgcagatagtactaactgtaaaatccatgccggtaaaacaatacctaatgcaataactagtattacaattcctaaaattaaaataaaatgcttcataaattaacctccttatttcttatactcttagtatataataaaagaaacctaatgtcaataggtttcttaaacttttttatttatttttaaaccctcttagagtaccactttcaagagtacctattttaacagattttttttctttacgttcttgctcttctaggtaaagaatacctaatgagttggctgttgtaactatagttcttgaaatttgacctgtgttataattctcttgcttaattttacctttcttgtacctaacaagtgctacaatatctaaagctaacattaaaaaattatttactgttagtgttaaaattttcttttctctactgttcatcttttgtttttcctccttcgtcttcaaaacgttctatttgttttttaagctctttattctctgcttcaagttcttttactttttgctctaactcttctttagtcatagaattcctcctactttataaaacctttaagttctaaaatagcataatactcagggttaattacatgtaaaccgccccaaataaaactagcaacaatactcactaacataattgagtacactacaaaagctgtcgggtgattaaaaggtacatggaatttttttactaatacccaaaccagtaaagataataccaatcctaataaaataaaccctaaccctactaatacttgatgccataccatagctgaaaatccttgacctaatgcattacttgcttgttctactgctttattcatattaatttcctccttattaaatatctaatttctcataacaatcttcacaaatcaaacctgattcctctttttcaatatactctttattatctcccatatatctatatccatcttgccacatatcaatataccctcgacacatatcacactcagtatcttcatgacttgtattattgagattaaatcactacttccacattcactacattcttcaggctcttcatcatgaccccagtcttcgaaatgcttacaatcctcacactcataagttaatacaaaatcctcttcaaattcaaagtctctttgtttcattttaatttcctcctttaacttgctcaattaaatcttttactgcataaacttccggattaattaatctcataatatcagttgggatacctgcaactattgcgtaaagagagaataacgttaatagtattaatagaattgtgtaacaccatccgataccacttaagtcatcgtaatcatcatacaataagctttcttttaaatccattttaaccttcttatattctttaaggtaaaccttacctaaaaatattgttagtgcaataactaaaatacttacaacaatatctataataccgtacgtaatttgatactttactaatgactccattacttgattacttaagttacctgctttatttccaagagaatcaattctctctaacactttatcttctaacttcattacaaccactccttatctagtcttctgagttccatccattcataaatccaagtacaaacagtactgccaatataaaaattacgaataacataggtcatacctcctctttatttgttaacttaagtataacctatgtatactcaattgtcaagacttatttcttctttttacttaattttttttgttgctcttttaatgaatccttttgttgaggtgttaacattaattatctttccttatcaaattcttttgttgttttaatcttgtattcttctttaggattttgttcttctaatctatcttttaaatcctctaatacagatacatccctgtgagctttaatttgtctaggtattttctttttacctattttgttataataacataaagcataccactcattcatttatataaccaccttacttaatattcttgatttctaactcaaactctactagtgagtttttagataaatctgtgatactgaacacattattatctcttttaataaagtgattactaggtgtaattctttccggttcatttttcttaatcaataattctaattgagagaataatctttcattaactaaattattaacatcctcttcaatatcttttctagttaaataagaatcatttaatttatagatatttttgttaacattacctttaccaataaacatattgtctacatgacaattacttcggtcaccatctttaaaacacacatgaccgttcttaaggtctcctcgtgattgactaatcatttcagggaatgtatgtgcaataactactcccctagaataacttgttttcttagggttaattaggtatacaatctcatgagcagtccacttaggatggttctttcttgtctgtgtacctttaattaactttttcgttactttatttctaattaaacccgtaggatgcgcttcataatacccattaaaaggtactgttctccatcctttctcaggctgtaatgagtggtctaaatgttttcttcttttaatcattatttttcctccttttgtaacttcttatctaaagttacgctataatcctctttataaaatcctttaattctacctttgcgccccctagcatgtttaacatacttataaacagtacctacaaattgaattctctttccttgaagctcctttagtttttcatagaaagagataccaacttcaatgttaacgtggtcaataggtataccattaatctttacatcagtaactgtgattttaacacacccatgttgattacctttatgtcttctacgctttgaatacttaacatcatctaagtaacccttaacattaacttgtttcctattaaaaggttttaactcttctctcatctttccacctcctactataattgtaatttatgatagttaaacatatctttagttaatttcttagcaaagtaattaatcatatattgtgttgaagttccttctagtttatcataatcaaaatcaatgtttgtaaaaactacaaagcttcctaaatttctactgccttctctgacttcaaatacaagtgtatcatttttaaaagttttaattttaaattctttattcgtatcaaaacttttagggaagttccaccattctttagttgttgccataatctcaattgcatttgaaaattgtcttctaattgtttctttactcatagtaatcatctcctgtacttgttatatactaagtataatacaaaagtacaaaggtgtcaatacttatttttaagaaaatgaaagaagatttattatcattcctagtgcacatattaatgaagcactggaatatagtattgactccttatcatctttaactttcctagctaatagtaagaaaagtaacattcctgcttgcagtaagaaaaaccatccataaaatcctgcccaaatatttattaagattgcagtaccagtaagcatatagctcatatcatcattaatcaaatcaataatgactacagctataaaagcaacagaagcaatagttaataagtccataggcattaatcccagttccagtcttccatcacaaaatcaatgtaagcttcattttcttcatctataatttggtctttaactacttctatttctgaaataacaaactgtaaagaatctgttaatctacctagcgcaatacgttctgttgttttaataggtaaacgtcctgagtgagggtgaactaagttatcaatgacttctactgcttttgtttcaccacttacaattttaatagtcccttcatcaaaaaatactttacttgattttcctgtacttgcgcttagtaaacttgcaatactattaatcatgtatacttttgtatcttccaggttttttagtttttcatttagcattctatttcctccttataacattaagtcttcatttagtttatttacagcttctataaaatcaggattcctatctagcatatctacaggattatcaacatcccattctattcctgcatgtatatctaaatatgaaattggtatgtctataataaaatctgcgtcttctacagcttcaccgtaatgataagaattatcagggtcatcttctgcaatgactatattatagttattaaatcttttctctatattctctctgctaagttttgttgctgtcatcttaaccaaaccatgttcatcatttaaatgattttgtattgtttgtattgtttcttttaaaacccgtttatttgtttctttataatgaataattagtatatccatctatacaacctcctaatacaagtatactactgtcatagcattatggatagatttcataatccaaaataagaatagtagtaaggctcctgctccgataacacctggcatccatctatctttccaaaactcaattccaaacatcaatacaaagtatgatacaaatccccatataaaccataaaattcctaataacataataatcattcctcttttctttattttatttatacttttagtatatattaaaatgcttcatctgtcaagcctttaatgtaaagttttttattaaaatattctaaagctttctgagccgtagatacttcttttgtgaactcacgtaaatctgtcatgttaagctctacagaacctatacgcattacaagctctacgccttctacaccattgtcaaatataataggtgtaaatgctgttttatgtttcttatctacttcagtaacaataatattccctgctaatcttacaactatagtataatctctatacttatctttgtaaagtacatttgtttctttagtcatttctttttctccttaaatcaatatataatgatttctttcttatctcttttaatttcttttccttatcttcaataggagtagaagaaggtagcttattctctaccttcttcttttgtttaggcttacctaccctatccgattcactgtttataaagtcattaaaatccatagtaaatctccttatagtaaaatattatttactgctagccatattaaaagtacaataattccaaacacaattgtactgatgactgccattatcatagagcaagcttctacatctaatgtagttttacttaataataacatcattagcttatctagtagaatagtcatacctttaaatactaagtagataattgccatagctactgcaaaagctataataatgtatatgacaagtagtatactatcaaatgagcttatcattgctttccacctctttaattactattgtattgtttatattcttctaaatcttcttgtttcctaatttcactttccttatctaatttctttaattttttatattctaaaattgttaaaggtctattagtaagcaattggtttttccagtggttaacatcttcaggtctttttacttcagcttgaaaagcccatgatatatttttttcttttacttcttctccattgtctctcttatagtaaaattctttaactcctaataaatctaaataccatttactatagtacttgtagttctcttctccaggtaatctgatgtaataggattcattccattcagtatagctccatcccggtgatacagctttatgaactagaataggtattaacgctgataggattccatatatagtaatgattatgaacatccctacgattatttgggctaaagtactctcaattgagtcagtaatagtacctattaaggttaaagttaaaataatagctattagtaaattaaataagtacactccgataaatttactcaccatatctctcctcttctagcaagttagtaacgtattcgttattttgttctaattcttggatgtaatcaaaagctaattctaacattttactttcactaatgtgaccttgtttataatactctaactcattcttgaatttgttttgtgctttaatactaggtataaccataatatctctcctcctttagtaatactatactacacttatttgttcttgtcaacagtttctttatcttttttaaaaagttctttagataatttagtatgttgttctactgccttattatatctcatatatttaagatgaagtatattaatctcactaaaagttatttgtcttaagttatgttttgatacaatatctacttcatcatatgaaaccaaccataatagaatctaatacctttgtagaaatataatttcttgttcctttagaacgtatgctgtccttataattatctacttcttttaactgtaactgtatgtctctattagcctcttcaatatctttaattaatttttccctttctgtcattgtcctatctcctttggtctatttgagctatcgacttaagtaagaatttatagtttcgtttaaaattttttgcttctaacttaccaaatttactcttaatacgttttttagccttgtaagctgtacctccttctagtatcgggtttagtaagtctacgcttaactccagtattctttcctccgaaagctcatcatctgaaattagtcttacaaatgcatcaaatctttctaaatctgccatttccatagttaatctctcctttctatatattaatataccataaaacaaaaagagagtcaagaattaacttgactcaatcactgttccataagtaataaatgatagcaataaagatgacgacaattaatgttgtcatttaagtcacctccttattactttttggttggtactgcttcaaacctatacttatgaattggattatcttctccatcttcagtatataccctaataagcttgaaatttaccccttccttaaatctacttgttttgtagtacctcatatctatgctgttatctcctgttctacctggtaggtaagtaggtaatgtttccataaaataagctttattatcttccacttttttcaatgtgtagtcaccgacaaattctacgttcttttcttctttaatctttcctgtgttcgataactcagtagagacttcattttttgctttgtcgtgatacatgaaagaagatatataataagatgagatagagaaagccaaaacaataaccactagtatatagaatatacttgcttttcttgacataattaacacctctatacaaaaatgtcccctcttacaattataatataagaggagactactattaatctataattttaacacatttatcgaggagtgaacaataaatcattttgtcactacctactaattctcgtaatttactatagtacttaagtaaatcattggaatccgttagttcagtgtatcggtcaagaacttcattaatacgactttcaaaaaacttattatcaacaatttctaaatgagaaacttcttctcctgttgctaatcttaagtcttctaactctacatcatatatctttttgtcatccttaatgtcttcaagtaactcatgttgttggtattcaatatttgtattattaatacgtatcaaaggttttaaaacatgagaagtttcatataagtttctaggcatttgacctcttaaatggctgaagtcttctgactctgataataatccagtactaataccaatatattgtcctggttgtacttcggtagactgatatcctttaggtattgtacagatatataacttattaggttcatataaatctttagatgcaggaaaatcagctacaacatcttttaatccttgagcatctagtaagctctcttttaactcatttaaagtatatagtttttcttcttctacttcaggttcttcaggaggagccacttcttcagtgaactcctcttcagatacccatttctctttattacgcatgtcatttaactgctcttgaaatgattttttatttctcatattataacctctttacttactcaacgacaatattatcctcttctaatgctttgtccttaagttcagattttctctccacatggtataaatctaagatgtaatataaaccattcttatgaagattatatttactataaatgtcctgtaaattcatatattggtaatccttaataatctctttgactaagtttttatcttttaagatatgtgcaattctttcttctacaggttatttcttattacgcttacctactttatagtagtttagaactgtgtaaattaatcccatacttaccttagcttcttttgcaatgtaatctaaaggtttaccttcattatagcttgttacaatgaattcttctttaggtgtgttaaactctcttgatgtttctaatggtgtgtatttactttcctctttagttaataattctttctcttcttgatttaatgaaataaacataattgttagctcctttacttttagtatacatatactatatcataaactataggagctgtcaaccctaatctttaactttatttaatttattttcatcaaagagtattgtttcccctgtaagtgcatagaaataattaccattattatctgtaagtatttctttaatcatagcaacatctttttcttggtcttctcttgtatattctacaatagtatctttaggatacttaaccttagttgtttctgaatattcttcatctagataaacatcaaatgttataagagagttaacagatacattatagagaacccaatgccttttatagtagaatgattctttatctaagtctctagatacttcagtaaatttgtgtttccctggactcattctaacaaggatatctccttggttgtcttgtttaactctcagtactttcccgttttttaagtatgtcatgatatctactatatcgtcaattggcttagcatcttgtattggatttaaactaatctcatctgccattactctaacctcactatctaaattgtatagcctactctatcaagagcttcttttaattttctactttcctcatctaaatctagatttttgtagaattcattttgtagttcttctaatcgttgtaatacctcttcatgtttctcttcaggtataaattctaataatacttcactcatagctgtatcttgagctgtttgttttaattgtaattctctaagtccaataaatgacataccttttaattgatttccggtaatcttatctttagtttccattgcacgtatcattaaagcagggtctaaagtttccacaaactctaatccttttgcacctttagaaataatcatatctagtacttgaatatcatcataaattgtttgtgtgtgatgttttgattgttcgaatggagaaagttgctcctcttctaatataggagtttctttttccttaatatcttttacacttgtttttttacgtttatcaattaattctcctaaatcccaaccattttcaatagcttcttttcttttacttttatatcttgtaatagctgatttagacaattctaaatcatattctttacataattctataatatcatcataactcatatcttcatctaatgcattatctacctttgaacgtagtaacttattatggtataacttagataatatactttctttttctttatcaggaacaacttttatatcactgtttttatttcttaaattcttttttctagccattatatagctctccttaactgtcatttatttatggtaactgatgttctgaaaccagtttaacgaacataaacaaaccctacaacatcaactgtttcaaggttacactatataatatatcacaatttctgttattatttcataatatataaatgtaatataattttattaactactttgtataaaattttaatatattttttaactatttacaaagcctttacctaaaagatattttaacttttcataaatataatttcacagaaactagagcaagtagaaaaaacttaactagtttctgtattaaaaacattatgaaaacttcttatataacgtatatttatacttaattatcacatatttataacattttcctagaataaaatagttaatgtgtcgtcaccctttaggttaattactagctcagttaatttatgagtcttatcttcaattttaacataactagacttaatatctttagtgttaactttaacatatgactgaccacaagcaatagtaataacatctgtgtcttctaaaaagaatatccctgtctcatgtgtattatataaattaaagtagtagtctttacttaataacttaacacaaaatacacttttacttattaaactattgtttctgaagaatgctaataatttatcagtatctcctttagataacttatcttttttagagagcataccgaagagtgtttgaccatcatatagtttttctgtagtaagtaagtatacaatatgagtgtaaccatttaatggtgattcttcttctactttatacaaacgtagtttaatatctttagctatagtaatattttggtatgttacctcaggctcaataaaagtacttacaatatctactttcttagtagaggttaagttagtacctctagcaagtaaatctgtcattagtccagtctcttccaactcttttttaagttgttcagttctcacatagccaagacctgaagaacctaattgattattcatacctactacagcctgtttcattgattctactacatttaccttattaacaaactgtagaccctcatcttcattacctatatcctcaatgtaagattgtactgtaacacctgaagggaagtaatcactatcgtaagatacacctagtaattctactttaccacttgttaaggtcttgtataaggtcttaatagttgtaggtttgtctacatgttctgaaaatccttttaacattatttatctttcctttctgctttaactctatgtcttgttttgaatataaatatcaaatgtttacccttaaatgttctttgttctctgatactacactttccaataagttttttacctctatgaacataataaactctatctgtatgcaaaatagattctgttttttttcctttttgcttaagagacaagtaagtttctttaacatcaagtgcttcactaataagatttagtgcatactttttctgtaatttcacatctgtacttgttacgttatcaaatcttttaataaatagtatacgatattcaataacagatgttgcatctttaggctctgaaggctttttagcaaagaaactattattagctcctgttaaagtattcgtaggggcaaagttaacaggttgatgctgtatctctttacgtctatttggtttgtctttgctcattaattactcttaccccattcttcatagtattaaagaaatcttcttctataacttcccacctattattataaccatagataacacctcttgtaaagagtttataagtattatctacccctacataatgaattatttggttaataatagcaggttttttcattcttttactttgagttgtttgggagaaattaccttcccctccactatatctaccactaacattatagctataccctgttctatacgtatgagggtctctaatcaatctaagtattgtcattaagtttgtcttccataatatattcattgtatgataagggcttctatgtggtgatggtgtagcatctttgtatttacctacaacaggtttgcctttaaaactaatgtaactcattatacctacatgattatgtttgtaccttttaagtatttgttctactttaggtacatgtttgtcatggcacattacatatacatacttacatactagactataatattttaattggttatttaatctttgtgtagagtctcgttctgtttttatctctatacccattactgtaccattacggtctaacactaaacaatctgttctacatttaccttgacaaatgactttctcattgaagattcgtatatcgtttatatcactagttatttcatcgtctttgaacatgtgttttttagttctgataaggtctttaatatctttttcatagaacttaactacatcattagacatttaacctatctcctttaacccttttgtagtactcaacaacactttctaagggggtatatgttctattccacttaacacttgttagacctataaaaacacctgtgtattgaccatgaacgactttagttggttccttttgagaatagatagttctatccttatctctttcaaaaaatcctttaccggttttacctgttactgtatctgttcctaaaggaataacattagctacaaatatagggtcttctgtaaccttcttaggatacttacgttgaggcatcatactcttaattaaattactaagaggtgttcctcccggtattgtacgttggtaaggttctattctatatttcttctgaggattttctttttcctctctgtcatgtatgtcatatacaataacatactcagacatttcttcttctacatgctcaataaagtaataaggcgtatcattgaaatagtatacaccctcactaattctcattgctaatgagctattatcaaaagcactaaaattaaaatccatttttaagtcacctctatattaatataccatatatagtaaaaagagtcaagtaaagacttgactctataagaaatccttttccatttttctaatagtaggtacaatatcttctacatctgttaaatattgtttcactgacatactaggtgtttctttattgattaaacttactgaatatggctgaccttcaggatggtctaccataccatttgtcatttctttcaagaatccatcattaataacattaaataaatacttatcattacctttatcaaactgtattgtataaatatttcctttgtcatcttgagactcatgaatgttttgtactgtatcatttttaattaattgctcataaatatcattgaacttcattattatcctcctgtgtttgatactgtctagcttttctagcttggaatattgtaagtacaaatgaccctacaagaccaattataacataaactagcatgtaaataaagcttgtaataataacaccgaaccctgattgtgcggaatacaaggaatccctaaaaataatactcagtataataacaataacacctactaagtaaggtactatcacatgatagtactcaaagttacttagtggaggttgtccttgacttaccatttttttctctgtagtccataaatcaaataatctaaagactatgttaaatgttgcaaaagttataaacgctacacctataataaaaattgtatccatttattaaccatccttaatctaaaaatactgttttaatatcaatatctttattttccttatttacaaataataactgctgtgaaggtttagtttttgataaatgtaactctttagcatagttattatatcccatcggagatgaagctacgatgtgcattctattaaaatcttcttgttttactgagaaatgatgtacatgacctgtaattaataagtcaatatgactatctaagataaatttattgatatgattacctttaccttttaacccatctccgtggttaatgataatagatttaccgccaaaggtatctctaatagtataaatatcttcacgattatcaataatatctacaccatttaatagcccttgttcttggaataacaataaagaatctaacactacataagcaatgttatcattataaatcttctgattcttgttaccttgcatacggtcatggttaccaccaataataccaaatcttagttctcctgaaactacattagatagtacatttaggatatcaataagtaatcgagtacctttagagatttgttctgctaaagtaaactctgtttcaaatgcttggttaacatctctcatattaatatgttctactaagtccccaacaaagtaaacagttacattagaaatttccctatcttcaatttctttaactgttgtattaataaaatgatttaatcttgatttaagaacttcaaaatcatatttaccattagtcatatcagatacaatacttccaatatggaaatctgatagtaacacaattagctctgtatcattaatattagctttacttccgtgatgttctactaatgtttccccttgtaattccttaaccatatgttctttaagttcatcaaacataatttgagggtatgcagtatgcctattgtgcttacgctgttctcttaagtgagacaattctttttctttagctactacataagtagatacttcttcatggtagttattaccttcgaatacttggttatctgcaacatcttttaaatcacctaacgttactttacctttaattgctttgtttttaagttcatttagataagcgtcttgtctaatatcctctacaatttctttaaggtcatctttaccgattctatctaaaccaaactctttagtaattttattaaacttagatgttacaattttaccttcatctttaaataataccccaataataactgcttgtacttcttctattgttttatagttactcataactgcttcctttctctcttataaacttagttatttgatttcctaaaatattaatgtgactgtttacaaatgtatggaaaatattagaatcataatcatgttctacattgaacatactagctattgccttagcatccatacctttaacataaaaagtcagtatatcattattagaacgtgtctgtgaaccgataataacaataccatgctccttgtttgaaagtaaactataagctagaggtaatttataatcaggcagaacctcatataataaatatacattaccattattatagcacttttctcttaccatgtcaacttcttttttgtaaaaactatttaaatcaaatacatcttcaaaatcttgaaagttataatacttagttaaaccttttgcatcatcctctacccatttaggaaaagggtctttacttaactcagttatctgttgtaataatgaataatattttctatcagaatcaacagtgataaaagtatcaggaagtaatcctatatcagagacataataaatatcattattcttaatataatcatcaatatctgaattagaattaatgaccttattaaatgtgatatcatcatagatgaatcctgctaaaacagttggctcatcctgtattgaaagcaatccttcttcaaatgtatctactttctcaatatcaaaaaagctatcatactcatataaagtgtctgattgaatatggttggagtttactataattcccataccctactcacctactctttcatataagtagcctttaacgtctctttcatcatctttagaagaattttcttgtccgtactctttaactctgtaaccatagatgtgtaggtaatcttggatgacattcatcataccttgaacatttaaattatgaacagaaacagaatcaaactttactacaatatactgctctttgttttccattaatttttttactttcgtataatcatatggatttctaaataatattgttggaatatccattactttcccctccttttcttaattcttatatagtgtatcatagatattataaattgtcaataaaaaaagaccaactttttagttggtcttaattattatgcgtctcctgaaggttcttcagtaggagttttagttgtaaattcaggaacatctactttttctgattcacctgaatcattagcaaatgctactttaaaatctcctttagcataagtttttccggcttctaagcctgtaatagatactgtagtagctccatcgttattagctacttgagagcccacaacttcaccattactataaacttttaatgtcttttccattctttattgtcctcctatacagtaatatctgcgctattttcaccaggttcaactgttacgtctgcaggtgcttcaggaataggttccttaactgttacaacaacaatatcagtgtgactaccatcttcagtagttacagtaacattagctgttcctgcactaactgcttcaattaagccttctgatgttacgctagcaacacctgatttgtctgattcaaatgacacatttttgttagatgcttcactaggcgtgatagttgttgatagttggtgagtatctccaactgtaaggtctaaactatcaacgtcaagggtaactgaaataactttaattgcttttgtcttaaactgaggaacatcgaccttctctgactctcctgaatcatttgagaatgatactttaaaagtacctttagggtaatccgtattagcatctaaattctcaatcgtgattgttgattttccttctgttttttctacgctgtttagtaactcattacctttgtataaatttaacttagccaaatttaccatccttatctatatattattctgctgatacgtctgctccgtcttccttaggagtaacttgaacgttcttaggtgctgatggttctgaagtgtctccttgaccttctcctgaatctacgcctttaacgtccttacgagcagggctagcagggaaatccgcaattacatcttcatgtgcgtgctctaaactatctttaacttcatctgcatcattatctgtagggtctacaatagataatacatcctctttgttgtttgcaacaaatacgttataaacaccaaatttgaaacggtcttcaacgaaagctaagaaagcttgaataagttgtttacctttatctaatcctttaatatcttcagtacggataaatgagtgattataagggtctactactgtgtttactacgaaatggatattgtctccatcagtgtaagcaataggtgatttcttcattacatttaatgtacctttaccatctgtttgtacagggtataagaataattcttctccctctgaaactactttgtacgcatattgtccttcatgtgtacgagtataaccgtcacggttaactttaacttgtaaatacttatctgctactgtaacagattcttgttgtaatacatcattaacattctttttagccataagcttaatctccaatctttattttagtaatctattttaaagcattctataagacatagaaaaaagccttccctatgctttattattgaattctcgataataatataacacaaggaaggcactgtaactagttttcccaaatatgattttcagtattaactctatggttatttaattctttcttagcatagtactcatggtatgcatttattttaaatctaacatagtctctgagttctgtcaactcacttgctacttcttttcttttcatatcaaacttttccgctacttgagaaacgataaaggcatcatcttctctttcagggttaataagtaattctttaagtaactcactttgaacctctgtaaattctatatcatcaaatacataactcataatttgattatcctcgaagtcttcatttaaatcttctgttaaagactctactgtataatcttttttaccgataatttcagtacgtttatatttttcattcttcttaacataactattttgaactcttaaagttagtttagctttaatatatccaggaaaatccactttacttctaatattatattctttaactaatttaataaattgctcatcaatatattctcttagttcttgtctttcaaaatcattagataaaatccctgcgtacctatgatataacgaccatctaaggtttttatacctattaagtaaactatctaaatccctttcaaaatcaacaggtataccatctatatctattacatatctattaccattattagctttcttcataaggattagccccctcgaagattattcttactttgtgtgatttatctttaacatatatatcagagaaatggatagtcttaggtactttaacttcttcaccttcatgtctaattaaggaatatcctcctgcccatctatctgttacatgttctatatacatcactgtagaatctgtataagggaatccaccttcttcaatgataattgattcacctttttcttttacttcttcaaatttctttgctatatctttataaggtaaaggaagaacatctgaggaatttgcattccccaaagattcttttacctgattcatgtttttatctattaaattatccatctattattcagaatccttttcttgagattctccttttagttcttcctcttgttcctcttgtttattatctgtagcccattctttatagtagttttcaagagcttgatactcttctaattgagttacgattactttgttattaagtaatggtgagtaaccattagggaagtgttctccataaactctactaaacaattctagatataactctttattatctttaagtacaggaacccattcagcatctctaagtttaatttcttcaccattcaatgtaacatagtttctccatgctcctttggtaattaaacctctttcaactgcttctttatatacagtatgttctaagtcgataccatttaataagatagggtcatcttctttacctaccatataatcagatagtaagtctgcttcagctttttgccctggtcgtgatagtttagatttcttagtttcaatacgcataatgtgaccatggtattcatctttaccagttaactcacttttctgttttaactgagatgctttatgaaccttaatacgtaaactagcactatgttcaaaagctctaccacctgtagatttaatagggtcaccatacatacctgcattcatatcatcacgggcttggttaataacaattaaccctgtgtttgtatcatttagtttaggtgttactgcattaattactttttgggtagctgatgccttaagacccatttgcttctcatcaataccgccatcaatctctttctgagttcttgtagcccctaatgagtcccaaataaatacaataggtactcccggaatcttttcattgaaagtgtcaatccagtactctaattctttacctacttgctctacagataattctactgtatttttaagtctaccttctcctgattgaatagagaatagttttgaaacatctacaccaagttgctccatacgattgttatctgctgttccttcaatatcaatccaaatagtgataacacctaattgtgttgcaattcgagataagtgaactgcaaaagtagatttaccactgccagttaaaccgtaaacttctgttaaccgacctaatggaatacctccacctaaaatatagtcatactgaggaaccattgtaggtataacgtttttaatatctgctctgtttgtatctgatagcaatgttaatcctaattctttacctaaatcaattgtatttaaatctgttaaatcgacttctttacctttttttgctcttgccatttaatatcctcctagtaattatatctcaaataaaagagtgctctaaggcactctatatcttataggttatctaagtctagaccacctaatacatcgtcaatactttgcgtttgtttttgttttggaggctcttgtgtagtttgttgtggttcaggtgcttgttgtgtattttgaggttgtgtctcaaaaggtacctgtgaatcatcaacactgctaacatcaaaatcattgaatgggtttgaatcaggttgtgattgttgttgcgctaagttatcccaatcaacagaacctgtgttacttggttgttgtcctgaaggagttccttgaccaaattgagtgttattgataggttgttgtgtaccttgtaactgctgtccttgaccattactttgaccttgttgtgcatactgacctacttgaccttgctgaggctgattaggttgtcctcccatattacttggcatttgactatctacatcttgttgtgtgggtgcttgtttaggctctgaaggttcttcacctaagacatttgtttcacggttaaatttaaagttatcatgacttaactctgtgttattaacgttattgattaagaagttaacaaaattaggattttgttcttctgttggttttgctaattggtctaagtcagataattgttgttcccaaccttgaggtaacgcacctaaaggggcattaggataaacacttactttccatgacatttcacctttcttagctttaacaatattaactaggaatgcttcagttgctgagataaagctatgaggtgcattaggtgatggttttaacatagtgtcttttaagttagctaataattctttatatcctgtattagataattccataggttgaatcattacatttccttgagcatccgtttttggttgaatttgaccattgttattaaagtattcaataacatgaatgtaatagcgtactcctggtttattagggaatctactgaactgaacaccatttgttatccaatcctgaatgtatgggtcaaggacagatgaaccctcttctgcaggtaatgttaaccctgtgaatccctgagaaccatctttcttagaatagttaatacctgttgttctaaattctttaaagaaaacatttgttccttgtgcaggaggtaatactcgtacaaaagcattttctttaacaaccttatttccgtttgcatctttaatattacctaaacgtagtacagggtttttaggtttgtagctctctacattattgttaaaagaactgttattgctttctaatttgcttgcctcattgttaataaattgattgaaatccataattatcttttctccttttaattaatttacttatttattataacatagttttttatatttgtcaattatattctttactttattttttaatgtgaaaatggattagtaattccaccatttttatttgtttgtcgtaattctgcacctaattgaatcatcatatctcttcttgtctcaaaagcttttacaacatattgaagttgtttaactttataattccaagcctcaacaactttaagttgttgttgataagaatcgtgaatataaacactagattcaatcatatctttagtaggctttttaccttgttgttcgtactcttgtctaattgtaaggtttagcttactaccaattgtttctaggtttctttgttccatttcagaatatgctcttaccttctctaatatagaagcccaaaaagcatatttagctgattgttgcataaaatcctcttgtattgtatactctgtaattcttaactcatctgtaatatcaaactcttgtgagttcccatttctatctttaatagtaaagtttgtaaaatctagggaatcaatgtgtatttccatttatcagttcctccttttaaatattaactgagatactacttaaaggtaaagctaaaggatattcttcttcaagacttgccaattcctctgttacttctccagtagatgactctgttggtaaaactgtagaaccatgtttattttcttctctattatagaatgtaacaacaatattcatcccacctagctcatcctggtacatgcttgttataatccattcatccatagaacgattaaatctaatctcccctgtattcactctttttcctccttatatttattacttatatagtataacataagttaactatattgtcaatacttttattaaatttcttgataaatatgtttttcacttttaacaacattagtagccttttcaaattgttcatctgttagtttacctgactctttataatcttctaaggtctgtaaattcatcatatacttaatataaccttggtaagaattaaatgtatctatttcttcctcatcatattcaaccatatcattatagtttaacccaatttccatatcggcttcaataggatattgtacttcttttccatcaatttctgcttttaagaaatcaaatggtaagttttccataatatgaattgtcactttagccataattttagcttcttcaggaggacaatcaattaagatactatcatgtactgtggcaataacttttgattttaaattacgagattgaataaaatcatctaagtaagttaaagacatgtttgttaagaaactacctgaaccttggatgatagtgttaaatgactgtcttagaccttcattttttatctttttatctgttgattgggctgaacggataaatcttctatgtccgtgcattgtatcaacataaccatattgttgcacaaactcatgtacattgtcaatagaagtttttacacttggttttgtttggaaatacttttcaaatatttcttctgcttcacttacttccatattatttttacctgcaaatgagaagggtgattcaccaaaggctaatccgaagttaactgctttagttgcttgtcgttcttctttagttacttcttctactggtttattgtaaacaatacttgcagtagccttatggatatcttccccatttaagaatgattgtagcatatcagggtctttagtaaataatccgataatacgcatctctagggcgctatagtcggaccctagcagtactccattttcaaatctagaaacaaatgaacgtttaattggatgtttgtaatcaaacttatttacatctgatgtgtgtgcaggtaagttttgtaaattctataatttgtaagctttttagtttacaatctgttactgtttaggtaacacaatagctctcactattgagtagactatatcatatatcatttctgtggtaggttttaattattatttttaaatttatatcctctgtattcaacaaagtcatctgtatttcttagtttatctcttaatgttttatgattattcatttctagtatacccataattttattcattgtcaaatcttcatataataaattattatttaaatcatacatattaatagacttatttaaacttctctttgaatccttatatacgtattcccaagtataacttctatgatgcttacgtttaccgtgagcaacggaactaatcttagtacttttaaagtacccattagattctttttcagcttcacttggtgaatcccataacttaatgatgttaccttccatatctttttgataaatggctttcttagatacagaactaagtttttctcttgcactataagtattcaatcttccttgcttattagcgtgagcactattacctttgtaacttacccattctaaattttcaacacagttatttaatttattattatcaatatggttaactatactatagctgtcggtaggtataaatgtcatagccactaatctatgtactaatacattagcaatacctatagatacttgctcatatccggaatttattatccacggctttaaaatatagttagtttttatattccttacttttcctttattacttacctcgtagttttcaaatcctactactttcttccaaatctctttcatataactctccttattattccctaccacagaaataatacctttgctttcgtgggtgtgtattgattgtattgtctcaacacctagtcgttgaaccttacccatacttttatatactttcagggtcttggatgcggattctctctattcttaacctttttactatatcaaacacgttactgtttgcccttatatatgtttccattataagttagtagttaagacctaacgagtctcttcccgcagttaacaaagttttacaaccccaatttaagggttactactacttagtcttgatgtctctgtgcctgtctcagaaaaagaaccatgtaatgttcgtttttgtttattaatcatattaagtaatttcttagtaaatgaattacgttttgtctgcatactagcatgatatttaagtaattcaagaagctctttaacatcatcttttaattctaaattatctaacgcataaccaatagctttcttgtctgttttatagtcctgccaagtaaggtctgcttctttaacattagcattaaatggtttttccttgacatattctttatcataaggtaattgaattcctagaatatcataaattactctacctttatggtctccggaacttggggaaaacatccatttaccatctttaaatttatctctgtactgatggatatctttatctctatcttttggctttttctcatgttcttctaacgccatttggtaaagattgtattggtattcttcaaattctttaacagcccagtgctctctcattgtagcatggttcttagccatctcagactcgtatgactcatcattttctttcatataatctaagtcacaatataaaccatttgattcaattctagctaaagacttagtaagtcttgggtaattaacttctaataaatgcattgacttaggtctatcttgttctttaagtttcttaattacatcacaataaattcttctacatacatcagtatctcctgatgcgtatggatgcattaactcaatagggaaccaatcataattaaagttacctccgtcaatatcattaactacatcttttacatctctatactcattaattaaattaattgcagtatttaacgtgtaagacttagcgtagtcagataaccccatgtactcaggtgattgttctgcaatatttatgaattcatcattatccataactaaattcatatttacaatttcaggtgaaagacctagttgaatatactttttctctaattcactaactctaaattttttctcagtatcatctagttctactactgtttcatttaatttattctctaaccattctttatattcaggagctttaacatcatactctttcttagcaatctttttattttctttctgtatctctttaattttatctgagaagaatcttaataacttagtaacaaaccataatttaaagtcttctaatggtttatcatagcctccgacatctgtaacctcataagctaaatcagataatcttaaagattctttaacttcttgggtaacagctaggtaccaacctactttagtatcctgaatactctcaaaatctttaaagttttcagtactcattaagaacttaatatcatatttcgttttagcctatatatttctatatagaacggactataccttcataagcccttaccattaggtttgtttgtagttacctaaatgggcttgatgtccctcatttacatagtgtgattattttttatgatgagtatcttcatgacaggatttacataaccattttatttctaaaggtttagtataatcaggatggtgagcttcaggtatgcaatctttattacactctgaacacttatcaggtcttatgagagaaccatttcttaatgcggttcttacagagcttctagctttcgattttaaagaattgttttctctgtatttcctagacctatttttattaagctctctacctctgtcagaagtattatactcattactttgtcttctatatttatctccgtttttctcataacattttttaatagtatttttagttttatctaacttattatattccttccgacaaggtatacacatactttgataataagttttatcattcttcttatatgttttaaagttagataaatctaattcttcgttgcatcttctacatacttttttgttatttttaactgatgtcatataatcttttttcactgtaccacctctattaagtcgatacactatgtctcaccttggtctacaaaacctcggctcagtctctacaccccatctattgtcaccaataatgtaagctcgggattaccactatccttattacaggacttaggttctcttatcagctacttaaagctatgcccgatatgagagggattttacaacagcaaatcatttaccgttatgtgctactttaatatcttctttactagctaaccaattcttaagcaattctaagacttcatcaatatcatcttgaccgttttcccagttaaagtctgatttgtataagggtatagttacaccttgaccatttctccatgatagagatagtactaaaggtttacttccttctttatcaggttttaatgagttagtctctaagtcccatgcggtaatatctaccccatcataattatcattctttacttccttattgaatatttctcttactctttcaatgttatctaccaactcgtaagatacttccttaggtttaaatgcctcttctccttgctctacaaacttaccaactgtttgtaaatctgatactacatgacgttcactatttttatttacattagtatattcaatgctataagtaggtaatacccacacatcatgagaagatgttccattttcaatagttactttacttggtacacctcttactttaccaatagctgtaacatttaataggtatttaacacctagtttacctacaggaattaccatatcataagaattatcaataataatattattcattctctcatagtatggctttacttctgcttgtttaacatcttggtatttaatgattttaccgtagtcatttctaataggtgtaggtactgcattgtagagaaaatcaatatcataatctctgtttgttttatctctctttaagttagaacacttctcaagtaatttcttaagtgtttttccgttagatgtatttagtacattagatttcacactcccatctttacttacagaaaaatgctcttctctaatgtggtcaaataagattaatactttcatttaattcctctccttttttaacttaataatactataccatatattttaaaccttgtcaataaaaaaagaagagaattatttctcttcttcattaagtctatcaataacctcttctagttcttttagtggtttgaattttatagcttttctattaggtaaatggaagtattctttattcagaccatcccacgcttttttaccttttctttcaataacttctatttgcataaatttatgattctttactttagtatacccttcttctagcaagtcagaaatagcatctgtctctgcacttagtatttcctctacatcttggatatagtaacctgtattctctgatatctttcttgctatatctcttctatttgctgtactcatataatcaccttatatcttaatctttcgttgttgttttaatttcgtaattacattgtctacatcctcaaaatcacgactacttgttaagatatagatatacattttccatactgataaagatactctaattttatctgcaaaatcgactttatattcgggttttaaatcataagcttcaccatcaaagaaataaaacttttcttgtttttcatctacccctaatattcgtggaaatgataaatgaacttcatctacatttgttttaatatcatgtaaacttctaataatatcataagggtttacataaggaaatactacagaaatatcaataataacttttgtacccatgaatgataaagctatattatctacttcttttgcagtaaaaccgttgcgcatacgataaacaacttctgagtcagggtgactagataagtcttttaaaacatagtaaggtaatactgaatcagtatagtatgtaactttaccaccgtgtttacatactttttctataagcctatcattattgtacttctcattaaatagcatgtaatgcttttcatccctcatgatatatcttctagacttagatattaattcatccatattcttttcaatttcttcttcattaccgtgtgttactttaatagcctcattaattttatctttatcttttgtgtgtatattaaaatatctatcactattatatatcgttaatgttttattctttgcgatatgtcatcttccttccattaaaaaatcctactaccttgtactattatagtatagcacagatagtaggaaattccaaattaaagtgactctaattcttgtaattgcataggtgtaacttggtcttcatactttttaactaactgaccatctttatacccaattaatactggtgtactcatgatatcgaataacttaacagctttttctctatctacatcttcagcatctagatttaatgttaagataggttttttaatgtcacctgactcttgaaacatagggattacactttttaaaatctcacacttaccacaattatcttgtgtaatcatgacaataacatctttcttttgtctaattgctgtatttaaatctactaaactattcattttttccatcttctgaatcatccttttctaatgttttaataatttcgggaaatgagttaactattgtagctttttgtgcttcataaccttctgctaaatcaataagttttagctgtcctgagccataagataaagtatacatggtcatatcatgcataaactctagctgaaaagaattagacgtatcatcagctttagcatccttagagttactgaataacttcatagggtcattgatatcacttacacatgaaaactcatcatgttttttaaataatatttctgctttacttagtaaatccattgcttcttttctatccataatatcttttctcctttttataggaattccttggcagtactgtattctttactatctaagaatgagaaatcttcatctttaatatctttgatatttgtagatttttcatagtctgctttaccactaaagaaatcaacatttcgtaatctgtctacattagtttgattttctacaatagggttataaggttcaggattaaaatagtcctctcttccaaggttttgaagagctctattaaaattatactgaacatagttaattacgtcttcacttaattctagtgggtcatatagactatgtgtatacgctacttcattagtgtaaagaatatctaataattcataagtttctttatctgcttgttttttctcttcttctgttagaagattataatcatattgagctgttaatcctactgccgaaccatggtaagcttcatcttgagtaattttataaataatagctcctgattgtgtcatctgacctcttcctgcaagaagtaaaggataataaaatcctgagtagaataatgcactttctaaaaaggcactagctactttagccatgtatctatcaaatatagtaggattaggttttaatagttggtcgtagtaatatccgataaactgagcttttacttttaaaaagtcgttttcttctacccaagtatctaataaataacttgtttctttattacttagtaatgttgtaaaaatatgactataagatttagcatgtatctcttccataccacccataaacgcaaatacagcttggtatttaggtcttggttcgtggtaggatactaagttcataccttctcctccttgctttgtatcgagccctgtgagccctgcaaggactttcttataagtgttcttttcactttcagttaaactattccaagaacttaaatctcttgatacatcaaactcttcaggagtccaaaattgggagattccttgctcccaataaatatcagacatcccgtcatcttcaatattccagtttgttgcttttaatacagcatttttattatgttgttttactttttgtgtaatatccatatctatttcctttctaaaccgaacatgtttcacattcaataacagaaagtttacgtgaacgtgtatagtataatgattttaatccttgttcccaagcataatagtataatgatactagtttattagtagggatttctgattctacataaagtattgtagacactgcttggtctgtatgtttctgaacaactgcgcttgtattaataatacgtttattgtcaatcttataagctgtttctccttcatagaagaactgagttataggacttaggtaaggcattgggtaatatgtctccatatctccatattgtctattctctatagcactaggaactggcataatagaacttgtagcattctgtacataagatatagattgagtaggtgcttctgcttttcggtaaccattatacaaaccatgtactttcactgctttagctaattcatcccaatcatcttgtgttggaatataaaccttatctaagactttcttagctttatctgtcttaggttgattggattctctaatgtatcttacaaagtattcacctgtagcgtaatcggacttctcaaagcctttaaatggttttcctgtttctttagccattaacatagatgtcttaatagactggaagttaatagcactatataaactgtttactaggtctaatgcttccctagaaccataactaatcatattcttagctaaaagtccatgtaaattcattgaacctaaaccgatagctctaatatcatcatttgctttttgaacactaggtaagtaaggtaaatccataatatcagtaacttttgttaatgctcttgtaccaatatctacagattcattcaataaacctttttcaaccacattaaccaagttaagagaacctaatgtacagacaacatctctattaataacattctgattactgtaagagtacggataaatttctgatacctcttgtaattgtgaaatttctgtacataagttactcattttaactttacctaggtttttcaatgggtgattatcattagcattatcaatatagaatacataagggtaccctgattctaattgtgttttagcaatatctgtcataacttctctagcatcatgttttactttaccgatactatcattatttagtaacttatcataccattcatcaatgtttagctcatctaaatacttaccgtacactttattaatgtcgtaagtatcaaaagtatagaaaggtttaccttctttaactaactccataaatttgttagggattgtaacaccaatagataatttatcgagtctaacagactcactagcattgattttcttagaacttaataaatccaggatatcattatggaatatatttagataaacagcaccactaccctgtcttacaccaccctggtcataaatactaactttatgttcaatagctttagcaaaagcaataacacctttacttgaattaggtctattacgtactggagctcctttaggtctaattctagttaaatcaattgcaactccaccaccattagaactagcattagctacactatcttcaacaaagtttaaagactcaattgaatcatctactacaaatagataacatgagcttagttcacctctattagcttgtcctgagttgttatacgtaggtgtagatggttggaatgtttgctccataagttgtactagtaattctctagcctttacatagtcattttgaaataaatataaagctactcgaacattatgttgttcatagtcttcaacaaaaataggttttttgttttcatcatactgttttgatgcgtaagattcataaaacttttgccctgccatgaaactagggaatttaaaattaaaactgtaagctaacttagtcatttcatcaataatagtgtcaggaacgttgtcaaatattttatcatatgttccttcttttgtcaatactctaattctttctacttcactattaaactttcttgtattgtcttcaatatattttaaatactcatctaaagcttcttggtctttatagagtttattttttccattgtcatctaattgagttatttcattatttaactcaatccattttccatatgttgccatatatctctcatcctttacgctactttatatttattactaaagtgttctaatattggttgtatctcgttaaagtgtcctgattgctctactttagcaattaaaggaatcttaaacatctctgaaatctttttacttgctccacagaaattagagccccattgtttatttcctgtgccaataacttctttagggctcccattaatttctaggaatctttctatttgtttaggtattgcacctttcttataagtaggtgttattagtataaaaggtttatttactttttcttttccactcttaaccctaaatgtttcaaatgaattattaatattaattttgtttactaatctttctgtttgtcctgtacctgaaaaatatacgattaatggtctttctttgcttgtcatacttatcttaccttccttatatatcactaagaaggtcattaatcaattcatcttcatgcttacttacctcagataaattagactcgaccttcttgtatggttttatatcttttttgtcgtagcaagatataatataagaaggtgtaaatgaggtcaacccattcatggtagttataaggacatcataaaaaaatttatcttttctaatattatcaataataaagtctgttctcatgacagggtattcattatttcttaaatcttcatatactttctcatcataaggtaaagcaacataactctgagctctatttctagtaaatattaagcaaggtgtcttattcaccttactactatcattaatgacttgctcccaccatgtgtgtggctctctgttgtttaaaagaacgttatctatagtccattcttctctatgtttacattctactactaaaggaaaattagcttcctgaggtactactatatctccgacagcattattatctttaccccatgaagcaccacctgattgaggagacctattgaattggtatccccaccaagcagttaattctttagctattttacgttcgaatgtatccccttttttcttactgttcgtcatttttcttcaactgaattactttttcattgctgtcagattcagtttttttctgtgcctcttcttgttgtttttgacgtaattcttcttgtttctcttttaggtaagcttctcgtttttcatttagtttatctttagctttacctactttttcttgagttactcctaactcatcttccatgataatcatcattaagtcaattgcattaaatgcatcattaaagtctcctgtaacctcttgagctgttacagcattaagaactttagcaatacgagttgcttcttctaaagaaacacttcgtgaaccaattctatctaattgtttagctgttttctcatcaacttgtttaccatcaacagttccttgtactacagcttcaatccatttctccatgtctgcttgactaataccttttgtgtgtgtgctaatatttttttccattattcttgttctcctttttcttcttcatttttaggttcttcttgtgatttatcaattgattcttgaatgtagtctaatacatcttcattaataagctctgctttatataaagcatcaattaaaatatttaagtttaactcgttattatattctaagtctgtcatctgctcattaacacctaacataaggttagtaatagctttagctacatctgttaatgttgctgtttcttctttacccatttcttctaattgatgttgtcgttgctcattaccaactcttaaagctgtaatgtattcatctacaaattctaaaatatcttttttattatttgacattattttttttctcctttattaattacttaattactagtataacataaaacttctctaaggtcaagcctaaagtttaagtttttgttgtattttgtaactctgtatactttcaggtgttactagcacccggttctgttttaataactcaaaagctttccttgttcccatatcatttgcatcttcatcaccatgaggtacaaagtaaactttattaaaatgtgtttttaacttataagctaagtctatattattatctaatgcatcagtgtctaacataatatatattgatgtatctatactaacataatcaattattttttttatttgattctcagttacttgtttacctaatgttgctacgccatactcatggaaggttaaagcatcaaatacaccctcagttataactaagaactttttctttcttgctatattcaaattaaatatgacatcttttctccctacttcatcttgtttagcaggagcattaatagatttaatatacgggttcttttctatacttcgtgtattccaatactggtaatttccatcattatcataagtaaaaaatataatactattccttaatgatactttggattccccgtcaactttataaaagctcccattaataacataacctatattattattaataatttgttctaaagttatacctctgccttttaagtattttaaaaagggtataatctctttattatttaagttatcttttaataatttatacccttcaggtaatctaggaggtttaatactagtatttcctttatcttggtgcacacctctaagcattaatataagtttctctgattctgtcaaatccttattattggtcgtaagtaaaggggctctctctatatctatattcttagactctaataaatcaaaagcttgtttacctgtaatgttataataagtcttcataaatgtaatagggttacctgattcatcacattttttacaatgatactgaccattactagagtctagggcttgcttaacatagaacttataacttttttctccacaaaacggacaacagtatcttagctcacctatagtattttcttttggttctcctaattcttgggttaaaaagtcttcaaacctcataatatccttccttactatttactaatttttttcattaataacaacatattataaatttgtcctaagtcactataagttaaatctacagaaaaatcactctctttaagtgcttcattcagttgtgactcttctgatttcatgttaaatattcttaattcttcttctgttttaccttctaggtatctagtaacaacaatagttttaagactttggaatataactccaatatctctaaatccttgctctacagtattagcatcatattcttctgttataataggtaatccatattgtttagctttctctataatctttaaattatctttgcgtactgcagggttgtaccaataacctattttacatctatggttattatctctaataatatcttggttattatcatctaaaccataagatgaaggtaaagtagaacgtaatctttttaattctaagtattccccgtttctacttaacttgtactcataagcattacttagagggtaccaatcaactaagttagcagttggtacattatctattgttttgtgctccttattacgatgaaacacattaagtgttgcattatctttatctaaaatctttaatttcattatttttcctccagttttgatactccattttcttttacgattttaattgtttgttcaaacagtggtttaagctcggtattatgagtaattacaaatattgttcctactgtattaagtctatcttttaataatttaatcacgttttcacaaccgatagtatctaatccatcaaaacactcatcgtaaagtgcaatattcgtagatatctcatctttactcataattaaatcctgaattgcaaaactaattgctaaatcaatacgtttttgttctcctgctgaattggatttgtacgaacctccgcccttgctattcttaacaataacatcaaacttatcttttagttctcctttagcattcttcacttgagtttggaactctatttcaatatctgaacctgataaagtttgaaggtactcatttgctttttcatttaagaatggtgtaataaagtctaacactactgaacgtatacctttattactaaaagcatctacagcttgctgatatttacctttcttagtttctaattgtactatattctcttcatgtttgtcaatagattcttgtaattctttcttatcttcctcatgtttttctttacttggttcttctctctctaatccttgaacagattgatgttgatatttaacttttaaatacgttttattattctctaatgaactaatgtctgatttaatcctagaagcctcttgaattatgctgttaatcttattattgtgctctcgttcttgttggtcttcctgtgacattttatctttaatttcttgtgatttatcttttactttatcaatagcttctttatacgtatctttcatttctaatacactagtaatctcttgtttcttattctcaatctgattattgatattttcctgttcttttaatttatgagaattatctataggggagccacaaacaggacaatggtcatttgtatctaactgatttagactattgcttaatgtagctatttcagattttagtacttgttctttggtattatactctgagtaaacttgatttaactccgttaatttattagaaagtaaatctaggctttgagaggctttattatactcctctgagtgcttaaattcatcttcaggtattgttggtatctgtgaagctagttgctctatttgagcttctatttgcttctcctcttgttctcttaatctatctttttccttattactgttttctatctgaaccaattgttctttatattgattgtactgctctatttctctttgaaagtactcatcttttgtcgataactgataacctagtttatagatttcctgtcttatgttattttgttgttcttccacttctttaactttctcttttgctacatcttgtgcttgtttatatacgtctgtcttagtaatagattcaagaatttctttcttacctttatctgttgcttgagagaacatagggatatctccttgaccatacatgatggcattaacgtaagtattaaactcaataccaaataaatcttgtatttgtttatcggtaacgtcatttgttgaacctgtaatctctttttcattaacgaataattttactttattcttattctctttatctttacgataacgttcaattaaataactatcttccccaatatcaaacttaagtttaacagatgtatttttcttctcaatattatttactacatcatctgcttttagtcctttttcagttttaccatataaagcgtaagtaatggcagatattaaagttgattttcctgagccgtttgaatgaaatgactcattagatttattctcaccttcaattagaattaatcctctattgtctaactctagcgtagcttctttaattgctaaaaaattattaacttctacttgtttaaatacaaccattacttatcccctttcgtaactctaaagaaacaatgatgagagccataatcaatccagtaatactcatcatggtctactactctattgtaataatgcttttcaaaattactatcatcaaaaatctttttcattttacttagaccatcttctaaaccttcaacattacaaatattctctattactccatctgaatcttggaattgtaaaataggtttacttttatctaagtctttacttactacattactgatatctaatgtattaattttaggtttcatatctttaaacactttccataaatcttcggttactttctcccaatcctctgctaaattttctgcatctgtcttatttagatattctctatattttttcattataaaacctccttcaaacactcaagtatttcttgttctgattcaggtgagtattgtttagcataggtactagcaattgttaaagggtcatcagacacatcactatcaatacgtttctctacagtatactcttttttaatttgaacttcaacattcttatctttcatactgtcatccatttctaaaaccttagcatgatttgctgtaccgataaccctaataaaattatcttcctcgattagctgttcaaagttctcaggaatatcttctccttgaatagtaataaatctacgtgtatggattgggatgaattctgtagtcattttttctgtatctattaaatgaacaccattagcttcttgctcatcagaaaatgattgttgcattaatgaaccaccataaaaatgattcggattttggaaatattgtctacggtgataatgacctagtaaaatgaaatcatatttatcaggtaataaatcctggtatccaaatgccccttctaatctatgagacccttttccagtcaaagagccttctacacctaaatgacctactaagatatttactctatcttctacatatttatttttaataaactctttaatttcttcagtctcgtctccataaggagccataacaatattaatatcctgttcttctcctaaaaaccctaaagtatcaaggtttttacaaacctctacattaggtaagtaaccgaaaggttctatactagaatctgtatataaactatttgtaactgaatcatgattacctcttaacattagtacttctatatctctattaagttggaatgtttcaaataccttattatatactctagtatctactgcgttacgtttgtggaataaatccccaccaaatataactgttgcatcctcttctcttgcaatatcaaacattttctgtaaagcttgtatctgttctctaaatctatcattcacatactgctcatcaggtttagcatagttagtaaataagtgaaagtggctatctgtaaagaatacaaatttcattattaatctctcctttattctttataaaataattataccacctaacatataaaatgtcaagtggtatattttaaaaaattaaactcccggtaatcctccgaatgtgttatttatagcttctattttattatctttattttgaaatttacttgtgtcttcttttccattatctgatagcaattgtatatgctcttgtttttcttcaggtgtttcatctcttacaatcatcttagttggttctactttaagattaacaaatcgttctcctgtgttagagctattacgaattttatctaaatacaatcttaagaaaccgctcttgaattcttcatctttttggtttactgctaaagacacctcaacagcattgacaatcttacgagaaccttctacatgctcacttgtaataacatctgaaccataagcaccacggttagtttgagctaacgtccaacaaacaaatccatattgctgtgataatctacgaatatcttcaaacaatttccctcctgcatctgattctgaatgatatttagcataaggatttctcattaagtgaggatagtcaataataacaacatcaatatttttatccttcttaattgttgtattgacaataatttgttctaattggtttggtgtaacttcacctggcatatgtttagaaatataaaagtcacctaataactttctattcttctgataatgattttgtattgcatcataaactttattatttaaagacatatcctgatttacaatttgactcttttctgctcctgccatttgttgctcagcacgcaaaaccattctatccattttttcctctaaggcaatatataaaacacttaatccacttttaacataattcttagctaagtttgaagccattaatgatttacctctaccggtaggagctatgattaatccaacctctcctcttgctataccaccttcaatatggttgtcaatagaagtaaatccagtagagaatttatttgtagctaaattactcaatagttcttgtttcttttctgtatcttcgaagaagtcaataaactccccattacctcctgagatgtcacttacttctatttgctttagtttgtctacaagttcatgtagattatcagagtcttcttgtttattttctgcaataaatttaactaaaacttcttttgacatttctgttttaatatacttctctatttcataattaacagattcatctttttcattaacgtctactttatataaatcttctaagtaagttaatgtcttggttactttctcttcatcttccttattattacccataagctgttctactttaattgctaatgcttcatttgacatcttatcggaaatatgtgctgttctctttatagctgtaaataagtaacccatgtcctcagactctgctgaaaatagattcttaggtaagttatctaatacttctcttgcaaaatgtatatccttcattgatttatgaaggataagctcctttatcttcttactcatagttattaacctcttcctctactaaatgtataagtttattataatccaacataccctcttttgtcaatggtattttatcaattaaattatatttctctacaatatattttacttctcttaagtttacctcataccctaaatagtcaccaaacattcttaatatataataagttgaagtatgttgtctcatatataggtattcattaccattctgttctaagttttgtatatccataccactcaataaatctttattcactatacctcctagacttaatggtaataaatcctctctcttgattcctttatttaactgcttattgtaaactatatgctctttctgcatagtaaacattgataaccttgcagaaggtgaaatacttgttggtgaataatcattaattatgcattgttttacatatttattaataatatccttttcttctctaggtaaattcttaatttcttcttcaatcatagaattatactgtagtaaaataatatgtttcatatctccaaataatccataggaggcattttcaaggtctaaggcttgttcaaacatagtatcaatatcatgtaaagtagaatttaatcctgtagtatatagttggtgtagatgtaatacagcagggtttttcacatagtctgatgaattaatgatgctgtctgtatcacctaggtgtctattagtactgttaacacctttttttattcctttaatataattattatagtttttaaaatacttatctgagctaaagaagttaggagaaggtattggattaggttgcataccgttctcgtaataaaatgttacatttttaaatacattttgcatatacttaaatacattaatatttttatcttttatttttaaataaaaattataaaaagtattaaacattttactaccaaagaaattatcactaacgctatttctaggtaaattttgaatcattttatttctatagtattcagacatatgttgatggtagtcgtattcatctttagttgttgccttactcattgctttaagatgacttgtatgtttagcatccatatgagaaatagcatattggtcgtataacttacataagatgtacgctttataaaacccttccgggtcataggacatattaaaaatatctttagaaggataaggaagaccttctgaatagaaattcatatctgctataattcttctcttctcatccttaatggcattgtattgtgccatttctatcttagtacgtctacgttgttcttttctctcatatctataagaagggaagtgcttttctcttaatgccttagcatactgtaaatcggaagtaataatattatctgtagaattattgaattctttcaattcttctgtgtcatactctctaactaaagttattattaccccgccgttttttcctcttttagaaacagtaacgactttattttcttcttctaactttttaaggttattggataaagcagagatggaaacacctagagaatctgctagctctttcttagtagaaatatgaaatttattttctgtagcattttcctctagatattggtatagctttctttgtgtttttgttgccatctcttaccctcctattttcctaagtcttttatttcaaaatcttcctcttcataaattttcctacgctcattagcatgagtatataaaaatctatttgtcatatcattaaaatcaaatatttgtgttgtattatcgtcttttttcttacgtaaagcacgaccaatacgttgcaatgtttgtcttaatgacttacctcctgcacctaatattagtgcattaatacctgatatatctacaccctcatcaataagtgatgtagctatcattactttaagcttaccacttctcatatcgtttaatttttctctcctagtttcagagtctatttctccatgtaagaagtaatgctctacatctaaatcatttaacatttctgatattgtgtctccatgttcaatgaagtttacaataatcaatgtacctttatcttgattataccacttttctgttagttttgcaataagtttattcctaaaatcattatttgttatacctttatcgtaagcttccctataatcatcaattctatctatgtcattaggattagctacaggtataatatttattgttggtctagcagaatgaccttcatcaattaaaaacttattagtagttcgtgcaataacattaccgaatagcgcctgcaatctcatccaaagtaattcatcttttttatctatagaccctgttaatgcaattcgatataaagctttttcacatgtcattaaattattataccaagaatcagatttagaatggtgtgcctcatcaactatcataactgtaactgagtctaaaaaatctctcatatcttgatatttatcatatttcttttcgttcttttctctaacaattttttgaaaatgtgcattatgatttcttaagtttaataaaacttctgcatctgtcttactattttggtaaattatctctaaggcacttaatacattttgttctacttttgttttaggtgttgtgttatcaagtaatacttttagtaatttcttttgatttgttccaccttcaaatttaggtaatatctcttgagcaatctttttactaatattttgtttaggcgtaacctttaccccttgtgttgggtctttaaggtttgcatttaaagtaggtatcattacaactgtaacctgcttaacatcaaacttacctgcacctactttaccaataggaatatttaaacgctcttggagcctatctgcagactgatggaatatctccgtagagcctgtgaagaatgctactctctcacctttttctaattgaggtaataattggtctataataccactagcaacctcagttttaccaccattagtagctaagtgagcaataccattgtaaaatgttaagctattgtacactgcttcatattggtaatctcttaaggtaatttgaccgacattattatcaagcaatgttatctcatcatcaatatcttcttcagataagaaactttcatcacgctcatcaattgtttcaaactggaaattataccttgattgtagctcacctaataataactcaattttaaataaaagtccagtggggaatttatcttcctcataaacataaaaatctacataaccatcccaactacctcttttgtacgcagggctgtactgaaatccttcttgtcttgctcctatagtcgtatggactctctttaaaaatatatcttttaagtatttatcatcctcctcaaattctacataggtatataagttcttaattcttaatctcattttaatctccttatatccttacacgagttatagtataccacagaatccttttcaagtcaagaaaaaaaaagactaggaaatttctcctagtctatggcatattaatacctataattcttgtaacgtattgagttttactattttgtactgcactatctacaagagtccatgtagaacctttaaaagatacacctactctataaaactccgtgatagggggttttgtatcggttctatccccggaaacaaatgtacctgaacaaacgtaagttccactaccatctaaataaaacttatgacatatagttcctgtattgtcattacctttagtcttctctgttgtcttatgtgtagtataaacttctacataagagtagttactaatatcatctgataaattcttagtagttcctgtagataagaatgattgtgctcctgtccataattctttatgagaaggggatgtttttggtgaagtccaattactacctaccactgaacctgttacatttctaccatctgtacctatagcattgtaaacaccatgtaaactagaagtattaggtgtgtctgacatgaataaccctcgacaactattcgacataggggaaccggatacaccgccttgaacatagaaagtaaaggttgtttgacctgttctatctatatggtctataatagcttgttgaaatgttttactatcactagtataaactaggagtgggaatgatgcactaccatcgtctataatactacttagtagatatttctccatatcttttggctcaaattcttttttccaggctgtgtatgttcctgtaggtttcttaatgctagtatattctttaccggtttcagcatcagttagtctagctaatctattatcattagcatctacacttaagtctaaaaatccggagcttactccactaggcattcctcctgtcattcccataacaaagtaaacacctggtgaagctatatctaaaatattagttcctgatataacagcatttttattactaggtaatccttgaataaattgccattctgaactagagtttcctgagacaaatctatataacattttaatttggttattgttagtagatagtgtatataaatgttgaactaccttactagaagtatcaagaggtactacatgtaaaacaacatcaagagattccatatcaggtctatctaataatgttgctaatgttctagcacctatatagtaataaccaggttcttttatatctctaagggctgttatgtttccataagatttagttaatcctttatcatcagttagtttaattttctgtgatttattaaaattataaactaaatcatcggtaatgtcaaatatatttctttcttttgttaattcttctccaatgttattgacctgagtagtcaggtctctaataacattgttttccgttattgtagtaaaatttaatgccatttataattaccccttttgacctaagagcttttgaagcatttccatcatagcttcttgttgttttctcataagctctagttcttcttttaaactatcatattgttctttaggtacagtaatttgttgggattcctcatcatcatcattgatttgtggtggtacatagtttttattttctataacctcttcaggtttactataatacaaatatctttcataggcaaaattatcaaagaagtaagcaggtaagatttctctattaacttttacttgtcctaactccttatcataccctcctactgttgtaaaccctgttatatatttaccttctcttaaatgtaatactatctcattattacctatatcatatttaacagccatagtttttttattccacctttctttatcctctattaattcctataatattgtatacctttatacgttccttttcattccatgatgctacttttgttgtatttgtagacccgtctaaagttatagattttaccataccaggttgtactgatgttctagtaggaaaagtgcaatacccttcaaaaaagtctacactagaacctttactatcatttgaaatattaaaatctctaatatacagtaaatttgatgtattttttatatctaatctttttgtagaaaaatgtcctgctgaacgagtccaataagttacctcaattaaatcgtaattatctaatgagtcatttaagttaacagcttccgtagacccaaaatctaaagtacctttccatagtaaagtagaagtttctgattgctttaagggtttccaaccctggttataaccaccataatataatgtgtaaggaataccctctgtgtcagtaaacaataatttaacaaacttattagctctatatgggtcacctttgtcataatctacctgaatggttcctctaatagaacgtcccttaggcgtcccggcaatagagccttgacaataaaatgtatgatatccttgaggtactgtcatagctaaatcttttaaactcttttcattaacttcagatttccaccactgatatttaaaataatcattatattcaatatctacaggggagtgaccttttaattcatctttactaataaattcttgccaattagaccaaatctcatcatatttacgaacatcattttcatgtgtaataaatggtgtaactttagatgatttttctaatttaggtaaggcatagtatactacagcaccattacctttactagatgaacaattcattcttagtagtaaagatacgtaattaactttaacgcctgatgctccgattgtattttcaggtatagtgaatgtgaaagagtattttttccactcagttgttatctcacttgctgttatttctctacgtcctcctgtataaggatttttactagtatcatctacagtatcgtatcctgctaattcaaagtaaactaagttatctttaagtaaagcagggtcattaattttagcatatactgataccgttacagtatcacctacagaaagttctttacccattttaaacctattagaaactactgaagggtacgtatcagacccatcatctatctgtactgtattctctcctctaaatctaactcctgaacgaccactattagaagaatatccccaaaatggaggcgtatcccagttatctgttttaatattagaatagctagtttcaggataaacaaataaactatctttcaataagttaggcttttgttcttgtactggtaactttcttatcatcttaaaagttgaagtggagttaaccgggttaaattctactaaagaagaaacaccatttttactttctttgtaattaacccaacctgtatttaagttctcttttggaggattaactagtgttgcatattgagttgtggattcagtaagttcctttacattcaaagtattaccttcattgtataagctaccttcatctaatttaaaactaatccaatcatatacagtaccattagcataagtcttgatatacattttgttactgctatatggtgaatagtaaattttataataatttacatttcttttatccaatcttacaaatcctgctgtagcttctccactaggagcaccttttgaattagttacataatagttaccactatcaggtatagatgctaaaatattattaaagtcgggttgtgatatctgttttacatcactaaggttaacttctgtttttaaaaagttacctatatcattaactttaggatacatatcttttaacttctgtgtttcagtaagaggcgtgtagttaaatgccatctatttttctcctctctctttctttaattcattgattaacttagtagcttcatcaattttttctttgtatgttttttctaattccttaattgatttttcctcaactgtaggtataaataatttagcttgagaataagggtctctaacttctctaggcatataaatctctccttattaaataaagaggggtcaatcaacccctctagtttattcatccctagtagtaaccataagtctacgaacacgaggacgtaaaaagctattttcagttgataaatctaatctaacttgtagtttagtatttgttcctgatgattttactttctcgtcaatgacatagcgtgtaaactcattattggctctagtagttgtaggggatttagtaaatgttttccaagtttttccatcatccgcagaatacttaggaacaactttagtacctttaggtaagaaagcttcataactaaatcttactgtattgtaaggagcctctgtcatatcaatagctctaccaacatatgaacctgttaactctgttaagaatgtagtgaatgttaaatcactagagctcattaatggtgagatatatctattagattcgaaagttgctcttaacttaacttgtcttgctagacctaaaacatctaagtcttgatagttaccgataggctcccatttcaattggtcgaatgttgtagaagatgccatatcatctagaattaatttcatttcccacgtacatcctgttctctcaggagttaagtacgtagacataagaactattctatccgctgatacatctttaattggttcgaattcaatcgttgctgtctcattaaatttagaagtataaataccaaatttaaggtcagagttttggtgaggagtccatgttgatgcgtttgatgaactgaataatacaccttgtaggtatggattacctgaaataacctcattaggtttatcaattttaggcttagttctagtacctacccacattgtataatcactgttctcagtaataataacaatagcgtactccttaccgccttcagccatcatagggtcatcaaagtatactctagtttcagcactagcattattagatactttaatatcatcagcattcataactgtttctgcatagattgttttattaggataaccttggtcacccatacctctaatttggataacaacattagattgtttatcacctttagaagcaaagtataatcctaatgatgatatagttctattctcatcatattggaatgattgtgctaacgggtctactaagtttactgttacacgagttctgataataatatcttgagcggtttttttacgtccttgggctgtgtaagttgtagcacttgtagagttagcattcttaagtgtaacttctcggttaccacaacgaatacctgcaggaatagtaaacttacctttagctgttcctttagcatctgtcattatcgtaccatcttcagagccttttctatagccagttgcaggtgttatagcacatcttactccatcaaataataaatataaattattatcattagggtttagtcctttaacttcgaaggatacatctctgattctaatgaattcaatcatttcttctagagtacgttgtcctcctgattccaataaagtaccggtacgaccatgctctctatcataagcgtaagtttcacctttccacttttgtcctgcatctaactgtaagttagaatacaagtaatgctcagtctcaccatagtaactttcattatgtctccaaaatcgtttcatagttacttttttagttttttgttcagtgattgtaacattttcagtatcaatccagttatcctcactaggtgttaattttaacacaccttgtttgttaggaatattataagggttaacatttaatgtttctgatgcttgaccttggtagattgtacgttcctcagtaaatggtgctgaaattaatctaccccaaatatgagctgttgtatctcctggaataatcttaggttggttaactgcttctgtataagctagagtagcttctgcatcttcaaaactaaatacaattccgaagtcaggatgtgtaatgtctgctttgtcaagactaatgaaaccttcactgaatactgaacgtaatgttagagggttctgtccttccatagcaccatcatctagagcatttactgcttggttatactctaagttatctactcttgttttaactttctgtaagtcttccatagacaatctagtgattgcaaatgaaatacatacggcttcatctgaatcaggtaatactgtaactgtacctaattgtaaattctcagggtctgtgtttaatggtggtgtaactaatctcataatattaggttcaccaggtaatattgcaatatcaccatacttattaataaacactgaatctttacgagccaagtagtaagtatagtctactaatactactgtttggtcaataggtttagcaccatttgaaggtgtaaagttaatgtaccatttcttacttaacccttcaccttgagttgttacttcataatccttaccggcttccatacgtttgttatatttataagaaacgtagtatgaagtacctcctgaaggttcttgaccttgaggtgaccaatcaattgtttgaccatctgttaatctgaagtcttctccttgtttatactcttttgtagtaactccagggcttgtttcagtccaaacttttacaatttcaaatgctgtattatttgaaagaaaatcttgaccatcaccttgagctcctcttgtaactcgttctttttcaataagtacttgacctgtaacacgtctaatttcttttacagggctattagctaaactaatagagttattagacttattaaagatagtactttcattttctgctgttcctaagtcataagatttaggtacactaattcttgttgatacgggtttgtctactttaaaaccttttacataggctttacctgcatctacaactacagaaacgtggtcatcatcttcagcattaccttctgaaaatagttcaaaaccatttactttatatgaacctgactcatcataagtacgttcagctaatactttgttgattttatccatctcagcattagttgattgaatatataaatccccatccatgaaagtataaatagttgctgatgtcgggtcattaactgttaatgacatcttttcttctaatctatctgcacctttagagaagtaacttggtactccactagtttggtctaataggctagcatcttcatcaggtgtaacaatacgttctgttaatttaataccaatagtttctttacctacgccagttattttaactgaatcgtcattatcgtaatatcttattttaccattgatatatacataaccaggatttactgtcaatacattatcttcagacaatgtgaatccaagccctgattgtttatctccgtctttaaaaatagcgtctcctagattttttaaataatattggtctatagactgcatttcatttaattctgcctgttgtagaggtctatcaggattaaataatacttttgttctatctttagacgggtcaaatctatctaaataaggtgaacctttaaaattaattgccatttagaattatctccctttctttaaacttctacaataaatctttctttagcagttgtctgctcacttctattttggaattgtttattatcaaagaataataatgttccagttgattctacttcactaggtactaagttaaatttactaataccactttttgctactaagtccataacaaatcctacttgtctatatgttccaagaggtagttcgtcaccaacaatactactttctaagtaaacccatttagctccttcagctttagcattttcaggtgttacttctacccacgatttattaccataagaaattaaattcttattatcatcttcaggtgatttagaaggtctaactaaagtaactttagtagcttttttatatccaataacctcctgtaatactgttgcattttcatcaggttgcggtgggtttgtttcattagaccacggtgtgctctttccaattgttaagtaaactgaatcagctttactaactagatattttgctaactcaacatgagaattatacgttgcaattgccatattttttaattcctttcatctgttgttttctttattaatataacatggtatacccactaaatattactttataagtataccatattattaactttctgtcaacttatttattaaactttccttaattctactcttcttactttagagatagttgaatcataaggtcgtatatatattctaacatctgtatttaaccttggtttgtcttcagtagatggttttgcaataaattctttttcagcaactccattaattatctctgatggatatataaatgtttcattaaagttagggttagaattgtataaagatataaattctatattatctttaaattctccttcgacaataagtttatatctagtaccatcttctataggctcatataaaccgtaatctaaatatttagaacctgtaacttctctttcaacaaatccatttttaataatgcttgtatttggtaactcctcatatattgagtcaattactttaattctattatcgacattaaatgattctgatgttagtagttcttcttttagctcaattattaatcgactagagcctaacgttaacggttcagtataattagtcaaagtataggctaacatatctaaatcagtataagcaccgacattaaatagagtagccttaataatttcacctgtagctccatcgaatactccgccctcagtgtctttaaccattctcatagaacctagctcaatatctgataatttatctatatcctgccatacagcattaaagtaatcattaggcatattatataaagaaccttcttgattgttaaattcacctagcttaacattccaaatggaacgtaatgatactttagacattggatttatattagaataatctactgtctgtaatccatataaatctattaagttattggtttctgttgtttctccagtactaatatttcctagagtctttagttctacccaactatcaccatagtaagaataatataaagttacattttttaagatagtatccgaattaatacttaaagtctgtatattagttaggatattattaaatttaattcctttttctattacgttaggtttatcttccggagcaggtgagtactctagcttagaagaaccctcagtcaattttaattccgtaattttaactgtattaaagaacactggaggagttgatgctttaccatcagatgtatatcttccagtatatattagtaatctatggtcagtaatagtaggggctgtaaaagtttgagttacggatactttgttacctatagtacgttccatagatttaacaccactaacaacaattcccttagtattactgtataaatatataccatgactatcagaataagatttaagagttgggtcattacctgttatttccatatcaaaagataacgtatacactttacctggttttaatactttagaaagataattagttgagtatatctctttagcccatcctgatacagtaaactcttttgtagaagttatttctctagctatcgtatttacacctttataagattgtaataaatttctattctctactgtattatatacatctaattcattagattctatagggttattatctgtatctttagctactatatttattgtgctaggtattgtattttcataattaccaactagttttaaataccctataggctgaaaacctgttttagttataatatctccattagataatgatgctattttaaatgcttctacataagtctctctagatatatctaaataattttctattaatgctttatacggttttattgtataaatacctcgttcatagtaataaaacattaaatctaagtaattaactttaatatctactgaatctcttttacctgcatttatctctaacctagtaaacatatttaattcactgtttagataatcttttatatatcctatattgctcccgatatttttttcatagaaagataaattattaattgatactgtaagccatctattaatagaaaaatcataaacttgtagtttaatttctatactttcatcaggaggtatcacagcactaagatagtaatcaatacttaattcttttatataatcagatacaattcttctagcctcatcagattgtaaatctacttgaggtctatatttactcatatagtccctaaaattaaagttattatataaataagacacaccggaagtagtttgcatactaagttctactgtattgttattcttagtactagtatacatataatagtcagtaggtacttcttgacctctaccttttgtactagcgcttatttgatttacagaagatgtcatacctggattataaacatatgatgttacatatccgtagttaatatactgtctccctacactagatgaacctgttaaaacatctaaactattaattaagctatggtttgttttaaaaatatctgatgaactgttatcagtatctttactttgattcatattaatatgaccatagaatgtatcatcataacctgtaaatctatcaaactcttggtatgtttctattttaggtaacccatctaaccacttaataattgctccacctctaatagtagaagccccatcataagtgacatatagagttacacctgcaggtttgaattcattaattacatctataatctctacagggaaataatcacctatagagacattaataacagcaaatctataatagtatcccattaagtggtcttcaccatttaaatgtgatttgttagtatagaaaatatttgtaaaaggttcatatacacttacatcaatatcatctctacctaaataatcttttatagcatctattatagcattattagttcctcttttcaagagtaaatattttataattcttgctctataaacatcatctttctcatcggtcttacgataaactccgaaccaatccccaaacttatctaaatattcactggtagatgtctttagagaagattgtaacttactttctatagcatctttctctacttgattcatctcttcattgagtgcatctatcagagcaaagttagggtcttgattatcttttttatttctatctcttcttaataatggatgaagattctttaaaaaattagccacgacttaagtttcacctctctttactttagttctacttttatttctcctgctctaataattccttgtggcggtactataatgttctcatctaggttatcaaatgacacatcatatattaatacatcatcaatattcattatagcttgaataaggtctgtaattattaggtcatcagaagtttttaaattatttaaatagcttctaataacactttcgatatgtttttgtaatgtatcaccaattctagatttattagatatagttactgtagcagaaacattaacttcttctttttctacacctgtaacatctaacattataccacttggtctatagtcttgtaaagcatcaattatatcttcttttaaggtatctgataaattaccgtttctatcatgagcaaatactgtaatatgtcctgtttcttcataaacataaacaccttctacatcaggtatctgcagtgcaccatatcttactgatttattagttgctctacctctagattctacaaaagagtggaatctgcgcttaaagtcttcttggctctcttctttagttcctgtattaaaagaatactcgttattaacacttctaatcaaactagaacctgatgctatagtattaattgttccttcaggaacattacctgcaacccctgtttctttacaataaacttcaacaacaatctcagtagaatcaggctctgcataataatcaactaatgtttcaaattgctgagggtattcttgtcgtgttgaagtaaaagttgttcctgcgggtatatacattctcatatctaagggttggtagaattgaatagtaacatcaccataagctcttttagattgtcttttttgaaaatcaaaagcttcaatgataccttcttgtataccccaatcaatattttcttttgttagaatatagaattgctctatctctaatgatacagcttctaataatgaacgagaagctgaaccaggagtaaagtctgttatcttgcttgtacctgccattgtcttatctattaattttgatagtatgttagttaattttctagttttcataatttaatcctttcctattcaaataaagcaaaaattccttcctcatcttgtcctaagacaaaatttattgattcttcaacagattttatttccactgaaaattgacctgaataaacattaccttgaattttccaatcaattagattagcagattttactctattatctgatgttaatgttctcaatacttccatttctattaatgtagcttgttcaggtatattaagaccaaataaattatgcaaatctgaaccgtaattaggatgtagcattaaagaacctctaggagttaataaacgtgcctgtaattgctgtttcatattatctatgccttttacagtatctaaatctccattaccatttgtgctgaatgctagtatattatcactagtaccatgttcattaaaatacttttcatctgcagtaatatttaaatctcttcctaaagctaattcaactagtacatctttatctctagaattaatttcttttgctgatacatctgttaaatcagattctataggtataatcagtgtatcacctgtagaagccaatcgttcagggtctttcattttttcttcatcagtttctactaaataggggtactttaaattattatgctctactaggtctatccaataactcacatcaccatagtatctctgtgctattgcttgcatcgtttcttcatgttgaactacgtgcttcttaaatctcattatacgttagcctcctttttgattagaggtaattgtaattctatagcactaaaacataagtctaaatctcttaagtctaagacaacactattatagtcatcatagttacctaagtagtcagctacataatttaaattttcccgtgttatactaacatcttcttcagttaagtattttaaagagctcggattttctacaaagcaattaacaatagcatatgcttcaagtacaatcgactgtagaatcatatacattcttggagcctgtacttttagtaatgtatcgttagctttatggataattgtattatcattaaattctctttctaatacaggtatatcatctgtctttatatttgtcaatactttttttgctaattcacttaattgtagctgtggtgtataaaatttagatacaaatgatacctcttcatccagtaaattcatatgagaagaagttaaatctgtatctagggcatttaaaaatctaagtagaccttgaggttgtggtataaaattattcattagctagatacacctccatcaccgtaacctattatcagcgccatattgtcaatattaccttttaatccgttagtagattgtctaggattatatatagaactactagggaagctttgcttaagtctattaccctcgttcgtattatctcctgtagatttaggtattgtattttctcttctagatatttcttgattgtttctatctctcgttttacgagcatttttatctaattcttttataccctcatctactctttgagaagcattaggtttaacgttaccaaactcttctgttgttacagcacttctatctgcttctgttaatgaaccaataactactaatgttatttcatatctaaaaagtaatggttcatctttagacctagttatctttaacccctgaggagctaaatgaactttataataactatcatctgtaaagttaaaaaattgtaagtaagaaccacttacattaccactgccaccttgcatagcatattcactaactctactttgtaactcttccattttctgcttacctgtttttaatccatctgcttctctaacaggtctaaaaccagttgtacctgtgaagttaataacttctatgtctttaccataatcttctattacaatatctgattttgttttaattgctgtcgtacgttgtggtgaatctattgtgtagttctcagggttaactttaaatctatacatatcataaccaccgccttctttagggaagcgtaaagctattctatgaagattacttattccatctgattgtggcattttatcctctccttttcagtatgtatataaatctcatactactaatatatcacgaactagtttaaattacaactaaaaaaagacctaaattaataggtctttaatattaaatctttttatattcatctaggtagtttgtaaagaatccatctacacccatttgtattaagctttgcatttcttcttttgtgtttaccgtccatgcatggactttaagccctttactatgagctaaatctactaattctttagttatagttgtatatttaggcgcaatagcataaaaaccataacttaaacaatcatccatttcactttcagaaaatgtacttgttaggtaagctaaaggtatattagagaattgattatgaatattaattaaagattctctagcaaatgattgaataattacttggaatctctctgaacctattcctattaatccttttgcttttaattgagttaataattctctatccatattagcatcaaacggacgtttagtttctatataatatttaacttttgttccaaaatgatttagtacatcatcaagagatggtatcggctcaccattgagagatgtaaagttagtttgtatataagataaggtcatatctcctaccttacctgttcctgttgttgttctgtctatagtagaatcatgcatacacaataacttattatcttttgttaattgtaaatccaattctatataatctgcacctttatctaaagccatttgatatccttttatcgtgtgctcatctgtgtatccggttgctcctctatgtcctataagcataaagtctttctccatagtttcagtttctaattctatagcttcaatactaaaccatgtataagggtctgctctgaattgtctgtcaccttctgagtcttggaaagcttcaaactcaaagtaatctccttctttaacaggaataactccactagtaccattctgaggtgctgtagatatagctacatctcttgtataaggtaatcctatactataagtaccatttttcaatattctcaacatacgttgtcctgtggcgttagagtcccacaaaacattacctgcaactcttacttttgttattcctttaggtactactaatcgagtaggattattagaatcccaaaactctgcattattataagtaactttgctccatggtattggcatatattttctcataggtactgtgacattttcctctaactgtatcatagcacctatatgttctcctctatctgagattattttatcagcattgttattattgttctctagttctgttaatctagttttaatatttggtatttcagttttctctaggttatcaactttagtctttaaggtcttaacatctgttttcatagtagggaaattctgatttacttcattaataacagtagaagcatcttcataaaaatctttaaaagtctcatctctaaatttttggaactgtgttataataccttcagttctaccactcacttctgctatctgagttgtaagtctattgacatctgaagttgcttttttagaagcttctatagactcttttgtagactctattaattcttctaaattgtctttacctacaccctcgagaaggtagtttatatcatcgagttctttttgtatttcattcaaattcttcatagcctctgctatactctcatctaagttttctaacataggtttatcatctatatagattccctcatcagtaaattcaaatttatgcttgtcatttttaatataaaatttattatcttcactaataccatactcaatccaaacttgagcttcatccaacattaaatcgtcatcttgtttgataactctataagacccatcacttgacatttcctgtgtagttcttttctgtgtttctgtatttaaaactgaggctcttatatcccctctttcagatataaataaagtagttatatgattatccggcgtgccatcatcataaaaaactccttgatgtttaaataacatattaggagccttttgtattctaggctcaattaatgtcttattaccataataagaagtaaataaatcttgatactcagttccagtataaaaatctgttgcctgaggtttctcttcttcacctgatgtcatagagaaaaatgatttaccattataagtcctaatacttgttccttctccatcatcatatttataatttaaagatggtaaaatttcatatagtgaactactatatttataaacactttctgtatcaaacttacctccatctagaggattggtattaatcattttattttgttcattatcaccataaacacttaaaataataggactatttatatcatcatttagaaaccctattaatactacagaaccttcagtaataagaggttttgtaccgaatacgcttccttcaggtgttcttcctatgaaagatttaggataaggtacagctaagctaccatcatcacctatacggcttcctagagttaaattgttaactttaacttctactgattgatatttataattaattttactaactgtagctaaaactaatccttttacatttaaatcatccgaatctactctttttaaacttctccccatggaagattggaatcttactaccattaaatatacacctcttcatcgaatctcataactcttccgttaaattcattccagtaatcatcaagtttatattttcctataggtgtttcatctttagggaatttacccatcatagatataaactccccatctcctacataaatacctatattagtatcacttctaccaaagaataatatatctccaactataaatttactaacatctaccttatacttatgtcctttaccgaatatagtaaagaatcttccctctgtaaagaagtagttaatattcataggatatgctaactcttcatcttcacctaaatataaccagtataataagttataagtatctatataaacatctttatctgttccaaatggattctctttaggagcacctttgaacatatactttatatttgataaggatttaagattatcatttaagtctaatgctagtatcgtacttaatggtacatcttcatcaaatacatattcttcttccatatattctccatacaacacatcttgtatctcatcaaagtaatctattaatagttcttgatttcgttctactaaatgaactgttaaagtagataaagaatctgtaggtatagaccctttatcaactaaataacttactttcctgaaacttaattctgcctctatatcataaccattattagaaaatgattcatactgaacatgctgagggttaaggctttgtattactccaacataaggtcgtatatacacaaccttttcatacatatcatctgtagttatttttttaatttcttcatctttttcttttattctttcttttgtaagtacatcaaaattaaatacattgtctataaatttatcaataacatctttagcttctttatctgttgccatagactatcctcctttatttcttatcgtaaatgaattgtgcattcttacctatggctgaactcatatctatcgttctagtaccatatccattaggggtagcatagttatgttcagagataaatatttttttacctccatctaatactttctctacaaacgctacgtgaccatattgtgggctacctccttgaactcctctttgccatataacacaagcaccttgtttaggtgttctacctacaccataacctgctcctttagcaccacctatccagtcggcggcgtccccccataaaggcacaggtatgcctaattgacctcttctattataagcataccatgtacactgatacttataatgtcggttaccaggttgaacaaaacttgggtcatgcttaggaagtttgccgttatattttttaagtgaatctaaagagccaccatcttgagctccaccactatcagaaccaccatctttatctccactactttgtttctctgctacacctttttctttaagttctttagaagtatcttcacccataagacctcccatgaagtctgatgattgattccataatcctgcaaatctatgcggactacctttaccatcttctagaatagcgtcttttaaacctctagttactcctacagttgtataatacccctgtttataatcgaatttatgttctacagattcaatatagaactcccaagtgtctcctcgttgcttatcttcaataaataatcttttacctaggtcatacttagggtctcctaaaacaataatatctcctgcatagaagttagggttaccatgataccaattaaatagcattctagaaaacataactaaagggctatcacttgcatctgaacctgtgtctgtagctacattactaacaccttcaatagctttaagatatttatcaaaagcctcatcgttaggtggctctcctttatatttaatatattcatctaataaagttgtagcatgtgttttattaccaaaacgatatttagttgtcaattctttaagtgcatctgtcttagcttttttctttttcttagaattattaaaatcatcttgtgttttaaacttctcttttagtatactctttaatttatcttttgtcaacttcggtctgttatctgatgttaattcatcatctaccttattacctgttattttttcatattctttttctgttacttttccttctttaacaaacttctctataatttttttagcttgggctttttttaagtttttatattttgaagataatttacttgtatacttatctatacctttagatatattatctcttccatagttacttaaatctttcataattttagaataagttcctcgttctgtgccattatcatcacctgaagaatctgaatcctcagtagctgaacctgattttgtactaagataaatattttctacttcaaatttagtataaccatatctatcagttaattcagggtggaattgtggtttagaaaatacatcaccgttaagctctttcaacatacctgcaggtgttgctgtaaatatagaatatgtctctacatcacttttacctacatcctcttctataaaatcctcagtaggtactttaatcatatctaaagctctccactcagtaggattaaaaggggtctttcttaatacaagttgagcctttccaggtgttttttctgaatttttgaagaataactcattaaaaggtctagctgttaccatatccattaactgttttaatgacccatcaaaattagtaaaggctgaaacttctgtaagtttttcaaactcatcccaactacttaaatcatcataatcaagatagttatcaattgtattatatgttttttcagtatagttatatttcatataaggtataaatctacgtataataccagtcattacttcatgagctgagctaccagtaaattttacttcattatctccatcaccatcaataagccaacctacttcaggtaatacagcttgaacttcctgaataacgcctaatccaaatttcataaaaggttttacaaaagattgacctgttattctaaattgagtttggtcattaccgtaactacctacttttgatacttgagaaaccatacctacctggataagtcgttcttgttttccttctttatcgttagggtcatcattaggtgtaataaatattcttatgatatcattagccataactagcttatcccaataagtatctcctgccatatttatttggaaaactgaactatcatcttccatagagtttttagtttggaatcctaaaagtttagctccaaactcatcaccattatagtcacgtgtatcttcaaatctcaatgtaaatgtattatcatctgtaacgatttctattcttaccttaggtcttcttattctacgcatatacacatacttccttttattttttatatgtataagagtagtatattatgatactactcttgtgtcaatacaaaattactaataacttctcttatgttcatttgcaaagatatccatattactatctagaacttctctaattcttcctccgataatgtctccagtttcttcaggattatttccaccttgaacattaacattgactgttattctgttagttgtagaagcattagaagcatcattagagcccctataattagaaccagtagttaatacaggattactcatagcttcttggtaagtagaggatagggctcctccaccacctccgccaccggagcctccgtatttcttcttgaattccttagcattgttaacacgagtacccatcatagcctcgtttgctcccatacgttcaaatccagtagcaaatgcttttgtattctgctctaagcttccacctttgctccaacctgcatttttaagattattgctttcataatcagactgcatttctttccataggtaatctaattgaacatccatgtcactagattttttacctttagatttagcaaaattttctaatcctgtttttctagcccctaaccactgagcaataccaaatgctccactagaagcattcttagcattagggtcaagattagattcttgttgtaagttccccataacagctcctacctgattatcagatagtccttttcctttaaggaacttgtacatcttctctgcgccttctccacctacatcagatgcagagcctccactatcagaaccactatcagaagttcctccatctatattaatacctttagcactctcaataattttctgagctctgtcaagtaaatcactatagattttaagattcttagtttcagattggttattcttttctctaagttgttctgctgtcattttttctttcttcttagtatctcctttaacgcctttaacaccttttttcttatctttactatcttcttcggatgcttcagcgtcattatcaaagagacctgttataccgctccatgctttagatataggttttcctaataaagaatcttggaatttattcttaccatccttatctccgttagtccatcctaggttccatccttctttagtgttagcttttttagccatgtctccgactttttcaccaacagctcctcctccgaaggaacctatagctccacctattaaagctccaggtattgcacctacaccaccaaataaagctcctatagaagctcctgctgatgcacctgccattcctccacccatagagcctattcctccgccgatagctttactatctccggaagctaaagcaggtattaatgataaggcagacgtagcaacacctaaaactccggctccttttccaagtccacctgcaagttttccaagtttccctatgccacttccacctgcttttggtgattgtgaaagaataccatttttacctccgtcaccaaacttatcagcaaacttaccatagagacccttagctttgccaccaaaacctttagctttttctcctaggttttgtcttctttctttaccaaatatactatcattatagatatctttaagaccactaccggtacctttagctatatctttagatttatccatggcttttctaccattatctctattaaatacttttctagtacctgaccaggcttctttagatgtttgtttaaagcctttacctttagactccccgccaaacatacctttccatcctgcgccacctactaggaatcctccaagacctttagcgctatcaagtatttttcctccagtagaacgagtgcccccacctttaggtgagcctccacccttaggtgaaccaccaccactaggcattccacctgccatagggttaccaccggagctaccgcctttatttctaccaaatttatttctaagtcctttggctcctttaccaattaagtgacctgctccaaattgacttgcagatgcaattagtgaagctgtaaatgctcctatagctcctatagctaaatatataggggcaggaagacctgctaaagcactatgagcatctcttagtggttgagccatatcataagtatcttctgctttatcatcagtcttcgatttattttggtcattttttcctgatttagattctttataatcagtggcgttatcttctccttcttttttaccttctttttccattttcttagctttcttagctaactcttctttagatagttttccggattgagcatctttaaatatttcatcagattgctccatagttaggttagcgcctatttctttcatacttctattaaataggtatttttgttctttttcactgccacccatttgagtagctatatcagccatatctgtcaagttttcaggattagatataccttcatccatacgtttttgtaaatcatatccaccttcaagaccttggtattgcgttccccatcccattgctatacgagcataagaactattcataccatttttaagtccttggtctatactgttaatagcattggcaccttgttcaccttgtaatcctttacttcctgactctgcaaaagtagattgcatggcagtaaggttactcatttggtctttagttagagttcttccttctcctgattgttccgctatagaacctagtgctttaagttgttcatcttgacgaccaaccatgcctgattgtttaataccacctaagaatgcatcttgcatttccttcatgttatcagaatttactccaccggtatgcatgatttgacccatagactcttgataagcttcttggtctttaatgcctaaagaacgtcctccaatagcaagctgtttagttcctgccattgtgtcctcatcacttttatgtcctactgatgcttcatatgaagtagccattttcaacatgtcagtactattataacctaatttgttatcaattgacaagtctccatatgcatttcttacagaatctatatccatattatcggaattttgacctaatgatgtaaccatcggtctattagtttcacttaatgaggcacccttcatgtataaacctgtagccatagctgtagtacccatcatagcatgagaaccaatagatggcaaacgttcttgaaccattctaccaaatgttcctctttgttggtctacgtctctagattggaaatccttttgaactgaacccttataatactgaactgttttatcaagttctgctcctaatttctcatagttagatataagttcttctctagctttcatttcctgtttaatagaagctaactcttgttcatattgttccttattaatttgaccgctagctctacgtttagtcaagtcttgttgttgatttctatagttgctatattgctctcttgcttgtcctaaattacgttggttttgttttctttggctctcataagaacttattttacctagctcacttctaactcgtgcaccttggtcatatgttaatcgtcctgttccttctgaacgctccatgtaacctctagtcatattacgattagctcttaatcgttttgtataatctttagttgcgctagaatctttacttattttattgtattcttcagttacttgatttaaggttttcatttccttaattctattttgaatactggcatcagaacctattttttgtgaagacattttctcaagtttttcttgagtttcatctacaactttacccatcttagcatatgctttttcaacttcagggtcaggtgcaatacctttagatatattctctttttgaaggtcatctaaagctttctttaattcatttatattctttgtagcagattgagtggatttaatagattcatccatgtctttagaagatataacagatttaccatcttgtttatccattgcgttctgtacatttttaacttctttagcaattacttttaatttttttaagaaattatctaaaggagcatcaatattcttagactctaaagaatccatataaagttctattgcctttagactattctctaaatccgaactatcaccggacaagaccaatctataatcgtcattcattgccatcattattcaccatccattttaaaaaaataaggatggtagtatcacctaccaccctttacatataccagtcatcatctaggtcttctagtgctttattaacgctatcttgtgatatagtagcgtcatcttcactagtcttaccatttcctacttcatcatctaattcttgttgtttctgtctgatgtattccatcatagctagatgttgtgtagtaagtccttctgtttcatcatttaactctgcatcgtttctcttagctctttcttccttatctctatcggataatttagcttccatctgttgtgctaaatcatcagcatctaagaaatcaggtactgggtcaaagtcttcatgagattcattccaccaactactatcatcatcttcgaattgactatcgaattttccacctttttgagcttgtttaagttgtttattatgttcataaacatctctattcatattcccaataataaactctatctgataatcgtctaagtccttaaatctttgctcggtaggtaaaacattaaactctttcataatagcccatagatttctagataaaggttgtttaacaattgattccataccgcccaatcgttctatatctttatctaatgtttctatactattagtaacgaaaggagtttaagaatcctaaccaatcttcatacataacagttaaaggatataagttataaatatcgtcagggtcttggaattccttaggaacttcaatacctacttcctgtaaggtagctaacatttgatatgctctaatcacttggtctgtttggtacatatccataccacctaagtaagcagaacgtaacgcaaagatattaccttgttctcgtgcattaggtaatttaatcttaactttaaatcttaagtctagttcttcaaagttatattctttttcccaagtatcattaacgcctttaataactttgttaataacttgttctttttcttgtttgtcttgatattttaatttatctatttcttccggtgtcattttactaacatcttttttttcttctttagttgtttcttgtacatcttgttcctttttaatttcttctgccataatatgatataccttccttttttaaatattaataaagcctacgcttatccgattcaatattaatataacacggactagtgtaggcttactactttaaacatagtatatcatattttgtttttaaatcaaaattactttacatattcacctgtacgcttagtaatttcttgtaaactttgttcaatcttttcactatttctaagtaggtaaatatagtattcttgtaatgaatcataatactcataatcaatagttaatatttcttgaccttctaaatattttctagtaatctttcctgcttgttctgttaatgttttgaatttatctaaaatctgattgattgcaattgcagttgatttacttgttatatctacctctttaggaaatttgtgtaattctactttcatttctgtgtaaattgctcttaatttatcttgtgacattttatatcttctcctttaatttgttatacctttattataatttattagttttaatttgtcaatacttttattctagttctactacataaattttagtgaccaataaataccattaataattaaacatgctaaaaattgaattgatgttattactactaatattttactcatagttaatgattcccttgtaactgtacccttaccatttaatatggttaatatgatgaggaaaatgctagacagaataaaataactatttactattgttatacccattataatacctccttattgaattgtcaagtctttactataataatttaaatctagtgttctatgtgtatcatctacactgttataccacaccgtaataactctagcatcttcaagactaattgtcaaaaactgatagctaggttttccttctacagttataactttaggatgtttaacaacaattctcctctcaatattaccattataatctgtgttattgtactctatgatattttcgtaggcaaacttatttttaaaaataaaacctattaatttaggtatatttgttgttggtttacctttaaccttttttgcactatgtcgtgtaaaattaataattctattttcagtaattgtctcttgtaataactctacacatttctttctttcatacatactcatttgagtataatgttttttaacttgcctatctttattagttgttcttctttctgaccaaaatgtactcatatacccatctcctttttttgttatactaataatataccttagttaattataattgtcaagacctttatgtttattttttctagaatattttgttttatcttttttaactttagttaccggattaatattccatgttgctcttgtgttactcatttttcctattgtttttctcttatttgccatctagtacatctcctgtttaaattattatatttataataacatataaattttaaagtgtcaataaaaaaagaccaactttttagttggtctaatctattaagtacgtgctttgtcacttgctgttaagtaactaaactcgatttcttctgttacaatttcatttgtctgccaagtttcattgtagttatttgcagagcaaccatgatatgagataataacttgtttcgttaagttatctactactaaaatatcaatgatgtctttcttaagaatttcttcaccaagtgaagcatatcctaaatctgcaaagttttcttttttcatacgtaaacgttctactgtaattgtaccttcataacgtaagtatacgtgttcttgaggcatgatagaaccgatttcgtatacaccagttgtaccgtattcacgttgacctgatgctgattgtgctcttcctaccggtttacctttaatcataagtaggacggtattaccagtatggacggtttgtttagcttctgatgccatctgtattcactctcctaaagtattcaaattatattaataggagtacctaatccaggtactccgtcacctagactaggcttgtaatgtctgttgcttgtaaaccaagctaacagagattttcttgaagcttctgattgggtaaactgtcattgaaattcttgcttcgttaccttcaacaataacttgtacgtcttcagcagggaagtcttgaatttcattatcacgtttcttacgacccaagtaagattggataaagtctttaatgattgaagcacttgtattaatagtacgagtaccaataaactggtcttcaagttgaactttaagctcacttactaagaagtcattagcttccccaacagccatttcagccttaactgggtctgatttatcgttgaatgtagttacatcgtcaacgattctgaagaatgtattagtacggttacgaacaaactctatactaataataccattttcatttaattcatctaagtctattgactcatagatttggtctaatgaacttacacgtagtggtttgaatgtgattgattcaccgatttctaaaccacttgcaagaccacctagagctacggctaccatgtaagcaggtacgtggtttttacgtccatcatccataacaaaagtacctgagttagctactaatgatactcgtggattagataatgatgcttgtctaccgaacaattgttctttagattcattgaatcctccaccaacaatagctctcattggttctcctgcatcagaacgttctttaacaaaagaagctacctctgcatgaactgattgtttagatgataatggaacaatgtagtatccgccttcatgtgcaaatttatctaacttgtctgcccatgtagcaggtggttcaccattagtaccgccttttaacttagttaactcaaacggttcaatagttttaataggtgaagtagcagttactgtagctgattcttctcctgcttcaacctctacattacttggtacttctccttctgcattaagttgctcgaaagatacgataccattgtaagctgtttgtttttctaagtcaccaaaaactgcttttacatatacagctttatcttttatatttgcattttcaattttatctaatttgctagattctaagttcttatctccgaaaggtgataatttagcttcgaaatcaggtaattgattaatgtctgtaataatagcattagtgtagtcataagctccaccagttaaatcatatgacttaacttcttggtctccaacttttaatactaaacgacttgctttttgagtttcttcatcatgttctacagagaaagttgcgttagcttcttctcctttgtacttgattgtgaagatattaccgatattatcataaacctcattgaaacggtcatcttggaatattactcttaaacgtaatgaatcacttagtgtattcttttctaatcctacttgaatgttgttagcaacattaccgtagattttagatgttatttttaatccgccaatttccgctgaagcaggtttagcatcttctatacgcatagctaaaatacgtcctgctgtatagttagggttagaaccccatgctaattctattgcatcaagtaattctcctgaacggaataaacgtttagcttgtgaatagttacgtaattcataaactgtatttggttctccgccttcagcctgaccgattaagcaaaatactttttcacttgaacctgctgagccaccgatacctgaagtatctacttcaatagatgcatgaggacgggtaataggtcttcttgggaatggttctactgccatatttaattctccttatctgaatgttattatatatctaatttcctacctagatacttctctaatgatggtaagaattcttgctcactaaacaaataatgactaccttccatataagctttaaatcctgctacttgtgaatccctcatattaaaaagggtttttgcagtatcaaggaatgtatctatatgaacataaccattaaatttaggagtgttattttcaggtgtcttttttctagccatcctacttccaatcctttctttctttaaaagttagtttattaatatcttgtgtaatagtataatccaaatctagagaacttgtgtacttaataattgttggtctaccaaaaatcattgagtcaccatcttctattattggtgcaatatcaccaaaagacaaattctgtaattggaatgtttgttgctcttctatactatctctcatagatattaaaatcattttcagtacagcatccaaacatcttgcaacgtctacattaaatgaaagacctacaactgttacttgttcttcaactgtgaatccttttactaaacctttagaatcatttttcttttcggtaaatataatgttagcattgtagttctcataatcttcatttgtttgatacttaaatgataccttattaccttcaacctgaagattatcgtatttagcaaactctatatcttctacctttattaactctcctattggtttagaaacagtaaaaactaacttatcatcttctcttattgctgtagaagattcgactaaagtatctcctgttgcctcaaagtaagatgactgaatactccctaaagagttttttgtctcttgtccttgacctaattgtatcaagtaatgagcttcatagttatttttaaattttgggaagttaaatcctaccgttacttcatgttgtgcattcttaccacagaaagcttctttaaagctctctctagtttgataatcaaagtcttttaaaacttcatctataatataacaattctctagcacagtgttaagtctaggctttatttctgataataaatatgaatcaactgatgttattgccatttaatactaaccctcctcttaatttaaatttttcatcttccagttcattaattgcttaacagtttttagagttgttttagagaagttatcttcattaactttatctctgttaagtatccaagaactagcaggtgacttactagaaacagttctaaatataaagtaagaagattgctttcctcttttaactttagtcatattatgagtcataggttcaggcttcattgaagggtggcttacattcttcctacgtccttctaggtaatcagttatcttcgaaactgaacctgttcctttatctacttttaaagttctcatatcttgataagtactgttattcattctactagttttgatgcggattggtacagttaaataccatccgccatctttactagttttctttttagatgagtgagcaaaagcttttttaaggtctactactccttctttttcaagctttttctctgttatttgtagataagtaggcatacgtttaatactagtattctctacttgctgtgctgttgcttgtaatttatcaagaacttctgaacgcataccatcaattgttctcttacctacctgcttatagtttttatttttaaataaattaggtctctttccatgaactggcattatttaaatgcacctccgaagaatccacctaaattactaggattagatgccgaagccttagggtcatctacttgaatttctaggtcttcttctataccatcattaactttataagggtcttgtagtacaatgacatcttcccttttaagaagtaatttttgaggtaagttttcaaattttgatttaggttgattaaacttagtatattgataacgactttcttttagtatatctgatactacatatcttaaagtcataagtatctttaaagttactgtatgattctcatatttttcatttaaatacaatctattattcttgatttcataatcttcttcatagactgtaccgtcttgagtggctatataagttatttcttttacatcgtagtataaaggtataccttttttaattctatctttattcacaaaataaatcatttgttggggcatcaatacctcaggaacagtaaacctgtctctataggaaattctcttttctaattgagtggtacctattgcagtacctgtatctaatatacctatatctaactggttagttcctttctcttgagactgtattgccattatcgtctctttaggaggtagatatgcaatacctttaccatgacatctagggcaatctactctaggatgtcctgtatcaggattaagacaaggacagaaatatgcttcttcccatagtacttttatacctcggtcattaataaacctaatcatgtcaatcgtatcaaactctagtctagtagtatacttaggtttatcttgttcatctgcttgtgttgtagtagtatatgttgttgacttattaataacattagggttagagttagctcctatcatatacggtttttccattttctaaccctccttataatcctatcatattatatccaaagtaagctcttaaaccatctaatagttcttttatatcttcatttatttgaagtatctgagcactagctccaccatacatagctgattgagtagtacctattgtctctgtaataccgtctacttctaatgttttattagcaataccggcaccaataattaagttaccccatacttggtatatttctttcaatgcatattttataactaactgttctaactcagggggcatttcccaaggtttatttcttcctgcttttttacgtggaagcatacctgatacatattctaatcgtatcatttgaggtgcaaatgttgctcctgatggtgggtatacacctgctaattgagggtatccattgaatactgcatcgtatgacattgattgtcctgtttgcataagtgctgtagggaataattgaacatgtcctgctagatgctctactttccaccagttagcagggtatttatatataggtctaccattaaactgtagctgtaagttctctacctgtaatataggttttctgtaagcatgtacaaacatgtaactattaaactctgtctcatgataatctctcatctcatgttgagtgtcaggtagtatagatatatctaatgctctttcagctttacctaccgctctttccaatatatggttatagaactcgtcacccataggctgtcctgtatcaggattttgaactacaataccgaacatatatgctttaactgcatccggtgaccatccataatcagctaatgttaaattatctatctcacttacatctatgtgtttaggattaccactaggatgataaggatattcatagtttaatgatttttcataagggtctaagtcccctccaaacattgagttaaccatttaattcaccctttacttattttcatcttttttggctgttgttttacgtgctgtagattttctagtagtatttgtagtttttctagtagaggctttcttttcctcttccttaggctcttcttctttactagtagaagctgattgttttctagtagttttcttttcttcttcaaattcaaaacctggaacattggcaaattctttttcttgttctttagttaatcctttgactactccatctttgtcaatctccactgtaccataaacagtagccatttttttatctaatagtttcttatagtataacatatttattaaatcccttcaagtttattttaataaaaaagggataaaccctaagtttatccctattctcttgtattcagtttttcttattctattaaactgcgatataacgaacgtttttaatacgagcccattttttaggagcacgtaatgctaatgcaccataccataatactgcaaatgtaatagaagcattaatttgagctaatggtaatttcatcattggaagtaattcgaataagtgaactacttgtggtgacatttcaccaacaaatacgtctgctgtttcaggcaatgtttcgttcttatctacgaatacgattgttccgtcttcttgtgcatctttaactggtacacgtttgattaggaagtacatacctgtttctttaccttgacggtagatagaaacgaattgtggttgttgttggtacatagcgttaacattaattgaaagtttaacaccatcgtctacgttagatactgtagctgttacttcttcagaaggagctgattgagcgtcatctgagttaactactactttatatgataatcctgcacggtcttcttcattttcaaaagcacctttttgcttagtttcaacagtagctgtaactttagcaggttgtggagcatttggtaatggttgtaatgattcatctaagattagttcattttccattactgtagaaccatgtaatttaatgaatccacgagatgagtagaaaccatttacgctgtaaccagtgttaacgttaccgctgttgtcttgcattaattgcatttgacgacctaagattgagttaacgaagtctgcgtgtacaccgataggcatgtaagcatctgtagctgtaccgaaacctttaccgatacgtaccgccgcctcatttaagtgtttctcagttaattgattacctttagcgttaattacgttatttttgtcaattaatttagctaaaccatcaaactctagaccttcaccttcaacttcagaagttaatgaagcgtcaccgtagaatgaagcccactcaattgtttttgcaacaactgcgatagcatcttctgtaaggatttgtgatgggtcagcaatgttatttactaaacctgatgcaattgacatatttttagtatcagaaacgtatttcattgatacagttttttgacggatatttgggtcagatactggtgctactccgatttctttaacgaaacgagagtgacctacgttaccatgacgtaaatattggtcgtattttactactgtagattgagcaggacggcgtgagatatcacgatagaagattaagtcttcattagtccatgttaacattgtgatttggtcatctaaaatttctctacgtaaagctcctgcgtcaatttgtgtatcaggagtgattccataaccagtttggaatgactttactacgtcttcttggaattggtcagcgtacttttgttgaacgtctgacaggttcttttcgatagtcatataaatttatcacctttcatttgtgttctttttattttatgtagggtacagtaaaatgtaccctatttatattctttagttacaccgtataatataacacaactttgtgcattaattaaatttgtgcaaagtcttttacagttttaatatctttctctgatgcattagtagggtcagtgttaatgttcaagtaagattggtaagctgattgtaagtcatgtttagaagctctagggtctttagcttgtgctttgtaagaatccatgaacttagttcgttcttcttgacttaatcctttaaatgtgttatcttcttgtacttcttcagcttcttctgattttgcttcaccagtttcagcttgttcttcagtgtctactgatttagaaacgtaaccttctactttttcttgctctccgtttgtagatgttactgatttttctacagcttcttcatcagtgtctactgatttagaaacatcttcagcttggatttcagaaatttttgctgataactcattaatagatttactaacttcacttaaatcttctttagtagcaaagttttctttctctttatttaagttttcaaaagatgttaagatagatttaaatactgttgtgatatcttcatcagaaattgatttagaagttttttccttcttatcttctttcttatctttatcatcgtcagtatctttatcttcatcgtcagaatcagaatcttcttttttatcttttttatttttacgtttctcgttgtctttattttctgtcttagtatctttttggtctacagggtcttttgattctttagctgatttttcaacttcctcatcttcattgccttctgctacctcttcagattcaactttttcaccttgctcttgtgcttctttagcatcttcctcagatactttttcaggttctttagcaggttcttcagttgtttctgctacttgctcttcggtagattctactgtattcttttcatcatcttctacagatttcgaaactgcctcttgagcatttaacttatcatactcttctaaaatattttgcatttctttactcatagaatgtattctccttacctattctagtttttgtttgtttatatccattactgctagttctgcatctttacgagataaacctttagctagttgtaacgtaaggactgattcctcatatcccatactattagatttactcaaatcttcaacaacgccattccatacatcattaaactcttttaaatctttaatcttagtgacgtaagctaaatttgtaatgctagatgctatttcttcttttcttaaagctcctgcatcaacttgagtgtcaggtgatgtaccatgaccagttaaaaatgatttcataaagctttcccatgttgcttcaggattagcagggtttttaactaatgcaactccggttatcataacttcatcaataactcgattgtcatttatattacgttttttaactgcaccttcaatagaaaaacctaaacgtcttccactacctgatttttctaatttctcagctaaatcaagcatcttaacaacattttcgtcattcttccatagctttgcttcaataaataaacctttttctatatccacatagcaattctctgtaggtatacctacaactttatcactttgatgctcataattaatgtacccttgtgacttaaaatactctatatctattcctttaggattaactatatcattctgtaaatccatacttggagtactagcccatcctgaaacgatagaatactcattagtatcagaagctgttgatacagatttcttcaaatccataggtacaaaagcattaaattttatttcttccaaaggtgtctacacctccacttgttattttatttacttttacgttaataatataacaataataacttgtaagtgaaatattaatattttttatgtattaatatcaattacatatagtatatcatagttattacatttatcaaaattaaattacgaaaaaaataaacaaagcctagatttattctaggcttattaattaatgtttaaagtcagaagatttttctccttttcttccttgacctttattagatgtttgagtacgataaacattatcgtcaccttttatttgtgcatctgttcctatctctttatcatcattactagaatctgttgattgcccttcttcagaatcatcattgtctccttctagtaaactcatcatcatttgtaaacgttctttttgtttaccatcattatattgtttatcttgttgtaattgggctgttccttgtaagaatgaagcatctagaataatgtctccaccttcaataggtttcttaccttgctcttctctagcctcattaactgttttaaatatttgagtctctagtttaagaatattaagtttatcagtagcactcttagtatctccacctacgaattggaatgtatacttatctccatattctgatataatatgtctattaactaagtcttcaataaatctaagtaaaggttgtaaacctttattttgagattgttgttgttttttacccgggtcagcctcatttaaagtagaaccacctttagaacctgtagctcctcctctattagggaaaccaatttctgcagggtcaataccatataaagcagatataatattgataaggtaatttaaccatttctcaaattgcatatcgttagcagtaggtgtcatatttacaaatttaatatcatctgccattaccactggaatttgccatgaaccattaatacctgataaactagatttccattcacgcttaaagttctctaatgcatgttgtgattgttgttggtctgaccgtatctgtaaaatacctctagtagtaccaccatgtgagaaaaatctatcattaaatgattccgtattattgtaggcaataaattctttcatagctatttctacttctgataatccgtatcctgaagaagataattcagttctagggtttcttatacccatagctaattctctagaagtaaaactagctactactcttttatctactacttgaacaaatctcttaccacccttaataattttaccttttttatctgttgcataaaaaatagtagaagggtctactgctatgaatttttctaatttagttttattattcttattaaatactttttcaaagttaacttggtcatagatataagtatctctaacaattttcttacagaaagtttgaaatgaatctctatctacatctttatctttacctgtattaacaataaaatcttctatgcgtttcatttcctctttttcttttctaccgggttccgcatctaggtctcttaatcttacctcaaaacctaaacctttctctgaatatcttgcaggttgacaatacatagctacttgatttgaacgtgtaagtatgatagcattaaggatagggttattaccaaactttttcaaaacatcatgtaagttatgttcgttcttcatgtaacttctcttatctctaaattcaggattcgtatccatcatttctataaatggctctgcataagcctgttgctgtccgtataaagacttagttaaatcttgatactctttattgtcttgttctatttttttaatgttagcttgtaatccttcatcaataggaacttgagcaatggtactattattaccatagtctttacctaatctgaattgcttaaataaatctgccaattactagtcacctctactccctcttactgtagttaatacaacagtattattataagttacatctctaatatgttgagggatatttattttaacctcaaacggttcttcaatgtaataaaacatcttcttactttccataacaatatcataatcttctaatagtatattattatctttatctactaatattactttattgtcttttgttgcttctagaatcatgtattttactttgtttaaagcaataaaaacagctacgtgttttccacctatctgtctataataatctacaatctctatccaattatcttgcataccttgacctttgatgaaatcttttgcttcatcccattttttatctttagagaatatcaacctattcaccgtcttttttattttttatctttaaagttactttttttaataattttcatcatcttgtaccaaattcctccaagtattgcacctataaaacttataataaatataattatagggtcattgaatccaatataatgttcagtattttctatggaatctattacatcattcttaataagtacagatgttgtttgaaacatagttaaaactatgtatgtacctattcctattatagcataaattacatacaaaacaaaaacgtttttactagagaacttactatgaacaaaaatcattaaagttaaaagacttaaacaggtaaatactactgatagtgacgttataatattcattcctaacaaagttttcaccaccgtaatgttcaatttttttatatttagctcttatttaatatagcaaacaaaaaaagaggagtaattactcctcttcagttttatcttcattatctatgttgttatcgtccggttgttcagtgggctgtttatcatctgtagtatcttcatttttatcctcttcgtccactttatcgtccggctctgaaggtgactcaggttcagttggttccgtttcccatcctccattttcatcaggattgtcagacggttcagatggttcaggttcaggttcaggttcaggttcaggttcaggttcaggttcaggttcaggttcaggcgtaacttctccaccatttcctcctgtattaccattatctgaaggttcttctcctgggtcaggtttttcttcccaatctcctcctccggtgttaccattatccgaaggttcttccgttgatggtggttcagaatttcctccaccaccggagcctcttccaccatcttcatctgtgtaccctggagggggaggatttggattatctgtatttccttgattattatctcctgggttctcaggagatatcggaactgaaggattatcaataggaggtataatatctgaaggtatagttggttcaactggatttgttataataggattatccgttggtaagttaatagaaggttttgtatcttctactatgctatctactgtatttggataaacattatcgttaataacacttaaaccatctatactatttgaaccaggtgataggaaaggagaacctaatacactagtaataatagaacctactgtttgtttttcatacccactatctacaggaacagttatcgtaccaattccatcttcactaataagtatttcgggcttgtctactttatcactaaactcaatattattttttactaatattaagtcatcatcaccagttacatcatatattttctctctagtttctttatcttctttttgtctttctttacttgacttcttattataatctttataatttgtttctttttttacttttttaagttcttcttcagccttatttaatcttttaacatatttctttttagtctcaccatcattatcatctgcttttttataaatctctactaatctttgatatctatctttatcttctttagcatcttgatatttattaaaaaatatggtcataatgactacaaatactgctatcataaccgttattatggttatgattacctcattgccttgtggtttctttttcaattatgatacctcattttatatagtctcataattcttcccttttataagttattaagtttctcttcttcttcttttctaattcgttctttttcttcaaagtcttttctaagcataaatgaaatacttccttggaactcattgcctgaaaggatgttacttaatgctgtatctataatagctacatcacctaactcaattgctttacctgcttctacatcttttctaaattgcttattagcttcgaaaatatcaatgcgttttgaaaaagcaagtttatacatcttatcatgtaaatctcctgttaaagggtcatttaatctactttgaaagaccattaaagagccatctagacgttgagtttcatccatagggtcttgtctaaatacattataagacttgtatagtctatttaccatttcacataataaactactaaattctttaatactatcttctttctcctctttactaagtggagttattgattcttctaatgttaatttattttcttctaaagtcatttatttcttccttccttttttatcttaccttcaatatcttcctgtaatatttttaataattcagttgtgttatctattctactcatattagactttatataaattgtttttaatacaatatataaaataaatacaatagaatagatgactatagtgctaaataaaatcactataatcacctaagaattgttttacatctttttttcttaaaatgtatccatctttattaggtttaaatcctaaagtttttaaatactctattgcttcaggtaattctatgtcatacacaactgcatttttattttctttaatacttaaacgtaataattccataatagctcttgctcctacaccatctgaatcaggtgaacttgcataagattctaatattatatcattttcagttcgtttaaaagatgtaattcctattgtatcaccgttatcatcaacaaactctttatacaatgatgagtttgtattaatatgggatacatctacaccttttttacttaaattagataatgaatttgctacttttttagggtttacctttccttcttcaccattagaaatagctcttgctcctactgcacttctactaacttcttcttttccttcttgtttttctttactatcgttctctttattaccatcttcgtaaactgtcatttccatttctttaccattacgaataacagtacgtttaaccttatttagtccaccaagatttttatctccacctttatggactatttcaggctctctattagcatataaaaaacaatcaaagaaattatcgaatcctagagatttataaacatgttcatatttaacatctaaaatatcactatttttagaaacataatctaaggataattcctcacccaataacaaattcaatacacttttattaatttcttcattactaattgtaccataaaataatttatctgtcaagtctatttcacctctattcatcaaagtagaattgtttatttccttcttgattgtaatcagtagaaacaaatgtagaaccaaagcttgtaccgtttccttctttaagaagttcttttattcttgttaatcctatataggcgtagacacttgcttgtgcatagtggtcgtcacctttacgtttgataacttggtacatttctccagttttttcatcttcttcatccataataacaacattttgccaatgttttaagaaagtttttaaatcatcatctactgtactataaacacttatatcctttgtcttaagtgcttgtacgtatcttttattctgcattaatttatccactgtaaccctgttattgttctcattaaattcaggtcttaattgtccggtagatttaggagaagatttataagtacatccaaatactttatcttttccaaaatgattaatgagttttaaaacattattacctgagtccccgttatctgcaattataatatcagggtcgtacttagatatttcccaaattattttttctaaatctgcttcaactaaatcaggtcttgtcatttttttaacagagaataatcgtattaagtctaccttaccattaggtaacataccatgaacagttatccagtgagtatttccccaatctataccaatagctatatgagaatatctatctcgtttcattaattgtgtttctgcaataggtgatttgttaccataaacatcttcttcattaactctaagtttaacatcttcaaaaggataacctaaaatatagttgtagaatgcttgcttagattctgtattcatttctttctcttttaattcatcagcagaaatccatacagcgttcatttgtgttattagataccctcgtacccctttattaccttttgtacgctcagggtacttacaatgccactcaccattataccatctatctagtggtttaccacatttttgacaaacaaattggtaactgccattttgtactgttttagcttgctcatctacaccttcaggattaacacataacatatttccactttcttcaagattatcagggttgtaatcattataactcatttcatttaagtaatcacaatgttgacatctatgaccgtaataccactggtctgattgttggtataatttgtgtatacccatcccaggtacagaaggtgtgctccatcttctcacaatcttaaaaggtgatgaagacattgattctaatgcagacgattctgctaataagtttaccctgtcatactcatctaaagataaataatcaatatccacaccctctacggtacttgctttagaacttgttctaaagaataaactagagtttcttatctttttaaaacctaacgagtctttatcccaatcaacaatgtctctaaaatattctttctctaaaacagggttcaaacgtgactgaacaaattttttcatttgttcatttgtagggaatgtatacagacactttgcgttagcataactatgcatatctgcaaaatgaaccatttccattacacccatttcactaagacctaactgacgtgattttattacagccttattaggatgagtatcatttacaatcctagtttgccaaggtctatgagcctgtgctttacttctatctctattaggaatactaaaagttataggatgccctctcagtgtatgatacttaagcatataggtagaaggatttagcatattgattatatggtctatctgttctcttgttattttttcagtttgaaatgtttcttgtgctatcttaattagttcttttccatccatttatatctttctccttagaatgtttctgaattttcctcattcattactttttccttttcagaaatcattgctgtaatatcttccgctgacatttctgatatcttctgtaaatcaactgtagattctccattactatcttctgttgtaatctcatcaaataattcctgttgaggtctagatagttgaggtattgctcctccttcagaagtttcattgaccatattttgcatttgagagaatatcacaaataatttgtataaatcattcgggtctttaacttctatttctttgttatctactttcttattgaattcgtacattaatttaccgataccattcataaacccatcagctatatattttcttgtatcctcttcttctaactttttcttgttaattatatctctaatattttctgcactcaatccatttcacctcgttttcttaaatttttataacaattgttgatatctgcgcaaatatcaaaataaatatacttagaatactgtaaatgaaaatgattaacttttgataatgcttttcctttagtgtaattcatacctgataactgctgaccacatagactacagattctactagggctagctattctcttatctcccttttttactaaagaaacagatgtagcaccatccttaagttctttcctttgttctttaatttctttctttgttttcattgtaatcttcctttttactatcttcctctactcttctttgtgctttagccctaacttttatctcttctgctgttataccataattactttctattattttcttgattattcttatttctttatcatttatctctacttttttaagaacatattcaggtagggggtcttctttcttcagtaagaacttatctgttacagtaagtgcgacattagaaacaataccaccaaagataaagtatgtcataactacactcggtcttattgagtaaataagggacaaggaattaataacagacaaaattagtagtaagaatataatgataaccagtgttttgaatatgaatatagctatgttattattcaaccactttaacctcctattgtctttttttattatattattaatctaacaattgacttttatgactggcttttaatgttatattagtggtgtaatctaaaagttctagaaataataggtcaaaaagaaataggtgtttaacaatgattatcgtatctttttttctgtctatacctcttttttgtttctattattttatacttagtggtgttacaacgtctttaggttattctatctatagaaaagataaaagtaaggtaactcttaatatagtatatctatcagctatctatgctttatctgtttttagtatttggcttattttctcagaaaaaataattattatcattattcttaatataattataataactgcctttatcataactattataccacaaaataagtaaatttaaaaattaatagtaaggaggttaccactttgactaaatataaggatatccttaagttagagtttaaagatgcactagcccattttaaaagagacagacgctatttccacgtctatagaatagacagagtattaataaatggctcaattatatacttcgactattattaccttccttctgatgaccctaatattgtaataaaagaattagacctccaaagttttggtaagcttaggtttgaaattgatacaaaaacatcttacggaaaagtcgttacggataattatatggagattatcaatgatttcttagaaaattatgatatccattctgaatctgaaacggttagaccttagaggtgattctagttggataaggagataaacaaccttgtttctcaagtagaaactattaagtctaagatacaagaaggtaattatatagatagaggaactttcaaagatttagaagttgaggtagctgaactaagaaaaatgatagttagtatagataaagatgtagctgtcaatagtgaaaaacaatcagcaatttacgtccaattagaaagacttgatgaaaagatttcagaacttgctgaaagtacaaaaactaaggatactaagaaaaaggatacaactgagaaggttcttttacttgttctaggagctatattatcctttgtctttaacaaatttgcataggagagattgtagatgtcaaaacatattgaaataacaatgtcaagtggagctaaatactttttagtatctactgatgaaaagagttacaataggcaagatatagattatatgttaagaggaatggatgaaacctctataaaagtatatactgaaagtgctataacttcacctcaagtatatattaatccaaacagaatagaatcttttaaaatagtattctaattattgagtgactttaagtagtcacttttttatttatactattgttgactttctaacattgttaattataattatatatttaaatagtaattaataatagtaataaatatatataactataataggagagctccttattttctaaatacgtacttttttgcctgtaagtactgatactaagggattttacttttatactgagtatctataagcagtatcagttatgtttatatcaattttcccaaccttattaattataattatatatttaatattatttattactctatatcagaatataattatatataataggagtactccttattttctaaatacgtactttttctcttgtaacccttgatattaaagtattcttcaaaataatttaaattatttttagaaaactgttgacttatttatcaatatagtatatagttaaattatcaaataaaagagaaaaggaattgattaattatgaaaagacaaaaaatgttttactcaagtttaatatgtaaagaatgtggaaatgtattcaaagtaccaagaaaaagagcaaataaaagagaagaaggtcatatcaaagatatatattgtatcaaatgttgtaaaactacaaaacatatagaagataatcgtagtgaagcagaaagaagatgggatgctattcaggaggaactaacaaaagataactaatgttttaaaaaaagtattaaattaacttgacaaaggtatcagtatatgctataatataattaacaataaaacaaataccttacacatcactgtttttattacattgttttccagtataaaacaataacaatgttatcatccttgtgtaaattaaatagaagtaggtaaacgtccctacttctataatttttttgaaattaacttgacaagtaataaaaattatgttataattaatatataaataatacaactaaatatctgagcatagtgactgaataatgatttggtagtcataaggtacaagataatcaaacgaatcaaaacctatcggagatatatcggtaggatgggtcgtcattagtattgttaaatacgattggtatacttggtacttacttataaggtctgtagagtatgataacggctagggtttaatttctttttagggtcttagcggaatcctagaaagatacgctgtaacgactaactgtagttggagcacagaaatgtgttagataatagaggttatcagtataagtagcataaatctacaggttcaaaagagctatatttgataaaaataatttggtaatcaaataaatttacttttcttcgaccatgtgaaagttattaactaatatgttaatatcacattatttaataaacttattaccgttttacggtttgtaagtcccttttaaattgcgtgagatggacttaaggtagctccttaatagagtcaatcgtagtgattcacttaataacaggtcttataggtaggttcctcgtgagacgggttatgcttggatatttagttgcattatatcagaatgtgactataaatattttcctctttatagtttattattaacaattatcagtttcctcctttgattgataattaaaaaaagaatctacagggagtaaactttcactctttcatgtatttattgttcacactcctttatgaaccaataacttactccttgtagattatacatatttcctatataagtaccaattaccacgttttgttcatttcacctcctttcacttaatatatagttgttatgagtggtaataatcgttgcttcgagcttatataaaaacctgctatacattaaggtaaggttagggtatagcattataattttggctcattggtgtaactggttaacacactgccctgtcacggcagagagtacgagttcgagtctcgtatgggtcgttttaaggtttcttagctcagatggtagagcactagattgaagctctaggtgtcattggttcaaatccaatagaaaccataataaacacccttagtataattagtagtacaagggtctccaaaacccttagtctttgtgcaaatcaaagagggtgtgttttacataaatgaaatctgagaattcatatgtgcggttgacgaaccggtttgctgtgtcctcgaatgagggtaggatttcattgagtatatagcgcaagtgtttgttcctaggatacgatactagctatatactttacatatttaaaaggagagatacaaatggcatcagcaaaacaattatattatacggaaagtttagttggtaaggcaattattaataataaagtatctaataaagaagaagtttgggataagttagagctacttcctgaaactaaattagaagatttagataacaaacaaatgtctgaagttatcaaaaaactaaaccaaattaatgagtaagtgttaaattaatatagataaacatgaccgacctactgttatattattgttagaaataaatataatagaaaggtcggttttttaatggctaatgaaactaaacaacctaaagttgttggaggaataaaccttagcacaagaactaagagcaaaacattttgggtagcaattatatcagcagtagcattatttgctaaccaaattataggtgctttcggtttagactactcagctcaaattgagcaaggtgtaaatattgtaggttctatactaacactattagcaggtttaggtattattgttgataataatactaaaggtcttaaagatagtgatattgttcaaacagactatcttaaacctcgtgatagtaaagaccctaatgaattcgttcaatggcaagcaaatgcaaataacactagtacttttgagatagacagctacgaaaacaatgcagaacctgacacagatgatagtgatgaagtacctgctattgaagatgaaattgatggtggttcagcaccttctcaagatgaagaagataccgaggaacatggtaaagtatttgcagaggaggaagttaagtaatggctaagactcaagcagaaataaataaacgtttagatgcttatgcaaaaggaacagtagatagcccttacagagttaaaaaagctacaagttatgacccatcatttggtgtaatggaagcaggagccattgatgcagatggttactatcacgctcagtgtcaagaccttattacagactatgttttatggttaacagataataaagttagaacttggggtaatgctaaagaccaaattaaacagagttatggtactggatttaaaatacatgaaaataaaccttctactgtacctaaaaaaggttggattgcggtatttacatccggtagttatgaacagtggggtcacataggtattgtatatgatggaggtaatacttctacatttactattttagagcaaaactggaatggttatgctaataaaaaacctacaaaacgtgtagataattattacggattaactcacttcattgaaatacctgtaaaagcaggaactactgttaaaaaagaaacagctaagaaaagcgcaagtaaaacgcctgcacctaaaaagaaagcaacactaaaagtttctaagaatcacattaactatacaatggataaacgtggtaaaaaacctgaaggaatggtaatacacaacgatgcaggtcgttcttcaggacaacaatacgagaattcattagctaatgcaggttatgctagatacgctaatggtattgctcattactacggctctgaaggttatgtatgggaagcaatagatgctaagaatcaaattgcttggcacacgggtaaataaattgccctgttatacagtaatgtataatgaaagttcttttaattgactgggaggctaaggctatcaagctatgctaatcagcatccaagaatattgttttaaaaaactacttgacaacataataactttcctatatacttaagtaaaggagatgttattatggaaaagaaattaaatgaaatacctggattagaaatatatgaaaattacactattactgataaaggagaagtaatatcttataaaggtaaagagcctaaaaagttaaaacttcaaaaaaataacaagggttacttgtttgtaaggttacgataccattcacctaaaatacatcgtttagttgctatggcttttatacctaatcctgataataaagaacaagttaaccatttaaatggtaaaaatgataatagtgtaggaaatttagaatgggtttctaattccgagaacagagaacatgcaataaagacaggattaaaaaatgaaataaattataatatagctcagtatgacttagaaggtaatttattgaatgtcttttacacagctcaagaggctttagagttcttaggtatttctaataaaagaagtggtaatataggaagatgtatcaaaggagagagaaaaacagcctacggatacatttggaaacaatattaaggttcaacgactatccctcggacgagtgccaataaaaataaatcgtcaataggagtagggctcaagttaatggagtgggtgagaatcccttaaatcgaaatggagaacccctaacaaaattagggtgaagatatagtctagtcttataggaaactataaggagttcataagagaaccggtaaaggagttacgtcttttattgaataatcggatggaacaggagcaaactcaggtaactttagatttgcaggtattgaagtctgtcaatcaatgagtgctagtgatgctcaattccttaaaaatgaacaagcagtattccaatttacagcagagaaatttaaagaatggggtcttactcctaaccgtaaaactgtaagattgcatatggaatttgtaccaactgcctgtcctcaccgttctatggttcttcatacaggatttaatccagtaacacaaggaagaccatcacaagcaataatgaataaattaaaagattatttcattaaacaaattaaaaactacatggataaaggaacttcaagttctacagtagttaaagatggtaaaacaagtagcgcaagtacaccggcaactagaccagttacaggttcttggaaaaagaaccagtacggaacttggtataaaccggaaaatgcaacatttgtcaatggtaaccaacctatagtaactagaataggttctccattcttaaatgctccagtaggcggtaacttaccggcaggggctacaattgtatatgacgaagtttgtatccaagcaggtcacatttggataggttataatgcttacaacggtaacagagtatattgccctgttagaacttgtcaaggtgttccacctaatcaaatacctggcgttgcctggggagtattcaaatagaatataaatttagacggattttaaatccgtctattttttttgcaaaaaagtgttgacaaaattaaatacatagtgtatagttatatatgtaatcaaataaaggaggaattacatggcactacttttaacatattttgctatttttattgtttttctagtccttgtaggttttggtataagttatttatttgattttctttcaatgaaagagaagaagagtaacataagaaaacaatacagggaattagttaggcaaggtacattagatgaatacggtttagaacaatatgtaaagtataaaaaacaattcttaaatgaccgtagacaatcaattgtaactagagccgataaacaagaaatagaccaagaggaaaaagctttaaatagcttaataaaagaaatagaaaaaggagaaatgtaatgagaaagtcagtagttatttcaggagtattagggtttttagcaattatagggtttattattttattaatgtgtattactaagattccacaaggtcatgttggagttgtatactcagtaaatggtgttaaggaagatactaaatcaccgggttggcatttaacagcaccttttgataaggtaaataaatacccaactaaaacacaaacacataaatataaagatttaaatgtagcaacttcagatggtaaaaatattaaattagacattgatgtatcttataaagtagatgcaactaaggcagtaaacctttttaatagattcggaagtgctgacatagaagaacttgaaaaagggtatcttcgttctagagtacaagataatgttagacaagcaatttctaagtactctgtaattgatgcttttggtgtaaaaacaggagaaattaaacaagatactttaaataaacttaatgataatttagaaaagcaaggttttattattgatgatattgcattatcaagcccaactgcagataaaaatactcaaaaagcaattgatgagagagtaaaagcaaaccaagaactagagcgcactaaagttgataagcaaatagcagaagaaaacgctaaaaagaaagaaattgaagcaaaaggtgaaaagaaagccaatgacattagaagtgaatccttaacagaggaagttttacaacaacaattaattgaaaaatggaatggaaaacaacctattagtataggttcagatagtgttattacaaacttaaataaataatatgtttaaccacctattgacataggtggttttttatgctatagttttattataggaggagatattatgattggagaaactattaataaactaaaagttataaaagagtctagtaagagggataaatcaagatgtaaaatgtatgaatgtttatgtgaatgcggagaagtaataatagttagaagctccactttaaggcaaggtaaaataaaatcttgtgggtgtgagagtaataaaattcatagtgagttaatgagagaaaggaatactacccatggattatccagtaacccaatgtaccaaagatggttaggtatgaaacagcgttgttacgatgttaacgctataaattataaaaactatggaggaaggggtatagaaatatgtgaagaatggaagaatgattttaaaaaattctacgactatatgggagaccctccgaatgaaaattatcaaatagatagaataaataatgatggtaattatgaacctggtaatgttaagtggtctactaggtctgaaaactcaactaatataagaaagaaaagtacacataatatatacaaaaaatcaaataatgtatataatatacaaattgtaagaaaaaataaagttaaatattttagtgcaaaatctttagaagaggctatagaattaagagataatgtaataaataaatataatgaaactggagagtggtaaatataattacctctctttttttgtatttaattatctgataactgtaatgtttgtaataaaaccttaataatattacactaattagtgctatattatacttgcgttatttaaagatatatgttatgattacttatgtcggtttacaaataaacaattctaaaaaattatttaaggagttttaaatttatgaagaaaacaatttttgcaacattagcattaggtacagctattacttttggaggtattgctacaaatgaagctagtgcagacgaaattgattataataagttagcagaacaagctaaatcaaattcagcagaagtaaatacaaaaccaattcaagcaggtaattatgatttctcttttagtgatggtgaatttacttatcatttctataattataatggtaactttggttatgaataccattcaggctcaactcaagtagataatacagtatctagattagcaggagaagaacaaacacctgaacaaaaagtagaccaacaacaagcacaatttgatactcaaaataaacaagatactaaaaaagaagtacaaacaacatcagcaccagttcaaaaggaaactaaacaacctacacaatcaactagttctacaggaggctctgtagcagaacagattagacaagcaggtggagacgaggcaatgattgaaattgctatgcgtgaatctacaatgaaccctaatgctgttaatgcatcatcaggagctcaaggattattccaaggattaggtaaatcatggagtggtggttctatagcagaacaaactaaaggtgcaaaacaatatatgattgaccgttacggttcaacatcaggagctcttgcataccacaacgcacataatagttattaatatattaagactaagattaatttcttagtctttttttgtatattatagttgacttcataagttaactatgctataattagtttaacaaataaatagaggaggataatgaatgaataaatttaaaagatggtttcgaattaatgttttaaaaaaagaaacactactttttaaagtttattggagatacgagtcaccgtctttaaaaaaacctcatgtatttcatatagagttatatgctaaaagtaaggcagaggcaagaaataaatcacaagagtatatactaaaaaatgcaaaagcatctgaggattttaaatttttaaaagtagaggagaaataattatggatgaaaaaaaggaaagtaaacctctaaaccttcaaaaaattagagtagaaaaaggacatacgttaagaagtctagcttctgagataggtgttcattactctcttatatcttattgggagtatggaaaaaagaaaccaagaagtgctaatttaatgcggttagagaaagcgttaaatactccaggaaaagagttatttaaagaattggaggaagacgatggagaataataatttagttaattttttaatgactacagatgatatagatgatactattgaaatggtagattcatttgaattacaagatataaacaaagttctaggtgaagatacgtttttaacaataatggaaattacagacagtcttcctgataaccaatataaaatagtattgttgtcctctttagacaagttattgaatacagatagaaaagaattagtagaatatgatgaagaatttcctactatacgaaaacataatgtatctgagctaaaaagagatacagttaactctgtaattgatagttatatgaatactaatgtagaaatactttatacagagtatcctactattagtaactacagtgtagttgtagattctgttaaagtgttaaatactttatatttaattgaaagtaaaaatggtaaaatagaagcaacactgtcagaagatggagaagacttacatgagtatatatcagaagagggttacagtgttacagacatattaaataaatttgatgatgttgaagatttatttgatgaggatgacagtttaattaatttcttttcagatattgatgaaggtaaaaataaaactattaaatcatttattgagttagttattaatttaaaataaaattagtggaggtatttgttttatgaaaaaaggagtatttacagtaatagctgatggttttaaatttaatgtgattgctaaagataaaaaagaagttcaagaacactgtttcaaatgttttgattttaactatatctcagtatctttttgcagagaagtctattcagattgtgaattccctcaatttatggaggattataagtatgcaggatagtgtaaatatatacacagacggtagctcctcatataataaaggtaaagtaggctcaggtgctgtcttggtaagtaaagaaggaaatataatatcggaaattagtaaaagtgttgacaaaccaggattaataaagtataataatgttgcaggtgaaatattggcttgttgttatggtattgaagaggctataaaactaggatacaatcaggcaatagtttatatagattatattggtttaatacattggtatgaaggtacttggtctgcaagaaatattctaagtaaaacatatattaatatgatacgagaataccaaaaagtaatagatataaactttgtaaaagtaaagagccattcaaatgacaaatggaatgactatgccgataatcttgcaaaaaaatcaattgatatataaaaaggagaaaatattatgaagaaaattaattcagtaattaaaggtgaaggtaagaaagtacaaacagcagatgttaggaaaattagttattatgttaaagattataacccttgcatgacagtagatgacgcaaacgactataatgcaactagtcaatatttggtaagtgacaatggtaaatttattgcaaaatataataaagatatgaatgcagtaggattctatgaagaatcaggggatactgtaaaacatttaacacatactacaccggaaagattagaaggaactgtattcactattgaagaagaaacagagattgatttaattaatgataccttacctcaaggtgatattttaattaaattttcagacggtagtatttatttacctgataatgaatcagtactagatagtgtaaattacttggcagataacgattgggattctgtggatgatattatttatacaggattatctaaaggtaatagtgaaaattgtattgtagattttaattataataattatgatattggttatgatgatgtagaagatgaagatgtttgtgataactaccctgaatgtgaatgtagtaattattgctcttcaacaggagaatatatcggaaattagtactatattaaaagagagtaaaactatttatagataggggaactttaaaatatggcaatacaactaaaagagttagactttaagttaaaagattatcctaatgtaagatacaacatgggagaacatctagtctttaatgaattccttgaaaaagctacaaccgagcagttagatttctgtgaggatttctttaatgataatgttgaaatactttggaatgagagtcaagccggtacaggtaaaacaatgtgctcagtagcctgtgcttacgcagactatcttaataaaaatagaaagctagtatttataatttcaccagtatcagaagatttaggaagcagaccaggtaatcagacagaaaaagaaatggcttatttcatgggattacatgatgcccttattgaacttaatatgaatcctgaacaacaaataactgaaatgttaatgatggaagataatgttaaagaagataaactaggagattgttgggtatctcaaatatcccatctattcctaagaggtggaaatctaagagatgctactataattataaatgaagcacagaactttaaacgtagtgaacttaaaaaagttcttacaagggttcatacaaaaaattctactgtaatagtagaaggtaattttaaacaaatagatttaaagaacgaaagtaaatcaggttttggagattatatggaatactttaaaaattatgaaggagcagtatttcataattttacagttaatttccgctctaagcttgcacaatatgcagataattttaaatggtaaaataatatgttgacaaatccccttagttatggtataattattctataataactaaggaggatttttttatgaattatttagctaaggtatttattaataacaattggttggtgaaacttataacaattgtattattaactttatttctaagcggtctagtttatgttataagtgcaatatcattattcttatcaacagttttaaatttacctggtttagtagtattagcatttttagcaagcgtaagtcttattttgttttctatagtacataattcaaaggaggataattaatgaaatcgtatactaaagtaaaaaataaaggtattgtactagataaatttaaagaaagaggtctagttgtacaagaaaaattagatggaagtaatgcaagcttcacagtagaaaatggtgaattagtatgtttttcacgtagaaaaaaattaaatgagaatgaaactttaaatggtttttatgattgggtacatgaaaatataaatgtaagaaatacgtacgtatcagccttagaaaaatacattatttttggtgaatggttagtcaaacataagattcagtacaaagaagaattttacaacaatttttatgtatttgatgtttatgataaagaaaatgaagtttatttatcagtagaagacatgaatgtaattgcacatcatttagggttaaaaacagttaaaactttgctagtatctaaaccatctcactacttaaatgatttaaaacctgaagaaattcaagaattagtaggaaaatctgacatgacagttaaacctgataagggtgaaggtatagtaattaaatacttagatggtaaatcagaatatgatgactactttaaattagtatctaatgagtttaaagaatttagtcgtcaaaaaatgaaaacagaagtaaaaaagaacgagtcagtggcagattatgccattacaagagcaagaatggaaaaaatgatttttagggctatagaagaagatagattatctgaagatgatttagaattagaaaacttcggtctaattatgaaacaagtaggtcaaaactttgttgatgatattatggaagaagaaaaagaaaatatactgaaaatagtagataaacaaattaagaaaaaaatgccacatattttaagagaaattttagaagaaaaaggagatactatagatgggtaataaaattaaagacaaagtaatttatatgggtggacatatcctaaatgaagctatggtagattacagagataaacaacataaagaagtagatggcattgtaggagtaactccttatagccctcacaaagataagtcaataaatgataaagctaatgcagaacaaactaagctagcagaacgtattttaactaatgactttaaggctatgcaagaatcagatatttttgtatttgatatccttaatgaaggattaggaacaattgcagaactcggtattttattaggtatgaaacatcaagcagaagaaacaattaaccatatatatgataatggagaagagtattttaattattttacaaataagtttgaaacgtcattaaatactgaagaagaattaatagtagataaacttgaaaacattgtaaataaacccgtattaatctactgctcggatattagacaaggacatggtaaaccatacaatgaccctgacagagctgaatttagtacgaaccaatttgtgtatggtatggttttagaactaacagacggagaaggatttatttcatgggaagaagttattaatagattagagaaattaggggaacaagatggataatttatcacattacttaagtatattatatgctatattaattacagtaggatatataccaggtttagtagcattagttaaagcagaaagtgtcaagggagttagtaactatttttggtatttaattgtagctacagtaggtataagtttttacaacttattattaactgatgcttcagtatttcaaatagtatctgtaggtcttaatttaaccttaggtattgtttgcttattagtagcttcatatagaaaaaaggactatttctctataccttttattattgtgttctcactgttactatttttattaagtgactttacagccttaacacaaactgtagcaactattactattatccttgcgtatgtaactcagataacaaccttttataaaactaaaagtgcagaaggaacaaatagatttctatttcttattattggattaggattagcttcattaattgtaagtatggttttaacacatacctatgttcatattatagctactgaatttgtaaactttgttcttatacttatatgttatctacaagctaattattactcaagagggtaggtaatattgaatgaccttatcaaagagggaaataaatattatcacaaagtaagagcaggagagacattatggactataagtaagaactatgatgtggaaattaagaaattacaagaattaaacaatattaaatcagtttctttaactaatttagaatacgtacttgtttgtgtagagtagaccaggaggtttataaatgagcatagaaaagaaagaagaagttatagcacataatgaggtagtatttaggagtttaactcaaggtctatatgtaaaagaagtagatatctattcagatgttgtaagctatactaaagatgttgatgaagctcttgctatgccaaatactatcaattttaaaaattcaagaaagtataaaaaacttattatgaatttagatttagaaccattaaataaaattcaaaaagttatatacgaaactcatttagaaggactttaaaaagttcttctttttttgttgacaaatattatttactatggtatgattagtacataacaaataaggagtgattttaaatggcaataacttataaacaaaaaggattaacagaacaagaaattattaatttacctaaggttaataaaggatgtatctatataggagaagaagatgtatttcttaagaaaaagaaaaataatataattaacttaggttctaaagaattatttagagatattcataatatatttagttttgatacagccacagaaatacatttatttttagccctatgtggtaataaagaagtaacaaactttgaaggtaacccttatgaaacagttgaaaaattagttgaaggtgtagttgatgataataaaggaagaagttacaaagagtatattgaatctaatagagaagaaagaaaagattttcctgtatatggtgataagagtagaagacgtatacaatcaaaaggttatgttgaagaaaaaattaaagaactagaaggtaatgaccatttatggaggaatgaatctagacaattagaagaatataaaaaagtagtagatagtttaaataatgatattatggatgtactagaccaaggtaaatatggtcttataaagtcatctattattgttatgaatgaagatatagaaaaaggttctagtgagtactatagtgctatgacagatgaattatatagccgtgtttggtacatgcatccaagtactgaaaactattcatcttttggtcttaaagttaaacatattagagataaacataatatgggtaacaaatgggttttagaaaataaaagttcatttgacgttaaaacaggagaagttaaggttttcttaacagatagtcttgttaataaagaaataactttaaacctatataaagatgatattagtaaaagtgaatataagaatgaattaactttatctgttttattaaatgttattttaaaaaactatgcacaacctaacttaactagaggaattataataaagataatagaacaaacattagagcaccataattttgatttttctagttggtgtcctgataatacagatgtttatggtcatataaattatagaggagataaatataggatttttataggagaaaattcaacttctaattacttaataactttaacagatattgttaaaaatattgataaaataaataacttagaagaatttgggttatttgaaagaaatgcattactattccatatacctaaaaaacctaaatggaaagttcatgaagccttcaatcttacaaaacagacttataaaaagttactaactttaaataaatttgagcaaggtaattacttaagatttgctaatattctctataagcactataatcatttacacaatgaagttaatttacaccaattgtttgacgatacctttttaatggttagagattcaagagatgttacagatgctttaaaagttaaacctattgtgaatcaaatattatctatatcttttgctaattacaaaaagatgacgcactatttagatgtagatgctcaagacagacaacgtataacaggatatgcactagataactattacttagattacttacatgatttatcaatattaataagagaaggttatagaacattagagagtgttagtttaacaccattttcactaaaactagaacatgatatagttacagatgagaaacaatctatacaacaacaattagatgatgcagaacttaaagctaaatatgataataaattagaaaaaataattgataaaacttataaattaaaagatggtagaaaagtaaaattccttcctgcagatactgtaagtaaacttaaagatgaaggtaaaatgttatctcattgtgttggtgggtatgctaacagaattctaaaaaatagttgtttaatattattagcaagattagaagaagatttagataattcatggtttacagtggaaatacgtattacagataatggttatgtattaggtcaacaacaatcaattgatgcctataaattacctaatgaattaaaagaagcattagaaaaagatatcaagaaaataaataaagaagaatttaaggaggttgcctaatggtaaaaccagttataactttagaacctgaggatgtaaaagtattattagattaccttagtttcttggaagatgatatgagaaactatgaaggtatgagagaattatatgaagaattacacaaaaagtatcaacttgctaaaggaaactactcagattaattgacaatagtatcataatatgatataataaaggaagttagaaatggaaaaattccaagaagattatgttaatatagatatcagagttaaagcttatgttcgtgtaggttataggtatgaagaagatattactaataatctacatgaattagttgaagataatttaaatgtaacaagtgactctgataacctaattatcaaagatacagaaattaaaggagatatagaataatggaaaaagtagaacttattaaacaatgggcaaaagatagaaatttacaaacaggtaaacctgaaggtcaaatgttaaagttattagaagaagcaggagaattagcttcaggtattgctaaaagtaatgaccatgtaacaagagatagtgttggggatatttttgtagtattaacagtactatgtttacagttagatatagatattgaagagtgtattgatatggcttatgatgagataaaagacagaaaaggtaagcttattaatggagtgtttgtcaaagaagaagaccttaaaaaataagtaataactagcaaggaaaactgttatattatgaggtgtacttagatagtacacttccataatatctttcctttttcaccttgcttgtagccaagcagggtgtttttttttatataatttaggagggtattacatgaacgcaaaagaatttatgaaaacacaagctcaagtagaagattatttagataaattaaaagtaacaattatagaagatgcactatcagtatctaaagaatggtctaatgattcaaatgatttaggttacgctttatctagtcttggtgaaagtataggtcttttagaagattattataatatacaagtagatgcacatttacctgaacactataaaggtagtaaggatgttatttcttttctagaagaacatttttcttatgatggctttgttgattctatgatatttaatattgtaaaatatactacaaggttaggaagaaaggatgcagtagataaagaagtacaaaaaattaaaacatattatgtacgattagaaagaaatataaagtacggagatagtactcgtgtctaaaagaacagacaattttatatatttctgtaaatattacttttcagaatatttaccttcactaggtgtagaagtacttaatcataatgaaacttctcatggaacaatggaaggtgttaggaaatattatatagcaaacatactttatgaaggtcaagaacttacggtaactattgatttagaggaattcaataatgcaacttctatgcataacatgttagaaataatgaataatcatacatataattgtatgtttatgtatgatatggatacacatgaaactaaagatattgatgatttttttaaattaatgtatttttaataaaagtgttgacaattaccttaccctatgttaagttataggtgtaaggtatttttttttattaacaactaaaaaaagttttagaaagttgttgacaaatacaaatacttgtaatataataaatgtataacaaatcaaaggagaatgataaatatggcaaagcaagatactattgaaagattagagagattggtagaacaacaaatggaaacaacaaaagacttggcagacaaactaggagagaaaaactctaatccgtatgaacaagcaattgtagatgcaattgttgagaaagcaggaactgagagtagagaaattattattactgacgttaaaaaacaaattgaagaatatgtagaagaacaacttagtaatttaccagttaaaattgaattacaacaagaaggaaaaacaattaaagatatctcaggaatcttccattatagataccaagatatactaaagttagttaaccaaaatattccagtatttttaaaaggtggagcaggttcaggtaagaaccatgtattagaacaagtagcagaagctctagatttagatttttatttcagtaatgcaattactcaagaatttaaattaacaggatttattgatgcaaatggtaagtttcatgaaacccaattctataaagcatttacaaaaggtgggttattcttcttagatgaaatggatgcatctattcctgaagtactattaattcttaattcagctattgcaaataaatactttgacttccctattggacgtgtaacagctcatgaagatttcagagttgtgtcagcaggtaatactatgggaacaggagcagaccatatttatgtaggtagacaacaattagacggagctacattagaccgctttgctcaagttgaatttgactatgatactaaggtggaacatcagttatcaagcaatgaagacctagttaactttgtacaacaattaagacatgagaatgatgaaaaaggattaccttatgtattctcaatgcgtgcaattattaatggtagtaaattagatggagtaatggaagatgagtttgttgtagaaagtattatctttaaatctgtaccgaaagatgagattaatcaatttattagctctttacctgagggtaacagatacacagaagcaacaagaaagcttttaggtatgcaacaagagcctaagcaggaacctagaaaatctgatagtacatcaaaagattcaatggactttgatacaattatggataaattaggattagaataggaggaaaatcaatgggtagacgattaatagataactcagaattaaatgtaattaaatatgatggtctacctgatttcttttctgctttaaaaaagaatagagtttcaggtagagataattcatcagatacaggtagctatgattttacagggactcatagttttcaagaagcctataacttaatggttaagggtgatagagagtcatatgatatggtagttaaacttaaaaaaatgacagatgcattatttagaatggataagtcagtaaaaagaaaacctgtcgtagctccggaagggtatcaacctcacgtacctaatgctataaaagggttacctaattctatgatgtctcagcaaagagttaaagcagagaagaaagttattgatgtattttataattctagtattagttggatggaagaccctgaaaatcttgcttacagaggggctataatgttaagtgctattcaaacattagaaacaaaaggatatagtataaacctttacttaggtaagttatcaaattcaggatatgaagataagttaacagggtttgttgttaatataaaacattcttatcaaagattaaatgtttttaaatcttccttttacttagtaaatccttcattcttacgtagaatatcttttagagtactagaagttgaacctgatatggttgacctaactaatcatggttatggtagtgtggtaagtaaaagtagttatggtaataaattaacagagcatatacttgataatgctgtaatttttgattctagcgtaggaattgatataaataacgattcatctgaaaacttaagagctgtaaaaaaactattcggaggtaggttgtaatggctaataatatttgggctgttgtattgagcattgttattttactcatcattttattaatactttggtttctttttagaaaaaaggtaaatggtagtagtaagaatgtagaaattcaaaaagcagaagaagataacgataataaagaacaagaggtagaagaagctcagtatagagaacttaatgaagaagagaaagaaaaaaatgaaaactctagtaaagattataagtacgataaagagaaagtaaaaaataaacttaaggagttagagtaactatgaaaaacctgattaaacttttatcaatggttgtagtaactatcttgactttttcactaacttatgttatacttaaaaaagaaacaaataataaaagaaatggtgtagcgccttttgatttttcattagaagaccacattcacctaaataaggagattaaataaatatggtatacgatagtattatttctcgaacaatggcagtatcaattttaaataaatggattgcagaattaattacagatgttgatttagataaatgtaagtttacagaagaagaatatgggaaagtagttacaaattcaatcaataaaatacaagatgtattaattgaaaagaactatgaggttacagatggtgaattgtatgatattgtttgtacagaattaattaacccaattaaaaataatacagaagaagaaaaacataatgaaaagaacgatttattagaacatttagaagatttggcttttagacatgatattgatttaggatatgttagtgatgggtcatataacttaactgtaacccattggttaatgcaagatgagtttacagatgttaacatcaaagttaataatgatgaagacttctatacagttacaattccggaaagtaaatatttttggttacctattacaaaagagaacttagaaatgttcttaacacaagaccctattaataaaggagaggttaagtaatggatagaattattggtaaacataacttaactcaagatttgagattaggtgataaagtagaagtttatgatgctcataaatttaaagaaaatgaagatggaactattgaattaggagataaaataactgaaggaattgttgtagattataaaggtgactttacaggtaatacaagtggtttagttacccttgactcttctgaaaaagaattaatcattggtgaatataattttaaacttattgaagaaggtaatttacaagcagtttatgattctgtatctaaaaacaaagtagaaagtctttctgaagattatgacatgtatagaaagttacttggagttaaatcaggagaactagcaggtatagaagatgaactagagtacttagtcagacaatataatagtaaagtagataattataatggactactaacattatctaaagaaaaagctagagaattatctctattaacaggagataaaaaaatgattcctcatatgaaaaatagaagattagaattaggtacagaagcagacttttaataagtctgcttttctcttatattagtaatagatataaggaggttggtatatggcaaatgaaaaagagattataagaatggttaattatcttattgataatatgtctatgtggcatataaactatgctagggctgtgttaataccaagtgaggtagaaaagataattaaagagcatgagaagtttgatgacctccttaagaagagaggagaatggttagtaaaaggttcagatacagataacattgatgatttagaaacttataatcaaataatgaataatcaaaaggatgaaatgatgatacaagaaattgatatctatacccaaggtaaaacaataacaatagataatgaacactattcttcagatgatttaggtgaagtcttaaataagctagaacaatcagaagatattaagataaaatctaattataagtcattatatgtaggatatactaacgtagtgggttatgaggttacttacgctagttcttacgaggaaacttttaaaaatgacttagaaaaagacctatgatatattagtactgtaagaataattaggaggaataaaccctatggaaaaagtaaatcatgagtttctagcagaattggcaaagagtaatagtcctgtactaaattcaaaaccacttcaagatggagactataatattgaatttgactatgatggttttcactttgagttctcacagaaaaatggttattggcgttggtcttataacgctaaataattaatatttaggctactttaattagtagcctttttttgttgacaaaatataaaaaatagtgtatagtaaagatataaataaaaaggagatgactaattatgtttaaaaaagcacctcaatacattatggaaaaagtagaaaaagaaaacaatattctaggagaagatttaagtcttgatatttattataaaggagttaaactaactgttaagagacaccctgaaactggtcatctaaatggatatataactttaccttcagatatcaatgaaaaagaatatgactccttagaaagacgtgcccatagaggtatcacttacgatgattatgactatgagggtaagagagtattagggtttgactgtgcacatgcctgggatatgacaccatatgctattataggttcgttagatgaccaatatagagatttagagtatgtactaagtattttaaaagatatggcagaatacgttaaaaaggatgagtaatatggacttctaccaatttctaaatcacgaaaatgtaagagttaattctataacaccaagtcaaaagaattttattagagaaaacttagaattaactaacctagaggatactgatattgattttattagttctaagcaagcaaaggaagaaatagagaaaattataagaattaaaaatgaagaagaatatgatatagctatggatgctttagcagggtgggtaactaaacatggttattaaggaggattgctatgaaatatattttaggattaataacactagggattatactatttaaagtctatgaacactttaagtataaacaagatgaagttgatacagaagaagatatataagtattgacattattatcaatatatgttattatttaaataacaaataaaaggaggatgttataatgaacttagaaaaaagtttcttattatcaacaatagaatttggaagtacttatcaaggaacatcagatgagcattcagataaagattacatgagtttagttgttcaacctttatcagatactatatttaggaataatgaaaaagcaagtaagcatacagaagtatcaagatactatgcagtagaaagatttatctctttagttctaaaaagtgggtttgataatgttcttaatttatgtgctcaattagagcaagctaaaaatactagattcaataaaactgttttagatttattttatgatgattttatatttttaacttatgttagagctaattttaagcctatagcatattctgttatcggtaatattaataatatactaaaaaaaggagaattaactggtaaagaccttgttaagttttatacattctataatcatttagaatactataatgatttattagatgatttagataacttaaatgttagctataaagactttgcaaaagttaaatatatgccaaaagaagtattagacaataagagaagcaatgtaagtattgaaaagaaaaaagatttggttaataaagtagagcccctaattcaagaagtaaaagataaacttaagtctaatgaatctaatatcaaacactataaagatgctatggaattagtagaaaaatctttgaaagacaaaactgtagaattccttacggaggtctataatgagagataaacgaatacactcagaattattatatgatatcataggtaaacacatacaagaagaggaaaatattacaccgtatatagaagctatatatgtagatatgatgaatattattgtagtagaatatactttttataatgaaaatggaacaagaatgctaggacaatatccgataggagaggttatgtaaatgaaaaatattattaattttttagtagactataacataaattttagttactcagaagatagtttaaatgttatgaataactcatacttagtagataaacatggtacacaagattatgaaattgtaggtaactatgaacatattacaggggtattttcttatcaaacagaagaagaggttatagctaagcttaaaaatcttatcggggtttgggagtaatatatgaaaaaaatttatatattagaagaagaaatagaggaaatggactatgacttgtgggaagaagatacagtgtatacaacaagttatgaaattttaggttatactgattccctagaggatgcagaatatattagagataactatggaacaagtaatcctatattcataaatgaatatccttatataacaaaagagaagttaatagaagaacaacgttactttagatacaatagttatattgaacttaaaagagttaatggttactttgaaatatctgaaataaatgacttacaggttactgaagactttagtattaataaagatgataaaaattttgattcacctttttctataaatatgttttcacataatagaaatagtataggtatagaattcattatgttttcagaatatgatgataaagaagatattatagaaaaagaaaaaaattcttttttaatgaaattaaaatatctccttaaacatagtaaagaagcagatatacgtagcacatcaaaaattatagattcaattgataaattaacttgacattaagtataatttatggtatattattagtatagtagttaactaaagcctcttatataatgctaaacacagttagcctttttaaccttataatgagaataagagcctaactatatgggtactttgaaatcattatattttacctatctaccaagcgtagaaaaggtctacggtaacgttataattctatttagcgttactagcaatcctgagtaatggcattggctagatgtagctacctagtcgttgaggttcgagtcctctctcaggatatttctaggactcttagcttaaaggtaaagccaaccgctcataacggtttgactgtaggttcgaatcctgcagagtccatataccaggctagcaaaaaacttataggtgttagacatatgtgcaatcacttgactatgatttaatattgtatgtggatttattgtttcatatctactaagtaaaacctatttctcttaagttactggttataagagaaggagtgaaaaactctaaaggtctagtcaacctgatgtacgagagataccgtatgttgcccggcagaggctttgtatctgaccgtagactccggatggtagtgcccctccattccgtatctatagttaattaattgattttggcttctagagcactcaagcttaattttagttttaacaataaagagtaggtaattgagaccgtagaacctttattgacgtaatagttagtacggacatagtgtcctgggtatcgaataggcttaaaaacgctaattgtacctttaaaaacaatcttcttccacaagctaccttctaaaatatcttgtggaataacgcagtgacaagagtttcaacgacagagggtctcatatagttgtcgtaaaatattattaaccgtaatctaagtacgggagctctgtcggacacctatttaaactaataggtgtttttttgttatattaattgtacaaaatatagtaaaggagataaaatttgtggagagaacattaaacttatatgattccaaaggtaaattattaaaatcttcagaaaaaattacaggtgcatccgctaaaattataattgaaaaactgacaccgaatacagtgtatagccaagggtcttttaaaatatcatggactattaatggtaaagagtctatattaacagatgttcccgaatttacaacaaaaagtaatgaagacaaacaagaaattgtttttaatactttaaacattgattctaactctttcgtagttagtgaaacagaaccaagtgataagagtaaattgtggtttaaaccaataaattaagaagaggagatttaaatgatggtaaacgtactacctagtgtttatgacgcagaaaaaggtgaatgggtaacactattagcaaaacctatagcagaagaagtacttaaaattatgaaagcagattatttagagcataaaggaaatattggattttttatatctaagtataaagacggagattctagtatagaacaacctaatgttgtagtattctataacgaaaaagattatgatacaatggagttaacagaatcagaacttactaatgcattaaatgaatatattgactataccttagatggcaagtataaaccattttctcttaataactttattaattatttggaagattatggatacagactacctgttaactttgaagttgatgttactattattttatctgacggtcaaaaatttacttaccctagaacaagctcaataactaataatgcttctatagtagatgcactgaaaagtgaagaccagtatatagaggttaaatatatttataatgaccatgcaatagatgataaaaaattagcacatggtaatgatactttaaaataacattaaatgataaacacctattaatttaataggtgtttttttattgactttttttactaagtatggtaagatagttatatacaataaggaggagattagatggctatatacgttgttcccgatatttacggagaataccaaaaattattaacaattatggataaaataaataatgaaagaaaacctgaagaaacaatagtatttttaggagattatgtagatagaggtaaaaggtcaaaagatgttgttaactatatatttgatttaatgtctaatgatgataatgtagtaactctgttagggaaccatgatgatgagttttataatattatggaaaacgtagaccgattaagtatctatgatattgaatggctctcaagatattgtatagaaacacttaactcttacggtgtgagtacagtaactttaaaatatagtagtgtagaggaaaatctaagaaataattatgattttattaaaagtgaactaaagaaacttaaagaatcagacgactatagaaaatttaaaatacttatggttaattgtagaaagtactataaagaagacaagtatatattctctcattcaggtggggttagttggaaacctgtagaagaacaaacaattgaccaattaatatggtcaagagactttcaacctagaaaagatggttttacctatgtatgtggtcacacccctacagacagtggagaagtagaaattaatggagacatgttaatgtgtgatgttggcgcagtatttagaaacattgattttccatttattaaattggaggttaagaaatgaagaaaaaatattttaaaggtcttaaattgaatgactttgaaaaagaagtttttgggataaaaaagaataaaaaatataagaaaatgaagaaaaaactaggtagaaatgaacctaagtattggaattatgacatgtccttttttattcagctatacgctgacctaaatgcatttatagagagtagtaatcatgtggatatggaataccatacttttgtagatgtagacggcaaagaaagaacacaaatagatatgataaaacatattttaagcttaataaaatattatcataaagaaatggatgattttgatatggataaatatgatgagcttgaacaggtacaaagtaaaatattagataattttaaaattgtattaccatcactatggaattaaaggagaaatattatgagagaaacaagggaatatattatgttttggggtaaagaggatatttattctaatttctaccctataaagtttaaacaccaaggaagaacatttaataactcagagcaagcctttatgtggcgcaaagcaagatactttaatgactttcaaatagcaggtgaaatactaaatgctaagaacccaaaccatgctaaaagtttaggtcgtaaagttcgtaattttaatgaagaacagtggaataaagtaagatataatattatggtagaagtagttaaagataaatttatgactacacacctaaagcaaagaatattagacacagatgtacgtaaagattttgtagaagcttcaccttatgataaaatatggggagtaggtcttaaagcaaatgaccctaaaatattagagcaaagtaactggaaaggtcagaatctattaggcaaagtaatggaagatgttcgagtacattgtatctacaataagtaggtaattataaaatggataagataaatcttaataaaaaacatgagggctctactgtagttaacatatcaaataatattacattaaaaatacaatgtacagacctaagaaaagaatgtgatgattcagaagcacctactacctatacccattttaaagcttatatcgtatataatatattcattgtagttaatgatagaaaacaaaagaaaaaagttaaatatgattgctataatgaccatgtaggcagaggtaatgttaaagacctattgaaagtaaaagatgttatcttccagttatccactcaattaaatactaatgaaattattaaaatatcaggtgcagatgaaagaagatataaaatatataaatattttatagaaaaagatataagatttgaagacaatatgtattatagtaaaagtaatatatggattataaataattttagcttattacaaaagttccaatggaacactgtagtaactaaagacggggactataataaaaaagaacttaaaaaggttgataaagaatggaaagaattattaatataaaaggagagttaaaatgaatatcaaatatattgatttagtattagaaaattgcgatgttgtaagattagagcctaaagacgtaagcaggttccatatatcaggtattacagaaggtatagattactatggtacatataaagggacttcaagtataagtcgaacacgtcactgtacttatttcggtattcttattgataagcctatggaaatacctcaagttggttttgcttatcctgataatacgaacgcttatgaaatgattacagcatattcagatattacagctatagatattatttatgaaaatgacgcaaatgaatatatttatgtagactttaatgaatacaatgataactataatatcaatcaaaagaatgattattacaataatatgttagaaattacaattacagaaagtaattccaaggaggaagaagatgaataaaacattttttaagttcctaggtaaaaacacattagagtattcaaaacaggggctaggttttcttgtagccctccctataatgttaattatattttctgtattcctagcattcattataggtattcctgcagttattatttacgctctacatgcattaaatgtagacaatgattttattatacagttagtacctgttatgtggttcataatactttatggtattgtaagaacaggtgagcacaaaaaaccatttgttaaactaaaattaaaggattacttattatctatattatatctaactactattacagctattagtgttttagaaaactatttgctctttcaatcattaccttttacaggggatgtaagagcagttataacactattatcatttatagtatttgttgctgtaaatagaggtatttgtaaaatagccattaagagctataaagaatataaggaggactcacaatgatagaaattagtatctcatggacttatttaatatcattcttgctactatggtcagccggtatcttatacattaactaccttgtttatagaataaggttaacaaataaggaacgtaaagaaatgagtaaggagcaccaccgcaatagagaagagataaaacaacggatagaaaataggagggataaataataatggaaaactataaaaactttattatagaggaaatgaataaagcccatatcttagtaactaaagcagaacaaattaaaaggaatagaaaattagcagaaacagaactagaagaagtatataaaaaagcagaagccttcgatgaaattgtaaatgagttactttatcaattacaaaatctagagagttgggatactctagaccaaaaagactgtcagacattaaaacaaatactagaggaaaacataaaggaggaaaaacagttgaaaagatacaaagtaaaacgtactattactacagaagaggtaagatatatagatgcagaaacagaggaggatgcatggtatagtgtagaatatgaagatgaaggtgcagatacagcacactataatgcagaatatggtacgtggtcttatgaagaggaggaaaaataatgggactagactttgaagtaatcggtgtgacattaagtaatagaaaagtggaacagaaaggcttacagcactttattaataacgctaggtatagacatatattagaaaagaattattataaaggatttaactttgaagatgactttagaaaaccaggatattttatggacttacttttaagagatgcagagacatattatgatgagtttgaagaatggtgtgaaggtgtattcgtactaactaaagacaagttagttaatctaatgaaaaatgagtttaatgaaaaaacgtttaaaggtacacatgatgcagaatattattatagattaatgtctcatatatacaatgtagaacaatatgaaggtaaattctatgacttttacttaatcatgagtgtaaatgtataaggaggattaagtatgaacaaagaacaagccaaacttaaactcgaaacaagtattattaattatgagaaccaaataaaattcttagaccctgcaagtatgtatactagagggcttatagatgcaaaaggttactcaaaattggcgttaaaagaattagaagatacaggtaaacactcttatgaagatactacatggaaagatagttatgcaaaggtatttacagatgaagaaatactagaattcttactatccaaaccaagagtcacatttaaaggtaatcaagaaaaactagatgaaattaaaaaagaaagagaaaaaatacaaaaagaagctactaaagacttacctaagggcagtccactaggtgacttatcaaaagaaaattatgagaaattttggggagcattacaatggtctagagaagaaagagaaaagttaacacaagaatcaagagcttattatgaaaactatctaaaaaaaatcaaggagaataagtaatatgaaactataccaagtagaacatgataattgtgaaccttatgaagataacttccattttagagaagataagatatatacagataaggagaacttaattaaacgtattaaagaagaaggttataaagaggaaacaaaccatagaggtgagcaagagttcattaaaggagacccaagagatttctatggaatggatatgattactatacatgaattagagtttgttaataatacctaagaggagggatttaatttccctctttttttattttaggtatgttgactttatatttagaggttgtctatttgtactataaattattgacttacagggaggggttataatgttgacttttaatatagggtgttgacttactttctatattttaggtgttgacttttaaatatttattttcataatttttcccaaaaaataagtacctgggtagaccataggggtatatagacccacccccattgagtggcattttacccctgcccccgtataatataaaaaaattatataataggataaaaataaaagagggattttattccctctatttgttatttaaaatattccattttgtttttttatttctacgattcttttttcttgtctttctaatttgttaactaaaatatcgtaagcttcagtgtattgttctcctttagcaatcattctatctaatacccaaataatgtcttttacatctttttctaattgttcctgctctaataagtttctcatgttcatcatttccttttcttttaatttgttaagttcattataccatacaaactaaaagattgtaaagcgttttgataaaataatttaaacaaatgtttgtattagttctttaatctcaccagtagataatgacgataggtcataagcttctctaagctcttctaagctaccatatgggctattactatatgaagtagtattattgttcatctgtattgttccgcttctctgtgtgcgtctgtgtagctcatcattgatgtactgctctatgaggttatccgttgggtgagcctgctcctgcaggagtttgctcttagcactcttaacgacattgtgaacctctctaatgttcattactcatcatcaccattcataacatcaactaataccattagctcttgcttagcctttatttgcttctctagttgtttattcaactcctgcatagtcttttgagtatctgctatttgttcatctaataatctaactgcttcgtttaacgctctgtttacccgtcttgtattcatcttcaacatctcctgattaattgtattagagaagcggttaagcttctctgtgtgctttctatagtgccattctttgagctacaatctcaaccgctagattaacatattgaaccttgtcagtttcttgtagttctaattcaacatcatccatagcctcatagattgcttccgcccggtctgcttcgtccatgtcttcaaatccttcaacctcatcagaagcaatgttttctgctaggtcaaattgaatgttattatattcgtcttcgtcttcaaattctacatttgcataattctcgaggtctctcataagctcatagttcaataagtcataatattgttgtcctgtaacctcttcaatgtattcagtgatgaccgcttcaatgtcctgctcgtaactatcaaagaagtttactacgtctgatgtgtagattaatttgccaattgctccgctttgtactccatgagttgctacgtccttaactgtgcttgcttcttgtccttgtaaaaatgcttgtattgtatttgtcataataatcaaatccttttctttttttattttttctatactcttatattaccatctatatgttaggatgtcaataccttttctaaactaatttccaaccgatgtcgtttacatattcttctagttcttccatagtgtccgcaaatttgagtgattgcaaagtgtctgaagtaataacaaccgcttcctggtcatgtatttctttgtctaggatgttattatctaagtggtcgatgtactcccaaattagggcttgttttacactctcaagtgttgctatgctaattacgatttcattatctctattgattaatttcatttgtatcaattccttttcagtttattttgtcgttgtgcttgattaccttacaatttaataataacattgtagggcttgaaagtcaagccctttatttaattaattttcgtaaatgaacaagtctttgcttggtaagtatctaaagtttggttcaattcctaagcaatatacaagtgtgtttaaactgtcacggtctgagttgctttcaccgtaatatgaaacaatctcatttatgcttaagtcaatctcaattgtttgtgtaccccaatgacgcataactaatttgttgccatcaatttcaattgcatatttttcttcaagttcacctttactatatccgtcaaagtaacgaccatttaaaattgcttgccctttgttttctgctttttctaataagttttcgatagttttgataaatttcattataatcaattccttttctgtttgttttttgttatcttaataatatcatggtttcaagtgactgtcaatagttttttttaaactttttttatttaggttggaagcggttgaccgcttcccttaccttgtatcttaatattaacatctattgtttaagttgtcaagccttattttgaatattttacaattttatttgctttcaagaaagtaacatcttgacggtagttttttaactcttctgtaaacctttcattatttttcatgccgattaagtttaaagttgttgtgatttttttcatttttatcatttcctttattgatttatttgttatcttaatattaacatgttgtttctaggttgtcaatagttatcttaaaaaaatatttatattatcttgtagtagtaaaaaccagtttacaagtttaggtgtgactgttgcaactaaaccccctataataagtaagagaatgttatctgttttttgtgttgatttgattgttttgtttttccatttaaattttatcattttgttcaatccctttcatttggtatacctttatattaaactataatgatattaaagtcaatagatttaatgaaattaatttaaaaaacttttttacatttatctattgacattaagaccgaattattatataatggaagtgataccaaaaaaggaggagtaaaaaaatggttgaaaataaaattaatgaaatttggaaacctataaaaaaagaatactttaataaatataatttttatgtttctaatttaggtagggtaaaaataaatgataggttgagtaaagtacatcaagaccgtgacggatatttaaccgtaagggtcaataataaaaaacacatggtgcatagacttgtatatgaatattttggcaatgattttattaaatcaaaccatgtacaccatatagacggtaacaaacaaaataattgtattgataacctagaatgtattagcccgtcagaacacaataaaaggcaccataaagacaatactttcaataggtataataggggttatgcattaacagaagatgaaagaaaagccatagcaagcaaatacaagcctagaaagtacactcaacctatgttagcaaaggaatataatataagtgaaataaccgttagaagaattataaaaaaatataaaaaagattaaagaaaagggttgaccttttcttttttctatagtatactataagtatagtaagaaagcggtaaggagcctacgtgtccgctatataagagaagaggttccgggaaagagtggttttaaaaccatttaatataaattttttatataataatttttttatactaaaaaaggtcttcacttcattataccataaaaaatacaaccttgtcaacagtcaattttacactaatttatttattaaataatttttttatataaattaaaataaataaaaatccctagaaatctaatcctagggaattgtataattttttttatattattgtattgcacttctattatatcttaatttcataaatttattataagtttgtttactgttaaatataataaattcatctgattcatttttaaatatattgtgtacagtttgatagctcatatatccattttttgctaaattccataaagtttttatatcactattatttttttctattttaaatttcatattatcatatcctttttatataatttttttatttaattcagaagcggttatattcaaccgcttcatatttaattttattgactaattctatttgttttaacttgtgtgtaagcatattctagagtatccgctatattataactttggaatactacaccaccgccaaaccatttagcagtatattttttaaagccgtatagtttattgatgttgtcatagtctgctagtttaatatctaaatccatgaaatgcactacataacgtgggttgccgtttgcgtcatttggaattctgaaagccgtaatgtcttgcccttgtactgtgtgagtttcttcgtataagtaatttttgtttgtcataataatcaaatcctattctataatttttttatttaatttcgttatttgctaggtggcttgtcttatttgttatcttaataataacatgtatcttgtaaggtgtcaacacttttctttaacttttttaaaaagttttttgaggtgtgttgctatcccctacacctattatattactatagatgcttgaggattgcaacccctatttttaaatttcttcaataaattttatttctttgttttgaactccgtacactacattttcatatacgattataattacatcgtcaatcacattaactaacatgctagccggttggctttgccctctcatgctcattataggcgttttgcggaacatctgcactgtgtcatagtttccattaatagaaacaattaacgccgtatatgtgcgtgtatggtttagtgtgctttcatataatggaggttctgcacatctgtagccgtccatttgccacaactccattatagtatttgcaatttcttttacatccatttaaatcatctccaattaaataatttttttatataattctaatatcttgttgctgttcccttgctgtacctttatattactaccatatgataataaagtcaataagaaaatgcaaattcttttaaaaacatttttagggctaaaaactactattatatagaagaaaaaagaatttaaaaaaagttactcaaaagtattgacttctgaataactatactgtaatatataaagtgtagtaaggcaagggacaaaccccaaccgcttctacaaatattatataaaaaaattataagaaaaggatttgatttaaatgaaaataaactatattccaatgtgggacaatgaagatgtattacaatatgcaaaatcacaattattagtaaatgaattagaaacaaaagaaattatattcaaaaattatcaaatatcagatgatttagatggcggaacagataaaaaatattatgaaatatatgaaagtaaattttatgtagatgaggaaacaacaaaagaagaatttaataatttaattatagaaaatgaaaaactaataaaagagtataaaacacaaaacggattaattaaaaatttaattaaatcacaacatgaagtaaatgaatttgaatacaatgttataaatattctataaaaaaattatataatacactaggaataatatcctagtgtatttatttttgcggtctgatattattccaaccgtctatatatatatagacacctgaagcggttaaaataaactagtagaaaacagagggagctacttagggtaaaatcataccgagctacttaggcaattcctagggagctacttagtgaaattagggagagctacttagtcaatccccacccatacgttgccaagcttttaacctaacaatttctcctaggtcatgtttactaatactttctcctctataatagaaataattactataattaccaaaaaactcaggaacacctaataaagctacaggttcattataactattatcaagaacaatgtcttttctttctaatagaccttccatatccttacagtatacaactgatacaatataagaggaatctatatcctcaggtaattcatctttatagtattctaagtatctatccctttcattaagccaaggtaagaactcgtcttttaaataatccttagaaacatctgtatatataactatcatcctatccctcctatcctacgtgccaaatgtgattgaaatcaaatacgccttgtcttctaatatctactagtaaattgactacatctatctttccatgtggtctgcgtttataatcatctaatgatatatcagtaactagattagtatttatcatcctttgaactaagttcatagtttctgtctcatttaaaccgctagctattaccctatcagattctttatgaataatagtgtatgttaaagtatccatcctaacttaaccccctatcctatcctatcctattgtaactcatcctaaattattttctatcctaagtttatcctatcctaccattctcttattgtaacataattacctctattgataagtaatataccttcctcattaaccgcttcaataagtttatctatatctatatcatcactcttactcccaaactctttattatagtgattagggaatacgttactaatatctttataccaagtatcatacaagaagtatattaaactacttctatctagcttatccttaatagttttcatactacctctatggtagactgtataatataccttaaacattagttacatctcctttacatactcttccatactatctacacctattgattcacccttaagatattcatgtatccacgtctccgctataaatacaagtatatcatcttcaaatggaggaagtgtactccattcttcaacttcgtctttagtacacctatagggattaacataaacatccaatcctggacttctattaacatcataagttagttctcttgaagcggtctctttaatccattttaattctgcttttagtaaattcttatcataattttcactaaggtatatctctatcattataatctctcctttacgaaggaaccaatattacatgatatacctctatagtagctacattctaaaccatgtctatttattagtatacaaagtttaccttcatcaataatgtttgagttaatccttacatcatctctttctctaaatacatttttctctatacatacatccgcttctccttttcccattaaatagctaagacctctactactacaattatcaatatgttctaatactttctttaaagccttggcatcatatttttcagttaaaaatattataatcattataacatctcctctacatacttacgtataataacaggttcaccattaacaaatgccttacagtatagctcttctataagaatacataagtcatccttgaacatgtttgtacctgataactcaattgtatcctcctcatctatagggtcatcggtttcaatcacgacatccttatcatagtctgtattaataccataagttaattccctacctacagtatcattaatatactcaagtaaactccttaatgtttctttattataatcctcttttacaaatatatgaatcattatatctctcctttcaattctaagacctctcagccttccatatagaagcggtatactattttaggacaaatcccctctaactacttaaccgcttcttaaatgagctctatattccaagtatagctaaaatacctgatgctattataactgtcataagtacaaatgttatgacgttaattaccttatcaaaactattatatctgattgactgaatcaagtctactaaaaaataaatatacatagagatagttgatatactcattatcgtaattaatattgttattcctatcatgattacttatcctccttaaaatatctcttctagtattcttaaaatactaatacctgaaatctgctcaccttggtggtatacaatagcatcatcatcaactatacaaataattaaatcacccgggaagtaataggatatatccttatatataaatctatcttcaggtttaacatataacattgtgtgaatattaacatccgcttctacattattaaaatcataacttaactccctaggagcatggtcattgattaatctgagtgctttagacaagtattctttattataaccttctcctaagtatatatcaatcatctatttcatctcctaaatttgactatacctaattatagcatacccgcttcatagttgtcaacaattaattacaattgaagcggattatctctcaatattctcaattatagaaccctaaatgataagtagctacttactattttaagtacattaaaactttgccctagggaaaacctaatactttttagtaattttaaaaataagaatcccatacctatagggttttatattttagtaccacttttcctagggtctaaagattaaaatcctagattagaatgattctaatgtaatatactagattaatgtaccattatataagattattatataataaccatagattaagaccgcttcagcatagattaacagtagaatactatagtttaagtattgtataataaatgaatattataactatagaataagtagttagggaagtcggtaataagtaataggctgagtgagagaggtaaaagaattcccaggtacttagtagtagtaatagtagtagtaatagtagtagtaatagtagtagtaatagtagtagtaatagtagtagtaatagtagtagtaatagta

SEQ ID NO: 3 - DNA, 42 bp, Kayvirus G1
  source 1..42: mol_type = genomic DNA
tactataaagtaggtaagcgtaataagaaatcacctgtagga

SEQ ID NO: 4 - DNA, 42 bp, synthetic construct
  source 1..42: mol_type = other DNA
tactataaagtaggtaagcgtaataagaaataacctgtagaa

SEQ ID NO: 5 - protein, 14 aa, Kayvirus G1
YYKVGKRNKKSPVE